(19) United States
(12) Patent Application Publication
(10) Pub. No.: US 2009/0105305 A1
(43) Pub. Date: Apr. 23, 2009

(54) Therapeutic Agents - 550

(75) Inventors: Roger John Butlin, Macclesfield (GB); Peter William Rodney Caulkett, Macclesfield (GB); Petra Johannesson, Molndal (SE); Laurent Daniel Knerr, Molndasl (SE); Andrew Leach, Macclesfield (GB); Nicholas John Newcombe, Macclesfield (GB); Charles John O'Donnell, Macclesfield (GB); Helen Pointon, Macclesfield (GB); James Matthew Wood, Macclesfield (GB)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 11/961,850

(22) Filed: Dec. 20, 2007

Related U.S. Application Data
(60) Provisional application No. 60/871,192, filed on Dec. 21, 2006.
(60) Provisional application No. 60/910,232, filed on Apr. 5, 2007.

(51) Int. Cl. A61K 31/451; C07D 211/60; A61P 35/00; A61P 3/10; A61P 3/04

(52) U.S. Cl. 514/330; 546/226

(57) ABSTRACT

A compound of formula I

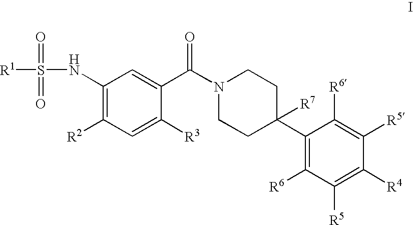

or a pharmaceutically acceptable salt thereof, processes for preparing such compounds, their use as Fatty Acid Synthase inhibitors, methods for their therapeutic use, particularly in the treatment of obesity and diabetes mellitus, cancer and infection and pharmaceutical compositions containing them.

This application claims the benefit under 35 U.S.C. § 119(e) of Application No. 60/871,192 (US sort No. 1) filed on 21 Dec. 2006, and Application No. 60/910,232 (US sort No. 2) filed on 5 Apr. 2007.

### FIELD OF INVENTION

The present invention relates to sulfonamides, particularly to substituted N-[3-[4-phenyl)piperidine-1-carbonyl]phenyl]sulfonamides, to processes for preparing such compounds, to their use as Fatty Acid Synthase inhibitors, to methods for their therapeutic use, particularly in the treatment of obesity and diabetes mellitus, and to pharmaceutical compositions containing them.

### BACKGROUND OF THE INVENTION

Obesity and diabetes are reaching epidemic proportions in the USA, EU, Japan and developing countries. Obesity is the major driver of the co-morbidities of the metabolic syndrome, particularly type 2 diabetes. Since no effective pharmacotherapies for obesity are available to date and current diabetes therapies do not stop the progression of the disease, there is a huge unmet medical need.

Fatty Acid Synthase (FAS) is a critical enzyme for endogenous lipogenesis and plays an important role in the modulation of key intermediates of lipid and carbohydrate cellular metabolism. FAS is highly expressed in the tissues with high metabolic activity (for example liver, adipose tissue and brain) and there are good reasons to believe that a FAS inhibitor would cause beneficial metabolic effects in peripheral tissues. In addition, inhibition of FAS in the hypothalamus may result in reduced food intake. The non-specific irreversible FAS inhibitors cerulenin and C-75 have been reported in the literature to decrease brain levels of orexigenic neuropeptides and to decrease food intake.

Therefore there is a need for an effective FAS inhibitor to treat obesity and diabetes.

### DESCRIPTION OF THE INVENTION

The present invention provides a compound of formula

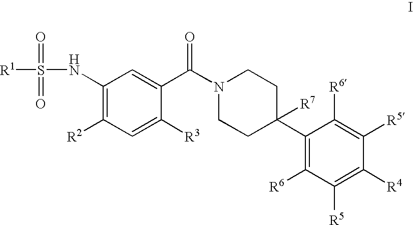

or a pharmaceutically acceptable salt thereof, in which
R¹ represents 1) a C₁₋₆alkyl group optionally substituted by one or two groups selected from A-X below and/or by one to five groups selected from Y below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) a group CONR^{e}R^{f} in which R^{e} and R^{f} are as defined below; vii) C₁₋₆alkanoyl; viii) benzoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl or benzyloxy; xix) nitro; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 10 membered heterocyclic group as defined in c) below; xxvii) phenylsulfonyl; xxviii) heteroarylsulfonyl;
xxix) a group of formula NR^{c}R^{d} in which R^{c} and R^{d} independently represent:

### a) H;

b) C₁₋₆alkanoyl optionally substituted by carboxy or by a C₁₋₆ alkoxycarbonyl group;
c) a carbon linked saturated or partially unsaturated 4 to 10 membered heterocyclic group containing one or more N, S or O, wherein the S may be in its oxidised form of SO or SO₂, which is optionally fused to a benz ring and any ring is optionally substituted by one or more of the following: hydroxy, halo, a C₁₋₆ alkoxycarbonyl group, oxo, carboxy, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulphonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 10 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulphonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl or nitro;
k) heteroaryl optionally substituted by one or more carboxy; fluoro; hydroxy; a C₁₋₆alkyl group (which is optionally substituted by one or more of the following: a C₁₋₆alkoxy group, hydroxy or a group of formula NR^{e}R^{f} in which R^{e} and R^{f} are as defined above); a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group optionally substituted by phenyl;
n) heteroarylcarbonyl;
o) sulfamoyl optionally substituted by one or two independently selected C₁₋₆alkyl groups or the terminal nitrogen is included in a 5 or 6 membered saturated or partially unsaturated heterocyclic ring optionally containing an additional N, S or O, wherein the S may be in its oxidised form of SO or SO₂;
B) a heteroaryl group which is optionally substituted by groups i) to xxix) as described for phenyl above;
C) a group of formula NR^{c}R^{d} in which R^{c} and R^{d} are as defined above;
D) a C₃₋₁₀cycloalkyl group optionally substituted by one or more hydroxy or a group of formula NR^{e}R^{f} in which R^{e} and R^{f} are as defined above;
E) a carbon linked saturated or partially unsaturated 4 to 10 membered heterocyclic group containing one or more N, S or O, wherein the S may be in its oxidised form of SO or SO₂, which is optionally fused to a benz ring or a heteroaryl ring and/or is optionally substituted by one or more of the following: hydroxy; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; a C₁₋₆alkyl optionally substituted by one or more hydroxy or C₁₋₆alkoxy; or a C₁₋₆ alkoxycarbonyl group;
F) a C₁₋₆ alkoxycarbonyl group;
G) a C₂₋₆alkynyl group;
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulphonyl group;
M) phenylsulfonyl;
N) heteroarylsulfonyl;
O) benzoyl;
P) a C₁₋₆alkanoyl group
Q) C₁₋₆alkylthio;
R) ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
S) phenoxy;
T) hydroxy;
U) oxo;
V) carboxy;
W) cyano;
X) sulfamoyl optionally substituted by one or two independently selected C₁₋₆alkyl groups or the nitrogen is included in a 4 or 7 membered saturated or partially unsaturated heterocyclic ring optionally containing an additional N, S or O, wherein the S may be in its oxidised form of SO or SO₂;
Y) sulfamoylamino optionally substituted by one or two independently selected C₁₋₆alkyl groups or the terminal nitrogen is included in a 4 or 7 membered saturated or partially unsaturated heterocyclic ring optionally containing an additional N, S or O, wherein the S may be in its oxidised form of SO or SO₂;
Z) fluoro or chloro;
or R¹ represents
2) a C₃₋₁₀cycloalkyl group optionally substituted by one or two groups selected from A to Y above and/or by one to five groups selected from Z above;
3) a C₂₋₆alkynyl group optionally substituted by one or two groups selected from A to Y above and/or by one to five groups selected from Z above;
4) a carbon linked saturated or partially unsaturated 4 to 10 membered heterocyclic group containing one or more N, S or O, wherein the S may be in its oxidised form of SO or SO₂, which is optionally fused to a benz ring and any ring is optionally substituted by one or two groups A to Y as defined above and/or by one to five groups selected from Z above;
5) a C₂₋₆alkenyl group optionally substituted by one or two groups selected from A to Y above and/or by one to five groups selected from Z above;
6) optionally substituted phenyl including optional fusion of the phenyl ring to a saturated or partially unsaturated 5 to 6 membered heterocyclic ring optionally containing one, two or three hetero atoms selected from oxygen, sulphur optionally in its oxidised forms of SO or SO₂ or nitrogen wherein the heterocyclic ring is optionally substituted by one or more of the following: a C₁₋₆alkoxy group; a C₁₋₆alkanoyl group; carboxy; a C₁₋₆alkylsulfonyl group; a C₁₋₆alkoxycarbonyl group; carbamoyl; N—C₁₋₆alkylcarbamoyl; N,N-diC₁₋₆alkylcarbamoyl; hydroxy; oxo; a C₁₋₆alkyl group (which is optionally substituted by one or more of the following: a C₁₋₆alkoxy group, hydroxy or a group of formula NR^{c}R^{d} in which R^{c} and R^{d} are as defined above) and wherein the phenyl ring is optionally substituted by one or more of the groups i to xxix listed above or by a heteroaryl group optionally substituted by one or more groups i) to xxix) above or by an ureido group of formula R^{m}RⁿN—C(O)—NH— in which R^{m} and Rⁿ independently represent H, a C₁₋₆alkyl group optionally substituted by a C₁₋₆alkoxy group, or R^{m} and Rⁿ together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and/or optionally substituted by one or more of the following: a C₁₋₆alkoxy group; hydroxy; oxo; carboxy; a C₁₋₆alkylsulfonyl group; or a C₁₋₆alkyl group optionally substituted by one or more hydroxy or C₁₋₆alkoxy;
7) optionally substituted heteroaryl including N-oxides and S-oxides thereof optionally substituted by one or more of the groups i to xxix listed above;
wherein any alkyl chain mentioned in any of the definitions from A to Z above or in any of the definitions i to xxix above is optionally substituted by 1) one group or two groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or by 2) from one to five fluoro; and further wherein any cycloalkyl, phenyl, heteroaryl ring or carbon linked saturated or partially saturated 4 to 10 membered heterocyclic group in the list of optional substituents from A to Y above or in any of the definitions i to xxix above, for which specific substitution has not been previously mentioned, is optionally substituted by one, two or three groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; a C₁₋₄alkanoyloxy group or a C₁₋₄alkyl optionally substituted by one or more hydroxy, C₁₋₃alkoxy or a group —NR^{e}R^{f} in which R^{e} and R^{f} are as defined above; and/or is optionally substituted by one to five fluoro;
R² represents H, cyano, halo, a C₁₋₃alkoxy group, a group C₁₋₆alkylS(O)ₐ(O)_{b}— wherein the C₁₋₆alkyl is optionally substituted by one or more fluoro and a is 0, 1 or 2 and b is 0 except when a is 2 then b may also be 1 or R² represents a C₁₋₃alkyl group optionally substituted by a C₁₋₃alkoxy group or by a group C₁₋₃alkylS(O)ᵤ— which is optionally substituted by one or more fluoro and in which u is 0, 1 or 2;
R³ represents H, cyano, halo, a C₁₋₃alkoxy group, a group C₁₋₆alkylS(O)_{c}(O)_{d}— wherein the C₁₋₆alkyl is optionally substituted by one or more fluoro and c is 0, 1 or 2 and d is 0 except when c is 2 then d may also be 1 or R² represents a C₁₋₃alkyl group optionally substituted by a C₁₋₃alkoxy group or by a group C₁₋₃alkylS(O)ₜ— which is optionally substituted by one or more fluoro and in which t is 0, 1 or 2;
R⁴ represents
i) a C₁₋₃alkyl group optionally substituted by cyano, hydroxy, a C₁₋₃alkoxy group or by one or more halo
ii) a C₁₋₃alkoxy group optionally substituted by one or more halo or optionally substituted by cyano, hydroxy, a C₁₋₃alkoxy group, an amino group of formula NR^{u}R^{v} in which R^{u} and R^{v} independently represent H, a C₁₋₃alkylsulphonyl group, a C₁₋₃alkanoyl group, a C₁₋₃alkoxycarbonyl group, or a C₁₋₃alkyl group optionally substituted by hydroxy or R^{u} and R^{v} together with the nitrogen atom to which they are attached represent azetidinyl, pyrrolidinyl, piperidinyl, piperazinyl or morpholinyl each of which is optionally substituted by one or more of the following: oxo, C₁₋₃alkyl or hydroxy; or
iii) halo, iv) nitro, v) cyano,
vi) a C₁₋₆alkylS(O)_{y}(O)_{z}— optionally substituted by one or more fluoro wherein y is 0, 1 or 2 and z is 0 except when y is 2 then z may also be 1
vii) a group -L-R^{g} in which L represents a bond, a C₃₋₆cycloalkylene group, a C₃₋₆cycloalkylidene group, a C₁₋₆alkylene group or a C₁₋₆alkoxyC₁₋₆alkylene group wherein each group is optionally substituted by one or more of the following: carboxy, hydroxy, a C₁₋₃alkyl group optionally substituted by hydroxy;
and R^{g} represents carboxy or a group NR^{u}R^{v} in which R^{u} and R^{v} are as defined above and additionally R^{v} represents cyano or R^{g} represents a group CO₂R^{w} in which R^{w} is a C₁₋₃alkyl group; or R^{g} represents a group CONR^{x}R^{y} in which R^{x} and R^{y} independently represent H, a C₁₋₃alkylsulphonyl group, a C₁₋₃alkyl group or a C₃₋₆cycloalkyl group wherein the alkyl and cycloalkyl groups are optionally substituted by one or more hydroxy, carboxy or NR^{u}R^{v} in which R^{u} and R^{v} are as previously defined, or R^{x} and R^{y} together with the nitrogen atom to which they are attached represent azetidinyl; pyrrolidinyl, piperidinyl or morpholinyl; or R^{g} represents tetrazolyl or thiazolidin-2,4-dion-5-yl; or R^{g} represents ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
viii) a group -L₁-N(R^{h})SO₂-L₂-Rⁱ in which L₁ and L₂ independently represent a bond or a C₁₋₆alkylene optionally substituted by one or more C₁₋₃alkyl groups, R^{h} is H or C₁₋₃alkyl and Rⁱ represents cyano or NR^{u}R^{v} in which R^{u} and R^{v} are as previously defined or Rⁱ represents a group CO—R^{j} in which R^{j} represents hydroxy, C₁₋₃alkoxy or a group NR^{u}R^{v} in which R^{u} and R^{v} are as previously defined;
ix) phenyl(O)_{f}— wherein f is 0 or 1 optionally substituted by one or more halo, C₁₋₃alkyl optionally substituted by one or more halo or C₁₋₃alkoxy optionally substituted by one or more halo;
x) phenylthio optionally substituted by one or more halo, C₁₋₃alkyl optionally substituted by one or more halo or C₁₋₃alkoxy optionally substituted by one or more halo;
xi) monocyclic heteroaryl(O)_{g}— wherein g is 0 or 1 optionally substituted by one or more halo, C₁₋₃alkyl optionally substituted by one or more halo or C₁₋₃alkoxy optionally substituted by one or more halo;
xii) a nitrogen containing 5 or 6 membered heteroarylCO— wherein the heteroaryl is linked through N to the carbonyl group and is optionally substituted by one or more halo, C₁₋₃alkyl optionally substituted by one or more halo or C₁₋₃alkoxy optionally substituted by one or more halo;
xiii) a C₂₋₆alkynyl group optionally substituted by one or more C₁₋₃alkyl, hydroxy, C₁₋₃alkoxy, C₁₋₃alkoxyC₁₋₃alkoxy, or a group —NR^{u}R^{v} as defined above;
xiv) a group -L₃-S(O)ₑC₁₋₆alkyl in which L₃ is a C₁₋₆alkylene optionally substituted by one or more of the following: hydroxy or a C₁₋₃alkyl group, and e is 0, 1 or 2;
xv) a group SO₂NR^{o}R^{p} in which R^{o} and R^{p} independently represent H; a C₁₋₆alkyl group optionally substituted by one or more of the following: hydroxy, C₁₋₆alkoxy or a group —NR^{u}R^{v} in which R^{k} and R^{l} are as defined above, or R^{o} and R^{p} together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and any ring is optionally substituted by one or more of the following: a C₁₋₃alkoxy group; carboxy; a C₁₋₃alkylsulfonyl group; C₁₋₃alkanoyl; benzoyl; hydroxy; oxo; carboxy; or by a C₁₋₃alkyl group optionally substituted by one or more of the following: hydroxy, C₁₋₃alkoxy or carboxy; or
xvi) —C(NH₂)═N—OH
R⁵ and R^{5′} independently represent H, halo, cyano, C₁₋₃alkyl optionally substituted by one or more halo or C₁₋₃alkoxy optionally substituted by one or more halo;
R⁶ and R^{6′} independently represent H, halo, cyano, C₁₋₃alkyl optionally substituted by one or more halo or C₁₋₃alkoxy optionally substituted by one or more halo; and

### R⁷ is H or OH.

In another aspect the present invention provides a compound of formula I

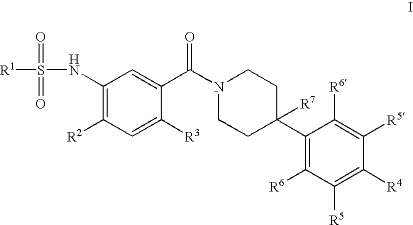

or a pharmaceutically acceptable salt thereof, in which
R¹ represents 1) a C₁₋₆alkyl group optionally substituted by one or two groups selected from A-W below and/or by one to five groups selected from X below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) a group CONR^{e}R^{f} in which R^{e} and R^{f} are as defined below; vii) C₁₋₆alkanoyl; viii) benzoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl or benzyloxy; xix) nitro; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 8 membered heterocyclic group as defined in c) below; xxvii) phenylsulfonyl; xxviii) heteroarylsulfonyl;
xxix) a group of formula NR^{c}R^{d} in which R^{c} and R^{d} independently represent:

### a) H;

b) C₁₋₆alkanoyl optionally substituted by carboxy or by a C₁₋₆ alkoxycarbonyl group;
c) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by one or more of the following: hydroxy, halo, a C₁₋₆ alkoxycarbonyl group, oxo, carboxy, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulfonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 8 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulfonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl or nitro;
k) heteroaryl optionally substituted by one or more carboxy; fluoro; hydroxy; a C₁₋₆alkyl group (which is optionally substituted by one or more of the following: a C₁₋₆alkoxy group, hydroxy or a group of formula NR^{e}R^{f} in which R^{e} and R^{f} are as defined above); a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group optionally substituted by phenyl;
n) heteroarylcarbonyl;
o) sulfamoyl optionally substituted by one or two independently selected C₁₋₆alkyl groups or the terminal nitrogen is included in a 5 or 6 membered saturated or partially unsaturated heterocyclic ring optionally containing an additional N, S or O, wherein the S may be in its oxidised form of SO or SO₂;
B) a heteroaryl group which is optionally substituted by groups i) to xxix) as described for phenyl above;
C) a group of formula NR^{c}R^{d} in which R^{c} and R^{d} are as defined above;
D) a C₃₋₁₀cycloalkyl group optionally substituted by one or more hydroxy or a group of formula NR^{e}R^{f} in which R^{e} and R^{f} are as defined above;
E) a carbon linked saturated or partially unsaturated 4 to 8 membered heterocyclic group containing one or more N, S or O, wherein the S may be in its oxidised form of SO or SO₂, which is optionally fused to a benz ring or a heteroaryl ring and/or is optionally substituted by one or more of the following: hydroxy; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; a C₁₋₆alkyl optionally substituted by one or more hydroxy or C₁₋₆alkoxy; or a C₁₋₆ alkoxycarbonyl group;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulfonyl group;
M) phenylsulfonyl;
N) heteroarylsulfonyl;
O) benzoyl;
P) a C₁₋₆alkanoyl group
Q) C₁₋₆alkylthio;
R) ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
S) phenoxy;
T) hydroxy;
U) oxo;
V) carboxy;
W) cyano;
X) fluoro; or R¹ represents
2) a C₃₋₁₀cycloalkyl group optionally substituted by one or two groups selected from A to X above;
3) a C₂₋₆alkynyl group optionally substituted by one or two groups selected from A to X above;
4) a carbon linked saturated or partially unsaturated 4 to 10 membered heterocyclic group containing one or more N, S or O, wherein the S may be in its oxidised form of SO or SO₂, which is optionally fused to a benz ring and any ring is optionally substituted by one or two groups A to X as defined above;
5) a C₂₋₆alkenyl group optionally substituted by one or two groups selected from A to X above;
6) optionally substituted phenyl including optional fusion of the phenyl ring to a saturated or partially unsaturated 5 to 6 membered heterocyclic ring optionally containing one, two or three hetero atoms selected from oxygen, sulphur optionally in its oxidised forms of SO or SO₂ or nitrogen wherein the heterocyclic ring is optionally substituted by one or more of the following: a C₁₋₆alkoxy group; a C₁₋₆alkanoyl group; carboxy; a C₁₋₆alkylsulfonyl group; a C₁₋₆alkoxycarbonyl group; carbamoyl; N—C₁₋₆alkylcarbamoyl; N,N-diC₁₋₆alkylcarbamoyl; hydroxy; oxo; a C₁₋₆alkyl group (which is optionally substituted by one or more of the following: a C₁₋₆alkoxy group, hydroxy or a group of formula NR^{c}R^{d} in which R^{c} and R^{d} are as defined above) and wherein the phenyl ring is optionally substituted by one or more of the groups i to xxix listed above or by a heteroaryl group optionally substituted by one or more groups i) to xxix) above or by an ureido group of formula R^{m}RⁿN—C(O)—NH— in which R^{m} and Rⁿ independently represent H, a C₁₋₆alkyl group optionally substituted by a C₁₋₆alkoxy group, or R^{m} and Rⁿ together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and/or optionally substituted by one or more of the following: a C₁₋₆alkoxy group; hydroxy; oxo; carboxy; a C₁₋₆alkylsulfonyl group; or a C₁₋₆alkyl group optionally substituted by one or more hydroxy or C₁₋₆alkoxy;
7) optionally substituted heteroaryl including N-oxides and S-oxides thereof optionally substituted by one or more of the groups i to xxix listed above;
wherein any alkyl chain mentioned in any of the definitions from A to R above or in any of the definitions i to xxix above is optionally substituted by 1) one group or two groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 10 membered heterocyclic group in the list of optional substituents from A to X above or in any of the definitions i to xxix above, for which specific substitution has not been previously mentioned, is optionally substituted by one, two or three groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; a C₁₋₄alkanoyloxy group or a C₁₋₄alkyl optionally substituted by one or more hydroxy, C₁₋₃alkoxy or a group —NR^{e}R^{f} in which R^{e} and R^{f} are as defined above; and/or is optionally substituted by one to five fluoro;
R² represents H, a C₁₋₃alkyl group; a C₁₋₃alkoxy group, cyano, halo or a group RS(O)x in which x is 0, 1 or 2;
R³ represents H, a C₁₋₃alkyl group; a C₁₋₃alkoxy group; cyano or halo;
R⁴ represents i) a C₁₋₃alkyl group optionally substituted by one or more halo ii) a C₁₋₃alkoxy group optionally substituted by one or more halo iii) halo, iv) nitro, v) cyano, vi) a C₁₋₆alkylS(O)_{y}(O)_{z}— wherein y is 0, 1 or 2 and z is 0 except when y is 2 when z is 0 or 1 vii) a group CH₂NR^{u}R^{v} in which R^{u} and R^{v} independently represent H; a C₁₋₃alkylsulfonyl group, a C₁₋₃alkanoyl group or a C₁₋₃alkyl group or R^{u} and R^{v} together with the nitrogen atom to which they are attached represent azetidinyl, pyrrolidinyl, piperidinyl or morpholinyl; viii) a group CO₂R^{u} in which R^{u} is a C₁₋₃alkyl group; or ix) a group CONR^{x}R^{y} in which R^{x} and R^{y} independently represent H; or a C₁₋₃alkyl group or R^{x} and R^{y} together with the nitrogen atom to which they are attached represent azetidinyl; pyrrolidinyl, piperidinyl or morpholinyl;
R⁵ and R^{5′} independently represent H, halo, cyano, C₁₋₃alkyl optionally substituted by one or more halo or C₁₋₃alkoxy optionally substituted by one or more halo;
R⁶ and R^{6′} independently represent H, halo, cyano, C₁₋₃alkyl optionally substituted by one or more halo or C₁₋₃alkoxy optionally substituted by one or more halo; and

### R⁷ is H or OH.

In another aspect the present invention provides a compound of formula I

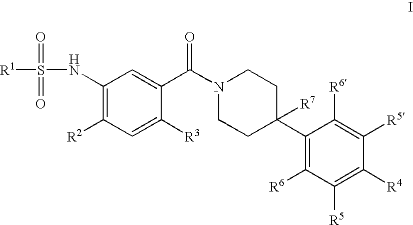

or a pharmaceutically acceptable salt thereof, in which
R¹ represents 1) a C₁₋₆alkyl group optionally substituted by one or two groups selected from A-S below and/or by one to five groups selected from T below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) carbamoyl; vii) N—C₁₋₆alkylcarbamoyl; viii) N,N-diC₁₋₆alkylcarbamoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl xix) benzyloxy; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 8 membered heterocyclic group as defined in c) below; xxvii) phenylsulfonyl; xxviii) heteroarylsulfonyl;
xxix) a group of formula NR^{c}R^{d} in which R^{c} and R^{d} independently represent:

### a) H;

b) C₁₋₆alkanoyl;
c) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by one or more of the following: hydroxy, oxo, carboxy, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulfonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 8 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulfonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl;
k) heteroaryl optionally substituted by one or more carboxy; fluoro; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group;
B) a heteroaryl group which is optionally substituted by groups i) to xxix) as described for phenyl above;
C) a group of formula NR^{c}R^{d} in which R^{c} and R^{d} are as defined above;
D) a C₃₋₇cycloalkyl group optionally substituted by one or more hydroxy or a group of formula NR^{e}R^{f} in which R^{e} and R^{f} are as defined above;
E) a carbon linked saturated or partially unsaturated 4 to 8 membered heterocyclic group containing one or more N, S or O, wherein the S may be in its oxidised form of SO or SO₂, which is optionally fused to a benz ring and/or is optionally substituted by one or more of the following: hydroxy; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; or a C₁₋₆alkyl optionally substituted by one or more hydroxy or C₁₋₆alkoxy;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulfonyl group;
M) phenylsulfonyl;
N) heteroarylsulfonyl;
O) benzoyl;
P) a C₁₋₆alkanoyl group
Q) hydroxy;
R) oxo;
S) carboxy;
T) fluoro
or R¹ represents
2) a C₃₋₇cycloalkyl group optionally substituted by one or two groups selected from A to T above;
3) a C₂₋₆alkynyl group optionally substituted by one or two groups selected from A to T above;
4) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by a group A to T as defined above one or more of the following: hydroxy, oxo, carboxy, a C₁₋₆alkoxy group, hydroxy, a C₁₋₆alkylsulfonyl group, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
5) a C₂₋₆alkenyl group optionally substituted by one or two groups selected from A to T above;
6) optionally substituted phenyl including optional fusion of the phenyl ring to a saturated or partially unsaturated 5 to 6 membered heterocyclic ring optionally containing one, two or three hetero atoms selected from oxygen, sulphur optionally in its oxidised forms of SO or SO₂ or nitrogen wherein the heterocyclic ring is optionally substituted by one or more of the following: a C₁₋₆alkoxy group; a C₁₋₆alkanoyl group; carboxy; a C₁₋₆alkylsulfonyl group; a C₁₋₆alkoxycarbonyl group; carbamoyl; N—C₁₋₆alkylcarbamoyl; N,N-diC₁₋₆alkylcarbamoyl; hydroxy; oxo; a C₁₋₆alkyl group (which is optionally substituted by one or more of the following: a C₁₋₆alkoxy group, hydroxy or a group of formula NR^{c}R^{d} in which R^{c} and R^{d} are as defined above) and wherein the phenyl ring is optionally substituted by one or more of the groups i to xxix listed above or by a heteroaryl group optionally substituted by one or more groups i) to xxix) above or by an ureido group of formula R^{m}RⁿN—C(O)—NH— in which R^{m} and Rⁿ independently represent H, a C₁₋₆alkyl group optionally substituted by a C₁₋₆alkoxy group, or R^{m} and Rⁿ together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and/or optionally substituted by one or more of the following: a C₁₋₆alkoxy group; hydroxy; oxo; carboxy; a C₁₋₆alkylsulfonyl group; or a C₁₋₆alkyl group optionally substituted by one or more hydroxy or C₁₋₆alkoxy;
7) optionally substituted heteroaryl including N-oxides and S-oxides thereof optionally substituted by one or more of the groups i to xxix listed above;
wherein any alkyl chain mentioned in any of the definitions from A to P above or in any of the definitions i to xxix above is optionally substituted by 1) one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 8 membered heterocyclic group in the list of optional substituents from A to P above or in any of the definitions i to xxix above, for which specific substitution has not been previously mentioned, is optionally substituted by one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or is optionally substituted by one to five fluoro;
R² represents H, a C₁₋₃alkyl group; a C₁₋₃alkoxy group, cyano or halo;
R³ represents H, a C₁₋₃alkyl group; a C₁₋₃alkoxy group; cyano or halo;
R⁴ represents i) a C₁₋₃alkyl group optionally substituted by one or more halo ii) a C₁₋₃alkoxy group optionally substituted by one or more halo iii) halo, iv) nitro, v) cyano, vi) a C₁₋₆alkylS(O)_{y}(O)_{z}— wherein y is 0, 1 or 2 and z is 0 except when y is 2 when z is 0 or 1 vii) a group CH₂NR^{u}R^{v} in which R^{u} and R^{v} independently represent H; a C₁₋₃alkylsulfonyl group, a C₁₋₃alkanoyl group or a C₁₋₃alkyl group or R^{x} and R^{y} together with the nitrogen atom to which they are attached represent azetidinyl, pyrrolidinyl, piperidinyl or morpholinyl; ix) a group CO₂R^{w} in which R^{w} is a C₁₋₃alkyl group; or x) a group CONR^{x}R^{y} in which R^{x} and R^{y} independently represent H; or a C₁₋₃alkyl group or R^{x} and R^{y} together with the nitrogen atom to which they are attached represent azetidinyl; pyrrolidinyl, piperidinyl or morpholinyl;
R⁵ and R^{5′} independently represent H, halo, cyano, C₁₋₃alkyl optionally substituted by one or more halo or C₁₋₃alkoxy optionally substituted by one or more halo;
R⁶ and R^{6′} independently represent H, halo, cyano, C₁₋₃alkyl optionally substituted by one or more halo or C₁₋₃alkoxy optionally substituted by one or more halo; and

### R⁷ is H or OH.

In another aspect the present invention provides a compound of formula II

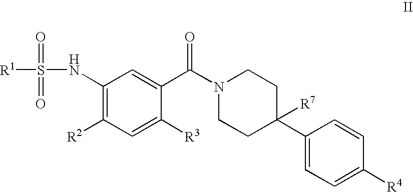

R¹ represents 1) a C₁₋₆alkyl group optionally substituted by one or two groups selected from A-S below and/or by one to five groups selected from T below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) carbamoyl; vii) N—C₁₋₆alkylcarbamoyl; viii) N,N-diC₁₋₆alkylcarbamoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl xix) benzyloxy; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 8 membered heterocyclic group as defined in c) below; xxvii) phenylsulfonyl; xxviii) heteroarylsulfonyl;
xxix) a group of formula NR^{c}R^{d} in which R^{c} and R^{d} independently represent:

### a) H;

b) C₁₋₆alkanoyl;
c) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by one or more of the following: hydroxy, oxo, carboxy, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulfonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 8 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulfonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl;
k) heteroaryl optionally substituted by one or more carboxy; fluoro; hydroxy a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group;
B) a heteroaryl group which is optionally substituted by groups i) to xxix) as described for phenyl above;
C) a group of formula NR^{c}R^{d} in which R^{c} and R^{d} are as defined above;
D) a C₃₋₇cycloalkyl group optionally substituted by one or more hydroxy or a group of formula NR^{e}R^{f} in which R^{e} and R^{f} are as defined above;
E) a carbon linked saturated or partially unsaturated 4 to 8 membered heterocyclic group containing one or more N, S or O, wherein the S may be in its oxidised form of SO or SO₂, which is optionally fused to a benz ring and/or is optionally substituted by one or more of the following: hydroxy; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; or a C₁₋₆alkyl optionally substituted by one or more hydroxy or C₁₋₆alkoxy;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulfonyl group;
M) phenylsulfonyl;
N) heteroarylsulfonyl;
O) benzoyl;
P) a C₁₋₆alkanoyl group
Q) hydroxy;
R) oxo;
S) carboxy;
T) fluoro
or R¹ represents
2) a C₃₋₇cycloalkyl group optionally substituted by one or two groups selected from A to T above;
3) a C₂₋₆alkynyl group optionally substituted by one or two groups selected from A to T above;
4) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by a group A to T as defined above one or more of the following: hydroxy, oxo, carboxy, a C₁₋₆alkoxy group, hydroxy, a C₁₋₆alkylsulfonyl group, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
5) a C₂₋₆alkenyl group optionally substituted by one or two groups selected from A to T above;
6) optionally substituted phenyl including optional fusion of the phenyl ring to a saturated or partially unsaturated 5 to 6 membered heterocyclic ring optionally containing one, two or three hetero atoms selected from oxygen, sulphur optionally in its oxidised forms of SO or SO₂ or nitrogen wherein the heterocyclic ring is optionally substituted by one or more of the following: a C₁₋₆alkoxy group; a C₁₋₆alkanoyl group; carboxy; a C₁₋₆alkylsulfonyl group; a C₁₋₆alkoxycarbonyl group; carbamoyl; N—C₁₋₆alkylcarbamoyl; N,N-diC₁₋₆alkylcarbamoyl; hydroxy; oxo; a C₁₋₆alkyl group (which is optionally substituted by one or more of the following: a C₁₋₆alkoxy group, hydroxy or a group of formula NR^{c}R^{d} in which R^{c} and R^{d} are as defined above) and wherein the phenyl ring is optionally substituted by one or more of the groups i to xxix listed above or by a heteroaryl group optionally substituted by one or more groups i) to xxix) above or by an ureido group of formula R^{m}RⁿN—C(O)—NH— in which R^{m} and Rⁿ independently represent H, a C₁₋₆alkyl group optionally substituted by a C₁₋₆alkoxy group, or R^{m} and Rⁿ together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and/or optionally substituted by one or more of the following: a C₁₋₆alkoxy group; hydroxy; oxo; carboxy; a C₁₋₆alkylsulfonyl group; or a C₁₋₆alkyl group optionally substituted by one or more hydroxy or C₁₋₆alkoxy;
7) optionally substituted heteroaryl including N-oxides and S-oxides thereof optionally substituted by one or more of the groups i to xxix listed above or by oxo;
wherein any alkyl chain mentioned in any of the definitions from A to P above or in any of the definitions i to xxix above is optionally substituted by 1) one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 8 membered heterocyclic group in the list of optional substituents from A to P above or in any of the definitions i to xxix above, for which specific substitution has not been previously mentioned, is optionally substituted by one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or is optionally substituted by one to five fluoro;
R² represents H, a C₁₋₃alkyl group; a C₁₋₃alkoxy group, cyano or halo;
R³ represents H, a C₁₋₃alkyl group; a C₁₋₃alkoxy group; cyano or halo;
R⁴ represents halo, cyano or a C₁₋₄alkylsulfonyl group;

### R⁷ is H or OH.

In another aspect the present invention provides a compound of formula IIA

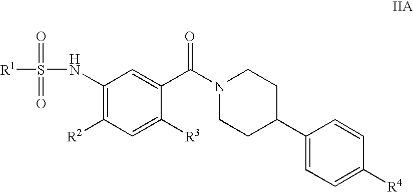

R¹ represents 1) a C₁₋₆alkyl group optionally substituted by one or more of the following: a) halo b) a C₁₋₆alkoxycarbonyl c) a C₃₋₆cycloalkyl group d) phenyl optionally substituted by one or more of the following: halo or a C₁₋₄alkylsulfonyl group; e) a C₁₋₄alkylsulfonyl group or f) an amino group of formula NR^{u}R^{v} in which R^{u} and R^{v} are as defined above;
2) C₃₋₆cycloalkyl group; or
3) phenyl optionally substituted by one or more of the following: a) halo; b) cyano; c) a C₁₋₆alkanoylamino group or d) a C₁₋₆alkoxy group;
4) thienyl optionally substituted by one or more halo;
5) 2-oxo-1,3-dihydroindol-5-yl;
6) 5-methyl-1,2-oxazol-4-yl;
R² represents H, a C₁₋₃alkyl group; a C₁₋₃alkoxy group, cyano or halo;
R³ represents H, a C₁₋₃alkyl group; a C₁₋₃alkoxy group; cyano or halo; and
R⁴ represents cyano or a C₁₋₄alkylsulfonyl group.

Particular values of the substituents R¹, R², R³, R⁴ and R⁷ are now given. It will be understood that such values may be used where appropriate with any of the definitions, claims or embodiments defined hereinbefore or hereinafter.

In a first group of compounds of formula I, one of R² and R³ is other than H.
In a second group of compounds of formula I, R² is methyl and R³ is H.
In a third group of compounds of formula I, R³ is methyl and R² is H.
In a fourth group of compounds of formula I, R² is methyl and R³ is methyl.
In a fifth group of compounds of formula I, R⁷ is H.
In a first group of compounds of formula II, one of R² and R³ is other than H.
In a second group of compounds of formula II, R² is methyl and R³ is H.
In a third group of compounds of formula II, R² is H and R³ is methyl.
In a fourth group of compounds of formula II, R² is methyl and R³ is methyl.
In a fifth group of compounds of formula II R⁷ is H.
In a first group of compounds of formula IIA, one of R² and R³ is other than H.
In a second group of compounds of formula IIA, R² is methyl and R³ is H.
In a third group of compounds of formula IIA, R² is H and R³ is methyl.
In a fourth group of compounds of formula IIA, R² is methyl and R³ is methyl.

In a particular groups of compounds of formulae I, II or IIA, R¹ represents a group —(CH₂)₃NR^{c}R^{d} in which R^{c} and R^{d} are as described above. Particularly R^{c} represents H or a C₁₋₄alkyl and R^{d} represents H or a C₁₋₄alkyl.

In a further particular group of compounds of formulae I, II or IIA, R¹ represents a carbon linked saturated or partially unsaturated 4 to 7 membered heterocyclic group, containing one or more N, S or O, wherein the S may be in its oxidised form of SO or SO₂, which is optionally fused to a benz ring and any ring is optionally substituted by a group A to W as defined above. Particularly R¹ represents piperidinyl or 1,1-dioxothiolanyl.

“Pharmaceutically acceptable salt”, where such salts are possible, includes both pharmaceutically acceptable acid and base addition salts. A suitable pharmaceutically acceptable salt of a compound of formula I is, for example, an acid-addition salt of a compound of formula I which is sufficiently basic, for example an acid-addition salt with an inorganic or organic acid such as hydrochloric, hydrobromic, sulphuric, trifluoroacetic, citric or maleic acid; or, for example a base-addition salt of a compound of formula I which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates including solvates of the free compounds or solvates of a salt of the compound. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions that will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of a compound of formula I that is compounds which are converted into a compound of formula I in vivo.

The following definitions shall apply throughout the specification and the appended claims.

The term “C₃₋₁₀cycloalkyl group which may be monocyclic, bicyclic or tricyclic and optionally may be bridged” includes cyclopropyl, cyclobutyl, cyclopentyl, cyclohexyl, cycloheptyl, cyclooctyl, cyclodecyl, bicyclo(2.2.1)heptyl, bicyclo(2.2.2)octyl, perhydroindanyl and adamantyl.

The term “heteroaryl” includes an aromatic 5- or 6-membered monocyclic ring or unless specified otherwise, an 8-, 9- or 10-membered bicyclic ring, with up to five ring heteroatoms selected from oxygen, nitrogen and sulfur, which may, unless otherwise specified be carbon or nitrogen linked. In one embodiment heteroaryl is an aromatic 5- or 6-membered monocyclic ring with up to five ring heteroatoms selected from oxygen, nitrogen and sulfur, which may, unless otherwise specified be carbon or nitrogen linked and includes pyrrolyl, thienyl, furyl, pyrazolyl, imidazolyl, oxazolyl, isoxazolyl, thiazolyl, isothiazolyl, thiadiazolyl, 1,3,4-oxadiazolyl, 1,2,4-oxadiazolyl, triazolyl, furazanyl, tetrazolyl, pyridyl, pyrimidyl, pyrazinyl, pyridazinyl, 1,3,5-triazinyl and imidazothiazolyl. Other heteroaryls include quinolyl, isoquinolyl, benzthienyl, benzofuranyl, benzofurazanyl, benzoxazolyl, benzimidazolyl, indolyl, benzthiazolyl, indazolyl, cinnolinyl, quinazolinyl, quinoxalinyl, phthalazinyl, 1,5-naphthyridinyl, 1,6-naphthyridinyl, 1,7-naphthyridinyl, 1,8-naphthyridinyl, pyrrolopyridinyl, pyrrolopyrazinyl, pyrazolopyridinyl or imidazopyridinyl.

The term “heteroaryl including N-oxides” includes heteroaryls as described immediately above and in addition N-oxides of such heteroaryls where such N-oxides are known to those skilled in the art to exist and are known to be stable at ambient conditions for example pyridine-N-oxides.

The term “a carbon linked saturated or partially unsaturated 4 to 10 (or 4 to 8) membered heterocyclic group containing one or more N, S or O, wherein the S may be in its oxidised form of SO or SO₂ which is optionally fused to a benz ring or a heteroaryl ring” includes oxiranyl, oxetanyl, tetrahydrofuranyl, tetrahydropyranyl, 2,3-dihydro-1,3-thiazolyl, 1,3-thiazolidinyl, 1,3-oxazolidinyl, oxepanyl, azetidinyl, pyrrolinyl, pyrrolidinyl, morpholinyl, thiamorpholinyl (perhydro-1,4-thiazinyl), (8-oxa-3-azabicyclo[3.2.1]octyl), (7-oxa-3-azabicyclo[3.1.1]heptyl), perhydroazepinyl, perhydrooxazepinyl, tetrahydro-1,4-thiazinyl, 1-oxotetrahydrothienyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl or tetrahydroquinolyl each of which may be optionally substituted as previously described.

When two substituents on an amine together with the nitrogen atom to which they are attached represent a saturated or partially unsaturated 4 to 10 membered heterocyclic ring optionally containing an additional oxygen, sulphur, SO, SO₂ or nitrogen O and/or optionally fused to a benz ring then such rings include azetidino, pyrrolidino, morpholino, piperidino, imidazolidinyl, imidazolinyl, piperazino, thiamorpholino (perhydro-1,4-thiazinyl), homopiperazino, perhydroazepino, perhydrooxazepino, (2,3-dihydro-1,3-thiazolyl, 1,3-thiazolidinyl, 1,3-oxazolidinyl, oxepanyl, oxazepanyl, dihydropyrimidinyl, tetrahydropyrimidinyl, and homopiperidinyl, each of which is optionally substituted as previously described.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl, t-butyl, pentyl, isopentyl, neopentyl, tert-pentyl, hexyl and isohexyl. Preferred alkyl groups are methyl, ethyl, propyl, isopropyl, butyl and tertiary butyl.

Unless otherwise stated or indicated, the term “alkoxy” denotes a group O-alkyl, wherein alkyl is as defined above.

Unless otherwise stated or indicated, the term “halogen” shall mean fluorine, chlorine, bromine or iodine.

An example of “C₁₋₆alkanoyloxy” is acetoxy. Examples of “C₁₋₆alkoxycarbonyl” include C₁₋₄alkoxycarbonyl, methoxycarbonyl, ethoxycarbonyl, n- and t-butoxycarbonyl. Examples of “C₁₋₆alkoxycarbonylamino” include methoxycarbonylamino, ethoxycarbonylamino, n- and t-butoxycarbonylamino. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)_{y}(O)_{z}— optionally substituted by one or more fluoro wherein y is 0, 1 or 2 and z is 0 except when y is 2 then z may also be 1” include methylthio, ethylthio, methylsulfinyl, ethylsulfinyl, mesyl, ethylsulfonyl methanesulfonyloxy and trifluoromethylsulfonyloxy. Examples of “C₁₋₆alkylsulfonylamino” include methylsulfonylamino, ethylsulfonylamino and propylsulfonylamino. Examples of “C₁₋₆alkylsulfonyl-N—(C₁₋₆alkyl)amino” include methylsulfonyl-N-methylamino, ethylsulfonyl-N-methylamino and propylsulfonyl-N-ethylamino. Examples of “C₁₋₆alkanoyl” include C₁₋₄alkanoyl, propionyl and acetyl. Examples of “N—(C₁₋₆alkyl)amino” include methylamino and ethylamino. Examples of “N,N—(C₁₋₆alkyl)₂-amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are N—(C₁₋₄alkyl)carbamoyl, methylaminocarbonyl and ethylaminocarbonyl. Examples of “N,N—(C₁₋₆alkyl)₂carbamoyl” are N,N—(C₁₋₄alkyl)carbamoyl, dimethylaminocarbonyl and methylethylaminocarbonyl. Examples of “N—(C₁₋₆alkyl)sulphamoylamino” are N-(methyl)sulphamoylamino and N-(ethyl)sulphamoylamino. Examples of “N—(C₁₋₆alkyl)₂sulphamoylamino” are N,N-(dimethyl)sulphamoylamino and N-(methyl)-N-(ethyl)sulphamoylamino. Examples of “C₁₋₆alkylsulfonylaminocarbonyl” include methylsulfonylaminocarbonyl, ethylsulfonylaminocarbonyl and propylsulfonylaminocarbonyl.

Specific compounds of the invention include one or more, including any number from 1 to 253, of the following compounds below labelled as List 1:
N-[5-[4-(4-cyanophenyl)piperidine-1-carbonyl]-2-methyl-phenyl]methanesulfonamide;
N-[5-[4-(4-cyanophenyl)piperidine-1-carbonyl]-2-methyl-phenyl]-2-fluoro-benzenesulfonamide;
4-chloro-N-[5-[4-(4-cyanophenyl)piperidine-1-carbonyl]-2-methyl-phenyl]benzenesulfonamide;
N-[5-[4-(4-cyanophenyl)piperidine-1-carbonyl]-2-methyl-phenyl]-1-phenyl-methanesulfonamide;
N-[5-[4-(4-cyanophenyl)piperidine-1-carbonyl]-2-methyl-phenyl]propane-1-sulfonamide;
N-[5-[4-(4-cyanophenyl)piperidine-1-carbonyl]-2-methoxy-phenyl]methanesulfonamide;
N-[3-[4-(4-cyanophenyl)piperidine-1-carbonyl]-4-methyl-phenyl]methanesulfonamide;
N-[3-[4-(4-cyanophenyl)piperidine-1-carbonyl]-4-methyl-phenyl]-1-phenyl-methanesulfonamide;
3-chloro-N-[5-[4-(4-cyanophenyl)piperidine-1-carbonyl]-2-methyl-phenyl]propane-1-sulfonamide;
N-[5-[4-(4-cyanophenyl)piperidine-1-carbonyl]-2-methyl-phenyl]benzenesulfonamide;
N-[5-[4-(4-cyanophenyl)piperidine-1-carbonyl]-2-methyl-phenyl]ethanesulfonamide;
N-[5-[4-(4-cyanophenyl)piperidine-1-carbonyl]-2-methyl-phenyl]butane-1-sulfonamide;
methyl 2-[[5-[4-(4-cyanophenyl)piperidine-1-carbonyl]-2-methyl-phenyl]sulfamoyl]-acetate;
N-[5-[4-(4-cyanophenyl)piperidine-1-carbonyl]-2-methyl-phenyl]cyclopropane-sulfonamide;
N-[5-[4-(4-cyanophenyl)piperidine-1-carbonyl]-2-methyl-phenyl]-1-cyclohexyl-methanesulfonamide;
N-[5-[4-(4-cyanophenyl)piperidine-1-carbonyl]-2-methyl-phenyl]-1-cyclopentyl-methanesulfonamide;
N-[5-[4-(4-cyanophenyl)piperidine-1-carbonyl]-2-methyl-phenyl]cyclohexanesulfonamide;
N-[5-[4-(4-cyanophenyl)piperidine-1-carbonyl]-2-methyl-phenyl]butane-2-sulfonamide;
5-chloro-N-[5-[4-(4-cyanophenyl)piperidine-1-carbonyl]-2-methyl-phenyl]thiophene-2-sulfonamide;
2-cyano-N-[5-[4-(4-cyanophenyl)piperidine-1-carbonyl]-2-methyl-phenyl]benzene-sulfonamide:
3-cyano-N-[5-[4-(4-cyanophenyl)piperidine-1-carbonyl]-2-methyl-phenyl]benzene-sulfonamide;
N-[4-[[5-[4-(4-cyanophenyl)piperidine-1-carbonyl]-2-methyl-phenyl]sulfamoyl]phenyl]-acetamide;
4-cyano-N-[5-[4-(4-cyanophenyl)piperidine-1-carbonyl]-2-methyl-phenyl]benzene-sulfonamide;
4-butoxy-N-[5-[4-(4-cyanophenyl)piperidine-1-carbonyl]-2-methyl-phenyl]benzene-sulfonamide;
N-[5-[4-(4-cyanophenyl)piperidine-1-carbonyl]-2-methyl-phenyl]-2-methoxy-benzene-sulfonamide;
N-[5-[4-(4-cyanophenyl)piperidine-1-carbonyl]-2-methyl-phenyl]-1-(2-fluorophenyl)-methanesulfonamide;
N-[5-[4-(4-cyanophenyl)piperidine-1-carbonyl]-2-methyl-phenyl]-1-(3-fluorophenyl)-methanesulfonamide;
N-[5-[4-(4-cyanophenyl)piperidine-1-carbonyl]-2,4-dimethyl-phenyl]methanesulfonamide;
N-[5-[4-(4-cyanophenyl)piperidine-1-carbonyl]-2,4-dimethyl-phenyl]-1-phenyl-methanesulfonamide;
N-[2-cyano-5-[4-(4-cyanophenyl)piperidine-1-carbonyl]phenyl]-1-phenyl-methanesulfonamide;
N-[3-[4-(4-cyanophenyl)piperidine-1-carbonyl]-4-methoxy-phenyl]-1-phenyl-methanesulfonamide;
N-[3-[4-(4-cyanophenyl)piperidine-1-carbonyl]-4-methyl-phenyl]propane-2-sulfonamide;
N-[3-[4-(4-cyanophenyl)piperidine-1-carbonyl]-4-methyl-phenyl]propane-1-sulfonamide;
3-chloro-N-[3-[4-(4-cyanophenyl)piperidine-1-carbonyl]-4-methyl-phenyl]propane-1-sulfonamide;
N-[3-[4-(4-cyanophenyl)piperidine-1-carbonyl]-4-methyl-phenyl]benzenesulfonamide;
N-[3-[4-(4-cyanophenyl)piperidine-1-carbonyl]-4-methyl-phenyl]ethanesulfonamide;
N-[3-[4-(4-cyanophenyl)piperidine-1-carbonyl]-4-methyl-phenyl]butane-1-sulfonamide;
methyl 2-[[3-[4-(4-cyanophenyl)piperidine-1-carbonyl]-4-methyl-phenyl]sulfamoyl]-acetate;
N-[3-[4-(4-cyanophenyl)piperidine-1-carbonyl]-4-methyl-phenyl]-5-methyl-1,2-oxazole-4-sulfonamide;
N-[3-[4-(4-cyanophenyl)piperidine-1-carbonyl]-4-methyl-phenyl]cyclopropane-sulfonamide;
N-[3-[4-(4-cyanophenyl)piperidine-1-carbonyl]-4-methyl-phenyl]-1-cyclohexyl-methanesulfonamide;
N-[3-[4-(4-cyanophenyl)piperidine-1-carbonyl]-4-methyl-phenyl]cyclohexanesulfonamide;
N-[3-[4-(4-cyanophenyl)piperidine-1-carbonyl]-4-methyl-phenyl]-1-cyclopentyl-methanesulfonamide;
N-[3-[4-(4-cyanophenyl)piperidine-1-carbonyl]-4-methyl-phenyl]butane-2-sulfonamide;
5-chloro-N-[3-[4-(4-cyanophenyl)piperidine-1-carbonyl]-4-methyl-phenyl]thiophene-2-sulfonamide;
2-cyano-N-[3-[4-(4-cyanophenyl)piperidine-1-carbonyl]-4-methyl-phenyl]benzenesulfonamide;
3-cyano-N-[3-[4-(4-cyanophenyl)piperidine-1-carbonyl]-4-methyl-phenyl]benzenesulfonamide;
N-[4-[[3-[4-(4-cyanophenyl)piperidine-1-carbonyl]-4-methyl-phenyl]sulfamoyl]phenyl]acetamide;
4-cyano-N-[3-[4-(4-cyanophenyl)piperidine-1-carbonyl]-4-methyl-phenyl]benzenesulfonamide;
N-[3-[4-(4-cyanophenyl)piperidine-1-carbonyl]-4-methyl-phenyl]-2-methoxy-benzenesulfonamide;
N-[3-[4-(4-cyanophenyl)piperidine-1-carbonyl]-4-methyl-phenyl]-1-(2-fluorophenyl)methanesulfonamide;
N-[3-[4-(4-cyanophenyl)piperidine-1-carbonyl]-4-methyl-phenyl]-1-(3-fluorophenyl)methanesulfonamide;
N-[3-[4-(4-cyanophenyl)piperidine-1-carbonyl]-4-methyl-phenyl]-2-oxo-1,3-dihydroindole-5-sulfonamide;
N-[3-[4-(4-cyanophenyl)piperidine-1-carbonyl]-4-methyl-phenyl]-1-(4-methylsulfonylphenyl)methanesulfonamide;
N-[3-[4-(4-cyanophenyl)piperidine-1-carbonyl]-4-methyl-phenyl]-2,2,2-trifluoro-ethanesulfonamide;
N-[3-[4-(4-cyanophenyl)piperidine-1-carbonyl]-4-methyl-phenyl]-1-(2,4-difluorophenyl)methanesulfonamide;
N-[3-[4-(4-cyanophenyl)piperidine-1-carbonyl]-4-methyl-phenyl]-2-methyl-propane-1-sulfonamide;
N-[3-[4-(4-cyanophenyl)piperidine-1-carbonyl]-4-methyl-phenyl]-2-phenyl-ethanesulfonamide;
N-[3-[4-(4-cyanophenyl)piperidine-1-carbonyl]-4-methyl-phenyl]pentane-2-sulfonamide;
N-[5-[4-(4-cyanophenyl)piperidine-1-carbonyl]-2-methyl-phenyl]-2,2,2-trifluoro-ethanesulfonamide;
N-[5-[4-(4-cyanophenyl)piperidine-1-carbonyl]-2-methyl-phenyl]-1-(2,4-difluorophenyl)methanesulfonamide;
N-[5-[4-(4-cyanophenyl)piperidine-1-carbonyl]-2-methyl-phenyl]-1-(4-methylsulfonylphenyl)methanesulfonamide;
N-[5-[4-(4-cyanophenyl)piperidine-1-carbonyl]-2-methyl-phenyl]pentane-2-sulfonamide;
N-[5-[4-(4-cyanophenyl)piperidine-1-carbonyl]-2-methyl-phenyl]-2-methyl-propane-1-sulfonamide;
N-[5-[4-(4-cyanophenyl)piperidine-1-carbonyl]-2-methyl-phenyl]-2-phenyl-ethanesulfonamide;
N-[3-[4-(4-cyanophenyl)piperidine-1-carbonyl]phenyl]-1-phenyl-methanesulfonamide;
N-[5-[4-(4-cyanophenyl)piperidine-1-carbonyl]-2-methyl-phenyl]-3-morpholin-4-yl-propane-1-sulfonamide;
N-[2-methyl-5-[4-(4-methylsulfonylphenyl)piperidine-1-carbonyl]phenyl]methanesulfonamide
N-[3-[4-(4-cyanophenyl)piperidine-1-carbonyl]-4-methyl-phenyl]-1,1-dioxo-thiolane-3-sulfonamide
N-[5-[4-(4-cyanophenyl)-4-hydroxy-piperidine-1-carbonyl]-2-methyl-phenyl]-1-phenyl-methanesulfonamide
N-[5-[4-(4-cyanophenyl)piperidine-1-carbonyl]-2-methyl-phenyl]-2-(1,3-dioxoisoindol-2-yl)ethanesulfonamide
2-amino-N-[5-[4-(4-cyanophenyl)piperidine-1-carbonyl]-2-methyl-phenyl]ethanesulfonamide
N-[5-[4-(4-cyanophenyl)piperidine-1-carbonyl]-2-methyl-phenyl]-3-(propan-2-ylamino)propane-1-sulfonamide
N-[5-[4-(4-cyanophenyl)piperidine-1-carbonyl]-2-methyl-phenyl]-3-dimethylamino-propane-1-sulfonamide
N-[5-[4-(4-cyanophenyl)piperidine-1-carbonyl]-2-methyl-phenyl]-3-methylamino-propane-1-sulfonamide
methyl 3-[[5-[4-(4-cyanophenyl)piperidine-1-carbonyl]-2-methyl-phenyl]sulfamoyl]benzoate
N-[5-[4-(4-cyanophenyl)piperidine-1-carbonyl]-2-methyl-phenyl]-2-methanesulfonamido-ethanesulfonamide
N-[2-[[5-[4-(4-cyanophenyl)piperidine-1-carbonyl]-2-methyl-phenyl]sulfamoyl]ethyl]acetamide
3-[[5-[4-(4-cyanophenyl)piperidine-1-carbonyl]-2-methyl-phenyl]sulfamoyl]benzoic acid
N-[2-[[5-[4-(4-cyanophenyl)piperidine-1-carbonyl]-2-methyl-phenyl]sulfamoyl]ethyl]propane-2-sulfonamide
3-[[5-[4-(4-cyanophenyl)piperidine-1-carbonyl]-2-methyl-phenyl]sulfamoyl]benzamide
3-[[5-[4-(4-cyanophenyl)piperidine-1-carbonyl]-2-methyl-phenyl]sulfamoyl]-N,N-dimethyl-benzamide
3-[[5-[4-(4-cyanophenyl)piperidine-1-carbonyl]-2-methyl-phenyl]sulfamoyl]-N-methyl-benzamide
3-[[5-[4-(4-cyanophenyl)piperidine-1-carbonyl]-2-methyl-phenyl]sulfamoyl]-N-(2-hydroxyethyl)benzamide
3-[[5-[4-(4-cyanophenyl)piperidine-1-carbonyl]-2-methyl-phenyl]sulfamoyl]-N-propan-2-yl-benzamide
N-[2-[[5-[4-(4-cyanophenyl)piperidine-1-carbonyl]-2-methyl-phenyl]sulfamoyl]ethyl]benzamide
N-[2-[[5-[4-(4-cyanophenyl)piperidine-1-carbonyl]-2-methyl-phenyl]sulfamoyl]ethyl]-2-methoxy-acetamide
N-[5-[4-(4-cyanophenyl)piperidine-1-carbonyl]-2-methyl-phenyl]-2-morpholin-4-yl-2-oxo-ethanesulfonamide
N-[5-[4-(4-cyanophenyl)piperidine-1-carbonyl]-2-methoxy-phenyl]-1-phenyl-methanesulfonamide
6-chloro-N-[5-[4-(4-cyanophenyl)piperidine-1-carbonyl]-2-methyl-phenyl]pyridine-3-sulfonamide
2-[[5-[4-(4-cyanophenyl)piperidine-1-carbonyl]-2-methyl-phenyl]sulfamoyl]-N,N-dimethyl-acetamide
2-[[5-[4-(4-cyanophenyl)piperidine-1-carbonyl]-2-methyl-phenyl]sulfamoyl]-N-(2-hydroxyethyl)acetamide
N-[5-[4-(4-cyanophenyl)piperidine-1-carbonyl]-2-methyl-phenyl]-6-morpholin-4-yl-pyridine-3-sulfonamide
N-[5-[4-(4-cyanophenyl)piperidine-1-carbonyl]-2-methyl-phenyl]-1-methyl-imidazole-4-sulfonamide
N-[5-[4-(4-cyanophenyl)piperidine-1-carbonyl]-2-methyl-phenyl]-5-methyl-1,2-oxazole-4-sulfonamide
N-[5-[4-(4-cyanophenyl)piperidine-1-carbonyl]-2-methyl-phenyl]-6-dimethylamino-pyridine-3-sulfonamide
N-[5-[4-(4-cyanophenyl)piperidine-1-carbonyl]-2-methyl-phenyl]-3-(morpholine-4-carbonyl)benzenesulfonamide
N-[5-[4-(4-cyanophenyl)piperidine-1-carbonyl]-2-methyl-phenyl]ethenesulfonamide
N-[5-[4-(4-cyanophenyl)piperidine-1-carbonyl]-2-methyl-phenyl]-6-(propan-2-ylamino)pyridine-3-sulfonamide
5-chloro-N-[5-[4-(4-cyanophenyl)piperidine-1-carbonyl]-2-methyl-phenyl]-1,3-dimethyl-pyrazole-4-sulfonamide
7-chloro-N-[5-[4-(4-cyanophenyl)piperidine-1-carbonyl]-2-methyl-phenyl]-4-thia-1,6-diazabicyclo[3.3.0]octa-2,5,7-triene-8-sulfonamide
N-[5-[4-(4-cyanophenyl)piperidine-1-carbonyl]-2-methyl-phenyl]-6-oxo-1H-pyridine-3-sulfonamide
N-[5-[4-(4-cyanophenyl)piperidine-1-carbonyl]-2-methyl-phenyl]-6-(2-methoxyethylamino)pyridine-3-sulfonamide
4-[1-[3-(ethenylsulfonylamino)-4-methyl-benzoyl]-4-piperidyl]benzamide
N-[5-[4-(4-cyanophenyl)piperidine-1-carbonyl]-2-methyl-phenyl]-2-methoxy-ethanesulfonamide
N-[5-[4-(4-cyanophenyl)piperidine-1-carbonyl]-2-methyl-phenyl]-2,4-dimethyl-1,3-thiazole-5-sulfonamide
N-[5-[4-(4-cyanophenyl)piperidine-1-carbonyl]-2-methyl-phenyl]pyridine-3-sulfonamide
methyl 2-[[5-[4-(4-cyanophenyl)piperidine-1-carbonyl]-2-methyl-phenyl]sulfamoyl]benzoate
N-[5-[4-(4-cyanophenyl)piperidine-1-carbonyl]-2-methyl-phenyl]-2-hydroxy-ethanesulfonamide
N-[5-[4-(4-cyanophenyl)piperidine-1-carbonyl]-2-methyl-phenyl]-3-methoxy-propane-1-sulfonamide
N-[5-[4-(4-cyanophenyl)piperidine-1-carbonyl]-2-methyl-phenyl]-2-(morpholine-4-carbonyl)benzenesulfonamide
4-[1-(3-methanesulfonamido-4-methyl-benzoyl)-4-piperidyl]benzamide
2-[[5-[4-(4-cyanophenyl)piperidine-1-carbonyl]-2-methyl-phenyl]sulfamoyl]benzamide
N-[5-[4-(4-cyanophenyl)piperidine-1-carbonyl]-2-methyl-phenyl]-2,3-dimethyl-imidazole-4-sulfonamide
N-[5-[4-(4-cyanophenyl)piperidine-1-carbonyl]-2-methyl-phenyl]piperidine-4-sulfonamide
N-[5-[4-(4-cyanophenyl)piperidine-1-carbonyl]-2,4-dimethyl-phenyl]cyclopropanesulfonamide
N-[5-[4-(4-cyanophenyl)piperidine-1-carbonyl]-2,4-dimethyl-phenyl]propane-1-sulfonamide
1-acetyl-N-[5-[4-(4-cyanophenyl)piperidine-1-carbonyl]-2-methyl-phenyl]piperidine-4-sulfonamide
N-[5-[4-(4-cyanophenyl)piperidine-1-carbonyl]-2-methyl-phenyl]-1-propan-2-ylsulfonyl-piperidine-4-sulfonamide
N-[5-[4-(4-cyanophenyl)piperidine-1-carbonyl]-2-methyl-phenyl]-1-propan-2-yl-piperidine-4-sulfonamide
N-[5-[4-(4-cyanophenyl)piperidine-1-carbonyl]-2-methyl-phenyl]-1-methylsulfonyl-piperidine-4-sulfonamide
N-[5-[4-(4-cyanophenyl)piperidine-1-carbonyl]-2-methyl-phenyl]-1-ethylsulfonyl-piperidine-4-sulfonamide
N-[5-[4-(4-cyanophenyl)piperidine-1-carbonyl]-2-ethyl-phenyl]methanesulfonamide
N-[5-[4-(4-cyanophenyl)piperidine-1-carbonyl]-2-ethyl-phenyl]-1,1-dioxo-thiolane-3-sulfonamide
N-[5-[4-(4-cyanophenyl)piperidine-1-carbonyl]-2-methyl-phenyl]-1-methyl-piperidine-4-sulfonamide
3-amino-N-[5-[4-(4-cyanophenyl)piperidine-1-carbonyl]-2-methyl-phenyl]propane-1-sulfonamide
N-[5-[4-(4-cyanophenyl)piperidine-1-carbonyl]-2-methyl-phenyl]-1,1-dioxo-thiolane-3-sulfonamide
N-[5-[4-(4-cyanophenyl)piperidine-1-carbonyl]-2-methyl-phenyl]propane-2-sulfonamide
N-[5-[4-(4-cyanophenyl)piperidine-1-carbonyl]-2-methyl-phenyl]-2-oxo-1,3-dihydroindole-5-sulfonamide
2-[[5-[4-(4-cyanophenyl)piperidine-1-carbonyl]-2-methyl-phenyl]sulfamoyl]acetic acid
N-[5-[4-(4-cyanophenyl)piperidine-1-carbonyl]-2-methyl-phenyl]-3-(propan-2-ylsulfonylamino)propane-1-sulfonamide
N-[5-[4-(4-cyanophenyl)piperidine-1-carbonyl]-2-methyl-phenyl]-3-methanesulfonamido-propane-1-sulfonamide and
N-[3-[[5-[4-(4-cyanophenyl)piperidine-1-carbonyl]-2-methyl-phenyl]sulfamoyl]propyl]benzamide
N-[5-[4-(4-cyanophenyl)piperidine-1-carbonyl]-2-methyl-phenyl]-3-(1,3-dioxoisoindol-2-yl)propane-1-sulfonamide
N-[5-[4-(4-cyanophenyl)piperidine-1-carbonyl]-2-methylsulfonyl-phenyl]-1-phenyl-methanesulfonamide
N-[3-[[5-[4-(4-cyanophenyl)piperidine-1-carbonyl]-2-methyl-phenyl]sulfamoyl]propyl]acetamide
methyl 2-[[5-[4-(4-cyanophenyl)piperidine-1-carbonyl]-2-methyl-phenyl]sulfamoyl]propanoate
N-[5-[4-(4-cyanophenyl)piperidine-1-carbonyl]-2-methyl-phenyl]-1-hydroxy-propane-2-sulfonamide
N-[5-[4-(4-cyanophenyl)piperidine-1-carbonyl]-2-methyl-phenyl]-1,3-dimethyl-pyrazole-4-sulfonamide
N-[5-[4-(4-cyanophenyl)piperidine-1-carbonyl]-2-methyl-phenyl]-1-methyl-pyrazole-3-sulfonamide
N-[5-[4-(4-cyanophenyl)piperidine-1-carbonyl]-2-methyl-phenyl]-3-(pyridin-3-ylmethylamino)propane-1-sulfonamide
N-[5-[4-(4-cyanophenyl)piperidine-1-carbonyl]-2-methyl-phenyl]-1-ethyl-5-methyl-pyrazole-4-sulfonamide
N-[5-[4-(4-cyanophenyl)piperidine-1-carbonyl]-2-methyl-phenyl]-1-ethyl-3-methyl-pyrazole-4-sulfonamide
methyl 3-[[5-[4-(4-cyanophenyl)piperidine-1-carbonyl]-2-methyl-phenyl]sulfamoyl]propanoate
N-[5-[4-(4-cyanophenyl)piperidine-1-carbonyl]-2-methyl-phenyl]-1-methyl-pyrazole-4-sulfonamide
N-[5-[4-(4-cyanophenyl)piperidine-1-carbonyl]-2-methyl-phenyl]prop-2-ene-1-sulfonamide
N-[5-[4-(4-cyanophenyl)piperidine-1-carbonyl]-2-methyl-phenyl]-1-[(4R)-4-methyl-2,5-dioxo-imidazolidin-4-yl]methanesulfonamide
N-[5-[4-(4-cyanophenyl)piperidine-1-carbonyl]-2-methyl-phenyl]-1-(4-ethyl-2,5-dioxo-imidazolidin-4-yl)methanesulfonamide
N-[5-[4-(4-cyanophenyl)piperidine-1-carbonyl]-2-methyl-phenyl]-3,5-dimethyl-1,2-oxazole-4-sulfonamide
N-[5-[4-(4-cyanophenyl)piperidine-1-carbonyl]-2-(methoxymethyl)phenyl]-methanesulfonamide
N-[5-[4-(4-cyanophenyl)piperidine-1-carbonyl]-2-methyl-phenyl]-3-dimethylamino-2-hydroxy-propane-1-sulfonamide
3-[[5-[4-(4-cyanophenyl)piperidine-1-carbonyl]-2-methyl-phenyl]sulfamoyl]propanoic acid
N-[5-[4-(4-cyanophenyl)piperidine-1-carbonyl]-2-methyl-phenyl]-1,3,5-trimethyl-pyrazole-4-sulfonamide
1-acetyl-N-[5-[4-(4-cyanophenyl)piperidine-1-carbonyl]-2-methyl-phenyl]pyrrolidine-3-sulfonamide
N-[5-[4-(4-cyanophenyl)piperidine-1-carbonyl]-2-methyl-phenyl]-1-methylsulfonyl-pyrrolidine-3-sulfonamide
N-[5-[4-(4-chlorophenyl)piperidine-1-carbonyl]-2-methyl-phenyl]methanesulfonamide
4-[1-(3-methanesulfonamido-4-methyl-benzoyl)-4-piperidyl]-N-methyl-benzamide
N-[2-methyl-5-[4-[4-(trifluoromethyl)phenyl]piperidine-1-carbonyl]phenyl]methanesulfonamide
N-[2-methyl-5-[4-(4-methylphenyl)piperidine-1-carbonyl]phenyl]methanesulfonamide
N-[5-[4-(4-cyanophenyl)piperidine-1-carbonyl]-2-methylsulfanylphenyl]methanesulfonamide
N-[5-[4-(4-cyanophenyl)piperidine-1-carbonyl]-2-methylsulfanylphenyl]-1-phenylmethanesulfonamide
N-[5-[4-(4-cyanophenyl)piperidine-1-carbonyl]-2-(methoxymethyl)phenyl]methanesulfonamide
N-[5-[4-(4-methoxyphenyl)piperidine-1-carbonyl]-2-methylphenyl]methanesulfonamide
N-[[4-[1-(3-methanesulfonamido-4-methylbenzoyl)piperidin-4-yl]phenyl]methyl]methanesulfonamide
N-[[4-[1-(3-methanesulfonamido-4-methylbenzoyl)piperidin-4-yl]phenyl]methyl]acetamide
N-[5-[4-[4-(hydroxymethyl)phenyl]piperidine-1-carbonyl]-2-methylphenyl]methanesulfonamide
N-[5-[4-(4-cyanophenyl)piperidine-1-carbonyl]-2,4-dimethylphenyl]ethanesulfonamide
N-[2-methyl-5-[4-[4-(5-methyl-1,2,4-oxadiazol-3-yl)phenyl]piperidine-1-carbonyl]phenyl]methanesulfonamide
N-[2-methyl-5-[4-[4-(1,3,4-oxadiazol-2-yl)phenyl]piperidine-1-carbonyl]phenyl]methanesulfonamide
N-[5-[4-(4-bromophenyl)piperidine-1-carbonyl]-2-methylphenyl]methanesulfonamide
N-[2-methyl-5-[4-[4-(1,3-thiazol-2-yl)phenyl]piperidine-1-carbonyl]phenyl]methanesulfonamide
N-[2-methyl-5-[4-[4-(1,2,4-oxadiazol-3-yl)phenyl]piperidine-1-carbonyl]phenyl]methanesulfonamide
N-[5-[4-(4-ethynylphenyl)piperidine-1-carbonyl]-2-methylphenyl]methanesulfonamide
N-[2-methyl-5-[4-(4-pyridin-2-ylphenyl)piperidine-1-carbonyl]phenyl]methane-sulfonamide
N-[2-methyl-5-[4-(4-pyrazin-2-ylphenyl)piperidine-1-carbonyl]phenyl]methanesulfonamide
N-[2-methyl-5-[4-(4-phenylphenyl)piperidine-1-carbonyl]phenyl]methanesulfonamide
N-[5-[4-(4-cyanophenyl)piperidine-1-carbonyl]-2-(methylsulfanylmethyl)phenyl]methanesulfonamide
N-[2-methyl-5-[4-[4-(trifluoromethyl)phenyl]piperidine-1-carbonyl]phenyl]-1,1-dioxothiolane-3-sulfonamide
N-[5-[4-(4-cyano-3-methoxyphenyl)piperidine-1-carbonyl]-2-methylphenyl]methanesulfonamide
N-[5-[4-(4-cyano-3-fluorophenyl)piperidine-1-carbonyl]-2-methylphenyl]methanesulfonamide
N-[2-methyl-5-[4-[4-(trifluoromethoxy)phenyl]piperidine-1-carbonyl]phenyl]methanesulfonamide
N-[5-[4-[4-(cyanomethoxy)phenyl]piperidine-1-carbonyl]-2-methylphenyl]methanesulfonamide
N-[2-methyl-5-[4-(4-methylsulfinylphenyl)piperidine-1-carbonyl]phenyl]methanesulfonamide
4-[1-(3-methanesulfonamido-4-methylbenzoyl)piperidin-4-yl]-N-methylbenzenesulfonamide
N-cyclopropyl-4-[1-(3-methanesulfonamido-4-methylbenzoyl)piperidin-4-yl]benzamide
[4-[1-(3-methanesulfonamido-4-methylbenzoyl)piperidin-4-yl]phenyl]methanesulfonate
N-[2-chloro-5-[4-(4-cyanophenyl)piperidine-1-carbonyl]phenyl]-2-fluorobenzenesulfonamide
4-chloro-N-[2-chloro-5-[4-(4-cyanophenyl)piperidine-1-carbonyl]phenyl]benzenesulfonamide
N-[2-methyl-5-[4-(4-methylsulfonylphenyl)piperidine-1-carbonyl]phenyl]-1-phenylmethanesulfonamide
N-[5-[4-(4-cyanophenyl)piperidine-1-carbonyl]-2-fluorophenyl]-1-phenylmethanesulfonamide
N-[5-[4-(4-cyanophenyl)piperidine-1-carbonyl]-2-methylphenyl]-1-methylsulfonylmethanesulfonamide
N-[2-cyano-5-[4-(4-cyanophenyl)piperidine-1-carbonyl]phenyl]-1-phenylmethanesulfonamide
N-[3-[4-(4-cyanophenyl)piperidine-1-carbonyl]-4-methoxyphenyl]methanesulfonamide
4-butoxy-N-[3-[4-(4-cyanophenyl)piperidine-1-carbonyl]-4-methylphenyl]benzenesulfonamide
N-[3-[4-(4-cyanophenyl)piperidine-1-carbonyl]-4-fluorophenyl]-1-phenylmethanesulfonamide
N-[5-[4-(4-bromophenyl)-4-hydroxypiperidine-1-carbonyl]-2-methylphenyl]methanesulfonamide
N-[5-[4-(4-bromophenyl)-4-hydroxypiperidine-1-carbonyl]-2-methylphenyl]-1-phenylmethanesulfonamide
N-[5-[4-(4-cyanophenyl)piperidine-1-carbonyl]-2-methylphenyl]-2-phenylethanesulfonamide
N-[3-[4-(4-cyanophenyl)piperidine-1-carbonyl]phenyl]methanesulfonamide
4-[1-[3-(benzylsulfonylamino)-4-methylbenzoyl]piperidin-4-yl]-N,N-dimethylbenzamide
N-[5-[4-(4-cyanophenyl)piperidine-1-carbonyl]-2-methylphenyl]-3-hydroxypropane-1-sulfonamide
2-[[5-[4-(4-cyanophenyl)piperidine-1-carbonyl]-2-methylphenyl]sulfamoyl]-N,N-dimethylbenzamide
2-[[5-[4-(4-cyanophenyl)piperidine-1-carbonyl]-2-methylphenyl]sulfamoyl]-N-(2-hydroxyethyl)benzamide
2-[[5-[4-(4-cyanophenyl)piperidine-1-carbonyl]-2-methylphenyl]sulfamoyl]-N-propan-2-ylbenzamide
N-[5-[4-(4-cyanophenyl)piperidine-1-carbonyl]-2-methylphenyl]-1,1,1-trifluoromethanesulfonamide
N-[5-[4-(4-cyanophenyl)piperidine-1-carbonyl]-2-methylsulfonylphenyl]methanesulfonamide
N-[5-[4-(4-cyanophenyl)piperidine-1-carbonyl]-2-methylsulfinylphenyl]-1-phenylmethanesulfonamide
1-cyano-N-[5-[4-(4-cyanophenyl)piperidine-1-carbonyl]-2-methylphenyl]methanesulfonamide
N-[5-[4-(4-fluorophenyl)piperidine-1-carbonyl]-2-methylphenyl]methanesulfonamide
N′-hydroxy-4-[1-(3-methanesulfonamido-4-methylbenzoyl)piperidin-4-yl]benzenecarboximidamide
N-[5-[4-hydroxy-4-[4-(trifluoromethyl)phenyl]piperidine-1-carbonyl]-2-methylphenyl]methanesulfonamide
N-[5-[4-(4-chlorophenyl)piperidine-1-carbonyl]-2-methylphenyl]-1,1-dioxothiolane-3-sulfonamide
N-[5-[4-(4-cyano-3-methylphenyl)piperidine-1-carbonyl]-2-methylphenyl]methanesulfonamide
N-[5-[4-(3-chloro-4-cyanophenyl)piperidine-1-carbonyl]-2-methylphenyl]methanesulfonamide
N-[5-[4-[4-(2-methoxyethoxy)phenyl]piperidine-1-carbonyl]-2-methylphenyl]methanesulfonamide
N-(2-hydroxyethyl)-4-[1-(3-methanesulfonamido-4-methylbenzoyl)piperidin-4-yl]benzenesulfonamide
N-[4-[1-(3-methanesulfonamido-4-methylbenzoyl)piperidin-4-yl]phenyl]methanesulfonamide
N-(2-dimethylaminoethyl)-4-[1-(3-methanesulfonamido-4-methylbenzoyl)piperidin-4-yl]benzamide
N-[2-methyl-5-[4-[4-(methylsulfonylmethyl)phenyl]piperidine-1-carbonyl]phenyl]methanesulfonamide
N-[5-[4-[4-[3-(2-methoxyethoxy)prop-1-ynyl]phenyl]piperidine-1-carbonyl]-2-methylphenyl]methanesulfonamide
3-[4-[1-(3-methanesulfonamido-4-methylbenzoyl)piperidin-4-yl]phenyl]-N-methylpropanamide
3-[4-[1-(3-methanesulfonamido-4-methylbenzoyl)piperidin-4-yl]phenyl]-N,N-dimethylpropanamide
N-[5-[4-[4-(5-hydroxypent-1-ynyl)phenyl]piperidine-1-carbonyl]-2-methylphenyl]methanesulfonamide
N-[5-[4-[4-(4-hydroxybut-1-ynyl)phenyl]piperidine-1-carbonyl]-2-methylphenyl]methanesulfonamide
N-[5-[4-[4-(3-hydroxy-3-methylbut-1-ynyl)phenyl]piperidine-1-carbonyl]-2-methylphenyl]methanesulfonamide or
2-[[4-[1-(3-methanesulfonamido-4-methylbenzoyl)piperidin-4-yl]phenyl]methylsulfamoyl]acetic acid
   or a pharmaceutically-acceptable salt thereof.

In another embodiment there is provided a compound selected from one or more of the following List 2:
N-[5-[4-(4-cyanophenyl)piperidine-1-carbonyl]-2-methylphenyl]methanesulfonamide;
N-[5-[4-(4-cyanophenyl)piperidine-1-carbonyl]-2-methylphenyl]propane-1-sulfonamide;
N-[5-[4-(4-cyanophenyl)piperidine-1-carbonyl]-2-methylphenyl]ethanesulfonamide;
N-[5-[4-(4-cyanophenyl)piperidine-1-carbonyl]-2-methylphenyl]-1-methylsulfonyl-piperidine-4-sulfonamide;
N-[5-[4-(4-cyanophenyl)piperidine-1-carbonyl]-2-methylphenyl]-1-ethylsulfonyl-piperidine-4-sulfonamide;
N-[5-[4-(4-cyanophenyl)piperidine-1-carbonyl]-2-methylphenyl]propane-2-sulfonamide;
N-[3-[[5-[4-(4-cyanophenyl)piperidine-1-carbonyl]-2-methylphenyl]sulfamoyl]propyl]-acetamide;
N-[5-[4-(4-cyanophenyl)piperidine-1-carbonyl]-2-methylphenyl]-1-methyl-pyrazole-3-sulfonamide; or
N-[5-[4-(4-cyanophenyl)piperidine-1-carbonyl]-2,4-dimethylphenyl]ethanesulfonamide or a pharmaceutically-acceptable salt thereof

A compound of the Formula I, or a pharmaceutically-acceptable salt thereof, may be prepared by any process known to be applicable to the preparation of chemically-related compounds. Such processes, when used to prepare a compound of the formula I are provided as a further feature of the invention and are illustrated by the following representative process variants. Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated that are within the ordinary skill of an organic chemist.

According to a further aspect, the present invention provides a process for preparing a compound of formula I or a pharmaceutically acceptable salt thereof (wherein R¹, R², R³, R⁴, R⁵, R^{5′}, R⁶, R^{6′} and R⁷ are, unless otherwise specified, as defined in formula

I) which process comprises:
(a) reacting a compound of formula VI

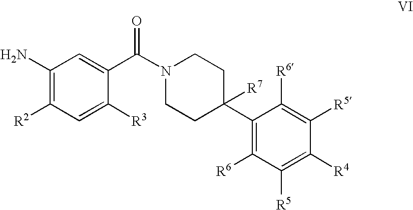

with a compound of formula VII

R₁SO₂X VII

in which X represents a leaving group for example halo, e.g. chloro, in the presence of a diluent for example a solvent e.g. dichloromethane and optionally in the presence of a base, for example an organic amine e.g DIPEA, at a temperature in the range of 0-150° C.; or
b) reacting a compound of formula IX

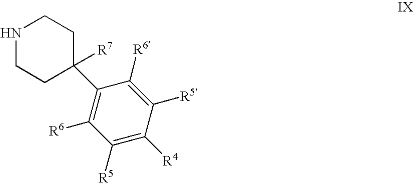

with a compound of formula X

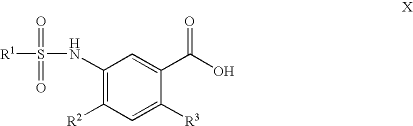

optionally in the presence of a coupling agent and optionally in the presence of a diluent for example a solvent at a temperature in the range of 0-150° C.; or
c) reacting a compound of formula IX

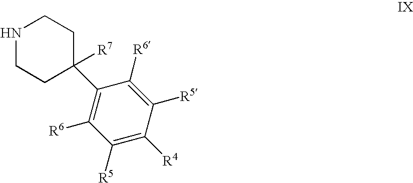

with a compound of formula XI

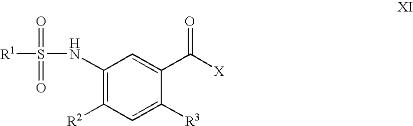

in which X represents a leaving group for example halo, e.g. chloro, in the presence of a diluent for example a solvent e.g. dichloromethane and optionally in the presence of a base, for example an organic amine e.g DIPEA, at a temperature in the range of 0-150° C.; or
d) reacting a compound of formula XII

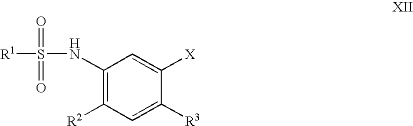

in which X represents a replaceable group, eg. Cl, Br, I, OMesyl, or OTriflyl with a compound of formula X in the presence of carbon monoxide and in the presence of a metal catalyst, eg. Pd or derivatives thereof, and in a solvent such as an alcohol, THF, toluene, or DMF, and in the temperature range 0-150° C. The carbon monoxide may be gaseous or in the form of a metal carbonyl, eg. Molybdenum hexacarbonyl.

It will be appreciated that the transformation in steps b and c above may be carried out with the use of different coupling agents, with or without additives, in various suitable diluents or solvents, and over a range of temperatures.

Examples of coupling agents are Dichlorotriphenyl phosphorane (DCTPP), 1-ethyl-3-(3-dimethylaminopropyl) carbodiimide hydrochloride (EDAC), O-benzotriazol-1-yl-N,N,N′,N′-tetramethyluronium hexafluorophosphate (HTBU), O-(7-azabenzotriazol-1-yl)-N,N,N′,N′-tetramethyluronium hexafluorophosphate (HATU) and 4-(4,6-dimethoxy-1,3,5-triazin-2-yl)-4-methylmorpholinium chloride (DMTMM).

Examples of optional additives are: 1-hydroxy benzotriazole (HOBt), 4-dimethylamino pyridine (DMAP), di-iso-propylethylamine (DIPEA), and triethylamine (TEA).

Examples of suitable solvents are: dimethyl formamide (DMF), chloroform, dichloromethane (DCM), and tetrahydrofuran (THF).

Certain compounds of formula I may be converted into other compounds of formula I by methods known to those skilled in the art. Compounds of formula I in which R¹ represents an optionally substituted pyridyl-N-oxide may be prepared by reacting a compound of formula I in which R¹ represents an optionally substituted pyridyl with an oxidising agent for example urea hydrogen peroxide or 3-chloroperbenzoic acid, in the presence of a diluent for example dichloromethane or acetonitrile at a temperature in the range of 0-150° C.

In other processes compounds of formula I containing a sulphide group may be oxidised to SO or SO₂ for example by use of potassium peroxymonosulfate, nitriles may be reduce to aminomethyl compounds, amines may be acylated or sulfonylated to give amides or sulfonamides, respectively, activated heteroaryl halides may be hydrolysed to hydroxy groups, and esters may be hydrolysed to acids.

It will be appreciated by those skilled in the art that certain functional groups may require protection before certain transformations are attempted followed by deprotection after the particular transformation. Such methods are well known to those skilled in the art and are described in “Protective Groups in Organic Synthesis”, 2^{nd} Edition (1991) by Greene and Wuts.

Certain intermediates of formula VI are believed to be novel and are herein claimed as another aspect of the present invention.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation comprising a compound of formula I, or pharmaceutically acceptable salt thereof, in admixture with pharmaceutically acceptable adjuvants, diluents and/or carriers.

### Pharmacological Properties

The compounds of formula (I) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, bulimia and compulsive eating), dyslipidaemia and the treatment of type 2 diabetes mellitus.

The present compounds of formula (I) are useful for the prophylaxis and/or treatment of clinical conditions associated with inherent or induced reduced sensitivity to insulin (insulin resistance) and associated metabolic disorders (also known as the metabolic syndrome). These clinical conditions will include, but will not be limited to, general obesity, abdominal obesity, arterial hypertension, hyperinsulinaemia, hyperglycaemia, type 2 diabetes and the dyslipidaemia characteristically appearing with insulin resistance. This dyslipidaemia, also known as the atherogenic lipoprotein profile, is characterised by moderately elevated non-esterified fatty acids, elevated very low density lipoprotein (VLDL) triglyceride rich particles, high Apo B levels, low high density lipoprotein (HDL) levels associated with low apoAI particle levels and high Apo B levels in the presence of small, dense, low density lipoproteins (LDL) particles, phenotype B.

The compounds of the present invention are expected to be useful in treating patients with combined or mixed hyperlipidemias or various degrees of hypertriglyceridemias and postprandial dyslipidemia with or without other manifestations of the metabolic syndrome.

Treatment with the present compounds is expected to lower the cardiovascular morbidity and mortality associated with atherosclerosis due to their antidyslipidaemic as well as antiinflammatory properties. The cardiovascular disease conditions include macro-angiopathies of various internal organs causing myocardial infarction, congestive heart failure, cerebrovascular disease and peripheral arterial insufficiency of the lower extremities. Because of their insulin sensitizing effect the compounds of formula I are also expected to prevent or delay the development of type 2 diabetes from the metabolic syndrome and diabetes of pregnancy. Therefore the development of long-term complications associated with chronic hyperglycaemia in diabetes mellitus, such as the micro-angiopathies causing renal disease, retinal damage and peripheral vascular disease of the lower limbs, is expected to be delayed. Furthermore the compounds may be useful in treatment of various conditions outside the cardiovascular system whether or not associated with insulin resistance, like polycystic ovarian syndrome, obesity, cancer and states of inflammatory disease including neurodegenerative disorders such as mild cognitive impairment, Alzheimer's disease, Parkinson's disease and multiple sclerosis.

The compounds of formula I may also be useful in the treatment of metabolic syndrome and Prader-Willi syndrome.

In another aspect the present invention provides a compound of formula I as previously defined for use as a medicament.

In a further aspect the present invention provides the use of a compound of formula I in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, bulimia and compulsive eating) and for the treatment or prophylaxis of dyslipidaemia and for the treatment or prophylaxis of type 2 diabetes mellitus.

In a still further aspect the present invention provides a method of treating obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, bulimia and compulsive eating) dyslipidaemia and type 2 diabetes mellitus comprising administering a pharmacologically effective amount of a compound of formula I to a patient in need thereof.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound for example sibutramine, phentermine, orlistat, bupropion, ephedrine, thyroxine;
an aldose reductase inhibitor;
a glycogen phosphorylase inhibitor;
a glycogen synthase kinase inhibitors;
a glucokinase activator;
a haemostasis modulator;
an antithrombotic;
an activator of fibrinolysis;
an antiplatelet agent;
a thrombin antagonist;
a factor Xa inhibitor;
a factor VIIa inhibitor;
an antiplatelet agents;
a 5 HT transporter inhibitor;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator; for example an NPY agonist or an NPY2 agonist or an NPY5 antagonist;
an Mc4r modulator for example an Mc4r agonist;
an Mc3r modulator for example an Mc3r agonist;
an orexin receptor modulator for example an antagonist;
a phosphoinositide-dependent protein kinase (PDK) modulator; or
modulators of nuclear receptors for example LXR, FXR, RXR, GR, ERRα, β, PPARα, β, γ, δ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a CB1 receptor modulator for example an inverse agonist or an antagonist;
a GLK receptor modulator;
a DPP-IV inhibitor;
a cholesterol absorption inhibitor;
a GLP-1 agonist;
an SGLT-2 inhibitor;
a DGAT1 inhibitor;
a DGAT2 inhibitor;
a DGAT2 anti-sense oligonucleotide;
a ghrelin antibody;
a ghrelin antagonist;
an 11β HSD-1 inhibitor;
an UCP-1, 2 or 3 activator;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

The compounds of the invention may also be useful as anti-cell-proliferation (such as anti-cancer) agents and are therefore useful in methods of treatment of the human or animal body.

Such properties are expected to be of value in the treatment of disease states associated with cell cycle and cell proliferation such as cancers (solid tumors and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine, N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

The compounds of the present invention may also be useful as anti-infective agents or as anti-bacterial agents. The compounds of the present invention may also be useful as in decreasing sebum production following topical application.

### Pharmacological Activity

The compounds of the present invention are Fatty Acid Synthase inhibitors. The activity of the compounds of the invention was demonstrated using the following assay. Human and Rat FAS Enzyme Assay.

Fatty acid synthase is an enzyme complex that harbours seven enzymatic activities catalysing the reductive synthesis of long chain fatty acids from acetyl CoA and malonyl CoA to palmitate. When acetyl CoA and malonyl CoA are forming palmitate NADPH is consumed forming NADP. Since NADPH is fluorescent but not NADP the reaction can be measured by analysing the decrease in fluorescence.

Compounds were added to a black 384 well plate (Matrix) in a volume of 5 μl consisting of 20% DMSO and 80% Tris buffer pH 7.5, at a top concentration of 1 mM. NADPH, 30 μl of 166.6 μM, formulated in assay buffer (0.1M Tris ph7.5, 0.1 mM EDTA, 1 mM glutathione, 0.05% BSA), was then added to all of the wells of the plate. Fatty acid synthase Human or rat enzyme (0.4 μg, produced in house), dissolved in 20 mM Tris/HCl pH 7.5, 5 mM BOG, 1 mM TCEP, 10% glycerol, 1 mM EDTA, 150 mM NaCl, was then added to the plate in a volume of 101. Enzyme was added to all but the last two columns of the plate, to which, 10 μl of assay buffer was added (0.1M Tris ph7.5, 0.1 mM EDTA, 1 mM glutathione, 0.05% BSA) to provide a no enzyme assay control. Following a 15-minute incubation period, at room temperature, the plates were read on an Envision plate reader using 340 nm excitation and 460 nm emission filters. This served as a time zero background read. Substrates (an equal mix of both malonyl and acetyl CoA) were then added to the plates in a total volume of 5 μl. The concentrations of malonyl and acetyl CoA in the mixture were 500 μM and 150 μM respectively. Both were prepared as 10 mM stock solutions in distilled water and were subsequently diluted to working concentrations in assay buffer. Plates were then incubated for a further 60 minutes, at room temperature, before being read again on the Envision reader using the same parameters as previously used. The data was analysed by subtracting the background time zero data from that generated following the final 60 minute incubate and the percent inhibition compared to the maximum and minimum assay controls was determined. Sigmoid curves were fitted using Origin 7.5 Client software and IC50 values were determined.

The compounds of the present invention were found to inhibit the activation of human (h) Fatty Acid Synthase with IC₅₀s in a range of about 0.0001 μM to about 30 μM in the above assay. The examples of the present invention inhibited the activation of human Fatty Acid Synthase with IC₅₀s in a range of about 0.001 μM to about 30.0 μM. In another embodiment the compounds inhibit the activation of human Fatty Acid Synthase with IC₅₀s in a range of about 0.001 μM to about 0.1 μM. The results are given in Table 1.

In Table 1 Ex stands for Example Number, Inhib (%) stands for the % inhibition at a concentration of 100 micromolar in the next column.

TABLE 1

| Ex | Inhib % |
|---|---|
| 1 | 86 |
| 2 | 82 |
| 3 | 96 |
| 4 | 81 |
| 5 | 92 |
| 6 | 75 |
| 7 | 78 |
| 8 | 82 |
| 9 | 95 |
| 10 | 78 |
| 11 | 88 |
| 12 | 90 |
| 13 | 76 |
| 14 | 89 |
| 15 | 93 |
| 16 | 95 |
| 17 | 94 |
| 18 | 92 |
| 19 | 96 |
| 20 | 85 |
| 21 | 90 |
| 22 | 95 |
| 23 | 89 |
| 24 | 83 |
| 25 | 87 |
| 26 | 94 |
| 27 | 96 |
| 28 | 93 |
| 29 | 93 |
| 30 | 96 |
| 31 | 83 |
| 32 | 85 |
| 33 | 91 |
| 34 | 89 |
| 35 | 89 |
| 36 | 88 |
| 37 | 91 |
| 38 | 83 |
| 39 | 80 |
| 40 | 89 |
| 41 | 96 |
| 42 | 90 |
| 43 | 93 |
| 44 | 89 |
| 45 | 83 |
| 46 | 79 |
| 47 | 85 |
| 48 | 84 |
| 49 | 83 |
| 50 | 87 |
| 51 | 94 |
| 52 | 91 |
| 53 | 84 |
| 54 | 84 |
| 55 | 79 |
| 56 | 89 |
| 57 | 94 |
| 58 | 92 |
| 59 | 89 |
| 60 | 83 |
| 61 | 93 |
| 62 | 82 |
| 63 | 85 |
| 64 | 83 |
| 65 | 90 |
| 66 | 80 |
| 67 | 92 |
| 68 | 74 |
| 69 | 82 |
| 70 | 87 |
| 71 | 100 |
| 72 | 93 |
| 73 | 94 |
| 74 | 95 |
| 75 | 93 |
| 76 | 97 |
| 77 | 98 |
| 78 | 95 |
| 79 | 95 |
| 80 | 90 |
| 81 | 90 |
| 82 | 93 |
| 83 | 98 |
| 84 | 92 |
| 85 | 95 |
| 86 | 100 |
| 87 | 82 |
| 88 | 89 |
| 89 | 78 |
| 90 | 79 |
| 91 | 80 |
| 92 | 80 |
| 93 | 79 |
| 94 | 86 |
| 95 | 73 |
| 96 | 98 |
| 97 | 88 |
| 98 | 110 |
| 99 | 92 |
| 100 | 90 |
| 101 | 98 |
| 102 | 81 |
| 103 | 98 |
| 104 | 76 |
| 105 | 89 |
| 106 | 95 |
| 107 | 92 |
| 108 | 78 |
| 109 | 94 |
| 110 | 86 |
| 111 | 80 |
| 112 | 79 |
| 113 | 77 |
| 114 | 85 |
| 115 | 97 |
| 116 | 92 |
| 117 | 98 |
| 118 | 85 |
| 119 | 94 |
| 120 | 94 |
| 121 | 89 |
| 122 | 98 |
| 123 | 85 |
| 124 | 82 |
| 125 | 98 |
| 126 | 87 |
| 127 | 92 |
| 128 | 96 |
| 129 | 95 |
| 130 | 77 |
| 131 | 91 |
| 132 | 88 |
| 133 | 91 |
| 134 | 97 |
| 135 | 100 |
| 136 | 94 |
| 137 | 97 |
| 138 | 100 |
| 139 | 83 |
| 140 | 100 |
| 141 | 93 |
| 142 | 98 |
| 143 | 86 |
| 144 | 88 |
| 145 | 97 |
| 146 | 90 |
| 147 | 98 |
| 148 | 100 |
| 149 | 85 |
| 150 | 88 |
| 151 | 81 |
| 152 | 87 |
| 153 | 97 |
| 154 | 99 |
| 155 | 84 |
| 156 | 74 |
| 157 | 78 |
| 158 | 81 |
| 159 | 80 |
| 160 | 89 |
| 161 | 80 |
| 162 | 86 |
| 163 | 85 |
| 164 | 83 |
| 165 | 76 |
| 166 | 96 |
| 167 | 87 |
| 168 | 75 |
| 169 | 90 |
| 170 | 74 |
| 171 | 85 |
| 172 | 85 |
| 173 | 64 |
| 174 | 92 |
| 175 | 71 |
| 176 | 78 |
| 177 | 91 |
| 178 | 81 |
| 179 | 85 |
| 180 | 96 |
| 181 | 100 |
| 182 | 80 |
| 183 | 90 |
| 184 | 84 |
| 185 | 80 |
| 186 | 99 |
| 187 | 100 |
| 188 | 110 |
| 189 | 88 |
| 190 | 88 |
| 191 | 63 |
| 192 | 67 |
| 193 | 70 |
| 194 | 69 |
| 195 | 72 |
| 196 | 96 |
| 197 | 71 |
| 198 | 71 |
| 199 | 79 |
| 200 | 48 |
| 201 | 68 |
| 202 | 90 |
| 203 | 71 |
| 204 | 78 |
| 205 | 75 |
| 206 | 76 |
| 207 | 75 |
| 208 | 75 |
| 209 | 76 |
| 210 | 91 |
| 211 | 92 |
| 212 | 69 |
| 213 | 71 |
| 214 | 85 |
| 215 | 56 |
| 216 | 89 |
| 217 | 60 |
| 218 | 67 |
| 219 | 87 |
| 220 | 85 |
| 221 | 74 |
| 222 | 64 |
| 223 | 74 |
| 224 | 108.5 |
| 225 | 89.1 |
| 226 | 81.0 |
| 227 | 87.4 |
| 228 | 85.5 |
| 229 | 80.2 |
| 230 | 73.2 |
| 231 | 86.1 |
| 232 | 88.9 |
| 233 | 81.8 |
| 234 | 78.3 |
| 235 | 72.8 |
| 236 | 59.8 |
| 237 | 85.2 |
| 238 | 81.3 |
| 239 | 89.1 |
| 240 | 63.9 |
| 241 | 81.5 |
| 242 | 95.7 |
| 243 | 69.9 |
| 244 | 92.3 |
| 245 | 103.8 |
| 246 | 92.8 |

The following compounds do not have IC₅₀s in the range of about 0.001 μM to about 30 μM in the above assay:
4-chloro-N-[2-methyl-5-(4-phenylpiperidine-1-carbonyl)phenyl]benzenesulfonamide
N-[2-chloro-5-[4-(4-cyanophenyl)piperidine-1-carbonyl]phenyl]methanesulfonamide
N-[5-[4-(4-cyanophenyl)piperidine-1-carbonyl]-2-(trifluoromethoxy)phenyl]methanesulfonamide
N-[5-[4-(4-cyanophenyl)piperidine-1-carbonyl]-2-(trifluoromethoxy)phenyl]-1-phenylmethanesulfonamide
N-[5-[4-(4-cyanophenyl)piperidine-1-carbonyl]-2-fluorophenyl]methanesulfonamide
N-[3-[4-(4-cyanophenyl)piperidine-1-carbonyl]-4-fluorophenyl]methanesulfonamide
4-[1-(3-methanesulfonamido-4-methylbenzoyl)piperidin-4-yl]-N,N-dimethylbenzamide
2-[[5-[4-(4-cyanophenyl)piperidine-1-carbonyl]-2-methylphenyl]sulfamoyl]benzoic acid
2-[[5-[4-(4-cyanophenyl)piperidine-1-carbonyl]-2-methylphenyl]sulfamoyl]-N-methylbenzamide
2-[[5-[4-(4-cyanophenyl)piperidine-1-carbonyl]-2-methylphenyl]sulfamoyl]-N-[(2,4-dimethoxyphenyl)methyl]benzamide
N-[5-[4-(4-cyanophenyl)piperidine-1-carbonyl]-2-methylsulfinylphenyl]methanesulfonamide
Benzyl 3-[[5-[4-(4-cyanophenyl)piperidine-1-carbonyl]-2-methylphenyl]sulfamoyl]pyrrolidine-1-carboxylate
N-[5-(4-hydroxy-4-phenylpiperidine-1-carbonyl)-2-methylphenyl]methanesulfonamide
N-[2-methyl-5-(4-phenylpiperidine-1-carbonyl)phenyl]methanesulfonamide
N-[5-[4-(4-chlorophenyl)-4-hydroxypiperidine-1-carbonyl]-2-methylphenyl]methanesulfonamide
N-[5-[4-(2-methoxyphenyl)piperidine-1-carbonyl]-2-methylphenyl]methanesulfonamide
N-[5-[4-hydroxy-4-[3-(trifluoromethyl)phenyl]piperidine-1-carbonyl]-2-methylphenyl]methanesulfonamide
N-[5-[4-(2-fluorophenyl)-4-hydroxypiperidine-1-carbonyl]-2-methylphenyl]methanesulfonamide
N-[5-[4-(3-cyanophenyl)piperidine-1-carbonyl]-2-methylphenyl]methanesulfonamide
N-[5-[4-(3-fluorophenyl)piperidine-1-carbonyl]-2-methylphenyl]methanesulfonamide
N-[5-[4-(3-chlorophenyl)piperidine-1-carbonyl]-2-methylphenyl]methanesulfonamide
N-[5-[4-(3-methoxyphenyl)piperidine-1-carbonyl]-2-methylphenyl]methanesulfonamide
N-[5-[4-(2-cyanophenyl)piperidine-1-carbonyl]-2-methylphenyl]methanesulfonamide
Methyl 4-[1-(3-methanesulfonamido-4-methylbenzoyl)piperidin-4-yl]benzoate
N-[5-[4-(3-bromophenyl)piperidine-1-carbonyl]-2-methylphenyl]methanesulfonamide
N-[5-[4-[4-(aminomethyl)phenyl]piperidine-1-carbonyl]-2-methylphenyl]methanesulfonamide
N-[2-methyl-5-[4-(3-phenylphenyl)piperidine-1-carbonyl]phenyl]methanesulfonamide
N-[2-methyl-5-[4-[3-(1,3-thiazol-5-yl)phenyl]piperidine-1-carbonyl]phenyl]methanesulfonamide
N-[5-[4-(3-ethynylphenyl)piperidine-1-carbonyl]-2-methylphenyl]methanesulfonamide
N-[5-[4-(4-cyanophenyl)piperidine-1-carbonyl]-2-(methylsulfonylmethyl)phenyl]methanesulfonamide
N-[2-methyl-5-[4-(4-pyrimidin-2-ylphenyl)piperidine-1-carbonyl]phenyl]methanesulfonamide
N-[5-[4-(4-cyano-2-methylphenyl)piperidine-1-carbonyl]-2-methylphenyl]methanesulfonamide
N-[5-[4-(4-cyano-3,5-difluorophenyl)piperidine-1-carbonyl]-2-methylphenyl]methanesulfonamide
N-[5-[4-(3,4-dicyanophenyl)piperidine-1-carbonyl]-2-methylphenyl]methanesulfonamide
N-[5-[4-[4-cyano-3-(trifluoromethyl)phenyl]piperidine-1-carbonyl]-2-methylphenyl]methanesulfonamide
N-[2-methyl-5-[4-[4-(2-oxopyrrolidin-1-yl)phenyl]piperidine-1-carbonyl]phenyl]methanesulfonamide
4-[1-(3-methanesulfonamido-4-methylbenzoyl)piperidin-4-yl]-N,N-dimethylbenzenesulfonamide
N-[5-[4-[4-[(3R)-3-hydroxypyrrolidin-1-yl]sulfonylphenyl]piperidine-1-carbonyl]-2-methylphenyl]methanesulfonamide
N-[2-methyl-5-[4-[4-(4-methylpiperazin-1-yl)sulfonylphenyl]piperidine-1-carbonyl]phenyl]methanesulfonamide and
N-[2-methyl-5-[4-[4-(4-methyl1,4-diazepane-1-carbonyl)phenyl]piperidine-1-carbonyl]phenyl]methanesulfonamide.
2-[4-[1-(3-methanesulfonamido-4-methylbenzoyl)piperidin-4-yl]phenyl]acetic acid
2-[4-[1-(3-methanesulfonamido-4-methylbenzoyl)piperidin-4-yl]phenyl]acetamide
2-[4-[1-(3-methanesulfonamido-4-methylbenzoyl)piperidin-4-yl]phenyl]-N,N-dimethylacetamide
2-hydroxy-N-[[4-[1-(3-methanesulfonamido-4-methylbenzoyl)piperidin-4-yl]phenyl]methyl]-2-methylpropanamide
N-[2-methyl-5-[4-(4-pyrrolidin-1-ylsulfonylphenyl)piperidine-1-carbonyl]phenyl]methanesulfonamide
2-[4-[1-[3-(cyclohexylsulfonylamino)-4-methylbenzoyl]piperidin-4-yl]phenyl]acetic acid and N-[5-[4-[4-(3-amino-3-methylbut-1-ynyl)phenyl]piperidine-1-carbonyl]-2-methylphenyl]methanesulfonamide

These compounds are excluded from the claims of the present application by means of a proviso.

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C., unless otherwise stated;
(ii) organic solutions were dried over anhydrous magnesium sulfate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and/or analytical LC-MS, and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard when the solvent is CDCl₃ (when the solvent is DMSO-d₆, it locks on to the 2.49 DMSO peak), determined at 300 MHz unless otherwise indicated; the following abbreviations have been used: s, singlet; d, doublet; t, triplet; q, quartet; m, multiplet; b, broad;
(viii) chemical symbols have their usual meanings; SI units and symbols are used;
(ix) solvent ratios are given in volume:volume (v/v) terms; and
(x) mass spectra (MS) were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is MH⁺;
[A] When Cl is present in the molecule, the m/z value for the (M+H)⁺ molecular ion is based on the ³⁵Cl isotope. When there are multiple chlorine atoms in the molecule, the m/z is based on the first peak of the isotope pattern.
[B] When Br is present in the molecule, the m/z value for the (M+H)⁺ and/or (M−H)⁻ molecular ions may be based either on the ⁷⁹Br isotope or the ⁸¹Br isotope. As the isotopes are of approximately equal abundance, in many cases both isotopes are seen in the spectrum, but only one is reported.
(xi) unless stated otherwise compounds containing an asymmetrically substituted carbon and/or sulphur atom have not been resolved;
(xii) where a synthesis is described as being analogous to that described in a previous example the amounts used are the millimolar ratio equivalents to those used in the previous example;
(xiii) The following methods were used for liquid chromatography (LC)/mass spectral

(MS) analysis:—

HPLC: Agilent 1100 or Waters Alliance HT (2790 & 2795)

Mass Spectrometer Waters ZQ ESCi

(xiv) the following abbreviations have been used:

### ABBREVIATIONS

### ACN Acetonitrile

### DIPEA Di-iso-propylethylamine

DMA Dimethyl acetamide
DMAP 4-dimethylamino pyridine
DMTMM 4-(4,6-Dimethoxy-1,3,5-Triazin-2-yl)-4-Methylmorpholinium Chloride
DMSO dimethyl sulphoxide (in NMR data the solvent is d₆-deuterioDMSO)
EDAC N-ethyl-N′-(3-dimethylaminopropyl)-carbodiimide hydrochloride
EtOAc Ethyl acetate

### EtOH Ethanol

HATU O-(7-Azabenzotriazol-1-yl)-N,N,N′,N′-Tetramethyluronium Hexafluoro-phosphate

### HOBT 1-Hydroxybenzotriazole

hrs hours
HTBU O-benzotriazol-1-yl-N,N,N′,N′-tetramethyluronium hexafluorophosphate

### MeOH Methanol

mins minutes

### TEA Triethylamine

### TFAA Trifluoroacetic Anhydride

### THF Tetrahydrofuran

### EXAMPLE 1

### N-[5-[4-(4-cyanophenyl)piperidine-1-carbonyl]-2-methyl-phenyl]methanesulfonamide

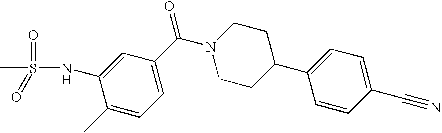

Pyridine (0.15 ml, 1.88 mmol, 3 eq) was added to a stirred suspension of 4-[1-(3-amino-4-methyl-benzoyl)-4-piperidyl]benzonitrile (Intermediate A, 200 mg, 0.63 mmol) and methane sulfonyl chloride (108 mg, 0.94 mmol, 1.5 eq) in DCM (5 mL), and the reaction mixture stirred at ambient temperature for 24 hrs. The reaction mixture was then diluted with more DCM (10 mL) and washed sequentially with dilute aqueous hydrochloric acid (10 mL of 1M), dilute aqueous sodium hydroxide solution (10 mL of 1M), brine (10 mL), dried (MgSO₄), filtered and the solvent removed in vacuo to give a brown oil. This was chromatographed (12 g silica cartridge, eluting with a gradient consisting of 20-70% EtOAc in isohexane) to give the title compound as a colourless solid, 71 mg, ¹H NMR (300.072 MHz, CDCl₃) δ1.66-2.00 (4H, m), 2.34 (3H, s), 2.79-2.93 (2H, m), 3.06 (3H, s), 3.09-3.26 (1H, m), 3.78-4.05 (1H, m), 4.70-5.08 (1H, m), 6.64 (1H, s), 7.20-7.28 (2H, m), 7.33 (2H, d), 7.50 (1H, s), 7.62 (2H, d), m/z 398 (M+H)⁺.

The following compounds were prepared in a manner essentially similar to that described for Example 1, starting from the appropriate intermediate and sulfonyl chloride

### EXAMPLE 2

### N-[5-[4-(4-cyanophenyl)piperidine-1-carbonyl]-2-methyl-phenyl]-2-fluoro-benzenesulfonamide

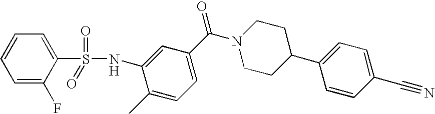

Prepared from Intermediate A

¹H NMR (300.072 MHz, CDCl₃) δ 1.58-1.95 (4H, m), 2.20 (3H, s), 2.77-3.15 (3H, m), 3.63-3.97 (1H, m), 4.60-5.00 (1H, m), 7.04 (1H, s), 7.12-7.22 (4H, m), 7.30-7.36 (3H, m), 7.48-7.58 (1H, m), 7.63 (2H, d), 7.77-7.84 (1H, m), m/z 478 (M+H)⁺.

### EXAMPLE 3

### 4-chloro-N-[5-[4-(4-cyanophenyl)piperidine-1-carbonyl]-2-methyl-phenyl]benzenesulfonamide

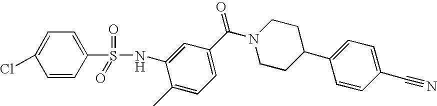

Prepared from Intermediate A

¹H NMR (300.072 MHz, CDCl₃) δ1.50-1.98 (4H, m), 2.04 (3H, s), 2.76-2.94 (2H, m), 2.97-3.15 (1H, m), 3.58-3.95 (1H, m), 4.81 (1H, m), 7.10-7.20 (4H, m), 7.30-7.41 (5H, m), 7.59-7.68 (3H, m), m/z 494 (M+H)⁺ [A].

### EXAMPLE 4

### N-[5-[4-(4-cyanophenyl)piperidine-1-carbonyl]-2-methyl-phenyl]-1-phenyl-methanesulfonamide

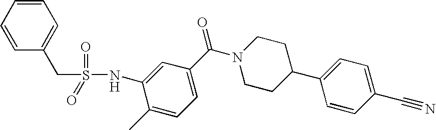

Prepared from Intermediate A

¹H NMR (300.072 MHz, CDCl₃) δ 1.65-1.98 (4H, m), 2.04 (3H, s), 2.79-2.95 (2H, m), 2.97-3.24 (1H, m), 3.78-4.04 (1H, m), 4.40 (2H, s), 4.63-5.00 (1H, m), 6.14 (1H, s), 7.16-7.25 (4H, m), 7.29-7.37 (5H, m), 7.58-7.66 (3H, m), m/z 474 (M+H)⁺.

### EXAMPLE 5

### N-[5-[4-(4-cyanophenyl)piperidine-1-carbonyl]-2-methyl-phenyl]propane-1-sulfonamide

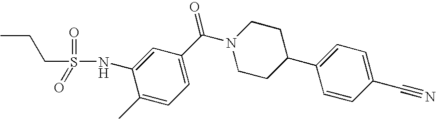

Prepared from Intermediate A

¹H NMR (300.072 MHz, CDCl₃) δ 1.04 (3H, t), 1.67-2.03 (6H, m), 2.33 (3H, s), 2.80-3.14 (5H, m), 3.79-4.08 (1H, m), 4.62-4.98 (1H, m), 6.48 (1H, s), 7.17-7.28 (2H, m), 7.33 (2H, d), 7.52 (1H, s), 7.61 (2H, d), m/z 426 (M+H)⁺.

### EXAMPLE 6

### N-[5-[4-(4-cyanophenyl)piperidine-1-carbonyl]-2-methoxy-phenyl]methanesulfonamide

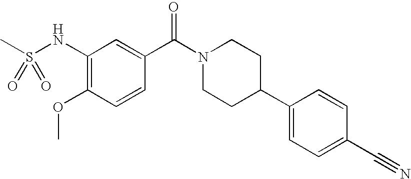

Prepared from Intermediate B

¹H NMR (300.072 MHz, CDCl₃, 30 C) δ 1.55-2.00 (5H, m), 2.78-2.92 (1H, m), 2.93-3.20 (4H, m), 3.93 (3H, s), 3.99-5.00 (2H, m), 6.86 (1H, s), 6.97 (1H, dJ=8.9 Hz), 7.28-7.37 (3H, m), 7.57-7.66 (3H, m), m/z 414 (M+H)⁺.

### EXAMPLE 7

### N-[3-[4-(4-cyanophenyl)piperidine-1-carbonyl]-4-methyl-phenyl]methanesulfonamide

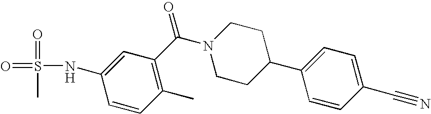

Prepared from Intermediate C

¹H NMR (300.072 MHz, CDCl₃) δ1.48-1.86 (3H, m), 1.94-2.08 (1H, m), 2.30 (3H, d), 2.75-2.92 (2H, m), 2.96 (3H, s), 3.02-3.18 (1H, m), 3.56-3.64 (1H, m), 4.90-5.01 (1H, m), 7.07 (1H, d), 7.15-7.21 (2H, m), 7.28-7.34 (2H, m), 7.52 (1H, s), 7.61 (2H, d), m/z 396 (M−H)⁻.

### EXAMPLE 8

### N-[3-[4-(4-cyanophenyl)piperidine-1-carbonyl]-4-methyl-phenyl]-1-phenyl-methanesulfonamide

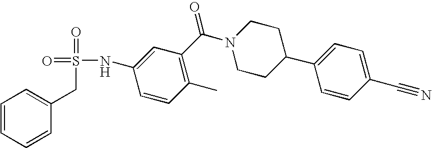

Prepared from Intermediate C

¹H NMR (300.072 MHz, CDCl₃) δ1.40-1.86 (3H, m), 1.97-2.02 (1H, m), 2.31 (3H, d), 2.79-2.91 (2H, m), 3.01-3.20 (1H, m), 3.54-3.69 (1H, m), 4.31 (2H, s), 4.87-5.03 (1H, m), 6.75-6.84 (1H, m), 6.95-7.13 (2H, m), 7.18 (1H, d), 7.23-7.37 (7H, m), 7.61 (2H, d), m/z 474 (M+H)⁺.

### EXAMPLE 9

### 3-chloro-N-[5-[4-(4-cyanophenyl)piperidine-1-carbonyl]-2-methyl-phenyl]propane-1-sulfonamide

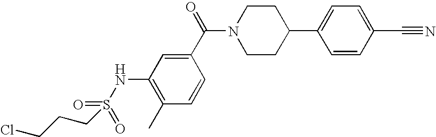

Prepared from Intermediate A

¹H NMR (400.13 MHz, DMSO-d₆) δ 1.61-1.70 (3H, br), 1.85 (1H, s), 2.12-2.20 (2H, m), 2.35 (3H, s), 2.84 (1H, s), 2.89-2.99 (1H, m), 3.15 (1H, s), 3.22-3.28 (2H, m), 3.71 (1H, s), 3.76 (2H, t), 4.62 (1H, s), 7.23 (1H, dd), 7.31-7.35 (2H, m), 7.51 (2H, d), 7.78 (2H, d), 9.38 (1H, s), m/z 460 (M+H)⁺ [A].

### EXAMPLE 10

### N-[5-[4-(4-cyanophenyl)piperidine-1-carbonyl]-2-methyl-phenyl]benzenesulfonamide

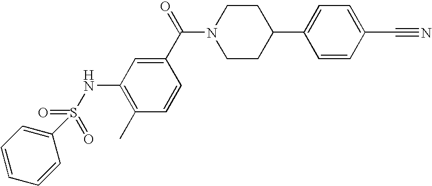

Prepared from Intermediate A

¹H NMR (300.073 MHz, DMSO-d₆) δ 1.52 (2H, s), 1.75 (2H, s), 2.02 (3H, s), 2.84-2.97 (3H, m), 3.55 (1H, s), 4.54 (1H, s), 6.98 (1H, s), 7.13-7.24 (2H, m), 7.46-7.61 (5H, m), 7.65 (2H, dd), 7.79 (2H, d), 9.71 (1H, s), m/z 460 (M+H)⁺.

### EXAMPLE 11

### N-[5-[4-(4-cyanophenyl)piperidine-1-carbonyl]-2-methyl-phenyl]ethanesulfonamide

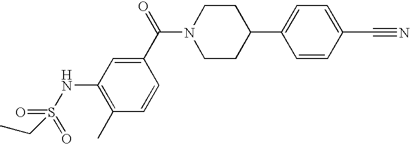

Prepared from Intermediate A

¹H NMR (400.13 MHz, DMSO-d₆) δ1.25 (3H, t), 1.66 (3H, br), 1.85 (1H, s), 2.34 (3H, s), 2.84 (1H, s), 2.89-2.98 (1H, m), 3.08-3.19 (3H, m), 3.73 (1H, s), 4.61 (1H, s), 7.22 (1H, dd), 7.31 (2H, d), 7.52 (2H, d), 7.78 (2H, d), 9.21 (1H, s), m/z 412 (M+H)⁺.

### EXAMPLE 12

### N-[5-[4-(4-cyanophenyl)piperidine-1-carbonyl]-2-methyl-phenyl]butane-1-sulfonamide

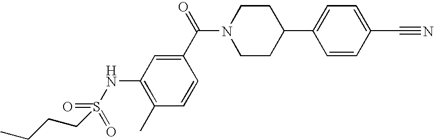

Prepared from Intermediate A

¹H NMR (400.13 MHz, DMSO-d₆) δ0.84 (3H, t), 1.37 (2H, q), 1.64-1.72 (5H, m), 1.85 (1H, s), 2.33 (3H, s), 2.90-2.99 (2H, m), 3.10 (2H, t), 3.17 (1H, s), 3.72 (1H, s), 4.62 (1H, s), 7.21 (1H, d), 7.31 (2H, d), 7.52 (2H, d), 7.78 (2H, d), 9.21 (1H, s), m/z 440 (M+H)⁺.

### EXAMPLE 13

### Methyl 2-[[5-[4-(4-cyanophenyl)piperidine-1-carbonyl]-2-methyl-phenyl]sulfamoyl]acetate

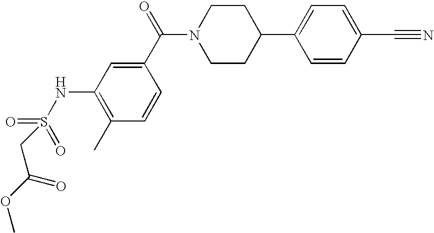

Prepared from Intermediate A

¹H NMR (400.13 MHz, DMSO-d₆) δ1.60-1.71 (3H, m), 1.86 (1H, s), 2.34 (3H, s), 2.85 (1H, s), 2.90-2.99 (1H, s), 3.17 (1H, d), 3.65 (3H, s), 3.74 (1H, s), 4.30 (2H, s), 4.62 (1H, s), 7.26 (1H, dd), 7.34 (1H, d), 7.39 (1H, d), 7.52 (2H, d), 7.79 (2H, d), 9.67 (1H, s), m/z 456 (M+H)⁺.

### EXAMPLE 14

### N-[5-[4-(4-cyanophenyl)piperidine-1-carbonyl]-2-methyl-phenyl]cyclopropanesulfonamide

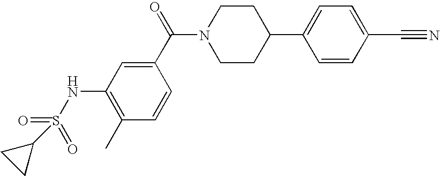

Prepared from Intermediate A

¹H NMR (400.13 MHz, DMSO-d₆) δ0.81-0.86 (2H, m), 0.91-0.96 (2H, m), 1.64 (2H, s), 1.85 (2H, s), 2.37 (3H, s), 2.60-2.69 (1H, m), 2.89-2.99 (2H, m), 3.17 (1H, d), 3.72 (1H, s), 4.61 (1H, s), 7.23 (1H, dd), 7.31-7.35 (2H, m), 7.52 (2H, d), 7.79 (2H, d), 9.26 (1H, s), m/z 424 (M+H)⁺.

### EXAMPLE 15

### N-[5-[4-(4-cyanophenyl)piperidine-1-carbonyl]-2-methyl-phenyl]-1-cyclohexyl-methanesulfonamide

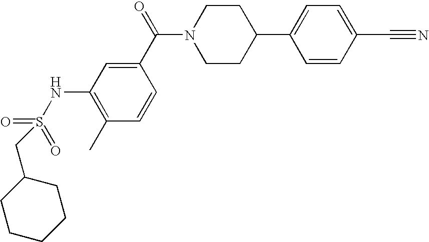

Prepared from Intermediate A

¹H NMR (400.13 MHz, DMSO-d₆) δ0.97-1.24 (6H, m), 1.53-1.66 (5H, m), 1.83 (4H, d), 2.33 (3H, s), 1.85 (1H, s), 2.90-2.96 (1H, m), 3.00 (2H, d), 3.14 (1H, s), 3.71 (1H, s), 4.62 (1H, s), 7.20 (1H, dd), 7.32 (2H, d), 7.52 (2H, d), 7.79 (2H, d), 9.22 (1H, s), m/z 480 (M+H)⁺.

### EXAMPLE 16

### N-[5-[4-(4-cyanophenyl)piperidine-1-carbonyl]-2-methyl-phenyl]-1-cyclopentyl-methanesulfonamide

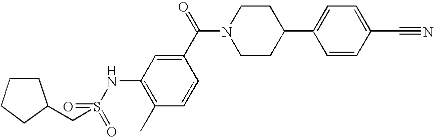

Prepared from Intermediate A

¹H NMR (400.13 MHz, DMSO-d₆) δ1.20-1.30 (2H, m), 1.42-1.51 (2H, m), 1.51-1.59 (2H, m), 1.66 (2H, s), 1.79-1.88 (4H, m), 2.21-2.26 (1H, m), 2.33 (3H, s), 2.85 (1H, s), 2.90-2.99 (1H, m), 3.14 (3H, d), 3.72 (1H, s), 4.61 (1H, s), 7.20 (1H, dd), 7.30-7.34 (2H, m), 7.52 (2H, d), 7.79 (2H, d), 9.19 (1H, s), m/z 466 (M+H)⁺.

### EXAMPLE 17

### N-[5-[4-(4-cyanophenyl)piperidine-1-carbonyl]-2-methyl-phenyl]cyclohexanesulfonamide

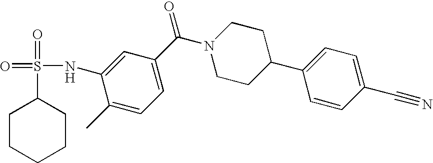

Prepared from Intermediate A

¹H NMR (300.072 MHz, CDCl₃) δ1.16-1.31 (4H, m), 1.57-1.76 (4H, m), 1.82-1.95 (4H, m), 2.08-2.19 (2H, m), 2.32 (3H, s), 2.80-2.90 (2H, m), 3.01-3.11 (2H, m), 3.83-4.03 (1H, m), 4.71-5.03 (1H, m), 6.14 (1H, s), 7.15-7.19 (1H, m), 7.22-7.26 (1H, m), 7.32 (2H, d), 7.56-7.63 (3H, m), m/z 466 (M+H)⁺.

### EXAMPLE 18

### (RS)—N-[5-[4-(4-cyanophenyl)piperidine-1-carbonyl]-2-methyl-phenyl]butane-2-sulfonamide

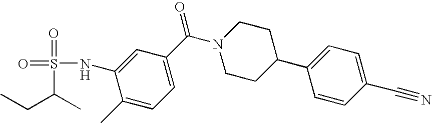

Prepared from Intermediate A

¹H NMR (300.072 MHz, CDCl₃) δ1.02 (3H, t), 1.39 (3H, d), 1.52-1.79 (4H, m), 1.84-2.12 (2H, m), 2.33 (3H, s), 2.79-2.92 (2H, m), 3.06-3.17 (2H, m), 3.78-4.03 (1H, m), 4.70-4.99 (1H, m), 6.16 (1H, s), 7.14-7.18 (1H, m), 7.22-7.25 (1H, m), 7.32 (2H, d), 7.55-7.63 (3H, m), m/z 440 (M+H)⁺.

### EXAMPLE 19

### 5-chloro-N-[5-[4-(4-cyanophenyl)piperidine-1-carbonyl]-2-methyl-phenyl]thiophene-2-sulfonamide

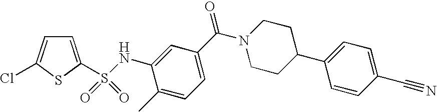

Prepared from Intermediate A

¹H NMR (300.072 MHz, CDCl₃) δ 1.60-1.95 (4H, m), 2.14 (3H, s), 2.79-2.94 (2H, m), 2.99-3.17 (1H, m), 3.73-4.03 (1H, m), 4.61-5.10 (1H, m), 6.80 (1H, s), 6.85 (1H, d), 7.19-7.27 (3H, m), 7.30-7.37 (3H, m), 7.63 (2H, d), m/z 500 (M+H)⁺ [A].

### EXAMPLE 20

### 2-cyano-N-[5-[4-(4-cyanophenyl)piperidine-1-carbonyl]-2-methyl-phenyl]benzenesulfonamide

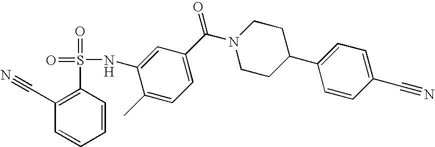

Prepared from Intermediate A

¹H NMR (300.072 MHz, CDCl₃) δ 1.66-1.95 (4H, m), 2.18 (3H, s), 2.77-2.91 (2H, m), 2.96-3.13 (1H, m), 3.76-4.01 (1H, m), 4.62-4.97 (1H, m), 7.17 (2H, d), 7.30-7.36 (4H, m), 7.60-7.75 (4H, m), 7.81-7.85 (1H, m), 8.08-8.13 (1H, m), m/z 485 (M+H)⁺.

### EXAMPLE 21

### 3-cyano-N-[5-[4-(4-cyanophenyl)piperidine-1-carbonyl]-2-methyl-phenyl]benzenesulfonamide

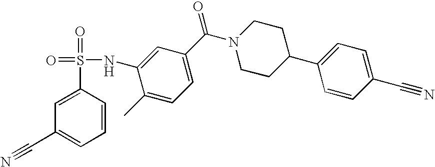

Prepared from Intermediate A

¹H NMR (300.072 MHz, CDCl₃) δ1.62-1.97 (4H, m), 2.02 (3H, s), 2.82-2.96 (2H, m), 3.08-3.23 (1H, m), 3.67-3.95 (1H, m), 4.59-5.10 (1H, m), 7.13-7.23 (4H, m), 7.33 (2H, d), 7.56-7.65 (3H, m), 7.78-7.83 (1H, m), 7.94-7.98 (2H, m), m/z 485 (M+H)⁺.

### EXAMPLE 22

### N-[4-[[5-[4-(4-cyanophenyl)piperidine-1-carbonyl]-2-methyl-phenyl]sulfamoyl]phenyl]acetamide

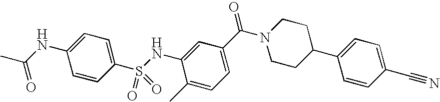

Prepared from Intermediate A

¹H NMR (300.073 MHz, DMSO-d₆) δ 1.39-1.82 (4H, m), 2.01 (3H, s), 2.06 (3H, s), 2.79-3.06 (3H, m), 3.40-3.65 (1H, m), 4.34-4.68 (1H, m), 6.95 (1H, s), 7.13-7.25 (2H, m), 7.47 (2H, d), 7.56 (2H, d), 7.69 (2H, d), 7.77 (2H, d), 9.55 (1H, s), 10.25 (1H, s), m/z 517 (M+H)⁺.

### EXAMPLE 23

### 4-cyano-N-[5-[4-(4-cyanophenyl)piperidine-1-carbonyl]-2-methyl-phenyl]benzenesulfonamide

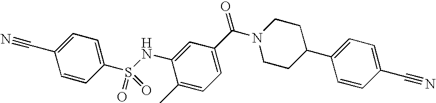

Prepared from Intermediate A

¹H NMR (300.072 MHz, CDCl₃) δ 1.65-1.93 (4H, m), 1.98 (3H, s), 2.82-2.92 (2H, m), 2.99-3.15 (1H, m), 3.67-3.94 (1H, m), 4.74-5.02 (1H, m), 7.10-7.21 (3H, m), 7.33 (2H, d), 7.47 (1H, s), 7.63 (2H, d), 7.71 (2H, d), 7.83 (2H, d), m/z 485 (M+H)⁺.

### EXAMPLE 24

### 4-butoxy-N-[5-[4-(4-cyanophenyl)piperidine-1-carbonyl]-2-methyl-phenyl]benzenesulfonamide

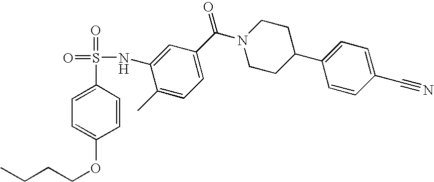

Prepared from Intermediate A

¹H NMR (300.072 MHz, CDCl₃) δ 0.96 (3H, t), 1.46 (2H, sextet), 1.65-1.96 (6H, m), 2.03 (3H, s), 2.79-2.90 (2H, m), 2.95-3.13 (1H, m), 3.80-3.91 (1H, m), 3.95 (2H, t), 4.65-4.93 (1H, m), 6.62 (1H, s), 6.85 (2H, d), 7.11-7.21 (2H, m), 7.33 (2H, d), 7.38-7.41 (1H, m), 7.60-7.65 (4H, m), m/z 532 (M+H)⁺.

### EXAMPLE 25

### N-[5-[4-(4-cyanophenyl)piperidine-1-carbonyl]-2-methyl-phenyl]-2-methoxy-benzenesulfonamide

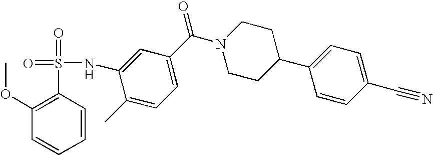

Prepared from Intermediate A

¹H NMR (300.072 MHz, CDCl₃) [symbol] 1.49-1.92 (m, 4H), 2.26 (s, 3H), 2.78-2.87 (m, 2H), 2.95-3.07 (m, 1H), 3.51-3.88 (m, 1H), 4.01 (s, 3H), 4.57-5.00 (m, 1H), 6.89 (s, 1H), 6.93-7.04 (m, 2H), 7.09-7.18 (m, 2H), 7.30-7.35 (m, 3H), 7.45-7.52 (m, 1H), 7.63 (d, 2H), 7.79-7.82 (m, 1H), m/z 490 (M+H)⁺.

### EXAMPLE 26

### N-[5-[4-(4-cyanophenyl)piperidine-1-carbonyl]-2-methyl-phenyl]-1-(2-fluorophenyl)methanesulfonamide

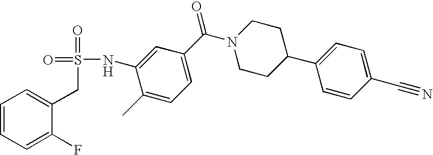

Prepared from Intermediate A

¹H NMR (300.072 MHz, CDCl₃) [symbol] 1.50-1.91 (m, 4H), 2.18 (s, 3H), 2.80-2.91 (m, 2H), 2.95-3.21 (m, 1H), 3.73-3.95 (m, 1H), 4.49 (s, 2H), 4.62-5.03 (m, 1H), 6.24 (s, 1H), 7.05 (t, 1H), 7.12-7.24 (m, 3H), 7.32-7.40 (m, 4H), 7.52 (d, 1H), 7.62 (d, 2H), m/z 492 (M+H)⁺.

### EXAMPLE 27

### N-[5-[4-(4-cyanophenyl)piperidine-1-carbonyl]-2-methyl-phenyl]-1-(3-fluorophenyl)methanesulfonamide

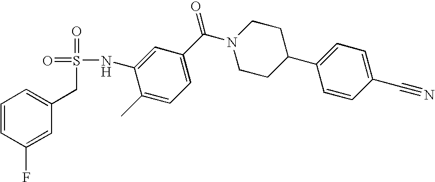

Prepared from Intermediate A

¹H NMR (300.072 MHz, CDCl₃) [symbol] 1.64-1.95 (m, 4H), 2.09 (s, 3H), 2.77-2.92 (m, 2H), 2.98-3.22 (m, 1H), 3.73-3.99 (m, 1H), 4.37 (s, 2H), 4.74-4.99 (m, 1H), 6.33 (s, 1H), 6.95-7.10 (m, 3H), 7.16-7.25 (m, 2H), 7.27-7.36 (m, 3H), 7.50-7.52 (m, 1H), 7.62 (d, 2H), m/z 492 (M+H)⁺.

### EXAMPLE 28

### N-[5-[4-(4-cyanophenyl)piperidine-1-carbonyl]-2,4-dimethyl-phenyl]methanesulfonamide

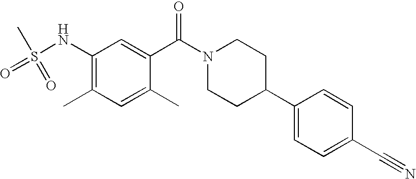

Prepared from Intermediate D

¹H NMR (300.072 MHz, CDCl₃) δ1.63-1.82 (2H, m), 1.98 (1H, s), 2.29 (6H, s), 2.84 (2H, m), 3.01 (3H, s), 3.11 (1H, m), 3.64 (1H, m), 4.95 (1H, m), 6.36 (1H, s), 7.09 (1H, s), 7.31 (2H, d), 7.61 (2H, d), m/z 412 (M+H)⁺.

### EXAMPLE 29

### N-[5-[4-(4-cyanophenyl)piperidine-1-carbonyl]-2,4-dimethyl-phenyl]-1-phenyl-methanesulfonamide

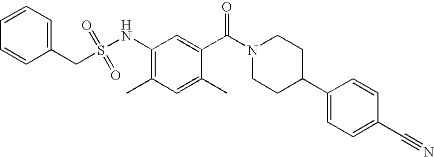

Prepared from Intermediate D

¹H NMR (300.072 MHz, CDCl₃) δ1.72-1.78 (1H, m), 2.00 (3H, s), 2.31 (3H, s), 2.85 (2H, m), 3.11 (1H, s), 3.63 (1H, d), 4.37 (2H, s), 4.98 (1H, d), 5.96 (1H, s), 7.06 (1H, s), 7.21-7.25 (2H, m), 7.32-7.40 (5H, m), 7.62 (2H, d), m/z 488 (M+H)⁺.

### EXAMPLE 30

### N-[2-cyano-5-[4-(4-cyanophenyl)piperidine-1-carbonyl]phenyl]-1-phenyl-methanesulfonamide

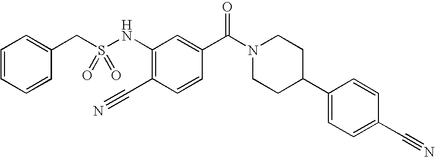

Prepared from Intermediate E

¹H NMR (400.132 MHz, CDCl₃) [symbol] 1.59-1.87 (m, 3H), 1.95-2.03 (m, 1H), 2.83-2.95 (m, 2H), 3.10-3.19 (m, 1H), 3.57-3.76 (m, 1H), 4.51 (s, 2H), 4.81-4.89 (m, 1H), 6.88 (s, 1H), 7.25-7.36 (m, 8H), 7.58-7.65 (m, 4H), m/z 485 (M+H)⁺.

### EXAMPLE 31

### N-[3-[4-(4-cyanophenyl)piperidine-1-carbonyl]-4-methoxy-phenyl]-1-phenyl-methanesulfonamide

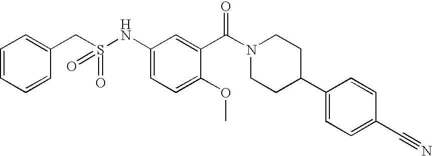

Prepared from Intermediate F

¹H NMR (400.132 MHz, DMSO-d₆) [symbol] 1.37-1.96 (m, 4H), 2.71-3.21 (m, 3H), 3.37-3.49 (m, 1H), 3.72-3.86 (m, 3H), 4.40 (s, 2H), 4.58-4.72 (m, 1H), 7.03-7.10 (m, 2H), 7.19-7.30 (m, 3H), 7.30-7.39 (m, 3H), 7.45-7.53 (m, 2H), 7.74-7.84 (m, 2H), 9.64-9.76 (m, 1H), m/z 490 (M+H)⁺.

### EXAMPLE 32

### N-[3-[4-(4-cyanophenyl)piperidine-1-carbonyl]-4-methyl-phenyl]propane-2-sulfonamide

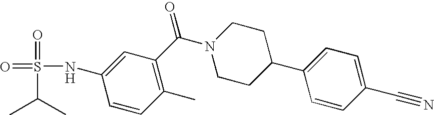

Prepared from Intermediate C

¹H NMR (300.072 MHz, CDCl₃) [symbol] 1.37 (d, 6H), 1.51-1.85 (m, 3H), 1.95-2.00 (m, 1H), 2.30 (d, 3H), 2.78-2.93 (m, 2H), 3.05-3.17 (m, 1H), 3.29 (septet, 1H), 3.56-3.63 (m, 1H), 4.89-5.03 (m, 1H), 7.01-7.19 (m, 4H), 7.29-7.34 (m, 2H), 7.61 (d, 2H), m/z 426 (M+H)⁺.

### EXAMPLE 33

### N-[3-[4-(4-cyanophenyl)piperidine-1-carbonyl]-4-methyl-phenyl]propane-1-sulfonamide

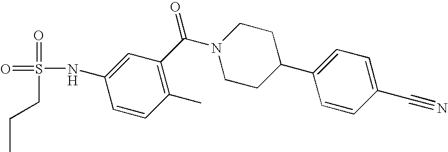

Prepared from Intermediate C

¹H NMR (300.072 MHz, CDCl₃) [symbol] 1.00 (t, 3H), 1.50-1.88 (m, 5H), 1.97-2.04 (m, 1H), 2.30 (d, 3H), 2.78-2.93 (m, 2H), 2.99-3.15 (m, 3H), 3.59 (d, 1H), 4.96 (d, 1H), 7.01-7.21 (m, 4H), 7.31 (d, 2H), 7.61 (d, 2H), m/z 426 (M+H)⁺.

### EXAMPLE 34

### 3-chloro-N-[3-[4-(4-cyanophenyl)piperidine-1-carbonyl]-4-methyl-phenyl]propane-1-sulfonamide

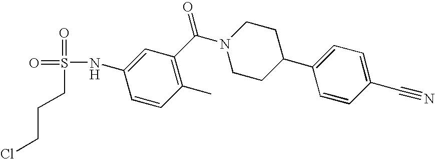

Prepared from Intermediate C

¹H NMR (300.072 MHz, CDCl₃) [symbol] 1.49-1.91 (m, 3H), 1.91-1.99 (m, 1H), 2.18-2.38 (m, 5H), 2.80-2.92 (m, 2H), 3.05-3.27 (m, 3H), 3.50-3.67 (m, 3H), 4.88-5.02 (m, 1H), 6.99-7.20 (m, 3H), 7.31 (d, 2H), 7.61 (d, 2H), 7.67 (s, 1H), m/z 460 (M+H)⁺ [A].

### EXAMPLE 35

### N-[3-[4-(4-cyanophenyl)piperidine-1-carbonyl]-4-methyl-phenyl]benzenesulfonamide

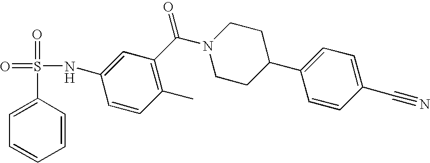

Prepared from Intermediate C

¹H NMR (300.072 MHz, CDCl₃) [symbol] 1.42-1.75 (m, 3H), 1.97-2.04 (m, 1H), 2.26 (d, 3H), 2.75-2.86 (m, 2H), 2.92-3.07 (m, 1H), 3.35-3.45 (m, 1H), 4.89-4.98 (m, 1H), 6.76-7.11 (m, 4H), 7.28-7.48 (m, 5H), 7.62 (d, 2H), 7.74 (d, 2H), m/z 460 (M+H)⁺.

### EXAMPLE 36

### N-[3-[4-(4-cyanophenyl)piperidine-1-carbonyl]-4-methyl-phenyl]ethanesulfonamide

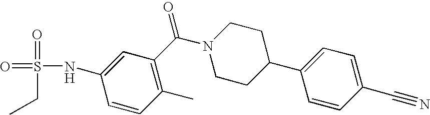

Prepared from Intermediate C

¹H NMR (300.072 MHz, CDCl₃) [symbol] 1.34 (t, 3H), 1.47-1.85 (m, 3H), 1.95-2.00 (m, 1H), 2.30 (d, 3H), 2.78-2.92 (m, 2H), 3.05-3.16 (m, 3H), 3.55-3.62 (m, 1H), 4.93-5.01 (m, 1H), 7.01-7.25 (m, 4H), 7.29-7.34 (m, 2H), 7.61 (d, 2H), m/z 410 (M−H)⁻.

### EXAMPLE 37

### N-[3-[4-(4-cyanophenyl)piperidine-1-carbonyl]-4-methyl-phenyl]butane-1-sulfonamide

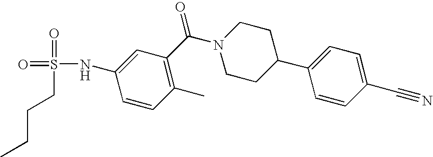

Prepared from Intermediate C

¹H NMR (300.072 MHz, CDCl₃) δ 0.88 (t, 3H), 1.40 (sextet, 2H), 1.68-1.83 (m, 5H), 1.95-2.00 (m, 1H), 2.30 (d, 3H), 2.78-2.90 (m, 2H), 3.02-3.16 (m, 3H), 3.59 (d, 1H), 4.93-5.01 (m, 1H), 7.02-7.20 (m, 4H), 7.31 (d, 2H), 7.61 (d, 2H), m/z 440 (M+H)⁺.

### EXAMPLE 38

### methyl 2-[[3-[4-(4-cyanophenyl)piperidine-1-carbonyl]-4-methyl-phenyl]sulfamoyl]acetate

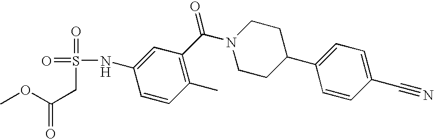

Prepared from Intermediate C

¹H NMR (300.072 MHz, CDCl₃) [symbol] 1.51-1.83 (m, 3H), 1.97-2.01 (m, 1H), 2.33 (d, 3H), 2.79-2.92 (m, 2H), 3.04-3.15 (m, 1H), 3.57-3.60 (m, 1H), 3.79 (s, 3H), 3.95 (s, 2H), 4.90-5.00 (m, 1H), 7.11-7.35 (m, 6H), 7.61 (d, 2H), m/z 456 (M+H)⁺.

### EXAMPLE 39

### N-[3-[4-(4-cyanophenyl)piperidine-1-carbonyl]-4-methyl-phenyl]-5-methyl-1,2-oxazole-4-sulfonamide

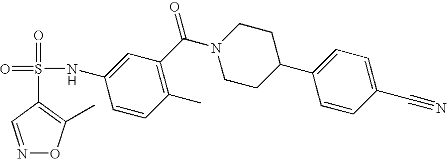

Prepared from Intermediate C

¹H NMR (300.072 MHz, CDCl₃) [symbol] 1.42-1.82 (m, 3H), 1.98-2.02 (m, 1H), 2.30 (d, 3H), 2.45 (d, 3H), 2.78-2.89 (m, 2H), 2.97-3.16 (m, 1H), 3.32-3.45 (m, 1H), 4.89-4.99 (m, 1H), 6.70 (d, 1H), 7.05 (d, 1H), 7.14 (d, 1H), 7.31 (d, 2H), 7.62 (d, 2H), 8.11 (d, 1H), 8.21 (s, 1H), m/z 463 (M−H)⁻.

### EXAMPLE 40

### N-[3-[4-(4-cyanophenyl)piperidine-1-carbonyl]-4-methyl-phenyl]cyclopropanesulfonamide

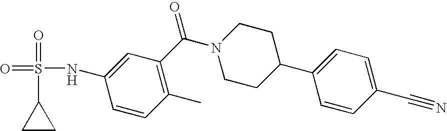

Prepared from Intermediate C

¹H NMR (300.072 MHz, CDCl₃) [symbol] 0.91-0.98 (m, 2H), 1.11-1.19 (m, 2H), 1.44-1.86 (m, 3H), 1.94-2.01 (m, 1H), 2.31 (d, 3H), 2.43-2.52 (m, 1H), 2.78-2.92 (m, 2H), 3.10 (t, 1H), 3.61 (d, 1H), 4.97 (d, 1H), 6.98-7.20 (m, 4H), 7.29-7.34 (m, 2H), 7.61 (d, 2H), m/z 422 (M−H)⁻.

### EXAMPLE 41

### N-[3-[4-(4-cyanophenyl)piperidine-1-carbonyl]-4-methyl-phenyl]-1-cyclohexyl-methanesulfonamide

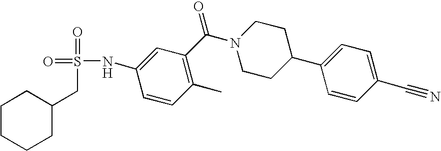

Prepared from Intermediate C

¹H NMR (300.072 MHz, CDCl₃) [symbol] 0.94-1.20 (m, 6H), 1.58-2.03 (m, 9H), 2.30 (d, 3H), 2.80-2.88 (m, 2H), 2.95 (d, 2H), 3.04-3.18 (m, 1H), 3.54-3.63 (m, 1H), 4.90-4.99 (m, 1H), 6.99-7.21 (m, 4H), 7.31 (d, 2H), 7.61 (d, 2H), m/z 480 (M+H)⁺.

### EXAMPLE 42

### N-[3-[4-(4-cyanophenyl)piperidine-1-carbonyl]-4-methyl-phenyl]cyclohexanesulfonamide

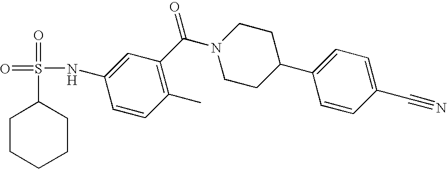

Prepared from Intermediate C

¹H NMR (300.072 MHz, CDCl₃) δ 1.12-1.21 (m, 3H), 1.48-1.91 (m, 8H), 1.96-2.00 (m, 1H), 2.09-2.17 (m, 2H), 2.30 (d, 3H), 2.79-2.91 (m, 2H), 2.95-3.16 (m, 2H), 3.56-3.65 (m, 1H), 4.91-5.03 (m, 1H), 6.84 (d, 1H), 7.01-7.20 (m, 3H), 7.31 (d, 2H), 7.61 (d, 2H), m/z 466 (M+H)⁺.

### EXAMPLE 43

### N-[3-[4-(4-cyanophenyl)piperidine-1-carbonyl]-4-methyl-phenyl]-1-cyclopentyl-methanesulfonamide

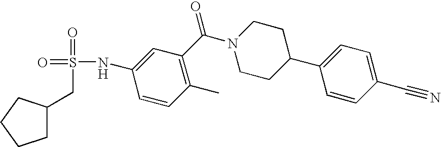

Prepared from Intermediate C

¹H NMR (300.072 MHz, CDCl₃) [symbol] 1.15-1.32 (m, 2H), 1.46-1.61 (m, 5H), 1.69-2.02 (m, 5H), 2.23-2.41 (m, 4H), 2.77-2.94 (m, 2H), 3.03-3.20 (m, 3H), 3.54-3.62 (m, 1H), 4.90-5.01 (m, 1H), 6.87-6.94 (m, 1H), 7.00-7.20 (m, 3H), 7.31 (d, 2H), 7.61 (d, 2H), m/z 466 (M+H)⁺.

### EXAMPLE 44

### (RS) N-[3-[4-(4-cyanophenyl)piperidine-1-carbonyl]-4-methyl-phenyl]butane-2-sulfonamide

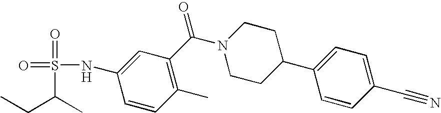

Prepared from Intermediate C

¹H NMR (300.072 MHz, CDCl₃) [symbol] 0.98 (t, 3H), 1.34 (d, 3H), 1.52-1.84 (m, 4H), 1.91-2.03 (m, 2H), 2.30 (d, 3H), 2.76-2.94 (m, 2H), 2.99-3.15 (m, 2H), 3.59 (d, 1H), 4.96 (d, 1H), 6.99-7.20 (m, 4H), 7.31 (d, 2H), 7.61 (d, 2H), m/z 440 (M+H)⁺.

### EXAMPLE 45

### 5-chloro-N-[3-[4-(4-cyanophenyl)piperidine-1-carbonyl]-4-methyl-phenyl]thiophene-2-sulfonamide

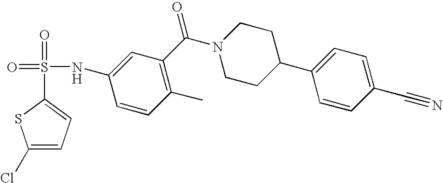

Prepared from Intermediate C

¹H NMR (300.072 MHz, CDCl₃) [symbol] 1.41-1.81 (m, 3H), 1.94-2.00 (m, 1H), 2.30 (d, 3H), 2.78-2.91 (m, 2H), 2.96-3.14 (m, 1H), 3.41-3.48 (m, 1H), 4.89-5.00 (m, 1H), 6.73 (s, 1H), 6.84 (d, 1H), 7.04-7.14 (m, 2H), 7.20-7.24 (m, 1H), 7.30 (d, 2H), 7.62 (d, 2H), 8.06 (s, 1H), m/z 500 (M+H)⁺ [A].

### EXAMPLE 46

### 2-cyano-N-[3-[4-(4-cyanophenyl)piperidine-1-carbonyl]-4-methyl-phenyl]benzenesulfonamide

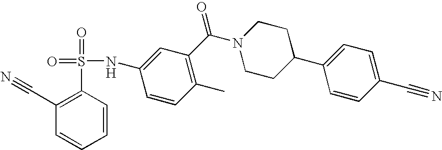

Prepared from Intermediate C

¹H NMR (300.072 MHz, CDCl₃) [symbol] 1.38-1.81 (m, 3H), 1.92-2.00 (m, 1H), 2.24 (d, 3H), 2.77-2.91 (m, 2H), 3.00-3.10 (m, 1H), 3.46-3.54 (m, 1H), 4.87-4.95 (m, 1H), 6.94-7.12 (m, 3H), 7.28-7.37 (m, 2H), 7.58-7.70 (m, 5H), 7.76-7.82 (m, 1H), 8.07 (d, 1H), m/z 485 (M+H)⁺.

### EXAMPLE 47

### 3-cyano-N-[3-[4-(4-cyanophenyl)piperidine-1-carbonyl]-4-methyl-phenyl]benzenesulfonamide

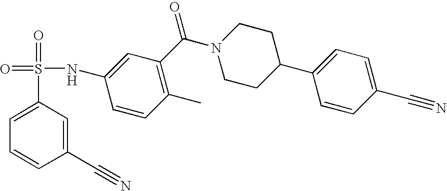

Prepared from Intermediate C

¹H NMR (300.072 MHz, CDCl₃) [symbol] 1.32-1.86 (m, 3H), 1.97-2.01 (m, 1H), 2.28 (d, 3H), 2.77-2.88 (m, 2H), 2.94-3.14 (m, 1H), 3.35-3.42 (m, 1H), 4.90-5.01 (m, 1H), 6.70 (d, 1H), 6.98-7.02 (m, 1H), 7.07-7.14 (m, 1H), 7.30 (d, 2H), 7.46-7.80 (m, 4H), 7.92-8.00 (m, 2H), 8.28 (d, 1H), m/z 485 (M+H)⁺.

### EXAMPLE 48

### N-[4-[[3-[4-(4-cyanophenyl)piperidine-1-carbonyl]-4-methyl-phenyl]sulfamoyl]phenyl]acetamide

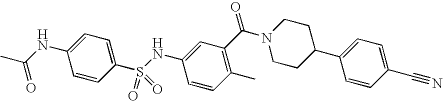

Prepared from Intermediate C

¹H NMR (300.073 MHz, DMSO-d₆) [symbol] 1.23-1.68 (m, 3H), 1.79-2.18 (m, 7H), 2.68-3.14 (m, 4H), 4.55-4.67 (m, 1H), 6.77 (d, 1H), 6.99-7.05 (m, 1H), 7.13 (d, 1H), 7.45 (d, 2H), 7.56-7.69 (m, 4H), 7.74-7.81 (m, 2H), 10.04 (s, 1H), 10.20 (d, 1H), m/z 517 (M+H)⁺.

### EXAMPLE 49

### 4-cyano-N-[3-[4-(4-cyanophenyl)piperidine-1-carbonyl]-4-methyl-phenyl]benzenesulfonamide

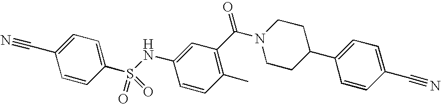

Prepared from Intermediate C

¹H NMR (300.072 MHz, CDCl₃) [symbol] 1.40-2.00 (m, 4H), 2.28 (d, 3H), 2.78-2.92 (m, 2H), 2.95-3.12 (m, 1H), 3.32-3.43 (m, 1H), 4.89-4.99 (m, 1H), 6.76 (d, 1H), 6.96-7.00 (m, 1H), 7.10 (d, 1H), 7.30 (d, 2H), 7.61-7.70 (m, 4H), 7.83 (d, 2H), 7.94 (s, 1H), m/z 483 (M−H)⁻.

### EXAMPLE 50

### N-[3-[4-(4-cyanophenyl)piperidine-1-carbonyl]-4-methyl-phenyl]-2-methoxy-benzenesulfonamide

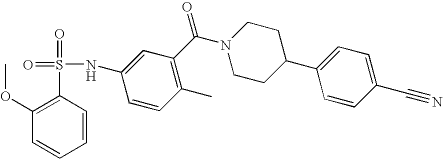

Prepared from Intermediate C

¹H NMR (300.072 MHz, CDCl₃) [symbol] 1.47-1.84 (m, 3H), 1.96-2.02 (m, 1H), 2.20 (d, 3H), 2.74-2.85 (m, 2H), 2.96-3.03 (m, 1H), 3.31-3.44 (m, 1H), 4.01 (s, 3H), 4.86-4.96 (m, 1H), 6.87-7.06 (m, 5H), 7.16-7.25 (m, 1H), 7.28-7.36 (m, 2H), 7.39-7.48 (m, 1H), 7.57-7.66 (m, 2H), 7.73-7.82 (m, 1H), m/z 490 (M+H)⁺.

### EXAMPLE 51

### N-[3-[4-(4-cyanophenyl)piperidine-1-carbonyl]-4-methyl-phenyl]-1-(2-fluorophenyl)methanesulfonamide

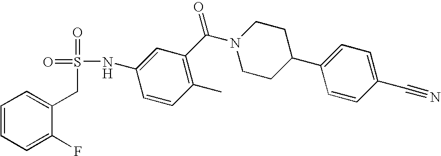

Prepared from Intermediate C

¹H NMR (300.072 MHz, CDCl₃) [symbol] 1.70-1.82 (m, 3H), 1.96-2.03 (m, 1H), 2.31 (d, 3H), 2.80-2.94 (m, 2H), 3.06-3.18 (m, 1H), 3.56-3.62 (m, 1H), 4.39 (d, 2H), 4.89-4.98 (m, 1H), 6.94-7.18 (m, 6H), 7.29-7.39 (m, 4H), 7.61 (d, 2H), m/z 492 (M+H)⁺.

### EXAMPLE 52

### N-[3-[4-(4-cyanophenyl)piperidine-1-carbonyl]-4-methyl-phenyl]-1-(3-fluorophenyl)methanesulfonamide

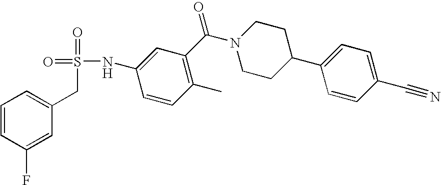

Prepared from Intermediate C

¹H NMR (300.072 MHz, CDCl₃) [symbol] 1.48-1.86 (m, 3H), 1.94-2.00 (m, 1H), 2.31 (d, 3H), 2.80-2.94 (m, 2H), 3.07-3.16 (m, 1H), 3.52-3.63 (m, 1H), 4.30 (d, 2H), 4.91-5.01 (m, 1H), 6.74 (s, 1H), 6.95-7.09 (m, 5H), 7.18 (d, 1H), 7.28-7.34 (m, 3H), 7.61 (d, 2H), m/z 492 (M+H)⁺.

### EXAMPLE 53

### N-[3-[4-(4-cyanophenyl)piperidine-1-carbonyl]-4-methyl-phenyl]-2-oxo-1,3-dihydroindole-5-sulfonamide

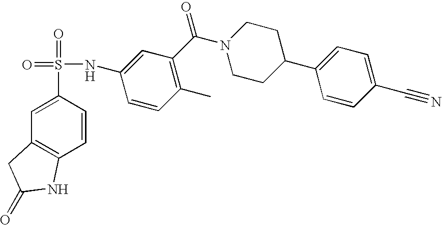

Prepared from Intermediate C

¹H NMR (300.072 MHz, CDCl₃) [symbol] 1.47-1.97 (m, 4H), 2.25 (d, 3H), 2.75-2.92 (m, 2H), 3.02-3.16 (m, 1H), 3.39 (s, 2H), 3.46-3.60 (m, 1H), 4.89-5.01 (m, 1H), 6.59 (d, 1H), 6.98-7.14 (m, 3H), 7.29-7.34 (m, 2H), 7.50-7.65 (m, 4H), 7.70 (s, 1H), 8.73 (s, 1H), m/z 515 (M+H)⁺.

### EXAMPLE 54

### N-[3-[4-(4-cyanophenyl)piperidine-1-carbonyl]-4-methyl-phenyl]-1-(4-methylsulfonylphenyl)methanesulfonamide

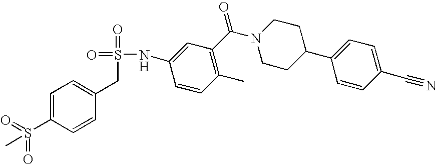

Prepared from Intermediate C

¹H NMR (300.072 MHz, CDCl₃) [symbol] 1.68-1.88 (m, 3H), 1.96-2.01 (m, 1H), 2.31 (d, 3H), 2.82-2.91 (m, 2H), 3.04 (s, 3H), 3.08-3.17 (m, 1H), 3.52-3.59 (m, 1H), 4.39 (d, 2H), 4.88-4.97 (m, 1H), 6.95-7.10 (m, 2H), 7.18 (d, 1H), 7.30-7.38 (m, 3H), 7.48 (d, 2H), 7.62 (d, 2H), 7.87 (d, 2H), m/z 552 (M+H)⁺.

### EXAMPLE 55

### N-[3-[4-(4-cyanophenyl)piperidine-1-carbonyl]-4-methyl-phenyl]-2,2,2-trifluoro-ethanesulfonamide

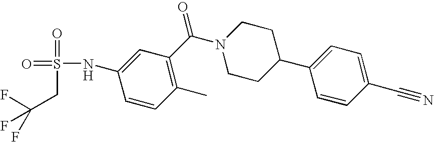

Prepared from Intermediate C

¹H NMR (300.072 MHz, CDCl₃) [symbol] 1.62-1.83 (m, 3H), 1.96-2.00 (m, 1H), 2.32 (d, 3H), 2.80-2.93 (m, 2H), 3.06-3.14 (m, 1H), 3.52-3.61 (m, 1H), 3.76 (q, 2H), 4.91-5.00 (m, 1H), 7.00 (d, 1H), 7.11-7.22 (m, 2H), 7.31 (d, 2H), 7.61 (d, 2H), 7.94 (s, 1H), m/z 466 (M+H)⁺.

### EXAMPLE 56

### N-[3-[4-(4-cyanophenyl)piperidine-1-carbonyl]-4-methyl-phenyl]-1-(2,4-difluorophenyl)methanesulfonamide

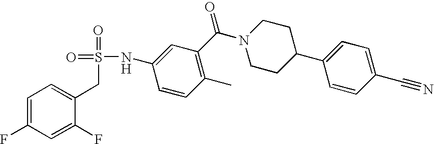

Prepared from Intermediate C

¹H NMR (300.072 MHz, CDCl₃) [symbol] 1.59-1.83 (m, 3H), 1.94-2.00 (m, 1H), 2.30 (d, 3H), 2.78-2.90 (m, 2H), 3.05-3.15 (m, 1H), 3.55-3.63 (m, 1H), 4.34 (d, 2H), 4.89-4.98 (m, 1H), 6.72-6.90 (m, 2H), 6.94-7.20 (m, 3H), 7.29-7.39 (m, 4H), 7.61 (d, 2H), m/z 510 (M+H)⁺.

### EXAMPLE 57

### N-[3-[4-(4-cyanophenyl)piperidine-1-carbonyl]-4-methyl-phenyl]-2-methyl-propane-1-sulfonamide

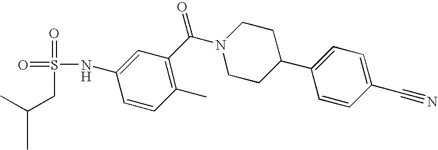

Prepared from Intermediate C

¹H NMR (300.072 MHz, CDCl₃) [symbol] 1.06 (d, 6H), 1.49-1.83 (m, 3H), 1.97-2.00 (m, 1H), 2.23-2.38 (m, 4H), 2.79-2.91 (m, 2H), 2.95 (d, 2H), 3.05-3.15 (m, 1H), 3.55-3.64 (m, 1H), 4.91-5.01 (m, 1H), 7.00-7.22 (m, 4H), 7.32 (d, 2H), 7.61 (d, 2H), m/z 440 (M+H)⁺.

### EXAMPLE 58

### N-[3-[4-(4-cyanophenyl)piperidine-1-carbonyl]-4-methyl-phenyl]-2-phenyl-ethanesulfonamide

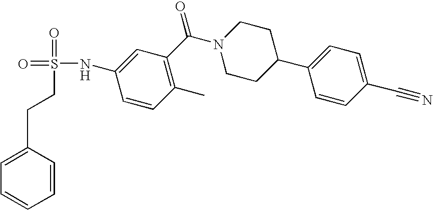

Prepared from Intermediate C

¹H NMR (300.072 MHz, CDCl₃) [symbol] 1.58-1.80 (m, 3H), 1.93-1.99 (m, 1H), 2.29 (d, 3H), 2.74-2.93 (m, 2H), 3.04-3.14 (m, 3H), 3.28-3.35 (m, 2H), 3.49-3.60 (m, 1H), 4.92-4.99 (m, 1H), 6.88-7.16 (m, 6H), 7.21-7.31 (m, 5H), 7.60 (d, 2H), m/z 488 (M+H)⁺.

### EXAMPLE 59

### N-[3-[4-(4-cyanophenyl)piperidine-1-carbonyl]-4-methyl-phenyl]pentane-2-sulfonamide

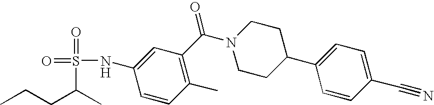

Prepared from Intermediate C

¹H NMR (300.072 MHz, CDCl₃) [symbol] 0.88 (t, 3H), 1.34 (d, 3H), 1.43-1.98 (m, 8H), 2.30 (d, 3H), 2.78-2.90 (m, 2H), 3.04-3.16 (m, 2H), 3.54-3.64 (m, 1H), 4.89-5.00 (m, 1H), 7.03-7.23 (m, 4H), 7.28-7.35 (m, 2H), 7.61 (d, 2H), m/z 454 (M+H)⁺.

### EXAMPLE 60

### N-[5-[4-(4-cyanophenyl)piperidine-1-carbonyl]-2-methyl-phenyl]-2,2,2-trifluoro-ethanesulfonamide

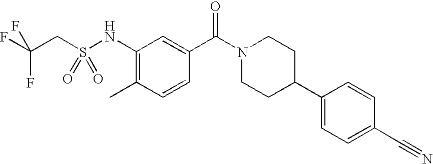

Prepared from Intermediate A

¹H NMR (300.072 MHz, CDCl₃) δ1.60-2.00 (4H, m), 2.34 (3H, s), 2.82-2.90 (1H, m), 3.88 (2H, q), 7.10 (1H, s), 7.22-7.25 (1H, m), 7.32 (2H, d), 7.44 (1H, d), 7.60-7.63 (2H, m), m/z 466 (M+H)⁺.

### EXAMPLE 61

### N-[5-[4-(4-cyanophenyl)piperidine-1-carbonyl]-2-methyl-phenyl]-1-(2,4-difluorophenyl)methanesulfonamide

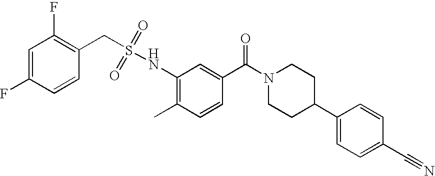

Prepared from Intermediate A

¹H NMR (300.072 MHz, CDCl₃) δ 1.60-2.00 (4H, m), 2.21 (3H, s), 2.85-2.91 (1H, m), 4.43 (2H, s), 6.32 (1H, s), 6.78-6.93 (2H, m), 7.24 (1H, m), 7.32-7.38 (3H, m), 7.51 (1H, d), 7.60-7.63 (2H, m), m/z 510 (M+H)⁺.

### EXAMPLE 62

### N-[5-[4-(4-cyanophenyl)piperidine-1-carbonyl]-2-methyl-phenyl]-1-(4-methylsulfonylphenyl)methanesulfonamide

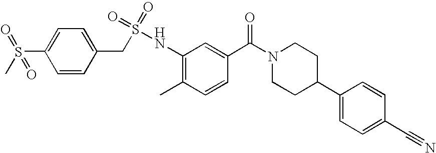

Prepared from Intermediate A

¹H NMR (300.072 MHz, CDCl₃) δ1.70-1.93 (4H, m), 2.16 (3H, s), 2.82-2.91 (1H, m), 3.07 (3H, s), 4.48 (2H, s), 6.30 (1H, s), 7.22 (2H, d), 7.30-7.35 (2H, m), 7.49 (2H, d), 7.60-7.63 (2H, m), 7.92 (2H, d), m/z 552 (M+H)⁺.

### EXAMPLE 63

### N-[5-[4-(4-cyanophenyl)piperidine-1-carbonyl]-2-methyl-phenyl]pentane-2-sulfonamide

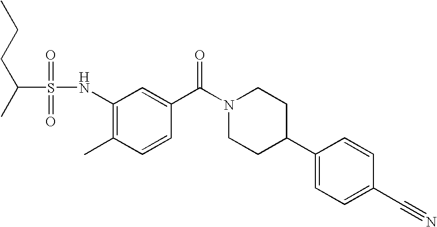

Prepared from Intermediate A

¹H NMR (300.072 MHz, CDCl₃) δ0.92 (3H, t), 1.39 (3H, d), 1.40-2.00 (8H, m), 2.34 (3H, s), 2.80-2.90 (1H, m), 3.13-3.23 (1H, m), 6.14 (1H, s), 7.15-7.18 (1H, m), 7.25 (1H, d), 7.33 (2H, d), 7.59 (2H, d), 7.63 (1H, s), m/z 454 (M+H)⁺.

### EXAMPLE 64

### N-[5-[4-(4-cyanophenyl)piperidine-1-carbonyl]-2-methyl-phenyl]-2-methyl-propane-1-sulfonamide

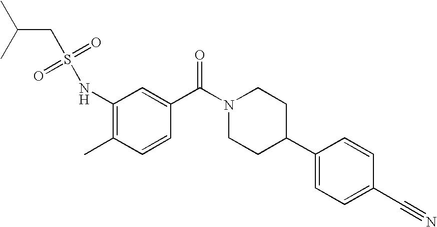

Prepared from Intermediate A

¹H NMR (300.072 MHz, CDCl₃) δ1.09 (6H, d), 1.60-2.00 (4H, m), 2.30 (1H, m), 2.32 (3H, s), 2.80-2.90 (1H, m), 3.02 (2H, d), 6.37 (1H, s), 7.18-7.21 (1H, m), 7.26 (1H, d), 7.32 (2H, d), 7.53 (1H, d), 7.60-7.63 (2H, m), m/z 440 (M+H)⁺.

### EXAMPLE 65

### N-[5-[4-(4-cyanophenyl)piperidine-1-carbonyl]-2-methyl-phenyl]-2-phenyl-ethanesulfonamide

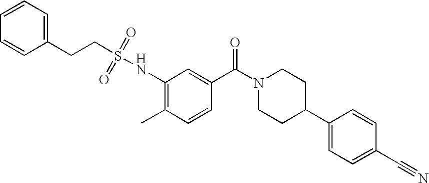

Prepared from Intermediate A

¹H NMR (300.072 MHz, CDCl₃) δ1.60-2.00 (4H, m), 2.25 (3H, s), 2.79-2.89 (1H, m), 3.14 (2H, t), 3.39-3.44 (2H, m), 6.33 (1H, s), 7.13-7.32 (8H, m), 7.50 (1H, d), 7.59-7.62 (2H, m), m/z 488 (M+H)⁺.

### EXAMPLE 66

### N-[3-[4-(4-cyanophenyl)piperidine-1-carbonyl]phenyl]-1-phenyl-methanesulfonamide

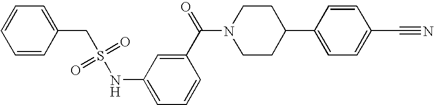

Prepared from Intermediate G

¹H NMR (300.073 MHz, DMSO-d₆, 30° C.) δ 1.53-1.94 (4H, m), 2.70-3.22 (3H, m), 3.50-3.85 (1H, m), 4.50 (2H, s), 4.53-4.73 (1H, m), 7.08-7.20 (2H, m), 7.21-7.45 (7H, m), 7.51 (2H, dJ=8.1 Hz), 7.77 (2H, dJ=8.5 Hz), 9.98 (1H, s), signals due to EtOAc also present

### EXAMPLE 67

### N-[5-[4-(4-cyanophenyl)piperidine-1-carbonyl]-2-methyl-phenyl]-3-morpholin-4-yl-propane-1-sulfonamide

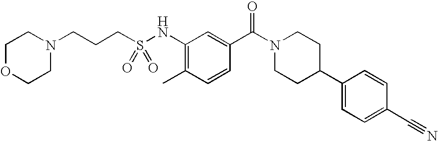

A stirred solution of in 3-chloro-N-[5-[4-(4-cyanophenyl)piperidine-1-carbonyl]-2-methyl-phenyl]propane-1-sulfonamide (Example 9, 150 mg, 0.33 mmol) in ethanol (2 mL) was treated with morpholine (86 μL, 0.98 mmol, 3 eq), and the reaction mixture heated in a microwave oven at temperatures from 80° C. to 120° C. until the reaction appeared to be complete by LCMS analysis. The reaction mixture was concentrated and purified by preparative HPLC (basic system) to give the title compound as a colourless solid, ¹H NMR (300.072 MHz, CDCl₃) δ1.71-1.90 (4H, m), 2.02 (3H, m), 2.26 (2H, s), 2.35 (3H, s), 2.44 (6H, m), 2.85 (1H, m), 3.28 (2H, t), 3.65 (4H, m), 7.18 (1H, d), 7.28 (1H, m), 7.32 (2H, d), 7.50 (1H, s), 7.61 (2H, d), m/z 511 (M+H)⁺.

### EXAMPLE 68

### N-[2-methyl-5-[4-(4-methylsulfonylphenyl)piperidine-1-carbonyl]phenyl]methanesulfonamide

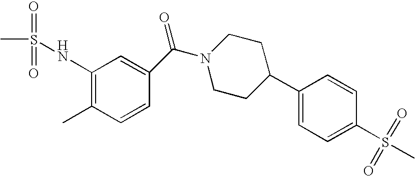

Prepared from Intermediate H

¹H NMR (300.073 MHz, DMSO-d₆) δ 1.44-1.98 (m, 4H), 2.33 (s, 3H), 2.82-3.12 (m, 6H), 3.18 (s, 3H), 3.57-3.97 (m, 1H), 4.39-4.79 (m, 1H), 7.16-7.25 (m, 1H), 7.28-7.37 (m, 2H), 7.57 (d, 2H), 7.85 (d, 2H), 9.16 (s, 1H), m/z 451 (M+H)⁺.

### EXAMPLE 69

### N-[3-[4-(4-cyanophenyl)piperidine-1-carbonyl]-4-methyl-phenyl]-1,1-dioxo-thiolane-3-sulfonamide

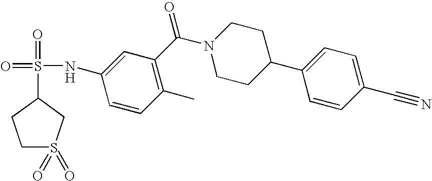

Prepared from Intermediate C

¹H NMR (300.072 MHz, CDCl₃) δ1.57-2.10 (m, 4H), 2.31 (d, 3H), 2.51-2.65 (m, 2H), 2.79-2.96 (m, 2H), 3.03-3.19 (m, 2H), 3.27-3.39 (m, 2H), 3.53-3.62 (m, 1H), 3.89-3.99 (m, 1H), 4.20-4.26 (m, 1H), 4.90-5.01 (m, 1H), 6.88-7.07 (m, 1H), 7.11-7.21 (m, 1H), 7.29-7.35 (m, 2H), 7.51-7.73 (m, 3H), 8.32-8.50 (m, 1H), m/z 502 (M+H)⁺.

### EXAMPLE 70

### N-[5-[4-(4-cyanophenyl)-4-hydroxy-piperidine-1-carbonyl]-2-methyl-phenyl]-1-phenyl-methanesulfonamide

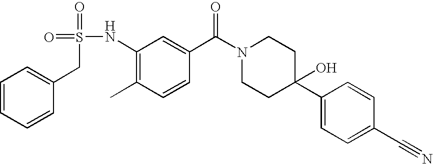

Prepared from Intermediate I

¹H NMR (300.072 MHz, CDCl₃) [symbol] 1.61-2.14 (m, 4H), 2.04 (s, 3H), 2.05 (s, 1H), 3.18-3.79 (m, 3H), 4.39 (s, 2H), 4.56-4.70 (m, 1H), 6.27 (s, 1H), 7.14-7.24 (m, 4H), 7.27-7.38 (m, 3H), 7.55-7.68 (m, 5H), m/z 490 (M+H)⁺.

### EXAMPLE 71

### N-[5-[4-(4-cyanophenyl)piperidine-1-carbonyl]-2-methyl-phenyl]-2-(1,3-dioxoisoindol-2-yl)ethanesulfonamide

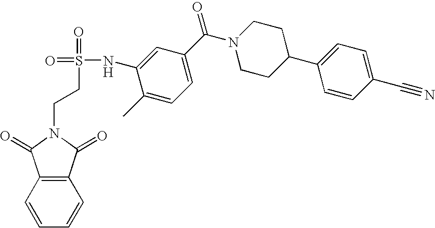

Prepared from Intermediate A

¹H NMR (300.072 MHz, CDCl₃) [symbol] 1.65-1.99 (m, 4H), 2.47 (s, 3H), 2.81-2.89 (m, 2H), 2.97-3.17 (m, 1H), 3.54 (t, 2H), 3.79-4.00 (m, 1H), 4.11 (t, 2H), 4.70-4.94 (m, 1H), 7.01 (s, 1H), 7.17 (d, 1H), 7.24-7.26 (m, 1H), 7.32 (d, 2H), 7.55 (s, 1H), 7.60 (d, 2H), 7.71-7.77 (m, 2H), 7.82-7.87 (m, 2H), m/z 557 (M+H)⁺.

### EXAMPLE 72

### 2-amino-N-[5-[4-(4-cyanophenyl)piperidine-1-carbonyl]-2-methyl-phenyl]ethanesulfonamide

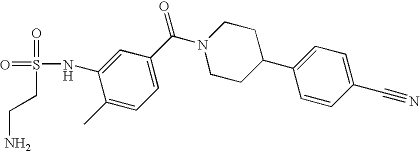

A solution of N-[5-[4-(4-cyanophenyl)piperidine-1-carbonyl]-2-methyl-phenyl]-2-(1,3-dioxoisoindol-2-yl)ethanesulfonamide (Example 71)(0.2 g, 0.36 mmol) in ethanol (6 ml) was treated with hydrazine monohydrate (0.07 mL, 1.44 mmol) and the reaction mixture heated at reflux for 1 hr. A white solid was removed by filtration and the filtrate reduced in vacuo; EtOAc (30 mL) and water (30 mL) were added to the fitrate and the resulting colourless solid was isolated by filtration to give the title compound as a colourless solid (0.1 g, 65%), ¹H NMR (300.073 MHz, DMSO-d₆) [symbol] 1.54-1.87 (m, 4H), 2.26 (s, 3H), 2.85-3.02 (m, 4H), 3.11 (m, 3H), 3.26-3.56 (m, 3H), 3.79 (s, 1H), 4.44-4.70 (m, 1H), 7.03 (d, 1H), 7.22 (d, 1H), 7.30 (s, 1H), 7.50 (d, 2H), 7.76 (d, 2H), m/z 427 (M+H)⁺.

### EXAMPLE 73

### N-[5-[4-(4-cyanophenyl)piperidine-1-carbonyl]-2-methyl-phenyl]-3-(propan-2-ylamino)propane-1-sulfonamide

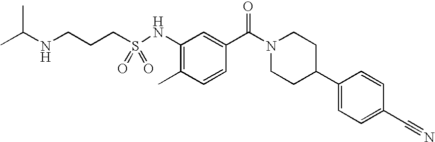

The title compound was prepared from 3-chloro-N-[5-[4-(4-cyanophenyl)piperidine-1-carbonyl]-2-methyl-phenyl]propane-1-sulfonamide (Example 9) by the method described in Example 67 (using THF as solvent), m/z 483 (M+H)⁺, Retention Time 1.96 min.

### EXAMPLE 74

### N-[5-[4-(4-cyanophenyl)piperidine-1-carbonyl]-2-methyl-phenyl]-3-dimethylamino-propane-1-sulfonamide

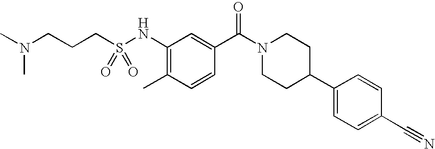

The title compound was prepared from 3-chloro-N-[5-[4-(4-cyanophenyl)piperidine-1-carbonyl]-2-methyl-phenyl]propane-1-sulfonamide (Example 9) by the method described in Example 67 (using THF as solvent), ¹H NMR (300.072 MHz, CDCl3) δ1.72-1.90 (4H, m), 2.01-2.07 (2H, m), 2.22 (6H, s), 2.34 (3H, s), 2.43 (2H, t), 2.81-2.89 (1H, m), 3.24 (2H, t), 7.12-7.24 (3H, m), 7.33 (2H, d), 7.48 (1H, s), 7.61 (2H, d), m/z 469 (M+H)⁺.

### EXAMPLE 75

### N-[5-[4-(4-cyanophenyl)piperidine-1-carbonyl]-2-methyl-phenyl]-3-methylamino-propane-1-sulfonamide

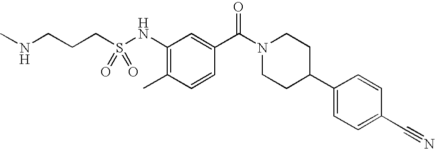

The title compound was prepared from 3-chloro-N-[5-[4-(4-cyanophenyl)piperidine-1-carbonyl]-2-methyl-phenyl]propane-1-sulfonamide (Example 9) by the method described in Example 67 (using THF as solvent), ¹H NMR (300.072 MHz, CDCl₃) δ1.80 (4H, m), 2.33 (2H, d), 2.40 (3H, s), 2.56 (3H, s), 2.82 (1H, m), 3.05 (2H, t), 3.38 (2H, t), 7.11-7.15 (2H, m), 7.23 (1H, d), 7.34 (2H, d), 7.51 (1H, d), 7.59 (2H, d), m/z 455 (M+H)⁺.

### EXAMPLE 76

### Methyl 3-[[5-[4-(4-cyanophenyl)piperidine-1-carbonyl]-2-methyl-phenyl]sulfamoyl]benzoate

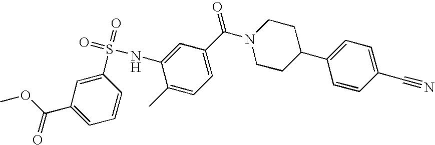

Prepared from Intermediate A

¹H NMR (300.072 MHz, CDCl₃) δ 1.63-1.96 (m, 4H), 2.02 (s, 3H), 2.80-3.13 (m, 3H), 3.92 (s, 3H), 3.92-3.96 (m, 1H), 4.66-4.94 (m, 1H), 6.53 (s, 1H), 7.14-7.19 (m, 1H), 7.21-7.24 (m, 1H), 7.34 (d, 2H), 7.38-7.41 (m, 1H), 7.53 (t, 1H), 7.64 (d, 2H), 7.88-7.91 (m, 1H), 8.20-8.22 (m, 1H), 8.40 (s, 1H), m/z 518 (M+H)⁺.

### EXAMPLE 77

### N-[5-[4-(4-cyanophenyl)piperidine-1-carbonyl]-2-methyl-phenyl]-2-methanesulfonamido-ethanesulfonamide

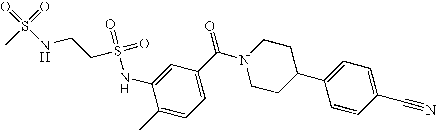

A solution of 2-amino-N-[5-[4-(4-cyanophenyl)piperidine-1-carbonyl]-2-methyl-phenyl]ethanesulfonamide (Example 72) (150 mg, 0.35 mmol) in pyridine (3 mL) was treated with methane sulfonyl chloride (0.04 mL, 0.53 mmol) and the reaction mixture stirred at ambient temperature for 24 hrs. It was then diluted with DCM (100 mL) and washed sequentially with dilute hydrochloric acid (100 mL of 1M), saturated sodium bicarbonate solution (100 mL) and brine (100 mL), dried (MgSO₄), filtered and evaporated in vacuo to give the crude product as a colourless oil. This was purified by chromatography on silica, eluting with 20-100% EtOAc in isohexane to give the title compound as a colourless solid (50 mg, 28%), ¹H NMR (300.072 MHz, CDCl₃) [symbol] 1.65-1.98 (m, 4H), 2.33 (s, 3H), 2.78-2.90 (m, 2H), 2.96 (s, 3H), 3.10-3.22 (m, 1H), 3.37 (t, 2H), 3.59-3.66 (m, 2H), 3.81-3.98 (m, 1H), 4.78-5.05 (m, 1H), 5.60 (t, 1H), 7.15-7.25 (m, 2H), 7.31-7.36 (m, 3H), 7.41-7.43 (m, 1H), 7.62 (d, 2H), m/z 505 (M+H)⁺.

### EXAMPLE 78

### N-[2-[[5-[4-(4-cyanophenyl)piperidine-1-carbonyl]-2-methyl-phenyl]sulfamoyl]ethyl]acetamide

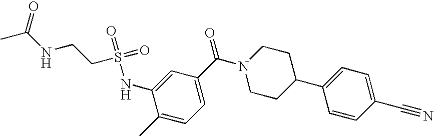

The title compound was prepared by the method described in Example 77 using acetyl chloride in place of methane sulfonyl chloride, ¹H NMR (300.072 MHz, CDCl₃) [symbol] 1.63-2.00 (m, 4H), 1.93 (s, 3H), 2.36 (s, 3H), 2.81-2.91 (m, 2H), 2.96-3.18 (m, 1H), 3.28 (t, 2H), 3.70 (q, 2H), 3.83-4.03 (m, 1H), 4.74-4.98 (m, 1H), 6.53 (t, 1H), 7.15-7.25 (m, 2H), 7.33 (d, 2H), 7.44 (d, 1H), 7.48 (s, 1H), 7.61 (d, 2H), m/z 469 (M+H)⁺.

### EXAMPLE 79

### 3-[[5-[4-(4-cyanophenyl)piperidine-1-carbonyl]-2-methyl-phenyl]sulfamoyl]benzoic acid

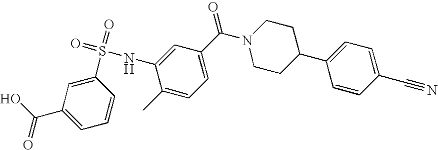

A solution of methyl 3-[[5-[4-(4-cyanophenyl)piperidine-1-carbonyl]-2-methyl-phenyl]sulfamoyl]benzoate (Example 76) (2.3 g, 4.44 mmol) in THF (24 mL) was treated with lithium hydroxide monohydrate (373 mg, 8.89 mmol) in water (12 mL) and the reaction mixture was stirred at ambient temperature for 20 hrs. The THF was evaporated in vacuo and the aqueous residue washed with EtOAc to remove any impurities. The aqueous portion was then adjusted to pH4 with citric acid solution and extracted with EtOAC. The extracts were combined and washed with brine, dried (MgSO₄), and evaporated in vacuo to give the title compound as a colourless solid (1.52 g, 68%), ¹H NMR (300.072 MHz, CDCl₃) [symbol] 1.57-1.89 (m, 4H), 2.09 (s, 3H), 2.72-2.93 (m, 2H), 3.02-3.20 (m, 1H), 3.69-4.01 (m, 1H), 4.63-4.92 (m, 1H), 5.14 (s, 1H), 7.13-7.22 (m, 2H), 7.25-7.28 (m, 2H), 7.31 (d, 2H), 7.51 (t, 1H), 7.60 (d, 2H), 7.89-7.94 (m, 1H), 8.16-8.21 (m, 1H), 8.44-8.46 (m, 1H), m/z 504 (M+H)⁺.

### EXAMPLE 80

### N-[2-[[5-[4-(4-cyanophenyl)piperidine-1-carbonyl]-2-methyl-phenyl]sulfamoyl]ethyl]propane-2-sulfonamide

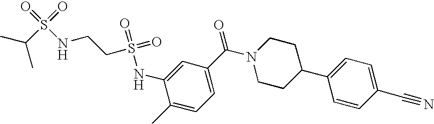

The title compound was prepared by the method described in Example 77, ¹H NMR (300.072 MHz, CDCl₃) δ1.34 (d, 6H), 1.69-1.99 (m, 4H), 2.33 (s, 3H), 2.79-2.92 (m, 2H), 3.06-3.19 (m, 2H), 3.34 (t, 2H), 3.62 (q, 2H), 3.77-3.94 (m, 1H), 4.79-4.96 (m, 1H), 5.40 (t, 1H), 7.16-7.23 (m, 2H), 7.34 (d, 2H), 7.40 (s, 2H), 7.61 (d, 2H), m/z 533 (M+H)⁺.

### EXAMPLE 81

### 3-[[5-[4-(4-cyanophenyl)piperidine-1-carbonyl]-2-methyl-phenyl]sulfamoyl]benzamide

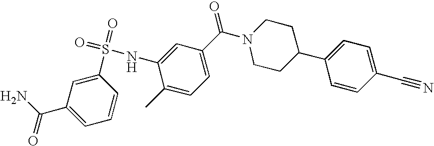

DIPEA (0.21 mL, 1.19 mmol) was added to a mixture of 3-[[5-[4-(4-cyanophenyl)piperidine-1-carbonyl]-2-methyl-phenyl]sulfamoyl]benzoic acid (Example 79) (0.15 g, 0.30 mmol), ammonia (1.49 mmol of a solution in dioxan) and HATU (0.24 g 0.63 mmol) in DMF (3 mL) and the reaction mixture stirred at ambient temperature for 72 hrs. EtOAc (30 mL) was added and the resulting solution was washed sequentially with water (30 mL) and brine (30 mL), dried (MgSO₄) and evaporated in vacuo to give the crude product as a brown oil which was purified by chromatography on silica, eluting with 0-10% MeOH in EtOAc to give the title compound as a colourless solid, ¹H NMR (300.073 MHz, DMSO-d₆) [symbol] 1.41-1.82 (m, 4H), 2.08 (s, 3H), 2.83-2.99 (m, 3H), 3.45-3.63 (m, 1H), 4.27-4.76 (m, 1H), 6.92 (s, 1H), 7.14-7.25 (m, 2H), 7.48 (d, 2H), 7.55 (s, 1H), 7.63 (t, 1H), 7.78 (d, 3H), 8.09 (d, 1H), 8.17 (d, 2H), 9.79 (s, 1H), m/z 503 (M+H)⁺.

### EXAMPLE 82

### 3-[[5-[4-(4-cyanophenyl)piperidine-1-carbonyl]-2-methyl-phenyl]sulfamoyl]-N,N-dimethyl-benzamide

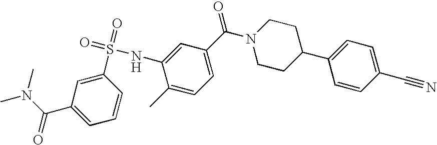

The title compound was prepared by the method described in Example 81, starting from 3-[[5-[4-(4-cyanophenyl)piperidine-1-carbonyl]-2-methyl-phenyl]sulfamoyl]benzoic acid (Example 79) and using dimethylamine in place of ammonia, ¹H NMR (300.072 MHz, CDCl₃) [symbol] 1.54-1.95 (m, 4H), 2.05 (s, 3H), 2.82-3.10 (m, 9H), 3.68-3.86 (m, 1H), 4.69-4.99 (m, 1H), 7.11-7.20 (m, 2H), 7.25-7.26 (m, 2H), 7.34 (d, 2H), 7.47 (t, 1H), 7.57-7.63 (m, 3H), 7.75-7.80 (m, 2H), m/z 531 (M+H)⁺.

### EXAMPLE 83

### 3-[[5-[4-(4-cyanophenyl)piperidine-1-carbonyl]-2-methyl-phenyl]sulfamoyl]-N-methyl-benzamide

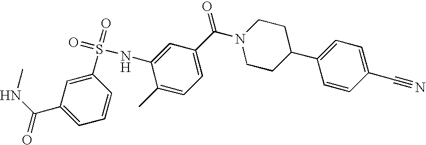

The title compound was prepared by the method described in Example 81, starting from 3-[[5-[4-(4-cyanophenyl)piperidine-1-carbonyl]-2-methyl-phenyl]sulfamoyl]benzoic acid (Example 79) and using methylamine in place of ammonia, ¹H NMR (300.073 MHz, DMSO-d₆) δ 1.44-1.80 (m, 4H), 2.06 (s, 3H), 2.74 (d, 3H), 2.81-2.97 (m, 3H), 3.38-3.64 (m, 1H), 4.35-4.61 (m, 1H), 6.92 (s, 1H), 7.15-7.23 (m, 2H), 7.47 (d, 2H), 7.63 (t, 1H), 7.78 (d, 3H), 8.04 (d, 1H), 8.15 (s, 1H), 8.62-8.64 (m, 1H), 9.80 (s, 1H), m/z 517 (M+H)⁺.

### EXAMPLE 84

### 3-[[5-[4-(4-cyanophenyl)piperidine-1-carbonyl]-2-methyl-phenyl]sulfamoyl]-N-(2-hydroxyethyl)benzamide

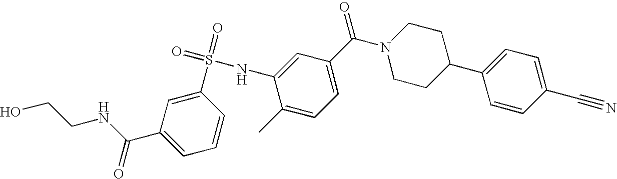

The title compound was prepared by the method described in Example 81, starting from 3-[[5-[4-(4-cyanophenyl)piperidine-1-carbonyl]-2-methyl-phenyl]sulfamoyl]benzoic acid (Example 79) and using ethanolamine in place of ammonia, ¹H NMR (300.072 MHz, CDCl₃) [symbol] 1.66-2.00 (m, 4H), 2.07 (s, 3H), 2.81-2.93 (m, 2H), 3.10-3.26 (m, 1H), 3.62 (q, 2H), 3.80 (q, 2H), 3.87-3.97 (m, 1H), 4.05 (t, 1H), 4.74-4.88 (m, 1H), 6.52 (s, 1H), 7.05 (d, 1H), 7.12 (d, 1H), 7.28-7.35 (m, 3H), 7.56-7.66 (m, 4H), 7.90 (s, 1H), 8.00 (d, 1H), 8.13 (d, 1H), m/z 547 (M+H)⁺.

### EXAMPLE 85

### 3-[[5-[4-(4-cyanophenyl)piperidine-1-carbonyl]-2-methyl-phenyl]sulfamoyl]-N-propan-2-yl-benzamide

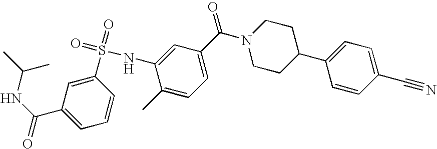

The title compound was prepared by the method described in Example 81, starting from 3-[[5-[4-(4-cyanophenyl)piperidine-1-carbonyl]-2-methyl-phenyl]sulfamoyl]benzoic acid (Example 79) and using isopropylamine in place of ammonia, ¹H NMR (300.073 MHz, DMSO-d₆) δ 1.11 (d, 6H), 1.40-1.86 (m, 4H), 2.07 (s, 3H), 2.84-3.05 (m, 3H), 3.43-3.64 (m, 1H), 4.04 (septet, 1H), 4.38-4.65 (m, 1H), 6.92 (s, 1H), 7.14-7.25 (m, 2H), 7.47 (d, 2H), 7.62 (t, 1H), 7.77 (d, 3H), 8.06 (d, 1H), 8.15 (s, 1H), 8.43 (d, 1H), 9.79 (s, 1H), m/z 545 (M+H)⁺.

### EXAMPLE 86

### N-[2-[[5-[4-(4-cyanophenyl)piperidine-1-carbonyl]-2-methyl-phenyl]sulfamoyl]ethyl]benzamide

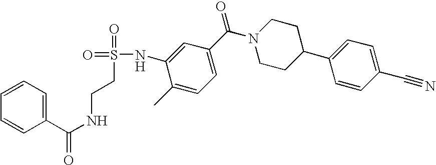

The title compound was prepared from the product of Example 72 by the method described in Example 77 using benzoyl chloride instead of methane sulfonyl chloride, ¹H NMR (300.072 MHz, CDCl₃) [symbol] 1.54-1.93 (m, 4H), 2.32 (s, 3H), 2.73-2.85 (m, 2H), 2.94-3.17 (m, 1H), 3.39 (t, 2H), 3.71-3.86 (m, 1H), 3.92 (q, 2H), 4.68-4.91 (m, 1H), 7.12-7.22 (m, 3H), 7.29 (d, 2H), 7.33-7.40 (m, 3H), 7.44-7.49 (m, 1H), 7.52 (s, 1H), 7.59 (d, 2H), 7.73 (d, 2H), m/z 531 (M+H)⁺.

### EXAMPLE 87

### N-[2-[[5-[4-(4-cyanophenyl)piperidine-1-carbonyl]-2-methyl-phenyl]sulfamoyl]ethyl]-2-methoxy-acetamide

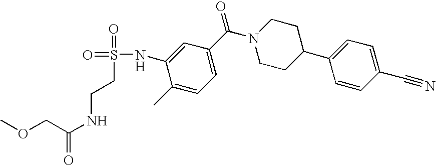

The title compound was prepared from the product of Example 72 by the method described in Example 77 using 2-methoxyacetyl chloride instead of methane sulfonyl chloride, ¹H NMR (300.072 MHz, CDCl₃) [symbol] 1.70-1.96 (m, 4H), 2.38 (s, 3H), 2.79-2.90 (m, 2H), 2.99-3.23 (m, 1H), 3.33 (t, 2H), 3.39 (s, 3H), 3.78 (q, 2H), 3.87 (s, 2H), 3.91-3.99 (m, 1H), 4.80-4.92 (m, 1H), 7.09 (t, 1H), 7.17-7.20 (m, 1H), 7.23 (d, 2H), 7.33 (d, 2H), 7.47 (d, 1H), 7.61 (d, 2H), m/z 499 (M+H)⁺.

### EXAMPLE 88

### N-[5-[4-(4-cyanophenyl)piperidine-1-carbonyl]-2-methyl-phenyl]-2-morpholin-4-yl-2-oxo-ethanesulfonamide

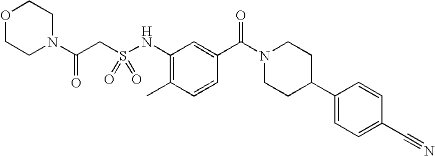

A solution of 2-[[5-[4-(4-cyanophenyl)piperidine-1-carbonyl]-2-methyl-phenyl]sulfamoyl]acetic acid (Intermediate J) (100 mg, 0.23 mmol) was stirred in DCM (4 mL) at ambient temperature under nitrogen and treated with 1-chloro-N,N, 2-trimethyl-1-propenylamine (0.039 mL, 0.29 mmol). The reaction mixture was stirred for 10 mins and then treated with morpholine (0.040 mL, 0.45 mmol) and pyridine (0.074 mL, 0.91 mmol), and the stirring continued for 1 h. The reaction mixture was then quenched with 0.5M HCl solution and stirred for a further 5 mins. The biphasic mixture was passed through a phase separation cartridge and the organic eluate concentrated in vacuo. The residue thus obtained was purified by chromatography on (12 g silica column, eluting with a gradient consisting of 50% EtOAc in isohexane to 10% MeOH in EtOAc); the compound was further purified by HPLC (acidic system) to give the title compounds as a colourless solid (21 mg, 18%), ¹H NMR (400.13 MHz, MeOD) δ 1.65-1.80 (4H, m), 2.32 (3H, s), 2.89 (1H, m), 3.53 (8H, m), 4.24 (2H, s), 7.16-7.18 (1H, m), 7.27 (1H, d), 7.39 (2H, d), 7.57 (1H, s), 7.57 (2H, d), m/z 511 (M+H)⁺.

### EXAMPLE 89

### N-[5-[4-(4-cyanophenyl)piperidine-1-carbonyl]-2-methoxy-phenyl]-1-phenyl-methanesulfonamide

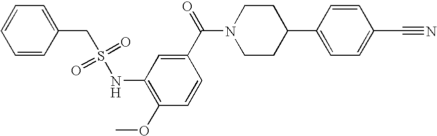

Prepared from Intermediate B

¹H NMR (300.073 MHz, DMSO-d₆, 30° C.) δ 1.52-1.71 (2H, m), 1.72-1.89 (2H, m), 2.76-3.20 (3H, m), 3.88 (3H, s), 4.44 (2H, s), [NB. Signals due to 2H, δ 3.5-5 approx. appear very broad], 7.08 (1H, dJ=9.0 Hz), 7.19-7.26 (2H, m), 7.27-7.35 (5H, m), 7.50 (2H, dJ=9.0 Hz), 7.77 (2H, dJ=9.0 Hz), 9.00 (1H, s), m/z 490 (M+H)⁺.

### EXAMPLE 90

### 6-chloro-N-[5-[4-(4-cyanophenyl)piperidine-1-carbonyl]-2-methyl-phenyl]pyridine-3-sulfonamide

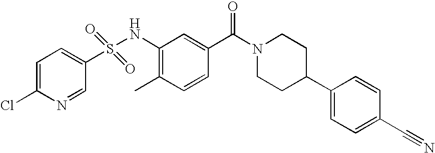

Prepared from Intermediate A

¹H NMR (300.073 MHz, DMSO-d₆, 30° C.) δ 1.39-1.95 (4H, m), 2.11 (3H, s), 2.68-3.14 (3H, m), 3.39-3.81 (1H, m appears as a broad singlet), 4.08-4.78 (1H, m appears as a broad singlet), 6.93 (1H, s), 7.18-7.33 (2H, m), 7.49 (2H, d J=7.0 Hz), 7.68-7.82 (3H, m), 8.01-8.10 (1H, m), 8.57-8.61 (1H, m), 10.08 (1H, s), m/z 493 (M−H)⁻ [A].

### EXAMPLE 91

### 2-[[5-[4-(4-cyanophenyl)piperidine-1-carbonyl]-2-methyl-phenyl]sulfamoyl]-N,N-dimethyl-acetamide

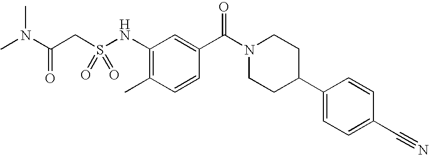

The title compound was prepared from Intermediate J by the method described in Example 88, ¹H NMR (300.072 MHz, CDCl₃) δ1.71-2.00 (4H, m), 2.44 (3H, s), 2.88 (1H, m), 3.02 (3H, s), 3.09 (3H, s), 4.10 (2H, s), 7.24-7.30 (2H, m), 7.33 (2H, d), 7.62 (1H, d), 7.60-7.67 (2H, m), m/z 469 (M+H)⁺.

### EXAMPLE 92

### 2-[[5-[4-(4-cyanophenyl)piperidine-1-carbonyl]-2-methyl-phenyl]sulfamoyl]-N-(2-hydroxyethyl)acetamide

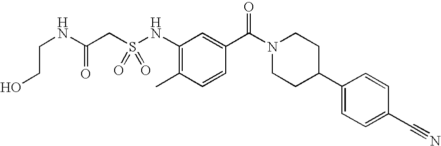

The title compound was prepared from Intermediate J by the method described in Example 88, ¹H NMR (300.072 MHz, CDCl₃) δ1.71-2.00 (4H, m), 2.40 (3H, s), 2.85 (1H, m), 3.29 (2H, t), 3.54 (2H, m), 4.00 (2H, s), 7.16-7.19 (2H, m), 7.34 (2H, d), 7.58-7.60 (2H, m), 7.63 (1H, s), m/z 485 (M+H)⁺.

### EXAMPLE 93

### N-[5-[4-(4-cyanophenyl)piperidine-1-carbonyl]-2-methyl-phenyl]-6-morpholin-4-yl-pyridine-3-sulfonamide

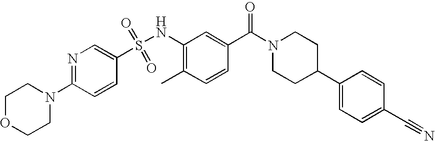

A stirred mixture of 6-chloro-N-[5-[4-(4-cyanophenyl)piperidine-1-carbonyl]-2-methyl-phenyl]pyridine-3-sulfonamide (Example 90)(50 mg, 0.1 mmol), triethylamine (0.07 ml, 0.51 mmol, 5 eq.) and morpholine (0.05 ml, 0.51 mmol, 5 eq.) in ethanol (2 mL) was heated in a Biotage Initiator microwave at 150° C. for 1 hr. The experiment was then repeated on the same scale and the reaction mixtures combined. The reaction solution was left to stand overnight and the material so formed was isolated by filtration to give the title compound (96.5 mg, 88%) as a crystalline solid, ¹H NMR (300.073 MHz, DMSO-d₆, 30° C.) δ 1.40-1.91 (4H, m), 2.13 (3H, s), 2.67-3.08 (3H, m), 3.36-3.70 (9H, m), 4.27-4.72 (1H, m appears as a broad singlet), 6.87 (1H, d J=9.0 Hz), 7.02 (1H, s), 7.15 (1H, d J=8.1 Hz), 7.23 (1H, d J=9.7 Hz), 7.49 (2H, d J=7.3 Hz), 7.63-7.70 (1H, m), 7.77 (2H, d J=9.0 Hz), 8.22-8.27 (1H, m), 9.41 (1H, s broad), m/z 546 (M+H)⁺.

### EXAMPLE 94

### N-[5-[4-(4-cyanophenyl)piperidine-1-carbonyl]-2-methyl-phenyl]-1-methyl-imidazole-4-sulfonamide

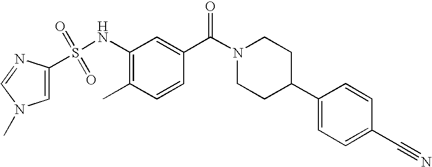

Prepared from Intermediate A

¹H NMR (300.073 MHz, DMSO-d₆, 30° C.) δ 1.44-1.94 (4H, m), 2.21 (3H, s), 2.70-3.20 (3H, m), 3.49-3.84 (4H, m), 4.34-4.73 (1H, m), 7.13 (1H, d J=6.7 Hz), 7.17-7.25 (2H, m), 7.49 (2H, d J=7.2 Hz), 7.61 (1H, s), 7.73-7.82 (3H, m), 9.48 (1H, s), m/z 464 (M+H)⁺.

### EXAMPLE 95

### N-[5-[4-(4-cyanophenyl)piperidine-1-carbonyl]-2-methyl-phenyl]-5-methyl-1,2-oxazole-4-sulfonamide

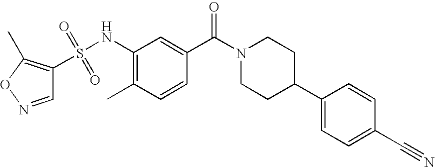

Prepared from Intermediate A

¹H NMR (300.073 MHz, DMSO-d₆, 30° C.) δ 1.48-2.02 (7H, m), 2.24 (3H, s), 2.66-3.25 (3H, m), 3.30-4.01 (1H, m), 4.38-4.80 (1H, m appears as a broad singlet), 7.02 (1H, d J=9.9 Hz), 7.19 (1H, d J=4.9 Hz), 7.31 (1H, s), 7.50 (2H, d J=7.8 Hz), 7.69-7.89 (3H, m doublet plus broad singlet), 8.63-8.85 (1H, m) [NB. Signals present due to water and MeOH], m/z 465 (M+H)⁺.

### EXAMPLE 96

### N-[5-[4-(4-cyanophenyl)piperidine-1-carbonyl]-2-methyl-phenyl]-6-dimethylamino-pyridine-3-sulfonamide

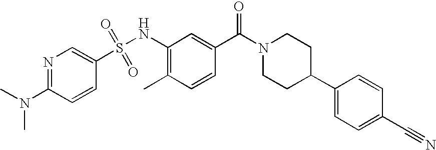

The title compound was prepared from the compound described in Example 90 by the method described in Example 2, ¹H NMR (300.073 MHz, DMSO-d₆, 30° C.) δ 1.30-1.93 (4H, m), 2.13 (3H, s), 2.65-3.11 (9H, m), 3.38-3.76 (1H, m appears as a broad singlet), 4.30-4.82 (1H, m appears as a broad singlet), 6.66 (1H, d J=9.9 Hz), 7.02 (1H, s), 7.15 (1H, d J=7.3 Hz), 7.23 (1H, d J=8.6 Hz), 7.49 (2H, d J=6.7 Hz), 7.62 (1H, d J=8.0 Hz), 7.78 (2H, d J=9.4 Hz), 8.21 (1H, s), 9.45 (1H, s), m/z 546 (M+H)⁺.

### EXAMPLE 97

### N-[5-[4-(4-cyanophenyl)piperidine-1-carbonyl]-2-methyl-phenyl]-3-(morpholine-4-carbonyl)benzenesulfonamide

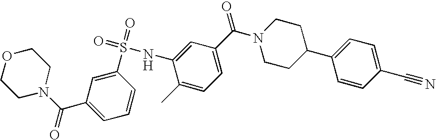

The title compound was prepared by the method described in Example 81, starting from 3-[[5-[4-(4-cyanophenyl)piperidine-1-carbonyl]-2-methyl-phenyl]sulfamoyl]benzoic acid (Example 79) and using morpholine in place of ammonia, ¹H NMR (300.072 MHz, CDCl₃) [symbol] 1.54-1.93 (m, 4H), 2.03 (s, 3H), 2.79-2.90 (m, 2H), 3.03-3.12 (m, 1H), 3.25-3.35 (m, 1H), 3.50-3.84 (m, 8H), 4.61-5.12 (m, 1H), 7.11-7.24 (m, 4H), 7.33 (d, 2H), 7.46-7.52 (m, 1H), 7.57-7.64 (m, 3H), 7.75-7.81 (m, 2H), m/z 573 (M+H)⁺.

### EXAMPLE 98

### N-[5-[4-(4-cyanophenyl)piperidine-1-carbonyl]-2-methyl-phenyl]ethenesulfonamide

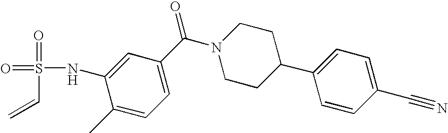

The title compound was prepared from Intermediate A by the method described in Example 1, using 2-chloroethanesulfonyl chloride. Elimination of HCl occurred during the work-up or purification procedures, ¹H NMR (300.072 MHz, CDCl₃) δ1.62-1.99 (m, 4H), 2.32 (s, 3H), 2.80-2.90 (m, 2H), 3.02-3.18 (m, 1H), 3.76-4.02 (m, 1H), 4.70-4.97 (m, 1H), 5.96 (d, 1H), 6.26 (d, 1H), 6.30 (s, 1H), 6.56-6.64 (m, 1H), 7.20-7.26 (m, 2H), 7.32 (d, 2H), 7.46 (s, 1H), 7.62 (d, 2H), m/z 410 (M+H)⁺.

### EXAMPLE 99

### N-[5-[4-(4-cyanophenyl)piperidine-1-carbonyl]-2-methyl-phenyl]-6-(propan-2-ylamino)pyridine-3-sulfonamide

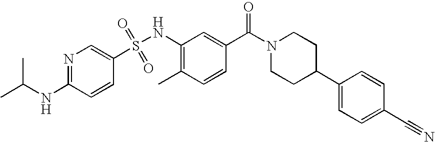

The title compound was prepared by the method described for Example 93, starting from 2-propylamine instead of morpholine. The crude product was purified by chromatography on silica (4 g column, eluting with a gradient consisting of 50-100% EtOAc in iso-hexane) to give the title compound as a colourless solid (88 mg, 85%), ¹H NMR (300.073 MHz, DMSO-d₆, 30° C.) δ 1.07 (6H, d J=7.0 Hz), 1.43-1.91 (4H, m), 2.13 (3H, s), 2.74-3.13 (3H, m), 3.39-3.84 (1H, m, appears as a very broad singlet), 3.86-4.08 (1H, m), 4.11-4.80 (1H, m, appears as a very broad singlet), 6.44 (1H, d J=9.5 Hz), 7.03 (1H, s), 7.16 (1H, d J=7.0 Hz), 7.23 (1H, d J=9.6 Hz), 7.29 (1H, d J=7.8 Hz), 7.77 (2H, d J=8.8 Hz), 7.44-7.54 (3H, m), 8.10-8.15 (1H, m), 9.38 (1H, s), m/z 518 (M+H)⁺.

### EXAMPLE 100

### 5-chloro-N-[5-[4-(4-cyanophenyl)piperidine-1-carbonyl]-2-methyl-phenyl]-1,3-dimethyl-pyrazole-4-sulfonamide

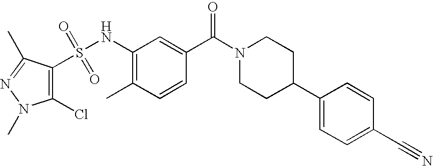

Prepared from Intermediate A

¹H NMR (300.073 MHz, DMSO-d₆, 30° C.) δ 1.44-1.92 (4H, m), 2.06 (3H, s), 2.12 (3H, s), 2.70-3.20 (3H, m), 3.50-3.79 (4H, m), 4.36-4.73 (1H, m appears as a broad flat singlet), 7.05 (1H, s), 7.17-7.28 (2H, m), 7.49 (2H, d J=7.6 Hz), 7.77 (2H, d J=6.9 Hz), 9.71 (1H, s), m/z 512 (M+H)⁺ [A].

### EXAMPLE 101

### 7-chloro-N-[5-[4-(4-cyanophenyl)piperidine-1-carbonyl]-2-methyl-phenyl]-4-thia-1,6-diazabicyclo[3.3.0]octa-2,5,7-triene-8-sulfonamide

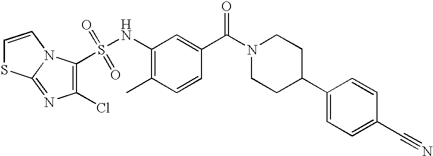

Prepared from Intermediate A

¹H NMR (300.073 MHz, DMSO-d₆, 30° C.) δ 1.41-1.93 (4H, m), 2.06 (3H, s), 2.66-3.15 (3H, m), 3.38-3.91 (1H, m appears as a broad singlet), 4.25-4.77 (1H, m appears as a broad singlet), 6.99 (1H, s), 7.17-7.29 (2H, m), 7.50 (2H, d J=12.0 Hz), 7.55-7.60 (1H, m), 7.71-7.83 (3H, m), 10.30 (1H, s), m/z 540 (M+H)⁺[A].

### EXAMPLE 102

### N-[5-[4-(4-cyanophenyl)piperidine-1-carbonyl]-2-methyl-phenyl]-6-oxo-1H-pyridine-3-sulfonamide

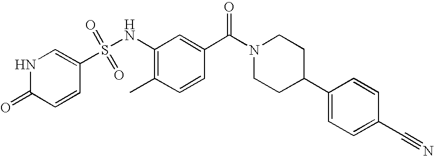

A mixture of 6-chloro-N-[5-[4-(4-cyanophenyl)piperidine-1-carbonyl]-2-methyl-phenyl]pyridine-3-sulfonamide (Example 90) (260 mg, 0.53 mmol) and potassium acetate (258 mg, 2.63 mmol) in a mixture of glacial acetic acid and water (2 mL of a 5:1 mixture) was heated in a Biotage Initiator microwave at 200° C. for 10 minutes. The bulk of the solvent was removed under reduced pressure and the residue purified by column chromatography (12 g silica cartridge, gradient eluting with 0-20% MeOH in DCM) to give the title compound as a colourless solid (42.1 mg, 17%), ¹H NMR (300.073 MHz, DMSO-d₆, 30° C.) δ 1.44-1.87 (4H, m), 2.19 (3H, s), 2.64-3.19 (3H, m), 3.39-3.83 (1H, m), 4.28-4.77 (1H, m), 6.45 (1H, d J=10.4 Hz), 7.03 (1H, s), 7.21 (1H, d J=8.5 Hz), 7.28 (1H, d J=8.5 Hz), 7.49 (2H, d J=7.8 Hz), 7.52-7.61 (2H, m), 7.77 (2H, d J=9.2 Hz), 9.62 (1H, s), 12.04 (1H, s), m/z 475 (M−H)⁻.

### EXAMPLE 103

### N-[5-[4-(4-cyanophenyl)piperidine-1-carbonyl]-2-methyl-phenyl]-6-(2-methoxyethylamino)pyridine-3-sulfonamide

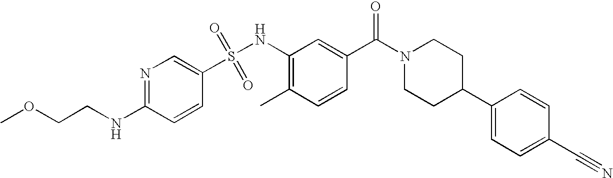

The title compound was prepared by the method described for Example 93, starting from 2-methoxyethylamine instead of morpholine. The crude product was purified by chromatography on silica (12 g column, eluting with a gradient consisting of 0-2.5% MeOH in DCM) to give the title compound as a colourless solid (170 mg, 80%), ¹H NMR (300.073 MHz, DMSO-d₆, 30° C.) δ 1.40-1.94 (4H, m), 2.13 (3H, s), 2.68-3.10 (3H, m), 3.22 (3H, s), 3.39 (4H, s), 3.44-3.84 (1H, m appears as a broad singley), 4.32-4.79 (1H, m appears as a broad singlet), 6.54 (1H, d J=9.2 Hz), 7.03 (1H, s), 7.16 (1H, d J=8.2 Hz), 7.23 (1H, d J=7.2 Hz), 7.43-7.55 (4H, m), 7.78 (2H, d J=7.7 Hz), 8.12 (1H, d J=2.5 Hz), 9.40 (1H, s), m/z 533 (M+H)⁺.

### EXAMPLE 104

### 4-[1-[3-(ethenylsulfonylamino)-4-methyl-benzoyl]-4-piperidyl]benzamide

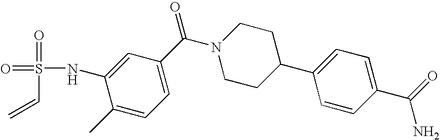

Benzyltrimethylammonium Hydroxide (Triton B, 40% solution in water) (1 mL) was added to a solution of N-[5-[4-(4-cyanophenyl)piperidine-1-carbonyl]-2-methyl-phenyl]ethenesulfonamide (Example 98) (0.1 g, 0.24 mmol) in THF (4 mL), and the reaction mixture stirred at ambient temperature for 20 hrs. The mixture was reduced in vacuo and EtOAc (20 mL) and aqueous citric acid (20 mL of a 1M solution) was added to the residue. The phases were separated and the organic portion washed with brine (20 mL), dried (MgSO₄), filtered and reduced in vacuo to give a colourless solid. This was purified by chromatography on silica, eluting with a gradient 0-10% MeOH in EtOAc to give a the title compound as a colourless solid, ¹H NMR (300.072 MHz, CDCl₃) [symbol] 1.60-1.91 (m, 4H), 2.33 (s, 3H), 2.81-2.89 (m, 2H), 3.02-3.25 (m, 1H), 3.81-4.00 (m, 1H), 4.70-4.93 (m, 1H), 5.94 (d, 1H), 6.06 (s, 2H), 6.23 (d, 1H), 6.56-6.65 (m, 1H), 6.77 (s, 1H), 7.21-7.24 (m, 2H), 7.29 (d, 2H), 7.44 (s, 1H), 7.77 (d, 2H), m/z 428 (M+H)⁺.

### EXAMPLE 105

### N-[5-[4-(4-cyanophenyl)piperidine-1-carbonyl]-2-methyl-phenyl]-2-methoxy-ethanesulfonamide

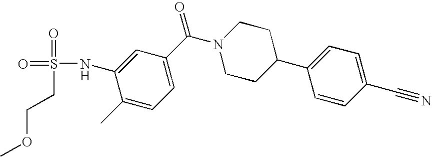

Sodium methoxide (0.5M in MeOH) (4 ml, 2 mmol) was added to a solution of N-[5-[4-(4-cyanophenyl)piperidine-1-carbonyl]-2-methyl-phenyl]ethenesulfonamide (Example 98) (0.1 g, 0.24 mmol) in THF (4 ml). The reaction mixture was stirred at ambient temperature for 20 hrs. More sodium methoxide (0.1 g, 1.85 mmol) was added and the reaction stirred for a further 5 days. The mixture was reduced in vacuo and EtOAc (20 mL) and aqueous citric acid (20 mL of a 1M solution) was added to the residue. The phases were separated and the organic portion washed with brine (20 mL), dried (MgSO₄), filtered and reduced in vacuo to give a colourless solid. This was purified by chromatography on silica, eluting with a gradient of 10-80% EtOAc in isohexane to give a the title compound as a colourless solid, ¹H NMR (300.072 MHz, CDCl₃) [symbol] 1.65-1.97 (m, 4H), 2.34 (s, 3H), 2.80-2.89 (m, 2H), 2.99-3.16 (m, 1H), 3.35 (t, 2H), 3.38 (s, 3H), 3.85 (t, 2H), 3.89-4.03 (m, 1H), 4.73-5.01 (m, 1H), 6.47 (s, 1H), 7.18-7.27 (m, 2H), 7.33 (d, 2H), 7.58-7.64 (m, 3H), m/z 442 (M+H)⁺.

### EXAMPLE 106

### N-[5-[4-(4-cyanophenyl)piperidine-1-carbonyl]-2-methyl-phenyl]-2,4-dimethyl-1,3-thiazole-5-sulfonamide

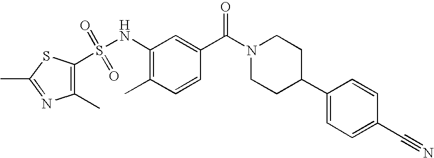

Prepared from Intermediate A

¹H NMR (300.073 MHz, DMSO-d₆, 30° C.) [symbol] 1.45-1.68 (2H, m), 1.69-1.92 (2H, m), 2.11 (3H, s), 2.17 (3H, s), 2.56 (3H, s), 2.67-3.19 (3H, m), 3.45-3.86 (1H, m appears as a broad singlet), 4.30-4.74 (1H, m appears as a broad singlet), 7.06 (1H, s), 7.21-7.32 (2H, m), 7.50 (2H, d J=8.4 Hz), 7.78 (2H, d J=6.0 Hz), 10.06 (1H, s), m/z 495 (M+H)⁺.

### EXAMPLE 107

### N-[5-[4-(4-cyanophenyl)piperidine-1-carbonyl]-2-methyl-phenyl]pyridine-3-sulfonamide

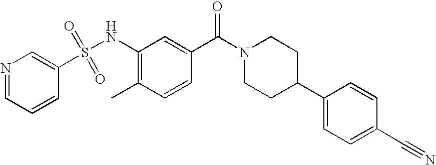

A solution of 6-chloro-N-[5-[4-(4-cyanophenyl)piperidine-1-carbonyl]-2-methyl-phenyl]pyridine-3-sulfonamide (Example 90) (200 mg, 0.40 mmol) in a mixture of THF and MeOH (10 mL of a 3:1 mixture) was stirred in an atmosphere of hydrogen with palladium on charcoal catalyst (50 mg of 10% Pd/C). A small quantity of triethylamine was added, the catalyst was changed several times, and the reaction stirred for 2 days. The catalyst was removes by filtration, and the filtrate evaporated under reduced pressure. The residue was purified by HPLC (basic system) to give the title compound as a colourless solid (20 mg, 11%), ¹H NMR (300.072 MHz, CDCl₃, 30° C.) δ 1.49-2.13 (7H, m), 2.69-3.26 (3H, m), 3.52-5.06 (2H, m), 7.10-7.24 (3H, m), 7.30-7.43 (3H, m), 7.63 (2H, d J=8.5 Hz), 7.98-8.07 (1H, m), 8.72-8.77 (1H, m), 8.87-8.91 (1H, m), m/z 461 (M+H)⁺.

### EXAMPLE 108

### methyl 2-[[5-[4-(4-cyanophenyl)piperidine-1-carbonyl]-2-methyl-phenyl]sulfamoyl]benzoate

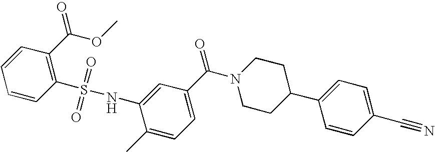

Prepared from Intermediate A

¹H NMR (300.072 MHz, CDCl₃) [symbol] 1.57-1.97 (m, 4H), 2.18 (s, 3H), 2.76-2.88 (m, 2H), 2.93-3.13 (m, 1H), 3.75-3.98 (m, 1H), 4.05 (s, 3H), 4.46-5.06 (m, 1H), 7.13-7.21 (m, 2H), 7.33 (d, 2H), 7.39 (s, 1H), 7.46-7.53 (m, 1H), 7.57-7.65 (m, 3H), 7.80-7.85 (m, 2H), 8.00 (s, 1H), m/z 518 (M+H)⁺.

### EXAMPLE 109

### N-[5-[4-(4-cyanophenyl)piperidine-1-carbonyl]-2-methyl-phenyl]-2-hydroxy-ethanesulfonamide

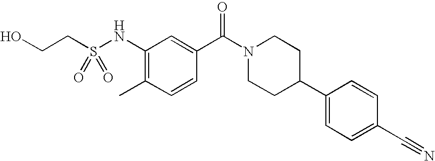

A solution of methyl 2-[[5-[4-(4-cyanophenyl)piperidine-1-carbonyl]-2-methyl-phenyl]sulfamoyl]acetate (Example 13) in THF (5 mL) was placed under an atmosphere of nitrogen and treated with lithium borohydride (0.44 mL of a 2M in THF, 0.88 mmol), and the reaction was stirred at ambient temperature for 1 hr. The mixture was reduced in vacuo and EtOAc (20 mL) and water (20 mL) was added to the residue. The phases were separated and the organic portion washed with brine (20 mL), dried (MgSO₄), filtered and reduced in vacuo to give a colourless solid. This was purified by chromatography on silica, eluting with a gradient of 20-100% EtOAc in isohexane to give a the title compound as a colourless solid (56 mg, 30%), ¹H NMR (300.072 MHz, CDCl₃) [symbol] 1.60-1.98 (m, 4H), 2.32 (s, 3H), 2.82-2.90 (m, 2H), 2.99-3.16 (m, 1H), 3.26 (t, 1H), 3.31 (t, 2H), 3.87-3.99 (m, 1H), 4.07 (q, 2H), 4.75-4.94 (m, 1H), 6.97 (s, 1H), 7.17-7.23 (m, 2H), 7.32 (d, 2H), 7.50-7.51 (m, 1H), 7.61 (d, 2H), m/z 428 (M+H)⁺.

### EXAMPLE 110

### N-[5-[4-(4-cyanophenyl)piperidine-1-carbonyl]-2-methyl-phenyl]-3-methoxy-propane-1-sulfonamide

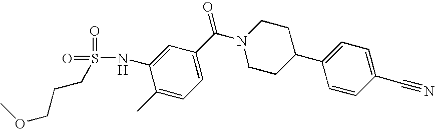

A mixture of tert-butyl N-(3-chloropropylsulfonyl)-N-[3-[4-(4-cyanophenyl)piperidine-1-carbonyl]phenyl]carbamate (Intermediate K) (0.2 g, 0.36 mmol), 1-butyl-3-methylimidazolium tetrafluoroborate (2.5 mL) and MeOH (2.5 mL) was heated in the microwave at 150° C. for 90 mins and then a further 20 hrs. The mixture was reduced in vacuo and EtOAc (30 mL) was added to the residue. The mixture was washed sequentially with saturated sodium bicarbonate solution (20 mL), water (20 mL) and brine (20 mL), then dried (MgSO₄), filtered and reduced in vacuo to give a colourless oil. This was purified by chromatography on silica, eluting with a gradient of 20-100% EtOAc in isohexane to give a the title compound as a colourless solid (23 mg, 14%), ¹H NMR (300.072 MHz, CDCl₃) [symbol] 1.63-1.96 (m, 4H), 2.09 (quintet, 2H), 2.34 (s, 3H), 2.79-2.92 (m, 2H), 3.01-3.12 (m, 1H), 3.23-3.27 (m, 2H), 3.29 (s, 3H), 3.47 (t, 2H), 3.79-4.02 (m, 1H), 4.72-4.91 (m, 1H), 6.33 (s, 1H), 7.18-7.28 (m, 2H), 7.32 (d, 2H), 7.53 (s, 1H), 7.61 (d, 2H), m/z 456 (M+H)⁺.

### EXAMPLE 111

### N-[5-[4-(4-cyanophenyl)piperidine-1-carbonyl]-2-methyl-phenyl]-2-(morpholine-4-carbonyl)benzenesulfonamide

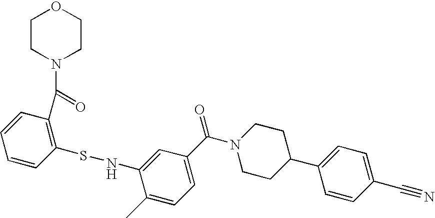

The title compound was prepared by the method described in Example 81, starting from 2-[[5-[4-(4-cyanophenyl)piperidine-1-carbonyl]-2-methyl-phenyl]sulfamoyl]benzoic acid (Intermediate L) and using morpholine in place of ammonia, ¹H NMR (300.072 MHz, CDCl₃) [symbol] 1.68-1.94 (m, 4H), 2.16 (s, 3H), 2.76-2.91 (m, 2H), 2.94-3.09 (m, 1H), 3.32-3.41 (m, 2H), 3.50-3.67 (m, 2H), 3.72-3.88 (m, 3H), 3.92-4.00 (m, 1H), 4.15-4.22 (m, 1H), 4.67-4.99 (m, 1H), 7.13-7.22 (m, 2H), 7.30-7.43 (m, 5H), 7.55-7.68 (m, 5H), m/z 456 (M+H)⁺.

### EXAMPLE 112

### 4-[1-(3-methanesulfonamido-4-methyl-benzoyl)-4-piperidyl]benzamide

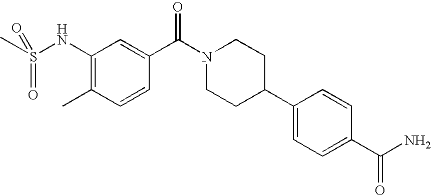

The title compound was prepared by the method described in Example 1, starting from Intermediate M, ¹H NMR (300.073 MHz, DMSO-d₆) δ 1.44-1.88 (m, 4H), 2.33 (s, 3H), 2.56-3.08 (m, 6H), 3.56-3.92 (m, 1H), 4.44-4.74 (m, 1H), 7.19-7.25 (m, 2H), 7.30-7.37 (m, 4H), 7.80 (d, J=8.2 Hz, 2H), 7.86 (s, 1H), 9.15 (s, 1H), m/z 414 (M−H)⁻.

### EXAMPLE 113

### 2-[[5-[4-(4-cyanophenyl)piperidine-1-carbonyl]-2-methyl-phenyl]sulfamoyl]benzamide

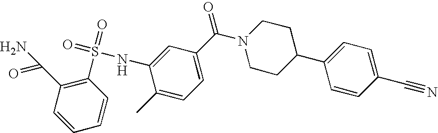

A solution of 2-[[5-[4-(4-cyanophenyl)piperidine-1-carbonyl]-2-methyl-phenyl]sulfamoyl]-N-[(2,4-dimethoxyphenyl)methyl]benzamide (Intermediate N) (81 mg, 0.12 mmol) and thioanisole (0.07 mL, 0.62 mmol) in DCM (0.5 mL) was treated with trifluoroacetic acid (0.05 mL, 0.62 mmol) and the reaction mixture was stirred at ambient temperature for 20 hrs. Further trifluoroacetic acid (2 mL) was added and the reaction heated at 60° C. for 1 hr. The reaction mixture was then reduced in vacuo and EtOAc (30 mL) added; the mixture was washed sequentially with saturated sodium bicarbonate solution (30 mL) and brine (30 mL), then dried (MgSO₄), filtered and reduced in vacuo to give a colourless solid. This was purified by chromatography on silica, eluting with a gradient of 0-8% MeOH in DCM to give the title compound as a colourless solid (21 mg, 35%), ¹H NMR (300.072 MHz, CDCl₃) [symbol] 1.59-1.94 (m, 4H), 2.18 (s, 3H), 2.76-2.90 (m, 2H), 2.95-3.06 (m, 1H), 3.81-4.03 (m, 1H), 4.64-5.01 (m, 1H), 6.02 (s, 1H), 6.22 (s, 1H), 7.13-7.19 (m, 2H), 7.30-7.36 (m, 3H), 7.46 (t, 1H), 7.54-7.67 (m, 4H), 7.74 (d, 1H), 8.21 (s, 1H), m/z 503 (M+H)⁺.

### EXAMPLE 114

### N-[5-[4-(4-cyanophenyl)piperidine-1-carbonyl]-2-methyl-phenyl]-2,3-dimethyl-imidazole-4-sulfonamide

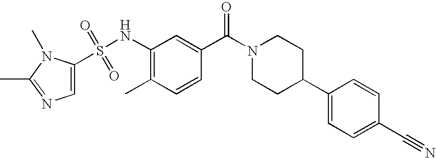

Prepared from Intermediate A

¹H NMR (300.073 MHz, DMSO-d₆, 30° C.) δ 1.41-1.93 (4H, m), 2.15 (3H, s), 2.30 (3H, s), 2.64-3.18 (3H, m), 3.40-3.87 (4H, m), 4.21-4.79 (1H, m), 6.99 (1H, s), 7.11 (1H, s), 7.20 (1H, dJ=8.3 Hz), 7.27 (1H, dJ=8.9 Hz), 7.50 (2H, dJ=7.7 Hz), 7.77 (2H, dJ=7.7 Hz), 9.86 (1H, s), m/z 478 (M+H)⁺.

### EXAMPLE 115

### N-[5-[4-(4-cyanophenyl)piperidine-1-carbonyl]-2-methyl-phenyl]piperidine-4-sulfonamide

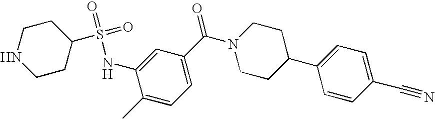

A solution of tert-butyl N-[5-[4-(4-cyanophenyl)piperidine-1-carbonyl]-2-methyl-phenyl]-N-(4-piperidylsulfonyl)carbamate (Intermediate O) (2.1 g, 3.7 mmol) in DCM (20 mL) was treated a solution of hydrogen chloride in dioxan (40 mL of 4M solution) and the reaction mixture was stirred at ambient temperature for 16 hrs. The reaction mixture was evaporated in vacuo and the residue azeotroped twice with chloroform to give the hydrochloride of the title compound as a colourless foam which was used without further purification, ¹H NMR (300.073 MHz, DMSO-d₆) δ 1.61-2.00 (6H, m), 2.17 (2H, d), 2.35 (4H, s), 2.75-3.20 (5H, m), 3.40-3.85 (4H, m), 4.59-4.65 (1H, m), 7.21 (1H, d), 7.31 (1H, d), 7.37 (1H, s), 7.51 (2H, d), 7.76-7.79 (2H, m), 8.85 (1H, m), 9.25 (1H, d), 9.41 (1H, s), m/z 467 (M+H)⁺.

### EXAMPLE 116

### N-[5-[4-(4-cyanophenyl)piperidine-1-carbonyl]-2,4-dimethyl-phenyl]cyclopropanesulfonamide

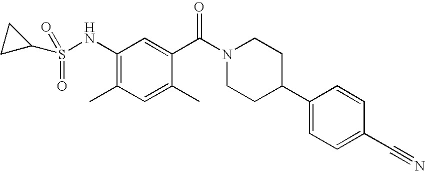

Prepared from Intermediate D

¹H NMR (300.073 MHz, DMSO-d₆) δ 0.72-0.99 (m, 4H), 1.31-1.94 (m, 4H), 2.09-2.28 (m, 3H), 2.31 (s, 3H), 2.52-2.63 (m, 1H), 2.75-2.99 (m, 2H), 3.04-3.19 (m, 1H), 3.34-3.50 (m, 1H), 4.59-4.76 (m, 1H), 6.98-7.19 (m, 2H), 7.47 (d, 2H), 7.76 (d, 2H), 9.07 (s, 1H), m/z 438 (M+H)⁺.

### EXAMPLE 117

### N-[5-[4-(4-cyanophenyl)piperidine-1-carbonyl]-2,4-dimethyl-phenyl]propane-1-sulfonamide

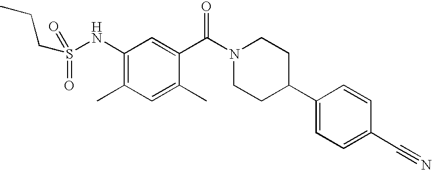

Prepared from Intermediate D

¹H NMR (300.073 MHz, DMSO-d₆) δ 0.82-1.04 (m, 3H), 1.29-1.95 (m, 6H), 2.09-2.24 (m, 3H), 2.28 (s, 3H), 2.75-3.18 (m, 5H), 3.35-3.50 (m, 1H), 4.58-4.76 (m, 1H), 6.96-7.19 (m, 2H), 7.47 (d, 2H), 7.76 (d, 2H), 9.03 (s, 1H), m/z 440 (M+H)⁺.

### EXAMPLE 118

### 1-acetyl-N-[5-[4-(4-cyanophenyl)piperidine-1-carbonyl]-2-methyl-phenyl]piperidine-4-sulfonamide

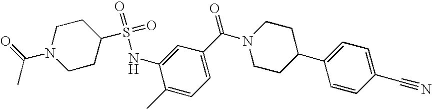

The title compound was prepared by the method described in Example 77, starting from N-[5-[4-(4-cyanophenyl)piperidine-1-carbonyl]-2-methyl-phenyl]piperidine-4-sulfonamide (Example 115) and using acetyl chloride in place of methane sulfonyl chloride, ¹H NMR (300.072 MHz, CDCl₃) δ 1.55-2.05 (6H, m), 2.04-2.25 (5H, m), 2.34 (3H, s), 2.52-2.61 (1H, m), 2.81-2.90 (2H, m), 3.06 (1H, d), 3.11 (1H, s), 3.22-3.32 (1H, m), 3.93 (2H, d), 4.66-5.00 (2H, m), 7.15 (1H, s), 7.13-7.16 (1H, m), 7.22 (1H, d), 7.32-7.34 (2H, m), 7.49 (1H, d), 7.60-7.63 (2H, m), m/z 509 (M+H)⁺.

### EXAMPLE 119

### N-[5-[4-(4-cyanophenyl)piperidine-1-carbonyl]-2-methyl-phenyl]-1-propan-2-ylsulfonyl-piperidine-4-sulfonamide

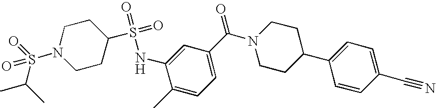

The title compound was prepared by the method described in Example 77, starting from N-[5-[4-(4-cyanophenyl)piperidine-1-carbonyl]-2-methyl-phenyl]piperidine-4-sulfonamide (Example 115) and propane-2-sulfonyl chloride, ¹H NMR (300.072 MHz, CDCl₃) δ 1.29 (6H, q), 1.55-2.00 (6H, s), 2.12-2.17 (2H, m), 2.33 (3H, s), 2.83-2.91 (3H, m), 2.94-2.95 (1H, m), 3.09-3.20 (2H, m), 3.89-3.96 (4H, m), 4.85 (1H, s), 7.06 (1H, s), 7.13-7.17 (1H, m), 7.21-7.23 (1H, m), 7.32-7.34 (2H, m), 7.43 (1H, d), 7.60-7.63 (2H, m), m/z 573 (M+H)⁺.

### EXAMPLE 120

### N-[5-[4-(4-cyanophenyl)piperidine-1-carbonyl]-2-methyl-phenyl]-1-propan-2-yl-piperidine-4-sulfonamide

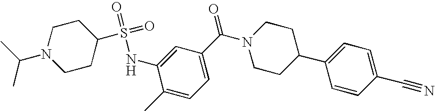

A solution of N-[5-[4-(4-cyanophenyl)piperidine-1-carbonyl]-2-methyl-phenyl]piperidine-4-sulfonamide (Example 115) (200 mg, 0.4 mmol) in THF (2 mL) and DMF (2 mL) was treated with acetone (0.073 mL, 0.99 mmol) and MacroPorous cationic polystyrene resin, with cyanoborohydride as the counterion (2.07 mmol/g, 483 mg, 0.99 mmol), and the reaction mixture was stirred at ambient temperature for 20 hrs. More MP cyanoborohydride (500 mg) was added and the reaction stirred at ambient temperature for a further 20 hrs. The reaction mixture was then diluted with EtOAc (20 mL) and the resulting mixture was washed sequentially with saturated sodium bicarbonate solution, water (3×) and brine, then dried (MgSO₄), filtered and reduced in vacuo to give a colourless foam. This was purified by chromatography on silica, eluting with a gradient of 0-20% MeOH in DCM, to give the title compound as a colourless foam, ¹H NMR (300.072 MHz, CDCl₃) δ 1.11 (6H, d), 1.50-2.50 (13H, m), 2.84-2.87 (2H, m), 2.94 (1H, d), 3.03 (1H, d), 3.09-3.14 (3H, m), 3.90 (1H, m), 4.85 (1H, m), 7.14-7.17 (1H, m), 7.24 (1H, d), 7.33 (2H, d), 7.53 (1H, s), 7.60-7.62 (2H, m), m/z 509 (M+H)⁺.

### EXAMPLE 121

### N-[5-[4-(4-cyanophenyl)piperidine-1-carbonyl]-2-methyl-phenyl]-1-methylsulfonyl-piperidine-4-sulfonamide

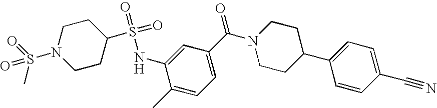

The title compound was prepared by the method described in Example 77, starting from N-[5-[4-(4-cyanophenyl)piperidine-1-carbonyl]-2-methyl-phenyl]piperidine-4-sulfonamide (Example 115) and methane sulfonyl chloride, ¹H NMR (300.072 MHz, CDCl₃) δ 1.60-2.05 (6H, d), 2.17-2.23 (2H, m), 2.33 (3H, s), 2.79 (5H, m), 2.71-2.90 (2H, m), 2.80-2.91 (1H, m), 3.14-3.23 (2H, m), 3.87-3.91 (3H, m), 4.85 (1H, m), 6.65 (1H, s), 7.14-7.17 (1H, m), 7.24 (1H, d), 7.32 (2H, d), 7.48 (1H, d), 7.60-7.63 (2H, m), m/z 545 (M+H)⁺.

### EXAMPLE 122

### N-[5-[4-(4-cyanophenyl)piperidine-1-carbonyl]-2-methyl-phenyl]-1-ethylsulfonyl-piperidine-4-sulfonamide

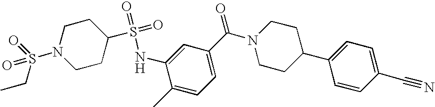

The title compound was prepared by the method described in Example 77, starting from N-[5-[4-(4-cyanophenyl)piperidine-1-carbonyl]-2-methyl-phenyl]piperidine-4-sulfonamide (Example 115) and ethane sulfonyl chloride, ¹H NMR (300.072 MHz, CDCl₃) δ 1.29-1.36 (6H, m), 1.50-2.00 (6H, m), 2.17 (2H, d), 2.33 (3H, s), 2.81-3.25 (8H, m), 3.63-4.00 (3H, m), 4.84 (1H, m), 7.13-7.22 (3H, t), 7.33 (2H, d), 7.43 (1H, s), 7.61 (2H, d), m/z 559 (M+H)⁺.

### EXAMPLE 123

### N-[5-[4-(4-cyanophenyl)piperidine-1-carbonyl]-2-ethyl-phenyl]methanesulfonamide

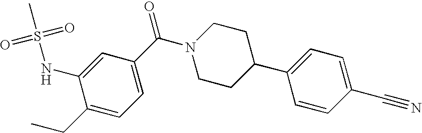

Prepared from Intermediate P

¹H NMR (300.073 MHz, DMSO-d₆, 30° C.) δ 1.17 (3H, tJ=7.4 Hz), 1.54-1.94 (4H, m), 2.74 (2H, qJ=7.4 Hz), 2.80-3.23 (6H, m), 3.58-3.88 (1H, m), 4.40-4.77 (1H, m), 7.27 (1H, dJ=8.2 Hz), 7.31-7.37 (2H, m), 7.50 (2H, dJ=7.6 Hz), 7.77 (2H, dJ=7.6 Hz), 9.14 (1H, s), m/z 412 (M+H)⁺.

### EXAMPLE 124

### N-[5-[4-(4-cyanophenyl)piperidine-1-carbonyl]-2-ethyl-phenyl]-1,1-dioxo-thiolane-3-sulfonamide

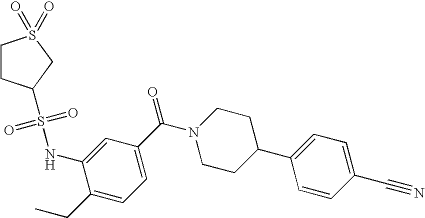

Prepared from Intermediate P

¹H NMR (300.072 MHz, CDCl₃, 30° C.) δ 1.23 (3H, tJ=7.0 Hz), 1.48-2.06 (4H, m), 2.46-2.73 (4H, m), 2.74-3.26 (4H, m), 3.28-3.49 (3H, m), 3.72-4.07 (2H, m), 4.70-5.01 (1H, m), 7.16-7.29 (3H, m), 7.34 (2H, dJ=8.3 Hz), 7.62 (2H, dJ=7.7 Hz), 8.10 (1H, s), m/z 516 (M+H)⁺.

### EXAMPLE 125

### N-[5-[4-(4-cyanophenyl)piperidine-1-carbonyl]-2-methyl-phenyl]-1-methyl-piperidine-4-sulfonamide

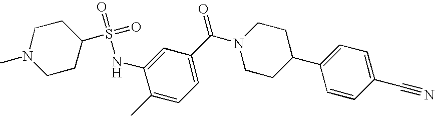

The title compound was prepared by the method described in Example 120, starting from N-[5-[4-(4-cyanophenyl)piperidine-1-carbonyl]-2-methyl-phenyl]piperidine-4-sulfonamide (Example 115) and using formaldehyde in place of acetone, ¹H NMR (300.072 MHz, CDCl₃) δ 1.50-2.10 (10H, m), 2.25 (3H, s), 2.33 (3H, s), 2.80-3.25 (6H, m), 3.89-3.95 (1H, m), 4.87 (1H, s), 7.15-7.18 (1H, m), 7.22-7.25 (2H, m), 7.31 (2H, d), 7.56-7.60 (1H, m), 7.62 (2H, d), m/z 481 (M+H)⁺.

### EXAMPLE 126

### 3-amino-N-[5-[4-(4-cyanophenyl)piperidine-1-carbonyl]-2-methyl-phenyl]propane-1-sulfonamide

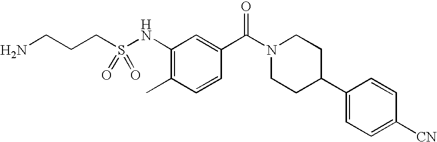

The title compound was prepared by the method described in Example 72 starting from N-[5-[4-(4-cyanophenyl)piperidine-1-carbonyl]-2-methyl-phenyl]-3-(1,3-dioxoisoindol-2-yl)propane-1-sulfonamide (Intermediate Q), ¹H nmr (300.072 MHz, DMSO-d₆) δ 1.55-1.83 (6H, m), 2.25 (3H, s), 2.68 (2H, t, J7.0), 2.89-3.17 (3H, m), 3.04 (2H, t, J7.0), 3.67-4.15 (1H, m), 4.43-4.85 (1H, m), 5.16-5.70 (2H, m), 6.97 (1H, dd, J7.7, 1.4), 7.19 (1H, d, J7.7), 7.28 (1H, s), 7.49 (2H, d, J8.3), 7.76 (2H, d, J8.3), m/z 441 (M+H)⁺, m/z 439 (M−H)⁻

### EXAMPLE 127

### N-[5-[4-(4-cyanophenyl)piperidine-1-carbonyl]-2-methyl-phenyl]-1,1-dioxo-thiolane-3-sulfonamide

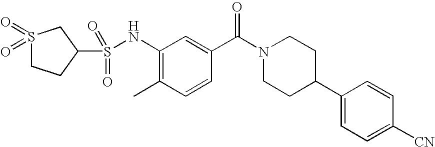

Prepared from Intermediate A

¹H NMR (CDCl₃) δ 1.64-1.97 (4H, m), 2.31 (3H, s), 2.59-2.66 (2H, m), 2.83-2.90 (2H, m), 3.06-3.16 (2H, m), 3.31-3.46 (3H, m), 3.81-3.98 (1H, m), 3.88 (1H, p, J8.0), 4.70-4.90 (1H, m), 7.14-7.19 (2H, m), 7.22-7.26 (1H, m), 7.33 (2H, d, J8.3), 7.62 (2H, d, J8.3), 8.03 (1H, bs), m/z 502 (M+H)⁺.

### EXAMPLE 128

### N-[5-[4-(4-cyanophenyl)piperidine-1-carbonyl]-2-methyl-phenyl]propane-2-sulfonamide

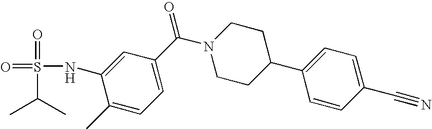

Prepared from Intermediate A

¹H NMR (300.072 MHz, CDCl₃) δ 1.41 (6H, d), 1.63-1.96 (4H, m), 2.33 (3H, s), 2.78-2.93 (2H, m), 3.03-3.20 (1H, m), 3.37 (1H, septet), 3.80-4.01 (1H, m), 4.61-5.05 (1H, m), 6.10 (1H, s), 7.13-7.18 (1H, m), 7.22-7.25 (1H, m), 7.32 (2H, d), 7.57-7.64 (3H, m), m/z 426 (M+H)⁺.

### EXAMPLE 129

### N-[5-[4-(4-cyanophenyl)piperidine-1-carbonyl]-2-methyl-phenyl]-2-oxo-1,3-dihydroindole-5-sulfonamide

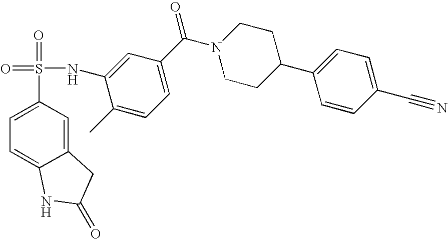

Prepared from Intermediate A

¹H NMR (300.072 MHz, CDCl₃) [symbol] 1.66-1.94 (m, 4H), 2.11 (s, 3H), 2.78-2.93 (m, 2H), 3.00-3.19 (m, 1H), 3.45 (s, 2H), 3.83-4.04 (m, 1H), 4.74-5.00 (m, 1H), 6.72 (d, 1H), 6.99 (s, 1H), 7.14-7.18 (m, 2H), 7.33 (d, 2H), 7.42 (s, 1H), 7.56-7.65 (m, 4H), 8.80 (s, 1H), m/z 515 (M+H)⁺.

### EXAMPLE 130

### 2-[[5-[4-(4-cyanophenyl)piperidine-1-carbonyl]-2-methyl-phenyl]sulfamoyl]acetic acid

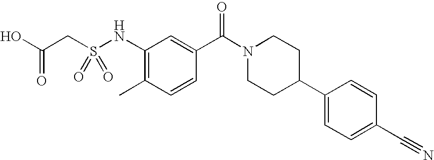

The title compound was prepared by hydrolysis in a manner analogous to that described in Example 79, starting from methyl 2-[[5-[4-(4-cyanophenyl)piperidine-1-carbonyl]-2-methyl-phenyl]sulfamoyl]acetate (Example 13) and using sodium hydroxide in place of lithium hydroxide, ¹H NMR (300.073 MHz, DMSO-d₆) δ 1.72 (4H, m), 2.34 (3H, s), 2.93 (1H, m), 4.11 (2H, s), 7.23 (1H, d), 7.31 (1H, d), 7.40 (1H, s), 7.50 (2H, d), 7.76 (2H, d), m/z 442 (M+H)⁺.

### EXAMPLE 131

### N-[5-[4-(4-cyanophenyl)piperidine-1-carbonyl]-2-methyl-phenyl]-3-(propan-2-ylsulfonylamino)propane-1-sulfonamide

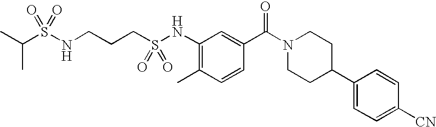

The title compound was prepared by the method described in Example 77, starting from 3-amino-N-[5-[4-(4-cyanophenyl)piperidine-1-carbonyl]-2-methyl-phenyl]propane-1-sulfonamide (Example 126) and propane-2-sulfonyl chloride, ¹H nmr (300.071 MHz, CDCl₃) 1.34 (3H, s), 1.36 (3H, s), 1.65-1.88 (4H, m), 2.08 (2H, p), 2.36 (3H, s), 2.81-2.89 (2H, m), 3.17 (2H, sextet), 3.26-3.33 (4H, m), 3.86-4.09 (1H, m), 4.78-4.83 (1H, m), 5.01 (1H, t), 6.82 (1H, s), 7.19 (1H, dd), 7.25-7.27 (1H, m), 7.33 (2H, d), 7.52 (1H, d), 7.61 (2H, d), m/z 545 (M−H)⁻, 547 (M+H)⁺.

### EXAMPLE 132

### N-[5-[4-(4-cyanophenyl)piperidine-1-carbonyl]-2-methyl-phenyl]-3-methanesulfonamido-propane-1-sulfonamide

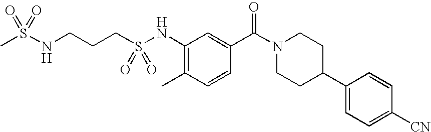

The title compound was prepared by the method described in Example 77, starting from 3-amino-N-[5-[4-(4-cyanophenyl)piperidine-1-carbonyl]-2-methyl-phenyl]propane-1-sulfonamide (Example 126) and methane sulfonyl chloride, ¹H nmr (300.072 MHz, CDCl₃) 1.66-2.00 (4H, m), 2.04-2.14 (2H, m), 2.35 (3H, s), 2.81-3.17 (3H, m), 2.94 (3H, s), 3.27-3.32 (4H, m), 3.85-4.09 (1H, m), 4.79-5.00 (1H, m), 5.25 (1H, t), 6.65 (1H, s), 7.17 (1H, dd), 7.25-7.27 (1H, m), 7.33 (2H, d), 7.52 (1H, d), 7.61 (2H, d), m/z 517 (M−H)⁻, 519 (M+H)⁺.

### EXAMPLE 133

### N-[3-[[5-[4-(4-cyanophenyl)piperidine-1-carbonyl]-2-methyl-phenyl]sulfamoyl]propyl]benzamide

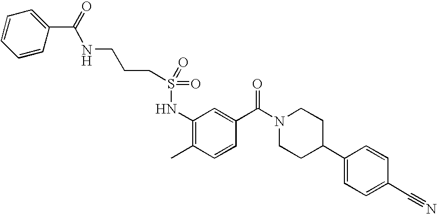

The title compound was prepared by the method described in Example 77, starting from 3-amino-N-[5-[4-(4-cyanophenyl)piperidine-1-carbonyl]-2-methyl-phenyl]propane-1-sulfonamide (Example 126) and benzoyl chloride, ¹H nmr (300.072 MHz, CDCl₃) 1.65-1.95 (4H, m), 2.17 (2H, p), 2.33 (3H, s), 2.72-3.08 (3H, m), 3.27 (2H, t), 3.60 (2H, q), 3.85-4.02 (1H, m), 4.69-5.00 (1H, m), 6.54 (1H, s), 6.71 (1H, bt), 7.16 (1H, dd), 7.23 (1H, d), 7.31 (2H, d), 7.42 (2H, d), 7.46-7.54 (2H, m), 7.60 (2H, d), 7.73 (2H, dd), m/z 543 (M−H)⁻, 545 (M+H)⁺.

### EXAMPLE 134

### N-[5-[4-(4-cyanophenyl)piperidine-1-carbonyl]-2-methyl-phenyl]-3-(1,3-dioxoisoindol-2-yl)propane-1-sulfonamide

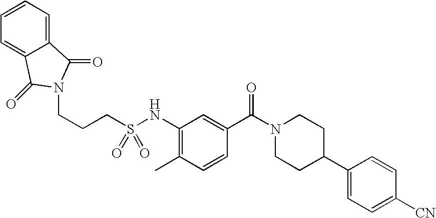

Prepared from Intermediate A

¹H nmr (300.071 MHz, CDCl₃) 1.47-1.82 (4H, m), 2.16-2.26 (2H, m), 2.32 (3H, s), 2.80-3.10 (3H, m), 3.20-3.25 (2H, m), 3.80 (2H, t, J6.8), 3.83-4.13 (1H, m), 4.64-4.99 (1H, m), 6.41 (1H, s), 7.15 (1H, dd, J7.8, 1.4), 7.21 (1H, d, J7.8), 7.33 (2H, d, J8.3), 7.50 (1H, d, J1.4), 7.61 (2H, d, J8.3), 7.71-7.75 (2H, m), 7.81-7.84 (2H, m), m/z 569 (M−H)⁻, 571 (M+H)⁺. The requisite 3-(1,3-dioxoisoindol-2-yl)propane-1-sulfonyl chloride (CAS Reg. No. 92605-69-0) may be prepared as described in Bioorganic & Medicinal Chemistry Letters (1996), 6(14), 1709-1714.

### EXAMPLE 135

### N-[5-[4-(4-cyanophenyl)piperidine-1-carbonyl]-2-methylsulfonyl-phenyl]-1-phenyl-methanesulfonamide

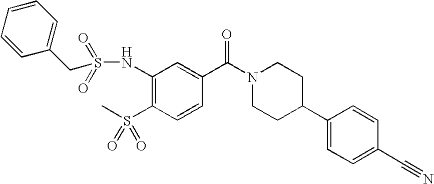

Prepared from Intermediate R

¹H NMR (300.072 MHz, CDCl₃) [symbol] 1.63-2.00 (m, 4H), 2.79-2.93 (m, 2H), 3.00 (s, 3H), 3.10-3.21 (m, 1H), 3.56-3.79 (m, 1H), 4.55 (s, 2H), 4.80-4.96 (m, 1H), 7.22-7.23 (m, 1H), 7.28-7.28 (m, 1H), 7.31-7.37 (m, 7H), 7.63 (d, 2H), 7.92 (d, 1H), 8.82 (s, 1H), m/z 536 (M−H)⁻.

### EXAMPLE 136

### N-[3-[[5-[4-(4-cyanophenyl)piperidine-1-carbonyl]-2-methyl-phenyl]sulfamoyl]propyl]acetamide

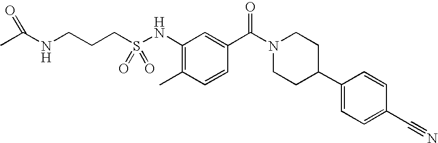

The title compound was prepared by the method described in Example 77, starting from 3-amino-N-[5-[4-(4-cyanophenyl)piperidine-1-carbonyl]-2-methyl-phenyl]propane-1-sulfonamide (Example 126) and acetic anhydride, ¹H NMR (300.072 MHz, CDCl₃) δ 1.83-1.49 (4H, m), 1.92 (3H, s), 2.04-1.92 (2H, m), 2.35 (3H, s), 3.14-2.82 (3H, m), 3.26-3.06 (2H, m), 3.36-3.26 (2H, m), 4.08-3.76 (1H, m), 5.00-4.58 (1H, m), 6.43 (1H, t), 7.16 (1H, d), 7.27-7.23 (2H, m), 7.33 (2H, d), 7.49 (1H, s), 7.61 (2H, d) ¹H NMR (300.072 MHz, CDCl₃) δ 1.83-1.49 (4H, m), 1.92 (3H, s), 2.04-1.92 (2H, m), 2.35 (3H, s), 3.14-2.82 (3H, m), 3.26-3.06 (2H, m), 3.36-3.26 (2H, m), 4.08-3.76 (1H, m), 5.00-4.58 (1H, m), 6.43 (1H, t), 7.16 (1H, d), 7.27-7.23 (2H, m), 7.33 (2H, d), 7.49 (1H, s), 7.61 (2H, d), m/z 483 (M+H)⁺.

### EXAMPLE 137

### Methyl 2-[[5-[4-(4-cyanophenyl)piperidine-1-carbonyl]-2-methyl-phenyl]sulfamoyl]propanoate

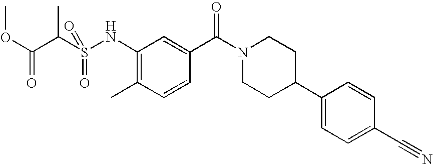

The title compound was prepared by a method analogous to that described in Example 115, starting from methyl 2-[[5-[4-(4-cyanophenyl)piperidine-1-carbonyl]-2-methyl-phenyl]-[(2-methylpropan-2-yl)oxycarbonyl]sulfamoyl]propanoate (Intermediate S) to give the title compound as the free base after chromatography on silica, ¹H NMR (300.073 MHz, DMSO-d₆) δ 1.48 (3H, d), 1.55-1.95 (4H, m), 2.34 (3H, s), 2.78-3.22 (3H, m), 3.58 (3H, s), 3.66-3.94 (1H, m), 4.24 (1H, q), 4.35-4.78 (1H, m), 7.22 (1H, d), 7.31 (1H, d), 7.39 (1H, s), 7.50 (2H, d), 7.76 (2H, d), 9.61 (1H, s), m/z 470 (M+H)⁺.

### EXAMPLE 138

### N-[5-[4-(4-cyanophenyl)piperidine-1-carbonyl]-2-methyl-phenyl]-1-hydroxy-propane-2-sulfonamide

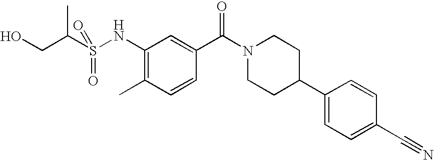

A stirred solution of methyl 2-(N-(5-(4-(4-cyanophenyl)piperidine-1-carbonyl)-2-methylphenyl)sulfamoyl)propanoate (Example 137) (69 mg, 0.15 mmol) in THF (2 mL) was placed under nitrogen and treated with a solution of lithium borohydride, (1.065 mL of a 2M solution in tetrahydrofuran, 2.13 mmol, 2 eq). The reaction mixture was stirred at ambient temperature for approximately 18 hrs. It was the diluted with EtOAc and the resulting solution washed sequentially with water and brine. The organic layer was dried (MgSO₄) and evaporated to afford crude product. This was purified by flash silica chromatography (4 g column, elution gradient 50 to 100% EtOAc in isohexane) to give N-(5-(4-(4-cyanophenyl)piperidine-1-carbonyl)-2-methylphenyl)-1-hydroxypropane-2-sulfonamide as a colourless solid (23 mg, 35.4%), ¹H NMR (300.073 MHz, DMSO-d₆) δ 1.29 (3H, d), 1.48-1.93 (4H, m), 2.32 (3H, s), 2.68-3.23 (4H, m), 3.39-3.94 (3H, m), 4.38-4.80 (1H, m), 5.52-5.66 (1H, m), 7.12-7.39 (5H, m), 7.49 (2H, d), 7.76 (2H, d), m/z 483 (M+H)⁺.

### EXAMPLE 139

### N-[5-[4-(4-cyanophenyl)piperidine-1-carbonyl]-2-methyl-phenyl]-1,3-dimethyl-pyrazole-4-sulfonamide

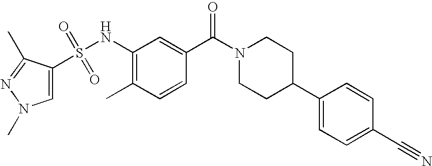

Prepared from Intermediate A

The reactants were heated by microwave heating at 100° C. for 30 minutes, using pyridine as solvent. ¹H NMR (300.073 MHz, DMSO-d₆) δ 1.45-1.90 (4H, m), 2.01 (3H, s), 2.13 (3H, s), 2.70-3.23 (3H, m), 3.41-3.88 (4H, m), 4.30-4.80 (1H, m), 7.04 (1H, s), 7.15-7.30 (2H, m), 7.50 (2H, d), 7.77 (2H, d), 7.98 (1H, s), 9.42 (1H, s), m/z (ESI+) (M+H)⁺=478.38, HPLC tR=2.13 min.

### EXAMPLE 140

### N-[5-[4-(4-cyanophenyl)piperidine-1-carbonyl]-2-methyl-phenyl]-1-methyl-pyrazole-3-sulfonamide

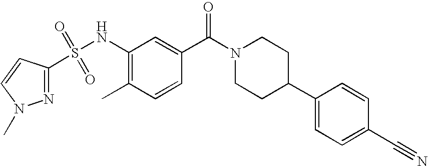

Prepared from Intermediate A

The reactants were heated by microwave heating at 100° C. for 30 minutes, using pyridine as solvent. ¹H NMR (300.073 MHz, DMSO-d₆) δ 1.43-1.94 (4H, m), 2.16 (3H, s), 2.67-3.18 (3H, m), 3.40-3.79 (1H, m), 3.86 (3H, s), 4.32-4.78 (1H, m), 6.46-6.51 (1H, m), 7.15 (2H, d), 7.23 (1H, d), 7.49 (2H, d), 7.73-7.84 (3H, m), 9.72 (1H, s), m/z (ESI+) (M+H)⁺=464.36, HPLC tR=2.12 min.

### EXAMPLE 141

### N-[5-[4-(4-cyanophenyl)piperidine-1-carbonyl]-2-methyl-phenyl]-3-(pyridin-3-ylmethylamino)propane-1-sulfonamide

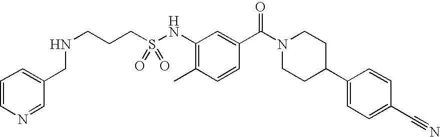

A solution of sodium cyanoborohydride (0.34 mL of a 10M solution in THF, 0.34 mmol) was added to a stirred solution of nicotinaldehyde (0.032 mL, 0.34 mmol) and 3-amino-N-(5-(4-(4-cyanophenyl)piperidine-1-carbonyl)-2-methylphenyl)propane-1-sulfonamide (Example 126) (150 mg, 0.34 mmol) in THF (2 mL) at ambient temperature under nitrogen. The resulting solution was stirred at ambient temperature for 17 hours. The reaction mixture was concentrated, diluted with EtOAc (50 mL) and washed with 1M NaOH (50 mL). The aqueous layer was extracted with EtOAc (2×50 mL) and the combined organic extracts were dried over MgSO₄, filtered and evaporated to afford crude product. This was purified by flash silica chromatography, elution gradient 0 to 15% MeOH in DCM. Pure fractions were evaporated to dryness to afford the title compound (39.0 mg, 0.07 mmol, 21.52%) as a yellow dry film, ¹H NMR (300.072 MHz, CDCl₃) δ 1.99-1.52 (4H, m), 2.08-2.01 (2H, m), 2.32 (3H, s), 2.83-2.77 (2H, m), 3.25-2.83 (3H, m), 3.27 (2H, t), 3.79 (2H, s), 4.08-3.79 (1H, m), 5.17-4.69 (1H, m), 7.17 (1H, d), 7.26-7.23 (2H, m), 7.32 (2H, d), 7.50 (1H, s), 7.60 (3H, d), 8.52-8.48 (2H, m), m/z 532 (M+H)⁺, 530(M−H)⁻.

### EXAMPLE 142

### N-[5-[4-(4-cyanophenyl)piperidine-1-carbonyl]-2-methyl-phenyl]-1-ethyl-5-methyl-pyrazole-4-sulfonamide

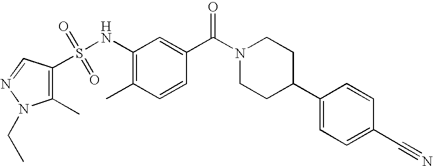

Prepared from Intermediate A

The reactants were heated by microwave heating at 100° C. for 30 minutes, using pyridine as solvent.

¹H NMR (300.073 MHz, DMSO-d₆) δ 1.23 (3H, t), 1.44-1.91 (4H, m), 2.07-2.11 (6H, m), 2.68-3.21 (3H, m), 3.45-3.83 (1H, m), 4.02 (2H, q), 4.41-4.71 (1H, m), 7.00 (1H, s), 7.17-7.28 (2H, m), 7.45-7.55 (3H, m), 7.77 (2H, d), 9.40 (1H, s), m/z 492 (M+H)⁺.

### EXAMPLE 143

### N-[5-[4-(4-cyanophenyl)piperidine-1-carbonyl]-2-methyl-phenyl]-1-ethyl-3-methyl-pyrazole-4-sulfonamide

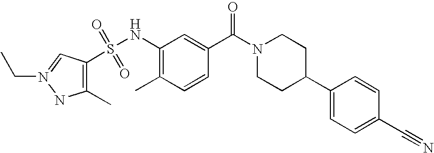

Prepared from Intermediate A

The reactants were heated by microwave heating at 100° C. for 30 minutes, using pyridine as solvent. ¹H NMR (300.073 MHz, DMSO-d₆) δ 1.25 (3H, t), 1.43-1.89 (4H, m), 2.01 (3H, s), 2.12 (3H, s), 2.66-3.18 (3H, m), 3.38-3.85 (1H, m), 4.00 (2H, q), 4.27-4.73 (1H, m), 7.02 (1H, s), 7.16-7.28 (2H, m), 8.00 (1H, s), 7.50 (2H, d), 7.77 (2H, d), 9.39 (1H, s), m/z 492 (M+H)⁺.

### EXAMPLE 144

### Methyl 3-[[5-[4-(4-cyanophenyl)piperidine-1-carbonyl]-2-methyl-phenyl]sulfamoyl]propanoate

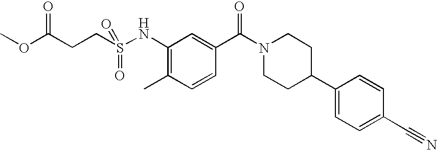

Prepared from Intermediate A

¹H NMR (300.073 MHz, DMSO-d₆) δ 1.52-1.93 (4H, m), 2.32 (3H, s), 2.78 (2H, t), 2.85-3.23 (3H, m), 3.38 (2H, t), 3.58 (3H, s), 3.62-3.90 (1H, m), 4.40-4.83 (1H, m), 7.19-7.26 (1H, m), 7.28-7.36 (2H, m), 7.49 (2H, d), 7.76 (2H, d), 9.34 (1H, s), m/z 470 (M+H)⁺.

### EXAMPLE 145

### N-[5-[4-(4-cyanophenyl)piperidine-1-carbonyl]-2-methyl-phenyl]-1-methyl-pyrazole-4-sulfonamide

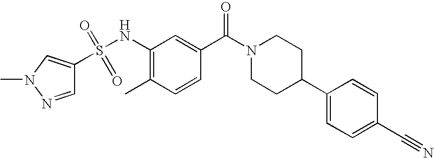

Prepared from Intermediate A

The reactants were heated by microwave heating at 100° C. for 30 minutes, using pyridine as solvent. ¹H NMR (300.073 MHz, DMSO-d₆) δ 1.44-1.92 (4H, m), 2.17 (3H, s), 2.63-3.21 (3H, m), 3.41-3.73 (1H, m), 3.80 (3H, s), 4.31-4.80 (1H, m), 7.03 (1H, s), 7.18 (1H, d), 7.26 (1H, d), 7.50 (2H, d), 7.58 (1H, s), 7.77 (2H, d), 8.11 (1H, s), 9.48 (1H, s), m/z 464 (M+H)⁺.

### EXAMPLE 146

### N-[5-[4-(4-cyanophenyl)piperidine-1-carbonyl]-2-methyl-phenyl]prop-2-ene-1-sulfonamide

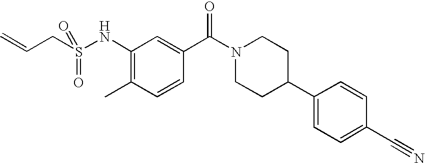

Prepared from Intermediate A

¹H NMR (300.073 MHz, DMSO-d₆) δ 1.44-1.91 (4H, m), 2.32 (3H, s), 2.65-3.22 (3H, m), 3.52-3.84 (1H, m), 3.90 (2H, d), 4.31-4.92 (1H, m), 5.31-5.43 (2H, m), 5.73-5.92 (1H, m), 7.20 (1H, d), 7.29 (1H, d), 7.35 (1H, s), 7.49 (2H, d), 7.76 (2H, d), 9.24 (1H, s), m/z 424 (M+H)⁺.

### EXAMPLE 147

### N-[5-[4-(4-cyanophenyl)piperidine-1-carbonyl]-2-methyl-phenyl]-1-[(4R)-4-methyl-2,5-dioxo-imidazolidin-4-yl]methanesulfonamide

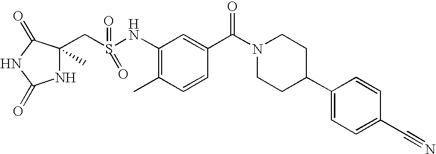

Prepared from Intermediate A

¹H NMR (300.072 MHz, CDCl₃) δ 1.53 (3H, s), 1.64-1.96 (4H, m), 2.31 (3H, s), 2.76-2.89 (2H, m), 3.02-3.32 (1H, m), 3.59 (2H, q), 3.81-3.99 (1H, m), 4.71-4.86 (1H, m), 7.12-7.22 (2H, m), 7.26-7.34 (3H, m), 7.45 (1H, s), 7.58 (2H, d), 8.05 (1H, s), 9.51 (1H, s), m/z 510 (M+H)⁺. The requisite (S)-(4-methyl-2,5-dioxoimidazolidin-4-yl)methanesulfonyl chloride may be prepared as described in Patent Application WO 2004/024698.

### EXAMPLE 148

### N-[5-[4-(4-cyanophenyl)piperidine-1-carbonyl]-2-methyl-phenyl]-1-(4-ethyl-2,5-dioxo-imidazolidin-4-yl)methanesulfonamide

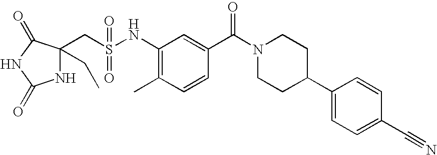

Prepared from Intermediate A

¹H NMR (300.072 MHz, CDCl₃) δ 0.88 (3H, t), 1.64-1.97 (6H, m), 2.32 (3H, s), 2.77-2.91 (2H, m), 3.07-3.22 (1H, m), 3.60 (2H, q), 3.81-4.03 (1H, m), 4.67-4.98 (1H, m), 7.11 (1H, s), 7.16-7.23 (2H, m), 7.33 (2H, d), 7.45 (1H, s), 7.61 (2H, d), 7.87 (1H, s), 9.36 (1H, s), m/z 524 (M+H)⁺. The requisite (4-ethyl-2,5-dioxo-imidazolidin-4-yl)methanesulfonyl chloride may be prepared as described in Patent Application WO 2004/024698.

### EXAMPLE 149

### N-[5-[4-(4-cyanophenyl)piperidine-1-carbonyl]-2-methyl-phenyl]-3,5-dimethyl-1,2-oxazole-4-sulfonamide

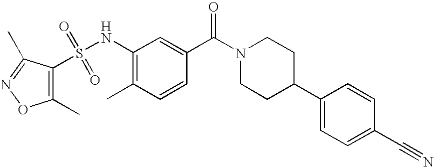

Prepared from Intermediate A

The reactants were heated by microwave heating at 100° C. for 30 minutes, using pyridine as solvent. ¹H NMR (300.073 MHz, DMSO-d₆) δ 1.49-1.90 (4H, m), 2.09-2.17 (6H, m), 2.26 (3H, s), 2.59-3.16 (3H, m), 3.45-3.86 (1H, m), 4.30-4.77 (1H, m), 7.06 (1H, s), 7.24-7.34 (2H, m), 7.50 (2H, d), 7.77 (2H, d), 9.98 (1H, s), m/z 479 (M+H)⁺.

### EXAMPLE 150

### N-[5-[4-(4-cyanophenyl)piperidine-1-carbonyl]-2-methyl-phenyl]-3-dimethylamino-2-hydroxy-propane-1-sulfonamide

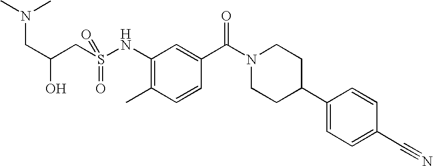

Titanium(IV) isopropoxide (161 μl, 0.55 mmol) was added to N-(5-(4-(4-cyanophenyl)piperidine-1-carbonyl)-2-methylphenyl)-1-(oxiran-2-yl)methanesulfonamide (Intermediate T) (141 mg, 0.32 mmol) and dimethylamine ((1.925 mL of a 2M solution in THF, 3.85 mmol). The resulting solution was stirred at room temperature for 18 hours. The reaction mixture was diluted with EtOAc and water, filtered, and the organic filtrate washed sequentially with water (×3) and saturated brine. The organic layer was dried over MgSO₄, filtered and evaporated to give crude product which was purified by flash silica chromatography, elution gradient 0 to 100% MeOH in EtOAc to give the title compound (37.0 mg, 23.80%) as a yellow solid, ¹H NMR (300.073 MHz, DMSO-d₆) δ 1.53-1.88 (4H, m), 2.11 (6H, s), 2.21-2.28 (2H, m), 2.31 (3H, s), 2.76-3.17 (3H, m), 3.32-3.42 (2H, m), 3.57-3.92 (1H, m), 4.02-4.15 (1H, m), 4.43-4.80 (1H, m), 7.15 (1H, d), 7.28 (1H, d), 7.37 (1H, s), 7.49 (2H, d), 7.76 (2H, d), m/z 485 (M+H)⁺.

### EXAMPLE 151

### 3-[[5-[4-(4-cyanophenyl)piperidine-1-carbonyl]-2-methyl-phenyl]sulfamoyl]propanoic acid

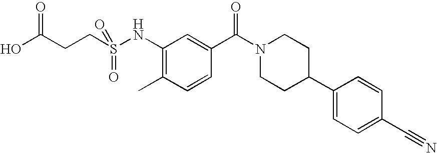

The title compound was prepared by hydrolysis in a manner analogous to that described in Example 79, starting from methyl 3-[[5-[4-(4-cyanophenyl)piperidine-1-carbonyl]-2-methyl-phenyl]sulfamoyl]propanoate (Example 144), ¹H NMR (300.073 MHz, DMSO-d₆) δ 1.49-1.88 (4H, m), 2.33 (3H, s), 2.69 (2H, t), 2.76-3.22 (3H, m), 3.34 (2H, t), 3.53-3.93 (1H, m), 4.38-4.79 (1H, m), 7.17-7.26 (1H, m), 7.31 (2H, d), 7.50 (2H, d), 7.76 (2H, d), 9.31 (1H, s), 12.45 (1H, s), m/z 456 (M+H)⁺.

### EXAMPLE 152

### N-[5-[4-(4-cyanophenyl)piperidine-1-carbonyl]-2-methyl-phenyl]-1,3,5-trimethyl-pyrazole-4-sulfonamide

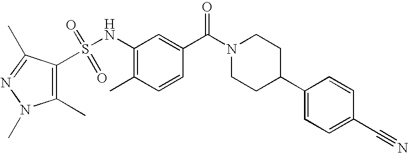

Prepared from Intermediate A

The reactants were heated by microwave heating at 100° C. for 3 hours, using pyridine as solvent. ¹H NMR (300.072 MHz, CDCl₃) δ 1.50-2.06 (4H, m), 2.16 (3H, s), 2.23 (3H, s), 2.27 (3H, s), 2.70-3.25 (3H, m), 3.67 (3H, s), 4.09 (1H, s), 4.60-5.03 (1H, m), 6.75 (1H, s), 7.16 (2H, s), 7.26 (1H, d), 7.34 (2H, d), 7.62 (2H, d), m/z 492 (M+H)⁺.

### EXAMPLE 153

### 1-acetyl-N-[5-[4-(4-cyanophenyl)piperidine-1-carbonyl]-2-methyl-phenyl]pyrrolidine-3-sulfonamide

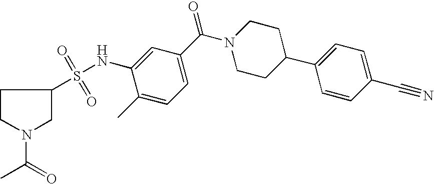

The title compound was prepared by the method described in Example 77, starting from N-(5-(4-(4-cyanophenyl)piperidine-1-carbonyl)-2-methylphenyl)pyrrolidine-3-sulfonamide hydrochloride (Intermediate U) and acetic anhydride, ¹H NMR (300.072 MHz, CDCl₃) δ 1.70-1.96 (4H, m), 2.03 (3H, s), 2.22-2.40 (1H, m), 2.34 (3H, s), 2.52-2.64 (1H, m), 2.79-2.92 (2H, m), 3.00-3.19 (1H, m), 3.45-3.58 (1H, m), 3.70-3.81 (3H, m), 3.87-3.96 (1H, m), 4.01-4.07 (1H, m), 4.76-4.94 (1H, m), 7.17-7.24 (2H, m), 7.33 (2H, d), 7.41 (1H, s), 7.45-7.49 (1H, m), 7.61 (2H, d), m/z 495 (M+H)⁺.

### EXAMPLE 154

### N-[5-[4-(4-cyanophenyl)piperidine-1-carbonyl]-2-methyl-phenyl]-1-methylsulfonyl-pyrrolidine-3-sulfonamide

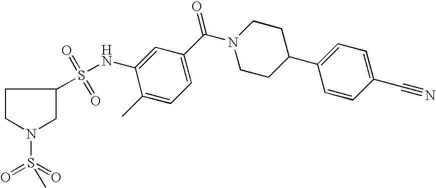

The title compound was prepared by the method described in Example 77, starting from N-(5-(4-(4-cyanophenyl)piperidine-1-carbonyl)-2-methylphenyl)pyrrolidine-3-sulfonamide hydrochloride (Intermediate U) and methane sulfonyl chloride, ¹H NMR (300.072 MHz, CDCl₃) δ 1.69-2.00 (4H, m), 2.29 (3H, s), 2.36-2.43 (1H, m), 2.50-2.58 (1H, m), 2.82-2.88 (2H, m), 2.91 (3H, s), 3.06-3.20 (1H, m), 3.47-3.55 (2H, m), 3.71-3.91 (4H, m), 4.75-5.00 (1H, m), 7.13-7.22 (2H, m), 7.32 (3H, d), 7.54 (1H, s), 7.62 (2H, d), m/z 531 (M+H)⁺.

### EXAMPLE 155

### N-[5-[4-(4-chlorophenyl)piperidine-1-carbonyl]-2-methyl-phenyl]methanesulfonamide

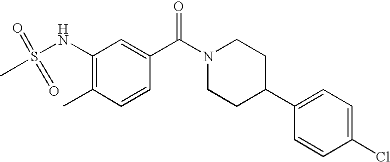

A solution of 3-methanesulfonamido-4-methyl-benzoic acid (Intermediate W) (200 mg, 0.87 mmol), DMAP (11 mg, 0.09 mmol) and EDAC (200 mg, 1.05 mmol) in DMF (5 mL) was added to 4-(4-chlorophenyl)piperidine hydrochloride (233 mg, 1.0 mmol) and the reaction was stirred overnight at ambient temperature. The solvent was evaporated in vacuo and the resulting gum was dissolved DCM and the solution washed with water. After drying (phase separating cartridge) the solvent was evaporated and the crude product purified by column chromatography (12 g silica cartridge, eluting with 0-5% MeOH in DCM) to give the title compound as a colourless solid (152 mg, 46% yield), ¹H NMR (300.073 MHz, DMSO-d₆) δ 1.48-1.97 (4H, m), 2.33 (3H, s), 2.68-3.20 (3H, m), 2.99 (3H, s), 3.58-3.89 (1H, m), 4.42-4.73 (1H, m), 7.16-7.44 (7H, m), 9.15 (1H, s), m/z 407 (M+H)⁺.

### EXAMPLE 156

### 4-[1-(3-methanesulfonamido-4-methyl-benzoyl)-4-piperidyl]-N-methyl-benzamide

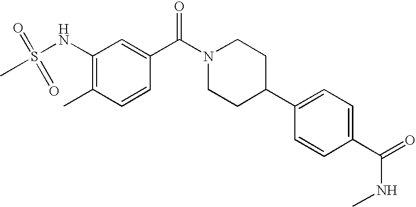

Prepared from Intermediate W

The title compound was prepared by a method analogous to that described in Example 155, using N-methyl-4-(4-piperidyl)benzamide in place of 4-(4-chlorophenyl)piperidine hydrochloride, ¹H NMR (300.073 MHz, DMSO-d₆) δ 1.52-1.96 (4H, m), 2.33 (3H, s), 2.76 (3H, d), 2.79-2.94 (2H, m), 3.00 (3H, s), 3.02-3.24 (1H, m), 3.59-3.90 (1H, m), 4.40-4.75 (1H, m), 7.22 (1H, d), 7.27-7.39 (4H, m), 7.76 (2H, d), 8.31 (1H, d), 9.15 (1H, s), m/z 430 (M+H)⁺.

### EXAMPLE 157

### N-[2-methyl-5-[4-[4-(trifluoromethyl)phenyl]piperidine-1-carbonyl]phenyl]methanesulfonamide

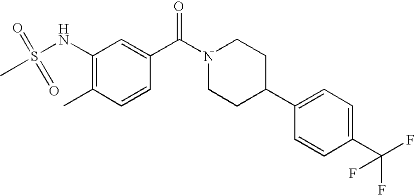

Prepared from Intermediate W

The title compound was prepared by a method analogous to that described in Example 155, using 4-[4-(trifluoromethyl)phenyl]piperidine hydrochloride in place of 4-(4-chlorophenyl)piperidine hydrochloride, ¹H NMR (300.073 MHz, DMSO-d₆) δ 1.52-1.97 (4H, m), 2.34 (3H, s), 2.69-3.24 (3H, m), 3.00 (3H, s), 3.56-3.94 (1H, m), 4.40-4.79 (1H, m), 7.22 (1H, d), 7.32 (2H, d), 7.51 (2H, d), 7.65 (2H, d), 9.16 (1H, s), m/z 441 (M+H)⁺.

### EXAMPLE 158

### N-[2-methyl-5-[4-(4-methylphenyl)piperidine-1-carbonyl]phenyl]methanesulfonamide

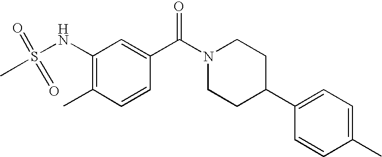

Prepared from Intermediate W

The title compound was prepared by a method analogous to that described in Example 155, using 4-(4-methylphenyl)piperidine in place of 4-(4-chlorophenyl)piperidine hydrochloride, ¹H NMR (300.073 MHz, DMSO-d₆) δ 1.47-1.91 (4H, m), 2.25 (3H, s), 2.34 (3H, s), 2.64-3.21 (3H, m), 2.99 (3H, s), 3.54-3.89 (1H, m), 4.40-4.71 (1H, m), 7.03-7.25 (5H, m), 7.27-7.36 (2H, m), 9.14 (1H, s), m/z 387 (M+H)⁺.

### EXAMPLE 159

### N-[5-[4-(4-cyanophenyl)piperidine-1-carbonyl]-2-methylsulfanylphenyl]methanesulfonamide

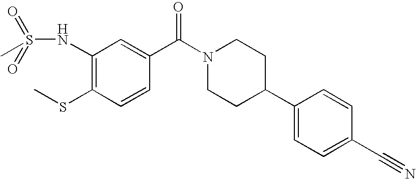

Prepared from Intermediate X

¹H NMR (300.072 MHz, CDCl₃) [symbol] 1.63-1.98 (m, 4H), 2.46 (s, 3H), 2.80-2.90 (m, 2H), 3.05 (s, 3H), 3.10-3.30 (m, 1H), 3.79-4.01 (m, 1H), 4.63-5.09 (m, 1H), 7.21 (s, 1H), 7.29 (d, 1H), 7.32 (d, 2H), 7.48 (d, 1H), 7.58-7.64 (m, 3H), m/z (ESI+) (M+H)+=430; HPLC tR=2.13 min.

### EXAMPLE 160

### N-[5-[4-(4-cyanophenyl)piperidine-1-carbonyl]-2-methylsulfanylphenyl]-1-phenylmethanesulfonamide

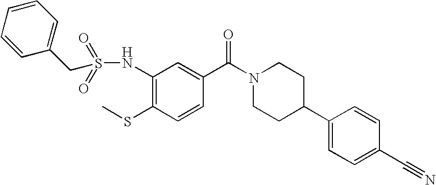

Prepared from Intermediate X

¹H NMR (300.072 MHz, CDCl₃) δ 1.62-1.98 (m, 4H), 2.34 (s, 3H), 2.81-2.93 (m, 2H), 3.00-3.20 (m, 1H), 3.72-3.96 (m, 1H), 4.42 (s, 2H), 4.72-4.95 (m, 1H), 7.18-7.37 (m, 9H), 7.51 (d, 1H), 7.56 (d, 1H), 7.62 (d, 2H), m/z (ESI−) (M−H)⁻=504; HPLC tR=2.56 min.

### EXAMPLE 161

### N-[5-[4-(4-cyanophenyl)piperidine-1-carbonyl]-2-(methoxymethyl)phenyl]methanesulfonamide

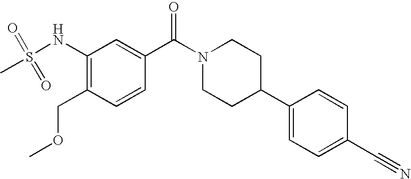

Prepared from Intermediate Y

¹H NMR (300.073 MHz, DMSO-d₆) δ 1.52-1.96 (4H, m), 2.69-2.98 (2H, m), 3.00 (3H, s), 3.24 (1H, s), 3.33 (3H, s), 3.54-3.82 (1H, m), 4.48-4.73 (3H, m), 7.32 (1H, d), 7.37 (1H, s), 7.43-7.55 (3H, m), 7.77 (2H, d), 9.10 (1H, s), m/z (ESI+) (M+H)⁺=428.38; HPLC tR=2.12 min.

### EXAMPLE 162

### N-[5-[4-(4-methoxyphenyl)piperidine-1-carbonyl]-2-methylphenyl]methanesulfonamide

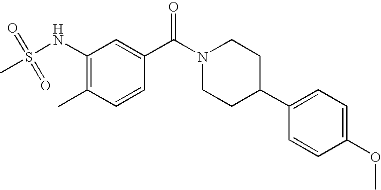

Prepared from Intermediate W

The title compound was prepared by a method analogous to that described in Example 155, using 4-(4-methoxyphenyl)piperidine in place of 4-(4-chlorophenyl)piperidine hydrochloride, ¹H NMR (300.073 MHz, DMSO-d₆) δ 1.45-1.90 (4H, m), 2.33 (3H, s), 2.67-3.24 (3H, m), 2.99 (3H, s), 3.60-3.84 (1H, m), 3.71 (3H, s), 4.40-4.69 (1H, m), 6.85 (2H, d), 7.13-7.23 (3H, m), 7.28-7.34 (2H, m), 9.15 (1H, s), m/z (ESI+) (M+H)⁺=403.32; HPLC tR=2.18.

### EXAMPLE 163

### N-[[4-[1-(3-methanesulfonamido-4-methylbenzoyl)piperidin-4-yl]phenyl]methyl]methanesulfonamide

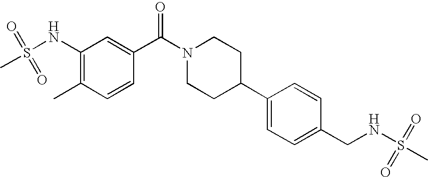

### Method 2

Methanesulfonyl chloride (0.040 mL, 0.52 mmol) was added dropwise to a solution of N-(5-(4-(4-(aminomethyl)phenyl)piperidine-1-carbonyl)-2-methylphenyl)methanesulfonamide (Intermediate Z) (173 mg, 0.43 mmol) in pyridine (2 mL) at ambient temperature over a period of 1 minute. The resulting solution was stirred at ambient temperature for 1 hour. Further methanesulfonyl chloride (0.040 mL, 0.52 mmol) was added and the solution was stirred at 100° C. for a further 1 hour. The reaction was still incomplete hence a further 2 equivalents of methanesulfonyl chloride was added and the mixture stirred for a further hour. The reaction mixture was then evaporated to dryness, redissolved in DCM (50 mL), and the resulting solution washed with water (50 mL). The organic layer was dried by passing through a phase separating cartridge and evaporated to afford crude product. This was purified by chromatography on silica (12 g column, elution gradient 0 to 10% MeOH in DCM) to give the title compound as a colourless solid (77 mg, 37.3%), ¹H NMR (400.132 MHz, DMSO-d₆) δ 1.43-1.87 (4H, m), 2.27 (3H, s), 2.68-2.85 (5H, m), 2.94 (3H, s), 3.00-3.15 (1H, m), 3.58-3.74 (1H, m), 4.05 (2H, d), 4.46-4.61 (1H, m), 7.14-7.23 (5H, m), 7.24-7.28 (2H, m), 7.44 (1H, t), 9.14 (1H, s), m/z (ESI+) (M+H)⁺=478.45; HPLC tR=1.79 min.

### EXAMPLE 164

### N-[[4-[1-(3-methanesulfonamido-4-methylbenzoyl)piperidin-4-yl]phenyl]methyl]acetamide

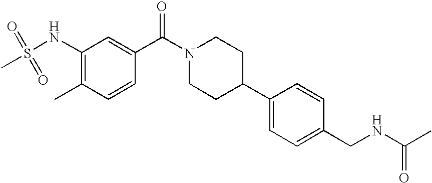

Method 2 from Intermediate Z

¹H NMR (400.132 MHz, DMSO-d₆) δ 1.50-1.94 (7H, m), 2.34 (3H, s), 2.73-2.94 (2H, m), 3.01 (3H, s), 3.05-3.24 (1H, m), 3.63-3.82 (1H, m), 4.20 (2H, d), 4.52-4.69 (1H, m), 7.15-7.26 (5H, m), 7.30-7.36 (2H, m), 8.30 (1H, t), 9.21 (1H, s), m/z (ESI+) (M+H)⁺=444.47; HPLC tR=1.61 min.

### EXAMPLE 165

### N-[5-[4-[4-(hydroxymethyl)phenyl]piperidine-1-carbonyl]-2-methylphenyl]methanesulfonamide

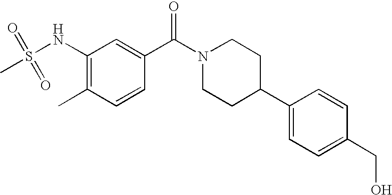

### Method 3

Lithium borohydride (0.174 mL of a 2M solution in THF, 0.35 mmol) was added dropwise to methyl 4-(1-(4-methyl-3-(methylsulfonamido)benzoyl)piperidin-4-yl)benzoate (Intermediate AA) (150 mg, 0.35 mmol) in THF (5 mL) at ambient temperature over a period of 2 minutes under nitrogen. The resulting solution was stirred at ambient temperature for 6 hours and then at 65° C. for 24 hrs. A further quantity of lithium borohydride (0.174 mL of a 2M solution in THF, 0.35 mmol) was added dropwise and maintained at 65° C. for 24 hrs. The reaction mixture was carefully diluted with water, acidified with 2M HCl and the aqueous mixture washed with EtOAc (2×75 mL portions). The solvent was dried and evaporated under reduced pressure to give crude product. This was purified by chromatography on silica (elution gradient 0 to 5% MeOH in DCM) to give the title compound as a colourless solid (59.0 mg, 42.1%), ¹H NMR (400.132 MHz, DMSO-d₆) δ 1.44-1.87 (4H, m), 2.27 (3H, s), 2.58-2.89 (2H, m), 2.94 (3H, s), 2.97-3.16 (1H, m), 3.51-3.85 (1H, m), 4.39 (2H, d), 4.42-4.63 (1H, m), 4.99 (1H, t), 7.12-7.21 (5H, m), 7.23-7.28 (2H, m), 9.09 (1H, s), m/z (ESI+) (M+H)⁺=403.39; HPLC tR=1.65 min.

### EXAMPLE 166

### N-[5-[4-(4-cyanophenyl)piperidine-1-carbonyl]-2,4-dimethylphenyl]ethanesulfonamide

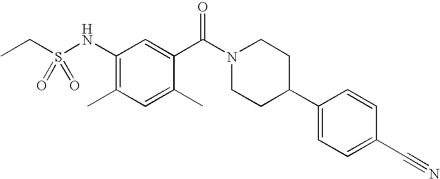

Prepared from Intermediate D

¹H NMR (400.132 MHz, DMSO-d₆) d1.24 (t, 3H), 1.40-1.81 (m, 3H), 1.82-1.95 (m, 1H), 2.08-2.27 (m, 3H), 2.27 (s, 3H), 2.78-2.99 (m, 2H), 3.00-3.19 (q, 2H+m, 1H), 3.36-3.49 (m, 1H), 4.67 (d, 1H), 6.97-7.22 (br m, 1H+s, 1H), 7.49 (d, 2H), 7.77 (d, 2H), 8.99 (s, 1H), m/z (ESI+) (M+H)⁺=426.28; HPLC tR=2.26 min.

### EXAMPLE 167

### N-[2-methyl-5-[4-[4-(5-methyl-1,2,4-oxadiazol-3-yl)phenyl]piperidine-1-carbonyl]phenyl]methanesulfonamide

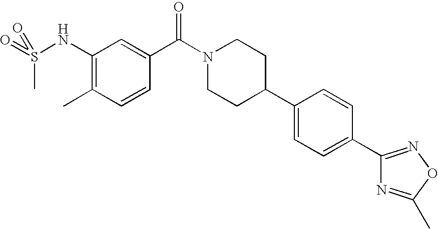

Acetyl chloride (0.018 mL, 0.25 mmol) was added to (Z)-N′-hydroxy-4-(1-(4-methyl-3-(methylsulfonamido)benzoyl)piperidin-4-yl)benzimidamide (Intermediate BB, Example 214) (0.072 g, 0.17 mmol) in toluene (2 mL) and the resulting suspension was heated to reflux and stirred for 3 days. The reaction mixture was evaporated to dryness to afford crude product which was purified by preparative HPLC using decreasingly polar mixtures of water (containing 0.1% formic acid) and MeCN as eluents to give the title compound as a colourless gum (0.012 g, 15.79%), ¹H NMR (400.132 MHz, DMSO-d₆) δ 1.58-1.94 (4H, m), 2.34 (3H, s), 2.66 (3H, s), 2.85-3.23 (6H, m), 3.67-3.92 (1H, m), 4.47-4.76 (1H, m), 7.24 (1H, d), 7.34 (2H, d), 7.48 (2H, d), 7.94 (2H, d), 8.95-9.24 (1H, m), m/z (ESI+) (M+H)⁺=455.27; HPLC tR=2.20 min.

### EXAMPLE 168

### N-[2-methyl-5-[4-[4-(1,3,4-oxadiazol-2-yl)phenyl]piperidine-1-carbonyl]phenyl]methanesulfonamide

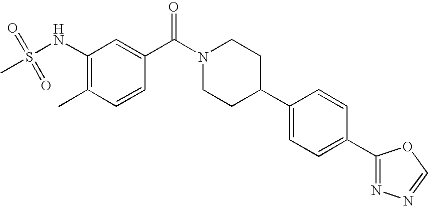

Hydrazine hydrate (0.14 mL, 2.9 mmol) was added to methyl 4-(1-(4-methyl-3-(methylsulfonamido) benzoyl)piperidin-4-yl)benzoate (Intermediate AA) (250 mg, 0.58 mmol) in DCM (5 mL) at ambient temperature over a period of 1 minute under argon and the resulting mixture was stirred at ambient temperature for 18 hours. It was then diluted with DCM (20 mL), and the mixture washed with water (25 mL). The organic layer was dried by passing through a phase separating cartridge and evaporated to afford crude intermediate hydrazide. To this was added triethyl orthoformate (0.290 mL, 1.74 mmol) in toluene (5 mL) and the mixture was stirred at 100° C. for 20 hours. The reaction mixture was concentrated and diluted with DCM (25 mL), and the solution was washed with water (25 mL). The organic layer was dried by passing through a phase separating cartridge and evaporated to afford crude product. This was purified by chromatography on silica (12 g column, elution gradient 0 to 5% MeOH in DCM) to give the title compound as a colourless solid (51.0 mg, 19.94%), ¹H NMR (400.132 MHz, DMSO-d₆) δ 1.57-2.02 (4H, m), 2.34 (3H, s), 2.62-2.98 (2H, m), 3.01 (3H, s), 3.05-3.24 (1H, m), 3.61-3.90 (1H, m), 4.42-4.77 (1H, m), 7.24 (1H, d), 7.33 (2H, d), 7.54 (2H, d), 7.98 (2H, d), 9.17 (1H, s), 9.31 (1H, s), m/z (ESI+) (M+H)⁺=441.42; HPLC tR=1.79 min.

### EXAMPLE 169

### N-[5-[4-(4-bromophenyl)piperidine-1-carbonyl]-2-methylphenyl]methanesulfonamide

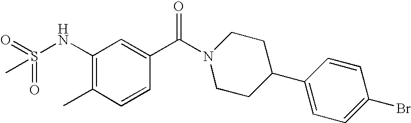

Prepared from Intermediate W

The title compound was prepared by a method analogous to that described in Example 155, using 4-(4-bromophenyl)piperidine hydrochloride in place of 4-(4-chlorophenyl)piperidine hydrochloride, ¹H NMR (400.132 MHz, CDCl₃) δ 1.60-1.95 (4H, m), 2.35 (3H, s), 2.71-2.79 (1H, m), 2.81-2.98 (1H, m), 3.05 (3H, s), 3.11-3.19 (1H, m), 3.79-4.02 (1H, m), 4.73-5.00 (1H, m), 6.35 (1H, s), 7.09 (2H, d), 7.20-7.23 (1H, m), 7.26-7.28 (1H, m), 7.43 (2H, d), 7.51-7.52 (1H, m), m/z (ESI+) (M+H)⁺=453; HPLC tR=2.48 min.

### EXAMPLE 170

### N-[2-methyl-5-[4-[4-(1,3-thiazol-2-yl)phenyl]piperidine-1-carbonyl]phenyl]methanesulfonamide

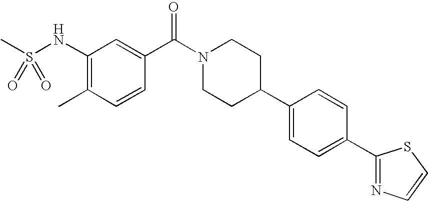

Tetrakis(triphenylphosphine) palladium(0) (0.064 g, 0.06 mmol) was added to N-(5-(4-(4-bromophenyl)piperidine-1-carbonyl)-2-methylphenyl)methanesulfonamide (Example 169) (0.25 g, 0.55 mmol) and 2-tributylstannylthiazole (0.518 g, 1.38 mmol) in toluene (6 mL). The resulting solution was de-gassed, heated to reflux and stirred for 24 hours under nitrogen. The reaction mixture was filtered through celite, diluted with EtOAc and the mixture was washed sequentially with water and saturated brine. The organic layer was dried over MgSO₄, filtered and evaporated to afford crude product. This was purified by chromatography on silica (elution gradient 30 to 70% EtOAc in isohexane) to give the title compound as a colourless solid (0.020 g, 7.93%), ¹H NMR (400.132 MHz, CDCl₃) δ 1.61-1.97 (4H, m), 2.34 (3H, s), 2.84 (1H, mult), 2.91-3.00 (1H, m), 3.06 (3H, s), 3.10-3.23 (1H, m), 3.80-4.02 (1H, m), 4.75-4.94 (1H, m), 6.34 (1H, s), 7.23-7.32 (3H, m), 7.43-7.48 (1H, m), 7.52-7.54 (1H, m), 7.64-7.70 (1H, m), 7.85 (1H, d), 7.92 (2H, d), m/z (ESI+) (M+H)⁺=456; HPLC tR=1.79 min.

### EXAMPLE 171

### N-[2-methyl-5-[4-[4-(1,2,4-oxadiazol-3-yl)phenyl]piperidine-1-carbonyl]phenyl]methanesulfonamide

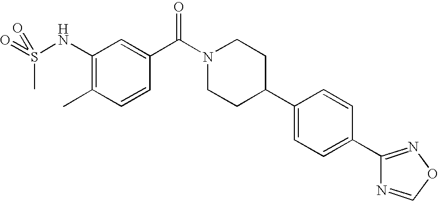

The title compound was prepared by a method essentially similar to that described for Example 167, starting from Intermediate BB and using triethyl orthoformate in place of acetyl chloride, ¹H NMR (500.133 MHz, DMSO-d₆) δ 1.62-1.74 (2H, m), 1.86-1.93 (2H, m), 2.36 (3H, s), 3.00-3.09 (6H, m), 4.16-4.25 (2H, m), 7.21 (1H, d), 7.31 (1H, d), 7.37 (1H, s), 7.47 (2H, d), 7.98 (2H, d), 8.65-8.85 (1H, m), 9.49 (1H, s), m/z (ESI+) (M+H)⁺=441.41; HPLC tR=2.13 min.

### EXAMPLE 172

### N-[5-[4-(4-ethynylphenyl)piperidine-1-carbonyl]-2-methylphenyl]methanesulfonamide

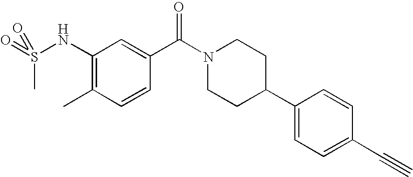

The title compound was prepared by a method essentially similar to that described in Tetrahedron Letters, 33 (23), p 3277 (1992), starting from N-[5-[4-(4-bromophenyl)piperidine-1-carbonyl]-2-methylphenyl]methanesulfonamide (Example 169) and trimethyl silyl acetylene, ¹H NMR (400.132 MHz, CDCl₃) δ 1.60-1.97 (4H, m), 2.34 (3H, s), 2.74-2.83 (1H, m), 2.86-2.97 (1H, m), 3.04 (1H, s), 3.05 (3H, s), 3.10-3.15 (1H, m), 3.79-4.09 (1H, m), 4.67-4.99 (1H, m), 6.40 (1H, s), 7.17 (2H, d), 7.21-7.23 (1H, m), 7.26-7.28 (1H, m), 7.44 (2H, d), 7.50-7.52 (1H, m), m/z (ESI+) (M+H)⁺=397; HPLC tR=2.36 min.

### EXAMPLE 173

### N-[2-methyl-5-[4-(4-pyridin-2-ylphenyl)piperidine-1-carbonyl]phenyl]methanesulfonamide

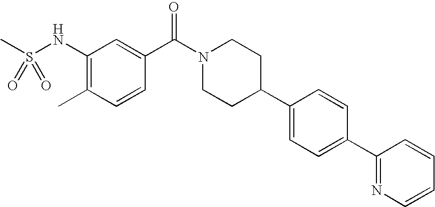

The title compound was prepared by a method essentially similar to that described for Example 170, starting from N-[5-[4-(4-bromophenyl)piperidine-1-carbonyl]-2-methylphenyl]methanesulfonamide (Example 169) and 2-(tributylstannyl)pyridine,

¹H NMR (400.132 MHz, CDCl₃) δ 1.66-1.96 (4H, m), 2.35 (3H, s), 2.77-3.00 (2H, m), 3.05 (3H, s), 3.10-3.20 (1H, m), 3.81-3.99 (1H, m), 4.73-4.96 (1H, m), 6.54 (1H, s), 7.19-7.25 (2H, m), 7.32 (2H, d), 7.43-7.56 (2H, m), 7.64-7.76 (2H, m), 7.95 (2H, d), 8.66-8.70 (1H, m), m/z (ESI+) (M+H)⁺=450; HPLC tR=1.65 min.

### EXAMPLE 174

### N-[2-methyl-5-[4-(4-pyrazin-2-ylphenyl)piperidine-1-carbonyl]phenyl]methanesulfonamide

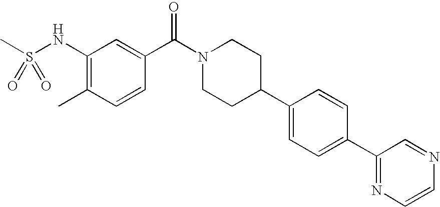

The title compound was prepared by a method essentially similar to that described for Example 170, starting from N-[5-[4-(4-bromophenyl)piperidine-1-carbonyl]-2-methylphenyl]methanesulfonamide (Example 169) and 2-(tributylstannyl)pyrazine, ¹H NMR (400.132 MHz, CDCl₃) δ 1.68-1.98 (4H, m), 2.34 (3H, s), 2.79-2.91 (2H, m), 3.05 (3H, s), 3.11-3.22 (1H, m), 3.81-4.01 (1H, m), 4.68-4.98 (1H, m), 6.52 (1H, s), 7.22-7.27 (2H, m), 7.37 (2H, d), 7.51-7.53 (1H, m), 7.98 (2H, d), 8.49 (1H, d), 8.61 (1H, s), 9.02 (1H, s), m/z (ESI+) (M+H)⁺=451; HPLC tR=2.04 min.

### EXAMPLE 175

### N-[2-methyl-5-[4-(4-phenylphenyl)piperidine-1-carbonyl]phenyl]methanesulfonamide

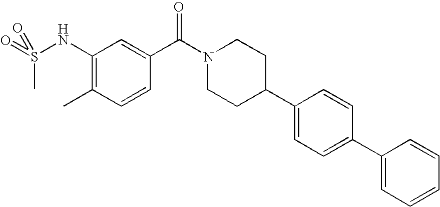

The title compound was prepared by a method essentially similar to that described in Journal of Organic Chemistry, 61(26), p 9582 (1996), starting from N-[5-[4-(4-bromophenyl)piperidine-1-carbonyl]-2-methylphenyl]methanesulfonamide (Example 169) and phenyl boronoic acid, ¹H NMR (400.132 MHz, CDCl₃) δ 1.67-2.09 (4H, m), 2.34 (3H, s), 2.79-2.96 (2H, m), 3.07 (3H, s), 3.13-3.25 (1H, m), 3.78-4.03 (1H, m), 4.70-5.04 (1H, m), 6.22 (1H, s), 7.23-7.32 (5H, m), 7.40-7.45 (2H, m), 7.54-7.59 (5H, m), m/z (ESI+) (M+H)⁺=449; HPLC tR=2.73 min.

### EXAMPLE 176

### N-[5-[4-(4-cyanophenyl)piperidine-1-carbonyl]-2-(methylsulfanylmethyl)phenyl]methanesulfonamide

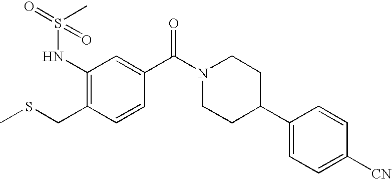

Prepared from Intermediate CC

¹H NMR (400.132 MHz, CDCl₃) δ 1.98-1.59 (4H, m), 2.01 (3H, s), 2.90-2.80 (2H, m), 3.24-2.91 (1H, m), 3.11 (3H, s), 3.75 (2H, s), 4.11-3.79 (1H, m), 5.00-4.70 (1H, m), 7.25-7.20 (2H, m), 7.35-7.30 (2H, m), 7.47 (1H, s), 7.64-7.59 (2H, m), m/z (EI+) (M+H)⁺=444.38; HPLC tR=2.26 min. m/z (EI−) (M−H)−=442.35; HPLC tR=2.26 min.

### EXAMPLE 177

### N-[2-methyl-5-[4-[4-(trifluoromethyl)phenyl]piperidine-1-carbonyl]phenyl]-1,1-dioxothiolane-3-sulfonamide

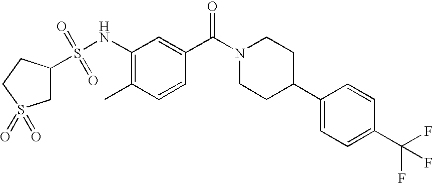

Prepared from Intermediate DD

¹H NMR (400.132 MHz, CDCl₃) δ 1.51-2.09 (4H, m), 2.31 (3H, s), 2.39-2.70 (2H, m), 2.78-2.98 (2H, m), 3.03-3.25 (2H, m), 3.29-3.49 (3H, m), 3.77-3.92 (1H, m), 3.92-4.05 (1H, m), 4.77-4.97 (1H, m), 7.14-7.22 (2H, m), 7.23-7.28 (1H, m), 7.34 (2H, d), 7.58 (2H, d), 8.21 (1H, s), m/z (ESI+) (M+H)⁺=545.39; HPLC tR=2.53 min.

### EXAMPLE 178

### N-[5-[4-(4-cyano-3-methoxyphenyl)piperidine-1-carbonyl]-2-methylphenyl]methanesulfonamide

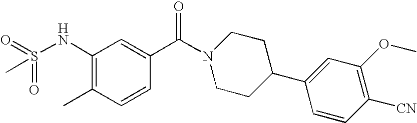

Prepared from Intermediates W and EE

The title compound was prepared by amide coupling of Intermediate W with 2-methoxy-4-(piperidin-4-yl)benzonitrile hydrochloride (Intermediate EE) H NMR (400.132 MHz, CDCl₃) δ 1.62-2.02 (9H, m), 2.34 (3H, s), 2.78-2.89 (2H, m), 3.07 (3H, s), 3.13-3.21 (1H, m), 3.90-3.95 (1H, m), 3.95 (3H, s), 4.83-4.91 (1H, m), 6.31 (1H, s), 6.81-6.89 (1H, m), 7.06-7.14 (1H, m), 7.23-7.30 (2H, m), 7.49-7.58 (2H, m), m/z (ESI+) (M+H)⁺=428; HPLC tR=2.15 min.

### EXAMPLE 179

### N-[5-[4-(4-cyano-3-fluorophenyl)piperidine-1-carbonyl]-2-methylphenyl]methanesulfonamide

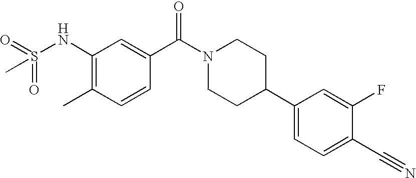

Prepared from Intermediates W and FF

The title compound was prepared by amide coupling of Intermediate W with 2-fluoro-4-piperidin-4-ylbenzonitrile hydrochloride (Intermediate FF), ¹H NMR (400.132 MHz, CDCl₃) δ 1.66-2.07 (4H, m), 2.34 (3H, s), 2.80-2.93 (2H, m), 3.05 (3H, s), 3.12-3.22 (1H, m), 3.87-4.00 (1H, m), 4.78-4.96 (1H, m), 6.67 (1H, s), 7.07-7.15 (2H, m), 7.20-7.29 (2H, m), 7.48-7.51 (1H, m), 7.58 (1H, t), m/z (ESI+) (M+H)⁺=416; HPLC tR=2.21 min.

### EXAMPLE 180

### N-[2-methyl-5-[4-[4-(trifluoromethoxy)phenyl]piperidine-1-carbonyl]phenyl]methanesulfonamide

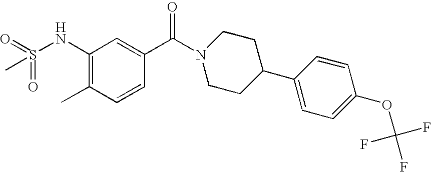

Prepared from Intermediates W and GG

¹H NMR 2.35 (3H, s), 2.77-2.88 (2H, m), 3.06 (3H, s), 3.12-3.20 (1H, m), 3.85-3.98 (1H, m), 4.81-4.95 (1H, m), 6.25 (1H, s), 7.15-7.18 (1H, m), 7.22-7.30 (5H, m), 7.53-7.55 (1H, m), m/z (ESI+) (M+H)⁺=457; HPLC tR=2.60 min.

### EXAMPLE 181

### N-[5-[4-[4-(cyanomethoxy)phenyl]piperidine-1-carbonyl]-2-methylphenyl]methanesulfonamide

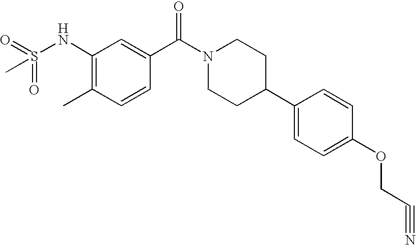

Prepared from Intermediates W and HH

¹H NMR (400.132 MHz, CDCl₃) δ 1.63-2.01 (4H, m), 2.35 (3H, s), 2.69-2.78 (1H, m), 2.83-2.90 (1H, m), 3.06 (3H, s), 3.10-3.19 (1H, m), 3.82 (2H, s), 3.86-3.91 (1H, m), 4.80-4.88 (1H, m), 6.24 (1H, s), 6.86 (1H, d), 6.95 (1H, d), 7.14 (1H, d), 7.19-7.30 (3H, m), 7.51-7.55 (1H, m), m/z (ESI+) (M+H)⁺=428; HPLC tR=2.12 min.

### EXAMPLE 182

### N-[2-methyl-5-[4-(4-methylsulfinylphenyl)piperidine-1-carbonyl]phenyl]methanesulfonamide

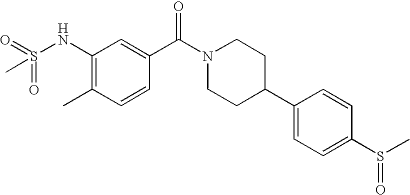

Prepared from Intermediates W and II

¹H NMR (400.132 MHz, CDCl₃) δ 1.68-2.04 (4H, m), 2.35 (3H, s), 2.73 (3H, s), 2.82-2.92 (2H, m), 3.05 (3H, s), 3.12-3.23 (1H, m), 3.82-3.97 (1H, m), 4.76-4.93 (1H, m), 6.33 (1H, s), 7.23-7.31 (2H, m), 7.39 (2H, d), 7.53-7.55 (1H, m), 7.61 (2H, d), m/z (ESI+) (M+H)⁺=435; HPLC tR=1.62 min.

### EXAMPLE 183

### 4-[1-(3-methanesulfonamido-4-methylbenzoyl)piperidin-4-yl]-N-methylbenzenesulfonamide

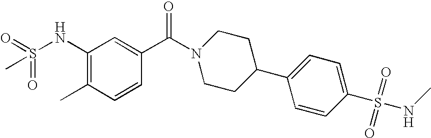

Prepared from Intermediates W and JJ

¹H NMR (400.132 MHz, CDCl₃) δ 1.64-2.05 (4H, m), 2.36 (3H, s), 2.68 (3H, d), 2.83-2.92 (2H, m), 3.07 (3H, s), 3.38-3.61 (1H, m), 3.84-3.99 (1H, m), 4.19-4.28 (1H, m), 4.79-4.99 (1H, m), 6.21 (1H, s), 7.23-7.31 (2H, m), 7.37 (2H, d), 7.54-7.56 (1H, m), 7.81 (2H, d), m/z (ESI+) (M+H)⁺=466; HPLC tR=1.87 min.

### EXAMPLE 184

### N-cyclopropyl-4-[1-(3-methanesulfonamido-4-methylbenzoyl)piperidin-4-yl]benzamide

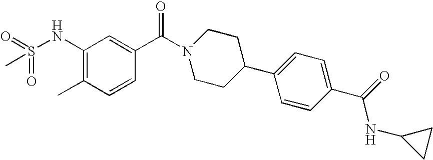

Prepared from Intermediates W and KK

¹H NMR (400.132 MHz, CDCl₃) δ 0.59-0.64 (2H, m), 0.85-0.90 (2H, m), 1.72-2.05 (4H, m), 2.34 (3H, s), 2.78-2.93 (3H, m), 3.06 (3H, s), 3.11-3.24 (1H, m), 3.83-3.98 (1H, m), 4.77-4.95 (1H, m), 6.20 (1H, s), 6.29 (1H, s), 7.21-7.30 (4H, m), 7.53-7.55 (1H, m), 7.69 (2H, d), m/z (ESI+) (M+H)⁺=456; HPLC tR=1.70 min.

### EXAMPLE 185

### [4-[1-(3-methanesulfonamido-4-methylbenzoyl)piperidin-4-yl]phenyl]methanesulfonate

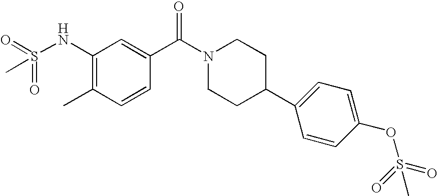

Prepared from Intermediates W and LL

¹H NMR (400.132 MHz, CDCl₃) δ 1.64-2.03 (4H, m), 2.34 (3H, s), 2.77-2.94 (2H, m), 3.06 (3H, s), 3.15 (3H, s), 3.16-3.21 (1H, m), 3.84-3.98 (1H, m), 4.81-4.93 (1H, m), 6.34 (1H, s), 7.22-7.31 (6H, m), 7.51-7.56 (1H, m), m/z (ESI+) (M+H)⁺=467; HPLC tR=2.08 min.

The following Examples were prepared in a similar manner.

### EXAMPLE 186

4-[1-(3-methanesulfonamido-4-methylbenzoyl)piperidin-4-yl]benzenesulfonamide

### EXAMPLE 187

N-[2-methyl-5-[4-(4-pyridin-2-yloxyphenyl)piperidine-1-carbonyl]phenyl]methanesulfonamide

### EXAMPLE 188

N-[2-methyl-5-[4-[4-(1,3-thiazole-2-carbonyl)phenyl]piperidine-1-carbonyl]phenyl]methanesulfonamide

### EXAMPLE 189

N-[2-methyl-5-[4-[4-[4-(trifluoromethyl)pyrimidin-2-yl]oxyphenyl]piperidine-1-carbonyl]phenyl]methanesulfonamide

### EXAMPLE 190

N-[2-methyl-5-[4-(4-pyrimidin-2-yloxyphenyl)piperidine-1-carbonyl]phenyl]methanesulfonamide

### EXAMPLE 191

N-[2-chloro-5-[4-(4-cyanophenyl)piperidine-1-carbonyl]phenyl]-2-fluorobenzenesulfonamide

### EXAMPLE 192

4-chloro-N-[2-chloro-5-[4-(4-cyanophenyl)piperidine-1-carbonyl]phenyl]benzene-sulfonamide

### EXAMPLE 193

N-[2-methyl-5-[4-(4-methylsulfonylphenyl)piperidine-1-carbonyl]phenyl]-1-phenyl-methanesulfonamide

### EXAMPLE 194

N-[5-[4-(4-cyanophenyl)piperidine-1-carbonyl]-2-fluorophenyl]-1-phenylmethane-sulfonamide

### EXAMPLE 195

N-[5-[4-(4-cyanophenyl)piperidine-1-carbonyl]-2-methylphenyl]-1-methylsulfonyl-methanesulfonamide

### EXAMPLE 196

N-[2-cyano-5-[4-(4-cyanophenyl)piperidine-1-carbonyl]phenyl]-1-phenylmethane-sulfonamide

### EXAMPLE 197

N-[3-[4-(4-cyanophenyl)piperidine-1-carbonyl]-4-methoxyphenyl]methanesulfonamide

### EXAMPLE 198

4-butoxy-N-[3-[4-(4-cyanophenyl)piperidine-1-carbonyl]-4-methylphenyl]benzene-sulfonamide

### EXAMPLE 199

N-[3-[4-(4-cyanophenyl)piperidine-1-carbonyl]-4-fluorophenyl]-1-phenylmethane-sulfonamide

### EXAMPLE 200

N-[5-[4-(4-bromophenyl)-4-hydroxypiperidine-1-carbonyl]-2-methylphenyl]methane-sulfonamide

### EXAMPLE 201

N-[5-[4-(4-bromophenyl)-4-hydroxypiperidine-1-carbonyl]-2-methylphenyl]-1-phenylmethanesulfonamide

### EXAMPLE 202

N-[5-[4-(4-cyanophenyl)piperidine-1-carbonyl]-2-methylphenyl]-2-phenylethanesulfonamide

### EXAMPLE 203

N-[3-[4-(4-cyanophenyl)piperidine-1-carbonyl]phenyl]methanesulfonamide

### EXAMPLE 204

4-[1-[3-(benzylsulfonylamino)-4-methylbenzoyl]piperidin-4-yl]-N,N-dimethylbenzamide

### EXAMPLE 205

N-[5-[4-(4-cyanophenyl)piperidine-1-carbonyl]-2-methylphenyl]-3-hydroxypropane-1-sulfonamide

### EXAMPLE 206

2-[[5-[4-(4-cyanophenyl)piperidine-1-carbonyl]-2-methylphenyl]sulfamoyl]-N,N-dimethylbenzamide

### EXAMPLE 207

2-[[5-[4-(4-cyanophenyl)piperidine-1-carbonyl]-2-methylphenyl]sulfamoyl]-N-(2-hydroxyethyl)benzamide

### EXAMPLE 208

2-[[5-[4-(4-cyanophenyl)piperidine-1-carbonyl]-2-methylphenyl]sulfamoyl]-N-propan-2-ylbenzamide

### EXAMPLE 209

N-[5-[4-(4-cyanophenyl)piperidine-1-carbonyl]-2-methylphenyl]-1,1,1-trifluoromethane-sulfonamide

### EXAMPLE 210

N-[5-[4-(4-cyanophenyl)piperidine-1-carbonyl]-2-methylsulfonylphenyl]methanesulfonamide

### EXAMPLE 211

N-[5-[4-(4-cyanophenyl)piperidine-1-carbonyl]-2-methylsulfinylphenyl]-1-phenylmethanesulfonamide

### EXAMPLE 212

1-cyano-N-[5-[4-(4-cyanophenyl)piperidine-1-carbonyl]-2-methylphenyl]methanesulfonamide

### EXAMPLE 213

N-[5-[4-(4-fluorophenyl)piperidine-1-carbonyl]-2-methylphenyl]methanesulfonamide

### EXAMPLE 214

N′-hydroxy-4-[1-(3-methanesulfonamido-4-methylbenzoyl)piperidin-4-yl]benzene-carboximidamide

### EXAMPLE 215

N-[5-[4-hydroxy-4-[4-(trifluoromethyl)phenyl]piperidine-1-carbonyl]-2-methylphenyl]methanesulfonamide

### EXAMPLE 216

N-[5-[4-(4-chlorophenyl)piperidine-1-carbonyl]-2-methylphenyl]-1,1-dioxothiolane-3-sulfonamide

### EXAMPLE 217

N-[5-[4-(4-cyano-3-methylphenyl)piperidine-1-carbonyl]-2-methylphenyl]methane-sulfonamide

### EXAMPLE 218

N-[5-[4-(3-chloro-4-cyanophenyl)piperidine-1-carbonyl]-2-methylphenyl]methane-sulfonamide

### EXAMPLE 219

N-[5-[4-[4-(2-methoxyethoxy)phenyl]piperidine-1-carbonyl]-2-methylphenyl]methane-sulfonamide

### EXAMPLE 220

N-(2-hydroxyethyl)-4-[1-(3-methanesulfonamido-4-methylbenzoyl)piperidin-4-yl]benzenesulfonamide

### EXAMPLE 221

N-[4-[1-(3-methanesulfonamido-4-methylbenzoyl)piperidin-4-yl]phenyl]methane-sulfonamide

### EXAMPLE 222

N-(2-dimethylaminoethyl)-4-[1-(3-methanesulfonamido-4-methylbenzoyl)piperidin-4-yl]benzamide

### EXAMPLE 223

N-[2-methyl-5-[4-[4-(methylsulfonylmethyl)phenyl]piperidine-1-carbonyl]phenyl]-methanesulfonamide

### EXAMPLE 224

2-chloro-N-[5-[4-(4-cyanophenyl)piperidine-1-carbonyl]-2-methylphenyl]propane-2-sulfonamide

### EXAMPLE 225

N—[[4-[1-(3-methanesulfonamido-4-methylbenzoyl)piperidin-4-yl]phenyl]methyl]propanamide

### EXAMPLE 226

N-[[4-[1-(3-methanesulfonamido-4-methylbenzoyl)piperidin-4-yl]phenyl]methyl]-2-methylpropanamide

### EXAMPLE 227

N-[[4-[1-(3-methanesulfonamido-4-methylbenzoyl)piperidin-4-yl]phenyl]methyl]ethanesulfonamide

### EXAMPLE 228

Ethyl N-[[4-[1-(3-methanesulfonamido-4-methylbenzoyl)piperidin-4-yl]phenyl]methyl]carbamate

### EXAMPLE 229

N-[2-methyl-5-[4-[4-(6-methylpyridazin-3-yl)oxyphenyl]piperidine-1-carbonyl]phenyl]methanesulfonamide

### EXAMPLE 230

N-[2-methyl-5-[4-[4-(methyl-methylsulfonylamino)phenyl]piperidine-1-carbonyl]phenyl]methanesulfonamide

### EXAMPLE 231

N-[2-methyl-5-[4-[4-(morpholine-4-carbonyl)phenyl]piperidine-1-carbonyl]phenyl]methanesulfonamide

### EXAMPLE 232

N-[2-methyl-5-[4-[4-(trifluoromethylsulfanyl)phenyl]piperidine-1-carbonyl]phenyl]methanesulfonamide

### EXAMPLE 233

1-[[4-[1-(3-methanesulfonamido-4-methylbenzoyl)piperidin-4-yl]phenyl]methyl]-3-methylurea

### EXAMPLE 234

1-ethyl-3-[[4-[1-(3-methanesulfonamido-4-methylbenzoyl)piperidin-4-yl]phenyl]methyl]urea

### EXAMPLE 235

N-[[4-[1-(3-methanesulfonamido-4-methylbenzoyl)piperidin-4-yl]phenyl]methyl]butanamide

### EXAMPLE 236

N-[[4-[1-(3-methanesulfonamido-4-methylbenzoyl)piperidin-4-yl]phenyl]methyl]-3-methylbutanamide

### EXAMPLE 237

N-[[4-[1-(3-methanesulfonamido-4-methylbenzoyl)piperidin-4-yl]phenyl]methyl]propane-1-sulfonamide

### EXAMPLE 238

N-[[4-[1-(3-methanesulfonamido-4-methylbenzoyl)piperidin-4-yl]phenyl]methyl]-2-methylpropane-1-sulfonamide

### EXAMPLE 239

Methyl N-[[4-[1-(3-methanesulfonamido-4-methylbenzoyl)piperidin-4-yl]phenyl]methyl]carbamate

### EXAMPLE 240

2-[4-[1-(3-methanesulfonamido-4-methylbenzoyl)piperidin-4-yl]phenyl]-N-methylacetamide

### EXAMPLE 241

2-hydroxy-N-[[4-[1-(3-methanesulfonamido-4-methylbenzoyl)piperidin-4-yl]phenyl]methyl]acetamide

### EXAMPLE 242

N-[5-[4-[4-[(dimethylsulfamoylamino)methyl]phenyl]piperidine-1-carbonyl]-2-methylphenyl]methanesulfonamide

### EXAMPLE 243

N-[2-methyl-5-[4-[4-(morpholin-4-ylmethyl)phenyl]piperidine-1-carbonyl]phenyl]methanesulfonamide

### EXAMPLE 244

3-[4-[1-[3-(cyclohexylsulfonylamino)-4-methylbenzoyl]piperidin-4-yl]phenyl]propanoic acid

### EXAMPLE 245

Methyl 2-[[4-[1-(3-methanesulfonamido-4-methylbenzoyl)piperidin-4-yl]phenyl]methylsulfamoyl]acetate

### EXAMPLE 246

4-[4-[[13-(cyclohexylsulfonylamino)-4-methylbenzoyl]piperidin-4-yl]phenyl]butanoic acid

### EXAMPLE 247

N-[5-[4-[4-[3-(2-methoxyethoxy)prop-1-ynyl]phenyl]piperidine-1-carbonyl]-2-methylphenyl]methanesulfonamide. Human Fatty Acid Synthase IC₅₀ 1.78.

### EXAMPLE 248

3-[4-[1-(3-methanesulfonamido-4-methylbenzoyl)piperidin-4-yl]phenyl]-N-methylpropanamide. Human Fatty Acid Synthase IC₅₀ 0.405.

### EXAMPLE 249

### 3-[4-[1-(3-methanesulfonamido-4-methylbenzoyl)piperidin-4-yl]phenyl]-N,N-dimethylpropanamide. Human Fatty Acid Synthase IC₅₀ 0.515.

### EXAMPLE 250

N-[5-[4-[4-(5-hydroxypent-1-ynyl)phenyl]piperidine-1-carbonyl]-2-methylphenyl]methanesulfonamide. Human Fatty Acid Synthase IC₅₀ 1.44.

### EXAMPLE 251

N-[5-[4-[4-(4-hydroxybut-1-ynyl)phenyl]piperidine-1-carbonyl]-2-methylphenyl]methanesulfonamide. Human Fatty Acid Synthase IC₅₀ 0.663.

### EXAMPLE 252

### N-[5-[4-[4-(3-hydroxy-3-methylbut-1-ynyl)phenyl]piperidine-1-carbonyl]-2-methylphenyl]methanesulfonamide. Human Fatty Acid Synthase IC₅₀ 3.33.

### EXAMPLE 253

2-[[4-[1-(3-methanesulfonamido-4-methylbenzoyl)piperidin-4-yl]phenyl]methylsulfamoyl]acetic acid. Human Fatty Acid Synthase IC₅₀ 12.7.

### Preparation of Intermediates

### Intermediate A

### 4-[1-(3-amino-4-methyl-benzoyl)-4-piperidyl]benzonitrile

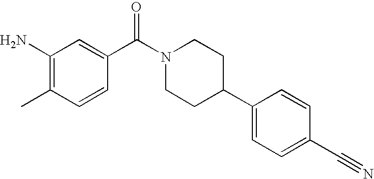

A solution of 3-amino-4-methyl benzoic acid (4.05 g, 26.792 mmol), 4-(4′-cyanophenyl)piperidine (5 g, 26.79 mmol), N-(3-Dimethylaminopropyl)-N′-ethylcarbodiimide hydrochloride [EDAC] (5.64 g, 29.47 mmol, 1.1 eq) and DMAP (328 mg, 2.68 mmol, 0.1 eq) in DMF (60 mL) was stirred at ambient temperature for 2 hrs. Ethyl acetate (200 mL) was added and the resulting solution was washed sequentially with KHSO₄ solution (100 mL of 2M), and brine (100 ml); a precipitate formed and was filtered off to give the title compound as a colourless solid (5.25 g), ¹H NMR (300.073 MHz, DMSO-d⁶, 30° C.) δ 1.47-1.67 (2H, m), 1.68-1.89 (2H, m), 2.05 (3H, s), 2.68-3.15 (3H, m), 3.59-4.13 (1H, m), 4.22-4.76 (1H, m), 4.97 (2H, s), 6.45-6.53 (1H, m), 6.63 (1H, s), 6.90-6.99 (1H, m), 7.44-7.54 (2H, m), 7.71-7.81 (2H, m), m/z 320 (M+H)⁺.

### Intermediate B

### 4-[1-(3-amino-4-methoxy-benzoyl)-4-piperidyl]benzonitrile

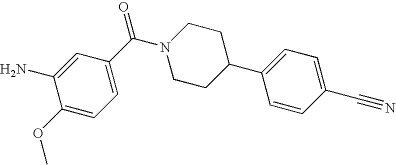

A stirred mixture of 4-(4′-cyanophenyl)piperidine (3 g, 16 mmol); 3-amino-4-methoxybenzoic acid (2.675 g, 16 mmol, 1 eq) and DIPEA (4.2 ml, 24 mmol, 1.5 eq) in DCM (100 mL) was blanketed with nitrogen and treated with N-(3-Dimethylaminopropyl)-N′-ethylcarbodiimide hydrochloride (EDAC) (3.4 g, 17.6 mmol, 1.1 eq). The reaction mixture was stirred for three days. Addition of water to the reaction mixture resulted in an emulsion and a colourless precipitate. The solid was isolated by filtration and washed with EtOAc (2×75 mL portions) to give a colourless solid (2.5 g). The ethyl acetate washings were combined, washed with water, dried (MgSO₄) and evaporated to give a further 2 g; the solids thus prepared were identical and combined to give the title compound as (4.5 g, 83%), ¹H NMR (300.073 MHz, DMSO-d₆, 30° C.) δ 1.48-1.67 (2H, m), 1.71-1.87 (2H, m), 2.80-3.08 (3H, m), 3.78 (3H, s), 3.88-4.63 (2H, m), 4.84 (2H, s), 6.56-6.64 (1H, m), 6.67-6.73 (1H, m), 6.80 (1H, dJ=8.1 Hz), 7.49 (2H, dJ=8.1 Hz), 7.76 (2H, dJ=9.4 Hz), m/z 336 (M+H)⁺.

### Intermediate C

### 4-[1-(5-amino-2-methyl-benzoyl)-4-piperidyl]benzonitrile

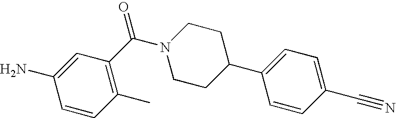

### Step 1: 4-[1-(2-methyl-5-nitro-benzoyl)-4-piperidyl]benzonitrile

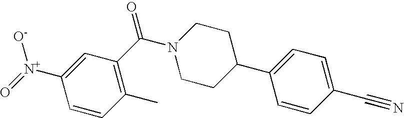

A mixture of 2-methyl-5-nitrobenzoic acid (5 g, 27.6 mmol), 4-(4′-cyanophenyl)piperidine (5.14 g, 27.6 mmol), N-(3-dimethylaminopropyl)-N′-ethylcarbodiimide hydrochloride (5.82 g, 30.36 mmol) and DMAP (338 mg, 2.76 mmol) in DMF (50 mL) was stirred at room temperature for 2 hrs. Ethyl acetate (200 ml) was added and the resulting solution washed with dilute hydrochloric acid (100 mL of 1M), NaHCO₃, brine (100 mL). At this point a colourless solid precipitated, which was isolated by filtration and dried. The filtrate was dried (MgSO₄), filtered and reduced in vacuo to give a white solid. This was chromatographed (120 g silica column, Companion, eluting with a gradient consisting of 0-50% ethyl acetate in isohexane) to give a colourless solid which was identical to that isolated previously. The solids were combined to give the title compound (4 g), ¹H NMR (300.072 MHz, CDCl₃) δ1.64-2.11 (4H, m), 2.42-2.53 (3H, m), 2.79-3.00 (2H, m), 3.09-3.23 (1H, m), 3.55 (1H, d), 4.98 (1H, d), 7.29-7.36 (2H, m), 7.42 (1H, d), 7.63 (2H, d), 8.04-8.18 (2H, m), m/z 348 (M−H)⁻.

### Step 2: 4-[1-(5-amino-2-methyl-benzoyl)-4-piperidyl]benzonitrile

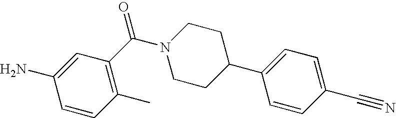

A solution of 4-[1-(2-methyl-5-nitro-benzoyl)-4-piperidyl]benzonitrile (4 g, 11.45 mmol) in ethanol/THF (200 mL of a 1:1 mixture) was placed under an atmosphere of argon and treated with 10% palladium on carbon catalyst (0.6 g, 15% by weight). The reaction mixture was stirred under a hydrogen atmosphere for 4 hrs. The catalyst was removed by filtration through celite, and the filtrate evaporated in vacuo to give a yellow foam. Some starting material still remained so the hydrogenation was repeated as above, with stirring for a further 1 hr. The isolation was repeated as above to give a yellow foam. EtOAc was added and the solution washed with water and brine; the solvent was removed in vacuo to give the title compound as a yellow solid (3.3 g), ¹H NMR (400.132 MHz, DMSO-d₆) δ 1.39-1.62 (2H, m), 1.69-1.78 (1H, m), 1.88 (1H, d), 1.99-2.12 (3H, m), 2.80 (1H, t), 2.90-2.99 (1H, m), 3.08 (1H, t), 3.45-3.51 (1H, m), 4.63-4.72 (1H, m), 5.01 (2H, s), 6.37 (1H, d), 6.48-6.52 (1H, m), 6.89 (1H, d), 7.46-7.52 (2H, m), 7.78 (2H, d), m/z 320 (M+H)⁺.

### Intermediate D

### 4-[1-(5-amino-2,4-dimethyl-benzoyl)-4-piperidyl]benzonitrile

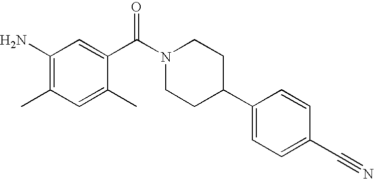

### Step 1: 4-[1-(2,4-dimethyl-5-nitro-benzoyl)-4-piperidyl]benzonitrile

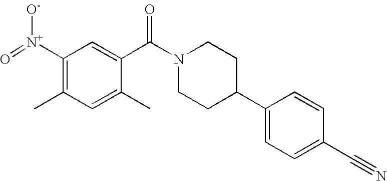

The title compound was prepared in a manner similar to that described for Intermediate C, Step 1, starting from 2,4-dimethyl-5-nitro-benzoic acid and 4-(4′-cyanophenyl)piperidine; ¹H NMR (300.072 MHz, CDCl₃) δ 1.44-1.64 (m, 1H), 1.66-1.91 (m, 2H), 1.95-2.09 (m, 1H), 2.30-2.49 (br s, 3H), 2.61 (s, 3H), 2.79-2.95 (m, 2H), 3.05-3.26 (m, 1H), 3.59 (d, 1H), 4.95 (d, 1H), 7.22 (s, 1H), 7.32 (d, 2H), 7.61 (d, 2H), 7.81 (br s, 1H), m/z 405 (M+MeCN+H)⁺.

### Step 2: 4-[1-(5-amino-2,4-dimethyl-benzoyl)-4-piperidyl]benzonitrile

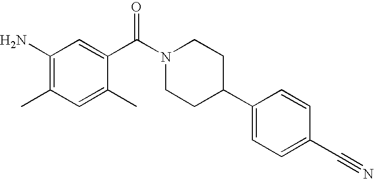

The title compound was prepared in a manner similar to that described for Intermediate C, Step 2, starting from 4-[1-(2,4-dimethyl-5-nitro-benzoyl)-4-piperidyl]benzonitrile, and using a methanol/THF mixture (1:1) as solvent; ¹H NMR (300.073 MHz, DMSO-d₆) δ 1.36-1.63 (m, 2H), 1.64-1.79 (m, 1H), 1.80-1.95 (m, 1H), 1.96-2.08 (br s, 3H), 2.02 (s, 3H), 2.69-2.85 (m, 1H), 2.85-2.97 (m, 1H), 2.98-3.12 (m, 1H), 3.40-3.56 (m, 1H), 4.59-4.70 (m, 1H), 4.73 (br s, 2H), 6.30-6.55 (br m, 1H), 6.78 (s, 1H), 7.47 (d, 2H), 7.77 (d, 2H); peak broadening is observed due to conformations of amide group, m/z 334 (M+H)⁺.

### Intermediate E

### 2-amino-4-[4-(4-cyanophenyl)piperidine-1-carbonyl]benzonitrile

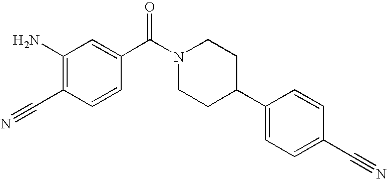

### Step 1: Ethyl 4-cyano-3-nitro-benzoate

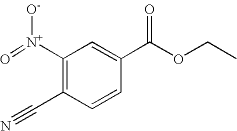

Water (0.01 mL) was added to a solution of 4-iodo-3-nitro benzoic acid ethyl ester (0.4 g, 1.25 mmol) and zinc cyanide (79 mg, 0.67 mmol) in NMP (5 mL) and nitrogen was bubbled through the mixture for 5 mins. Bis(dibenzylideneacetone)palladium(0) (29 mg, 0.05 mmol) and 1,1′-Bis(diphenylphosphino)ferrocene (83 mg, 0.15 mmol) were added and the vessel sealed and filled with nitrogen. The reaction was heated in the microwave oven at 150° C. for 5 mins. EtOAc (50 ml) was added and the resulting mixture was filtered through celite and then washed sequentially with dilute aqueous hydrochloric acid (50 mL of 1M), saturated aqueous sodium bicarbonate solution (50 mL), water (50 mL) and brine (50 mL), dried (MgSO₄), filtered and reduced in vacuo to give a brown oil which was chromatographed (40 g silica column, Companion, eluting with a gradient consisting of isohexane containing 0-20% EtOAc to give the title compound as a yellow solid (200 mg), ¹H NMR (300.072 MHz, CDCl₃) [symbol] 1.45 (t, 3H), 4.49 (q, 2H), 8.01 (d, 1H), 8.42-8.47 (m, 1H), 8.92 (d, 1H), m/z 220 (M^{.+}).

### Step 2: Ethyl 3-amino-4-cyano-benzoate

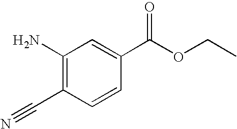

This was prepared by hydrogenation of ethyl 4-cyano-3-nitro-benzoate (Step 1) using a procedure similar to that described in Intermediate C, Step 2, to give the title compound as a yellow solid, ¹H NMR (300.072 MHz, CDCl₃) δ 1.39 (3H, t), 4.38 (2H, q), 4.57 (2H, s), 7.34-7.47 (3H, m), m/z 190 (M^{.+}).

### Step 3: 3-amino-4-cyano-benzoic acid

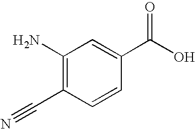

A solution of ethyl 3-amino-4-cyano-benzoate (Step 2) (140 mg, 0.74 mmol) in THF (6 mL) was treated with a solution of lithium hydroxide monohydrate (47 mg, 1.10 mmol) in water (3 ml), and the mixture stirred at ambient temperature for 2 hrs. The THF was removed in vacuo and the aqueous residue washed with EtOAc (30 mL) to remove any unreacted starting material. The aqueous was then adjusted to pH3 with citric acid solution (1M), and extracted with EtOAc (20 mL). The organic extracts were washed with brine (20 mL), dried (MgSO₄), filtered and reduced in vacuo to give the title compound as a yellow solid (60 mg), ¹H NMR (300.073 MHz, DMSO-d₆) [symbol] 6.27 (s, 2H), 7.04-7.08 (m, 1H), 7.38-7.40 (m, 1H), 7.47 (d, 1H), 13.03 (s, 1H), m/z 161 (M−H)⁻.

### Step 4: 2-amino-4-[4-(4-cyanophenyl)piperidine-1-carbonyl]benzonitrile

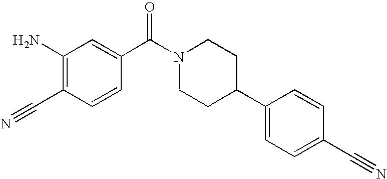

The title compound was prepared in a manner similar to that described for Intermediate C, Step 1, starting from 3-amino-4-cyano-benzoic acid (Step 3) and 4-(4′-cyanophenyl)piperidine, ¹H NMR (300.072 MHz, CDCl₃) [symbol] 1.53-2.06 (m, 4H), 2.78-2.92 (m, 2H), 3.05-3.22 (m, 1H), 3.71-3.96 (m, 1H), 4.61 (s, 2H), 4.79-4.97 (m, 1H), 6.71-6.75 (m, 1H), 6.78-6.81 (m, 1H), 7.32 (d, 2H), 7.42 (d, 1H), 7.62 (d, 2H), m/z 331 (M+H)⁺.

### Intermediate F

### 4-[1-(5-amino-2-methoxy-benzoyl)-4-piperidyl]benzonitrile

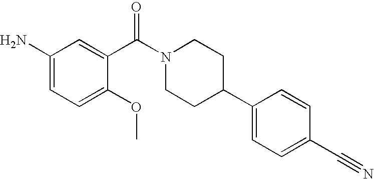

### Step 1: 2-methoxy-5-nitro-benzoic acid

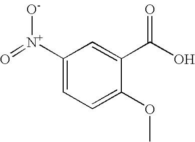

The title compound was prepared by hydrolysis of methyl 2-methoxy-5-nitro-benzoate in a manner similar to that described for Intermediate E, Step 3, ¹H NMR (400.132 MHz, DMSO-d₆) δ 3.97 (s, 3H), 7.36 (d, 1H), 8.36-8.43 (m, 1H), 8.46 (d, 1H), 13.33 (s, 1H), m/z 196 (M−H)⁻.

### Step 2: 4-[1-(2-methoxy-5-nitro-benzoyl)-4-piperidyl]benzonitrile

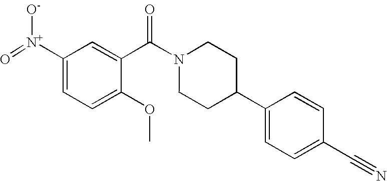

The title compound was prepared in a manner similar to that described for Intermediate C, Step 1, starting from 2-methoxy-5-nitro-benzoic acid (Step 1) and 4-(4′-cyanophenyl)piperidine, m/z 366 (M+H)⁺.

### Step 3: 4-[1-(5-amino-2-methoxy-benzoyl)-4-piperidyl]benzonitrile

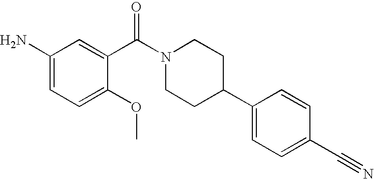

This was prepared by hydrogenation of 4-[1-(2-methoxy-5-nitro-benzoyl)-4-piperidyl]benzonitrile (Step 2) using a procedure similar to that described for Intermediate C, Step 2, to give the title compound as a pale yellow foam, which was used without further purification, m/z 336 (M+H)⁺.

### Intermediate G

### 4-[1-(3-aminobenzoyl)-4-piperidyl]benzonitrile

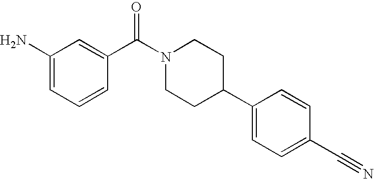

The title compound was prepared in a manner similar to that described for Intermediate A, starting from 3-amino benzoic acid and 4-(4′-cyanophenyl)piperidine. After work-up of the reaction, the crude product was triturated with ether and recrystallised from EtOAc to give a pink solid, ¹H NMR (300.072 MHz, CDCl₃) δ1.50-2.04 (4H, m), 2.79-2.89-3.20 (3H, m), 3.76-3.97 (2H, s), 4.00 (1H, s), 4.90 (1H, s), 6.70-6.78 (3H, m), 7.15-7.20 (1H, m), 7.32 (2H, d), 7.60-7.63 (2H, m), m/z 306 (M+H)⁺.

### Intermediate H

### (3-amino-4-methyl-phenyl)-[4-(4-methylsulfonylphenyl)-1-piperidyl]methanone

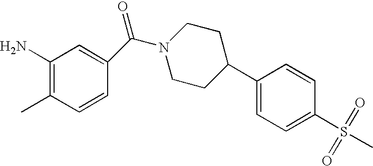

The title compound was prepared in a manner similar to that described for Intermediate A, starting from 3-amino-4-methyl benzoic acid and 4-(4-methylsulfonylphenyl)piperidine, m/z 373 (M+H)⁺.

### Intermediate I

### 4-[1-(3-amino-4-methyl-benzoyl)-4-hydroxy-4-piperidyl]benzonitrile

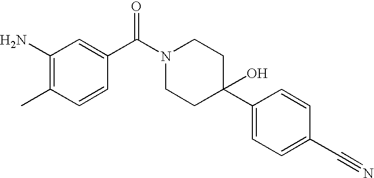

### Step 1: [4-(4-bromophenyl)-4-hydroxy-1-piperidyl]-(4-methyl-3-nitro-phenyl)methanone

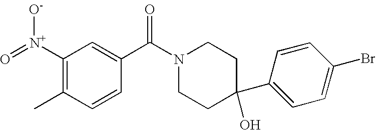

A solution of 4-(4-bromophenyl)-4-piperidinol (2.5 g, 9.76 mmol) and 4-methyl-3-nitrobenzoyl chloride (1.42 mL, 9.76 mmol) in DCM (30 mL) was treated with DIPEA (2.04 mL, 14.05 mmol) and the reaction mixture stirred at ambient temperature for 20 hrs. It was then washed sequentially with aqueous citric acid (40 mL of 1M), saturated sodium bicarbonate solution (40 mL) and brine (40 mL), dried (MgSO₄), and evaporated in vacuo to give a yellow oil. DCM was added and a colourless solid filtered off (2.1 g). The filtrate was purified by chromatography (120 g silica column, gradient eluting with 20-70% EtOAc in isohexane) to give a colourless solid (0.97 g). This was combined with the product isolated previously to give the title compound (3.07 g), ¹H NMR (300.072 MHz, CDCl₃) [symbol] 1.67 (s, 1H), 1.73-2.20 (m, 4H), 2.65 (s, 3H), 3.25-3.74 (m, 3H), 4.51-4.81 (m, 1H), 7.34-7.39 (m, 2H), 7.42 (d, 1H), 7.49-7.54 (m, 2H), 7.57-7.61 (m, 1H), 8.06 (d, 1H).

### Step 2: 4-[4-hydroxy-1-(4-methyl-3-nitro-benzoyl)-4-piperidyl]benzonitrile

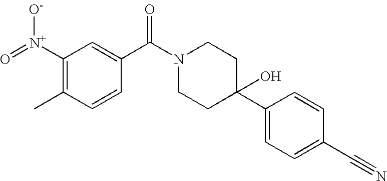

A mixture of [4-(4-bromophenyl)-4-hydroxy-1-piperidyl]-(4-methyl-3-nitro-phenyl)methanone (1.57 g, 3.74 mmol) and copper (I) cyanide (504 mg, 5.62 mmol) in NMP (20 mL) was stirred in the microwave at 190° C. for 12 hrs. EtOAc (30 mL) was added and the resulting mixture was washed sequentially with water (30 mL) and brine (30 mL), dried (MgSO₄) and evaporated in vacuo to give a brown oil which was purified by chromatograph (12 g silica comumn, eluting with 20-70% EtOAc in isohexane to give the title compound as a colourless solid (0.2 g), ¹H NMR (300.072 MHz, CDCl₃) [symbol] 1.49-2.30 (m, 5H), 2.66 (s, 3H), 3.22-3.85 (m, 3H), 4.53-4.89 (m, 1H), 7.43 (d, 1H), 7.58-7.64 (m, 3H), 7.67-7.71 (m, 2H), 8.07 (d, 1H), m/z 366 (M+H)⁺.

### Step 3: 4-[1-(3-amino-4-methyl-benzoyl)-4-hydroxy-4-piperidyl]benzonitrile

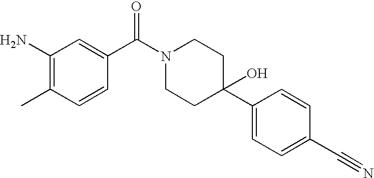

A mixture of 4-[4-hydroxy-1-(4-methyl-3-nitro-benzoyl)-4-piperidyl]benzonitrile (Step 2) (0.2 g, 0.55 mmol), iron (III) chloride hexahydrate (444 mg, 1.64 mmol) and zinc dust (360 mg, 5.5 mmol) in DMF (6 mL) and water (3 mL) was heated at 100° C. for 4 hrs. The reaction mixture was filtered through celite and the filtrate evaporated in vacuo. EtOAc (30 ml) was added to the residue and the resulting solution was washed sequentially with water (2×30 mL) and brine (30 mL), dried (MgSO₄) and evaporated in vacuo to give the title compound as a colourless solid (0.17 g), ¹H NMR (300.072 MHz, CDCl₃) [symbol] 1.59-2.10 (m, 5H), 2.18 (s, 3H), 3.10-3.48 (m, 3H), 3.60-4.02 (m, 2H), 4.47-4.73 (m, 1H), 6.69-6.75 (m, 2H), 7.06 (d, 1H), 7.57-7.68 (m, 4H), m/z 336 (M+H)⁺.

### Intermediate J

### 2-[[5-[4-(4-cyanophenyl)piperidine-1-carbonyl]-2-methyl-phenyl]sulfamoyl]acetic acid

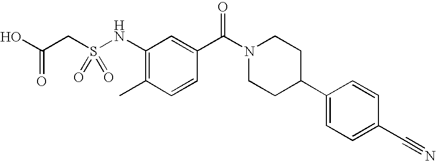

A solution of methyl 2-[[5-[4-(4-cyanophenyl)piperidine-1-carbonyl]-2-methyl-phenyl]sulfamoyl]acetate (Example 13) (1.335 g, 2.93 mmol) in MeOH (10 mL) was treated with aqueous sodium hydroxide solution (7.32 mL of a 2M solution, 14.65 mmol), and the mixture was stirred at ambient temperature for 1 h. The yellow suspension was acidified with 2M aqueous hydrochloric acid, and the organic solvent evaporated in vacuo. The resulting precipitate was isolated by filtration, washed with water and dried to give the title compound as a cream solid (1.08 g) which was used without further purification, ¹H NMR (300.073 MHz, DMSO-d₆) δ 1.72 (4H, m), 2.34 (3H, s), 2.93 (1H, m), 4.11 (2H, s), 7.23 (1H, d), 7.31 (1H, d), 7.40 (1H, s), 7.50 (2H, d), 7.76 (2H, d), m/z 442 (M+H)⁺.

### Intermediate K

### tert-butyl N-(3-chloropropylsulfonyl)-N-[3-[4-(4-cyanophenyl)piperidine-1-carbonyl]phenyl]carbamate

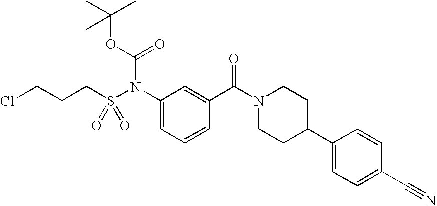

A solution of 3-chloro-N-[5-[4-(4-cyanophenyl)piperidine-1-carbonyl]-2-methyl-phenyl]propane-1-sulfonamide (Example 9) (1.35 g, 2.93 mmol) in THF (40 mL) was treated with DMAP (36 mg, 0.29 mmol) and (2-methylpropan-2-yl)oxycarbonyl tert-butyl carbonate (1.93 g, 8.80 mmol) and stirred at ambient temperature for 2 hrs. The solvent was evaporated in vacuo and EtOAc (30 mL) added to the residue. The resulting solution was washed sequentially with water (30 mL) and brine (30 mL), dried (MgSO₄), filtered and evaporated in vacuo to give a colourless solid which was purified by chromatography on silica, eluting with a gradient of 20-70% EtOAc in isohexane, to give the title compound as a colourless solid (1.45 g, 88%), ¹H NMR (300.072 MHz, CDCl₃) [symbol] 1.46 (s, 9H), 1.60-1.95 (m, 4H), 2.37 (s, 3H), 2.39-2.46 (m, 2H), 2.78-2.92 (m, 2H), 2.95-3.09 (m, 1H), 3.71 (t, 2H), 3.76-3.83 (m, 1H), 3.88-4.03 (m, 2H), 4.64-4.98 (m, 1H), 7.23-7.25 (m, 1H), 7.29-7.36 (m, 3H), 7.39-7.43 (m, 1H), 7.61 (d, 2H), m/z 560, 562 (M+H)⁺ [A].

### Intermediate L

### 2-[[5-[4-(4-cyanophenyl)piperidine-1-carbonyl]-2-methyl-phenyl]sulfamoyl]benzoic acid

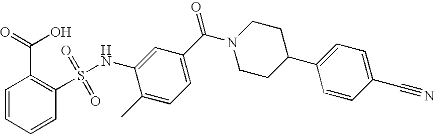

The title compound was prepared by the method given in Example 79, starting from methyl 2-[[5-[4-(4-cyanophenyl)piperidine-1-carbonyl]-2-methyl-phenyl]sulfamoyl]benzoate (Example 108), ¹H NMR (300.072 MHz, CDCl₃) δ 1.57-1.99 (m, 4H), 2.14 (s, 3H), 2.80-2.91 (m, 2H), 2.99-3.13 (m, 1H), 3.77-3.98 (m, 1H), 4.77-4.96 (m, 1H), 6.05 (s, 1H), 7.20 (s, 2H), 7.31-7.37 (m, 3H), 7.41-7.48 (m, 1H), 7.52-7.58 (m, 1H), 7.62 (d, 2H), 7.74-7.78 (m, 1H), 7.84-7.88 (m, 1H), 8.08 (s, 1H), m/z 504 (M+H)⁺.

### Intermediate M

### 4-[1-(3-amino-4-methyl-benzoyl)-4-piperidyl]benzamide

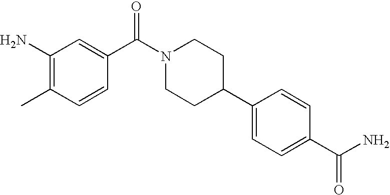

### Step 1: 4-[1-(4-methyl-3-nitro-benzoyl)-4-piperidyl]benzamide

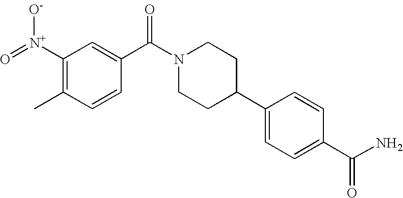

The title compound was prepared from 4-[1-(4-methyl-3-nitro-benzoyl)-4-piperidyl]benzoic acid (Intermediate H, Step 2) using the procedure described in Heterocycles, 2006, 68 (6), 1149-1162, ¹H NMR (300.073 MHz, DMSO-d₆) δ 1.57-1.94 (m, 4H), 2.54 (s, 3H), 2.66-3.03 (m, 3H), 3.56 (s, 3H), 3.59-3.72 (m, 1H), 4.50-4.71 (m, 1H), 7.24 (s, 1H), 7.35 (d, J=8.1 Hz, 2H), 7.57 (d, J=7.9 Hz, 1H), 7.70 (d, J=7.8 Hz, 1H), 7.80 (d, J=8.1 Hz, 2H), 7.87 (s, 1H), 8.02 (s, 1H), m/z 368 (M+H)⁺.

### Step 2: 4-[1-(3-amino-4-methyl-benzoyl)-4-piperidyl]benzamide

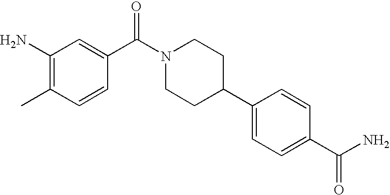

The title compound was prepared from 4-[1-(4-methyl-3-nitro-benzoyl)-4-piperidyl]benzamide (Step 1) using the hydrogenation procedure described in Intermediate H, Step 4, ¹H NMR (300.073 MHz, DMSO-d₆) δ 1.47-1.67 (m, 2H), 1.70-1.88 (m, 2H), 2.06 (s, 3H), 2.69-2.98 (m, 3H), 3.66-3.98 (m, 1H), 4.37-4.74 (m, 1H), 4.97 (s, 2H), 6.49 (d, J=7.5 Hz, 1H), 6.64 (s, 1H), 6.95 (d, J=7.5 Hz, 1H), 7.24 (s, 1H), 7.33 (d, J=8.2 Hz, 2H), 7.80 (d, J=8.1 Hz, 2H), 7.87 (s, 1H), m/z 368 (M+H)⁺.

### Intermediate N

### 2-[[5-[4-(4-cyanophenyl)piperidine-1-carbonyl]-2-methyl-phenyl]sulfamoyl]-N-[(2,4-dimethoxyphenyl)methyl]benzamide

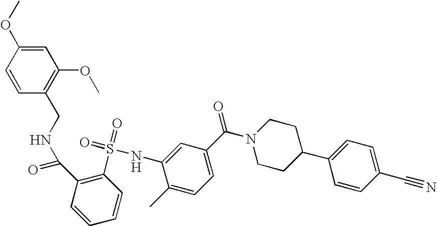

### Step 1: 4-{1-[3-(1,1-dioxido-3-oxo-1,2-benzisothiazol-2(3H)-yl)-4-methylbenzoyl]piperidin-4-yl}benzonitrile

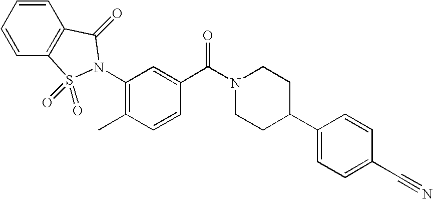

A mixture of 2-[[5-[4-(4-cyanophenyl)piperidine-1-carbonyl]-2-methyl-phenyl]sulfamoyl]benzoic acid (Intermediate L) (0.4 g, 0.79 mmol), ammonia (8 mL of a 0.5M solution in dioxan, 3.97 mmol), N-(3-Dimethylaminopropyl)-N′-ethylcarbodiimide hydrochloride (229 mg, 1.19 mmol) and DMAP (10 mg, 0.08 mmol) in DMF (8 mL) was stirred at ambient temperature for 20 hrs. EtOAc (30 mL) was added and the resulting solution washed sequentially with potassium bisulfate solution (30 mL of 2M), brine (30 mL), dried (MgSO₄), filtered and reduced in vacuo to give an off-white solid which was purified by chromatography on silica, eluting with a gradient of 0-10% MeOH in DCM, to give a the title compound as a colourless solid.

¹H NMR (300.072 MHz, CDCl₃) δ 1.62-1.97 (m, 4H), 2.34 (s, 3H), 2.80-2.90 (m, 2H), 2.97-3.18 (m, 1H), 3.89-4.11 (m, 1H), 4.62-5.05 (m, 1H), 7.32 (d, 2H), 7.46-7.53 (m, 2H), 7.59 (d, 3H), 7.87-8.03 (m, 3H), 8.14-8.18 (m, 1H).

### Step 2: 2-[[5-[4-(4-cyanophenyl)piperidine-1-carbonyl]-2-methyl-phenyl]sulfamoyl]-N-[(2,4-dimethoxyphenyl)methyl]benzamide

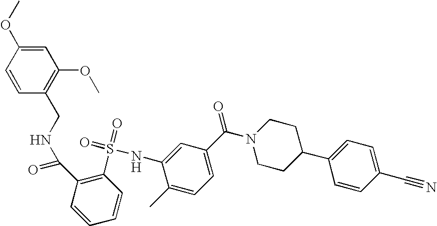

A mixture of 4-{1-[3-(1,1-dioxido-3-oxo-1,2-benzisothiazol-2(3H)-yl)-4-methylbenzoyl]piperidin-4-yl}benzonitrile (Step 1) (0.2 g, 0.41 mmol) and 2,4-dimethoxybenzylamine (138 mg, 0.82 mmol) in THF (5 mL) was heated in the microwave at 140° C. for 30 mins then a further 30 mins. The solvent was evaporated in vacuo and EtOAc (30 mL) added to the residue. The resulting solution was washed sequentially with aqueous citric acid (20 mL of a 1M solution), water and brine, dried (MgSO₄), filtered and reduced in vacuo to give a colourless solid which was purified by chromatography on silica, eluting with a gradient of 0-4% MeOH in DCM to give the title compound as a colourless solid (91 mg, 34%), ¹H NMR (300.072 MHz,) [symbol] 1.52-1.92 (m, 4H), 2.21 (s, 3H), 2.77-2.88 (m, 2H), 2.94-3.08 (m, 1H), 3.81 (s, 3H), 3.86 (s, 3H), 3.89-4.02 (m, 1H), 4.63 (d, 2H), 4.71-4.87 (m, 1H), 6.46-6.51 (m, 2H), 6.59 (t, 1H), 7.15-7.18 (m, 2H), 7.28-7.33 (m, 4H), 7.36-7.42 (m, 1H), 7.47-7.52 (m, 2H), 7.60 (d, 2H), 7.70 (d, 1H), 8.45 (s, 1H), m/z 653 (M+H)⁺.

### Intermediate O

### tert-butyl N-[5-[4-(4-cyanophenyl)piperidine-1-carbonyl]-2-methyl-phenyl]-N-(4-piperidylsulfonyl)carbamate

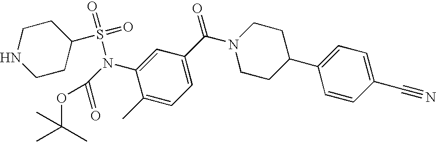

### Step 1: tert-butyl N-[5-[4-(4-cyanophenyl)piperidine-1-carbonyl]-2-methyl-phenyl]carbamate

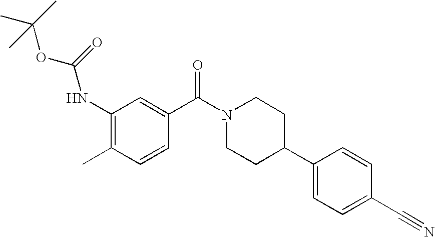

The title compound was prepared by the method described in Intermediate K, starting from 4-[1-(3-amino-4-methyl-benzoyl)-4-piperidyl]benzonitrile (Intermediate A) and (2-methylpropan-2-yl) oxycarbonyl tert-butyl carbonate, ¹H NMR (300.073 MHz, DMSO-d₆) ä1.45 (s, 9H), 1.53-1.70 (m, 2H), 1.70-1.91 (m, 2H), 2.21 (s, 3H), 2.68-3.00 (m, 1H), 3.00-3.23 (m, 1H), 3.61-3.94 (m, 1H), 4.21-4.83 (m, 1H), 7.04-7.12 (m, 1H), 7.17-7.25 (m, 1H), 7.38 (s, 1H), 7.50 (d, 2H), 7.75 (d, 2H), 8.59 (s, 1H) [NB signals due to piperidyl 4-H v. broad and obscured by adjacent signals], m/z 420 (M+H)⁺.

### Step 2: benzyl 4-[[5-[4-(4-cyanophenyl)piperidine-1-carbonyl]-2-methyl-phenyl]-[(2-methylpropan-2-yl)oxycarbonyl]sulfamoyl]piperidine-1-carboxylate

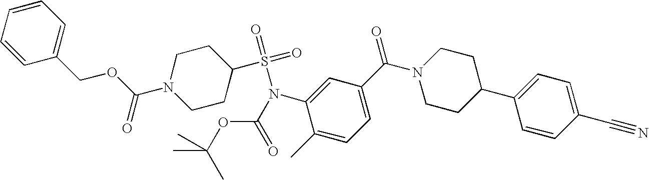

An ice-cooled solution of tert-butyl N-[5-[4-(4-cyanophenyl)piperidine-1-carbonyl]-2-methyl-phenyl]carbamate (Step 1) (6.1 g, 14.52 mmol) in THF (100 mL) was treated with lithium hexamethyl disilazide (16 mL of a 1M solution in THF, 16 mmol) and stirred at 0-5° C. for 30 mins before addition of benzyl 4-chlorosulfonylpiperidine-1-carboxylate (4.62 g, 14.52 mmol); the reaction mixture was then stirred for 2 hrs, allowing to warm to ambient temperature. The solvent was evaporated in vacuo and EtOAc (200 mL) added to the residue. The resulting solution was washed sequentially with water (2×100 mL) and brine (100 mL), dried (MgSO₄), filtered and evaporated in vacuo to give a colourless solid which was purified by chromatography on silica, eluting with a gradient of 0-100% EtOAc in isohexane, to give the title compound as a colourless foam (6.1 g, 60%), ¹H NMR (300.072 MHz, CDCl₃) δ1.42 (9H, s), 1.50-2.00 (6H, m), 2.18-2.28 (2H, m), 2.38 (3H, s), 2.81-3.20 (5H, m), 3.95 (2H, m), 4.20-4.50 (3H, m), 4.85 (1H, m), 5.13 (2H, s), 7.19 (1H, s), 7.30-7.40 (9H, m), 7.61 (2H, d), m/z 701 (M+H)⁺.

### Step 3: tert-butyl N-[5-[4-(4-cyanophenyl)piperidine-1-carbonyl]-2-methyl-phenyl]-N-(4-piperidylsulfonyl)carbamate

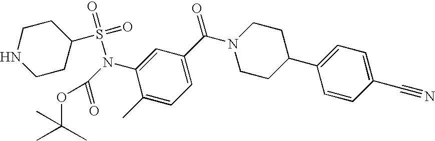

A solution of benzyl 4-[[5-[4-(4-cyanophenyl)piperidine-1-carbonyl]-2-methyl-phenyl]-[(2-methylpropan-2-yl)oxycarbonyl]sulfamoyl]piperidine-1-carboxylate (Step 2) (6.1 g, 8.7 mmol) in EtOAc (300 mL) was stirred in an atmosphere of hydrogen with palladium on charcoal catalyst (610 mg of 10% Pd/C). The catalyst was removed by filtration, and the filtrate evaporated under reduced pressure. The residue was purified by chromatography on silica, eluting with a gradient of 0-10% MeOH in DCM, to give the title compound as a colourless solid (2.1 g, 43%), ¹H NMR (300.073 MHz, DMSO-d₆) δ 1.39 (9H, s), 1.45 (1H, s), 1.53-1.63 (2H, m), 1.71 (2H, s), 1.98-2.02 (1H, m), 2.13 (1H, s), 2.27 (3H, s), 2.50 (4H, m), 2.70-3.20 (4H, m), 3.65 (1H, m), 4.60 (1H, m), 7.28 (1H, s), 7.35-7.42 (2H, m), 7.51 (2H, d), 7.76 (2H, d), m/z 567 (M+H)⁺.

### Intermediate P

### 4-[1-(3-amino-4-ethyl-benzoyl)-4-piperidyl]benzonitrile

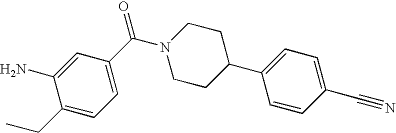

### Step 1: 4-ethyl-3-nitro-benzoic acid

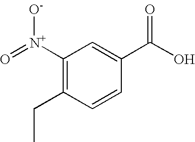

Concentrated nitric acid (80 mL) was cooled to approximately 0-5° C. in an ice bath and 4-ethyl benzoic acid (10 g, 66.59 mmol) was added portionwise. The resultant mixture was allowed to warm up to ambient temperature and the reaction mixture was stirred for approx. 72 hrs. It was then warmed to 60° C. at which it was maintained overnight at this temperature.

The reaction mixture was quenched into ice/water (200 mL) and the resulting precipitate isolated by filtration and washed with water to give the title compounds as a colourless solid (9.52 g, 73%), ¹H NMR (300.073 MHz, DMSO-d₆, 30° C.) δ 1.22 (3H, t J=8.3 Hz), 2.87 (2H, q J=7.8 Hz), 7.65 (1H, d J=8.3 Hz), 8.13 (1H, d J=9.0 Hz), 8.34 (1H, s 13.00-13.80 (1H, m), m/z 194 (M−H)⁻.

### Step 2: 4-[1-(4-ethyl-3-nitro-benzoyl)-4-piperidyl]benzonitrile

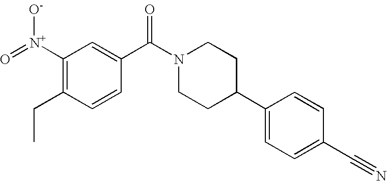

The title compound was prepared by an amide coupling reaction starting from 4-ethyl-3-nitro-benzoic acid (Step 1) and 4-(4-piperidyl)benzonitrile as described for Intermediate A, ¹H NMR (300.073 MHz, DMSO-d₆, 30° C.) δ 1.22 (3H, tJ=7.4 Hz), 1.56-1.96 (4H, m), 2.77-3.01 (4H, m—contains q from ethyl), 3.04-3.25 (1H, m), 3.63 (1H, br s), 4.61 (1H, br s), 7.50 (2H, dJ=9.1 Hz), 7.59 (1H, dJ=7.4 Hz), 7.67-7.81 (3H, m), 7.94-7.98 (1H, m).

### Step 3: 4-[1-(3-amino-4-ethyl-benzoyl)-4-piperidyl]benzonitrile

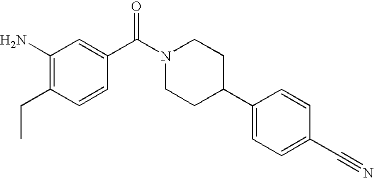

The title compound was prepared by hydrogenation of 4-[1-(4-ethyl-3-nitro-benzoyl)-4-piperidyl]benzonitrile (Step 2) as described for Intermediate I, Step 3, ¹H NMR (300.073 MHz, DMSO-d₆, 30° C.) δ 1.13 (3H, tJ=6.8 Hz), 1.46-1.67 (2H, m), 1.67-1.91 (2H, m), 2.38-2.48 (2H, m), 2.64-3.21 (3H, m), 3.59-4.05 (1H, m), 4.33-4.72 (1H, m), 4.98 (2H, s), 6.53 (1H, dJ=7.3 Hz), 6.64 (1H, s), 6.95 (1H, dJ=6.1 Hz), 7.49 (2H, dJ=7.2 Hz), 7.76 (2H, dJ=9.5 Hz), m/z 334 (M+H)⁺.

### Intermediate Q

### N-[5-[4-(4-cyanophenyl)piperidine-1-carbonyl]-2-methyl-phenyl]-3-(1,3-dioxoisoindol-2-yl)propane-1-sulfonamide

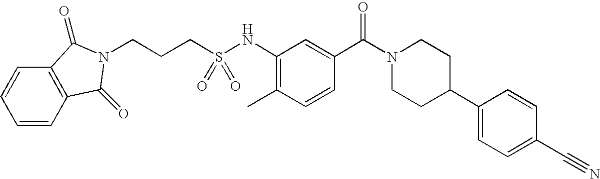

### Step 1: 3-(1,3-dioxoisoindol-2-yl)propane-1-sulfonyl chloride

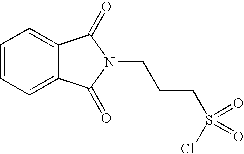

The title compound was prepared according to the procedure reported in Bioorganic and Medicinal Chemistry Letters, Vol. 6 (14) p 1709 (1996).

### Step 2: N-[5-[4-(4-cyanophenyl)piperidine-1-carbonyl]-2-methyl-phenyl]-3-(1,3-dioxoisoindol-2-yl)propane-1-sulfonamide

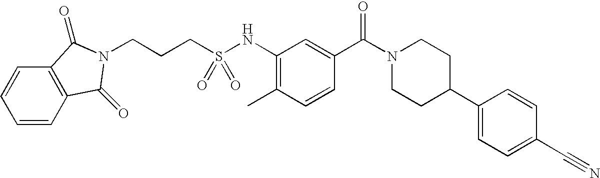

The title compound was prepared by the method described in Example 1, starting from Intermediate A and 3-(1,3-dioxoisoindol-2-yl)propane-1-sulfonyl chloride (Step 1), ¹H nmr (300.071 MHz, CDCl₃) δ 1.47-1.82 (4H, m), 2.16-2.26 (2H, m), 2.32 (3H, s), 2.80-3.10 (3H, m), 3.20-3.25 (2H, m), 3.80 (2H, t, J6.8), 3.83-4.13 (1H, m), 4.64-4.99 (1H, m), 6.41 (1H, s), 7.15 (1H, dd, J7.8, 1.4), 7.21 (1H, d, J7.8), 7.33 (2H, d, J8.3), 7.50 (1H, d, J1.4), 7.61 (2H, d, J8.3), 7.71-7.75 (2H, m), 7.81-7.84 (2H, m), m/z 571 (M+H)⁺, 569 (M−H)⁻.

### Intermediate R

### 4-[1-(3-amino-4-methylsulfonyl-benzoyl)-4-piperidyl]benzonitrile

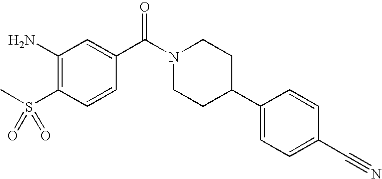

### Step 1

### 4-[1-(4-methylsulfonyl-3-nitro-benzoyl)-4-piperidyl]benzonitrile

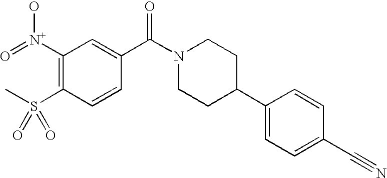

Oxalyl chloride (2.18 ml, 24.47 mmol) was added to a mixture of 4-methylsulfonyl-3-nitro benzoic acid (5 g, 20.39 mmol) in dichloromethane (50 ml); to this stirred mixture was added DMF (2 drops). The reaction mixture was stirred at ambient temperature for 2 hrs, the volatiles removed in vacuo, and the residue redissolved in dichloromethane (25 ml). This solution was added to a stirred solution of 4-(4′-cyanophenyl)piperidine (3.79 g, 20.39 mmol) and DIPEA (7.82 ml, 44.86 mmol) in DCM (25 ml) and the resulting mixture stirred for 20 hrs. It was then diluted with DCM and the mixture washed sequentially with 0.5M HCl solution, saturated NaHCO₃ solution and brine. The organic phase was dried and concentrated in vacuo to give a yellow solid which was purified by chromatography on silica (120 g column, eluting with 10-100% ethyl acetate in isohexane) to give the title compound as a yellow solid (4.0 g), ¹H NMR (300.072 MHz, CDCl₃) [symbol] 1.62-2.12 (m, 4H), 2.84-2.97 (m, 2H), 3.11-3.34 (m, 1H), 3.45 (s, 3H), 3.61-3.82 (m, 1H), 4.72-5.08 (m, 1H), 7.33 (d, 2H), 7.63 (d, 2H), 7.79-7.82 (m, 1H), 7.89 (d, 1H), 8.27 (d, 1H), m/z 455 (M+MeCN+H)⁺.

### Step 2

### 4-[1-(3-amino-4-methylsulfonyl-benzoyl)-4-piperidyl]benzonitrile

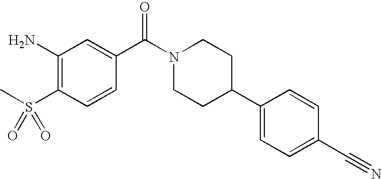

A mixture of 4-[1-(4-methylsulfonyl-3-nitro-benzoyl)-4-piperidyl]benzonitrile (Step 1) (4.0 g, 9.67 mmol), iron (III) chloride hexahydrate (7.85 g, 29 mmol, 3 eq) and zinc dust (6.32 g, 96.7 mmol, 10 eq) in DMF (100 ml) and water (50 ml) was heated at 100° C. for 4 hrs. The reaction mixture was filtered through celite and the filtrate evaporated in vacuo. EtOAc (30 mL) was added and the resulting solution was washed sequentially with water (2×30 ml) and brine (30 ml). An insoluble beige solid was filtered off at this point; this was discarded as an impurity. The remaining solution was dried (MgSO₄) and evaporated in vacuo to give a yellow foam. This was purified by chromatographed on silica (40 g column, eluting with 20-80% EtOAc in isohexane) to give the title compound as a colourless solid (1.5 g), ¹H NMR (300.072 MHz, CDCl₃) [symbol] 1.51-2.08 (m, 4H), 2.79-2.93 (m, 2H), 3.06 (s, 3H), 3.11-3.24 (m, 1H), 3.74-3.91 (m, 1H), 4.74-4.92 (m, 1H), 5.16 (s, 2H), 6.79-6.83 (m, 2H), 7.32 (d, 2H), 7.62 (d, 2H), 7.78 (d, 1H), m/z 382 (M−H)⁻.

### Intermediate S

### Methyl 2-[[5-[4-(4-cyanophenyl)piperidine-1-carbonyl]-2-methyl-phenyl]-[(2-methylpropan-2-yl)oxycarbonyl]sulfamoyl]propanoate

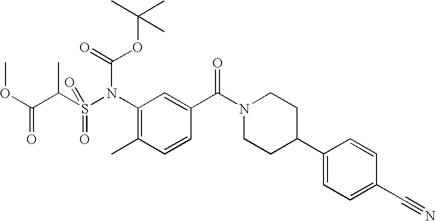

### Step 1

### Methyl 2-[[5-[4-(4-cyanophenyl)piperidine-1-carbonyl]-2-methyl-phenyl]-[(2-methylpropan-2-yl)oxycarbonyl]sulfamoyl]acetate

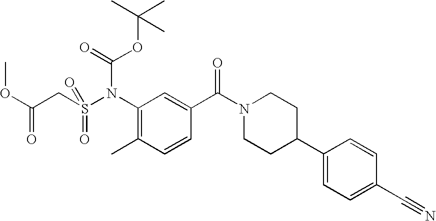

A solution of methyl 2-[[5-[4-(4-cyanophenyl)piperidine-1-carbonyl]-2-methyl-phenyl]sulfamoyl]acetate (Example 13) (2.99 g, 6.56 mmol) in THF (35 ml) was treated with (2-methylpropan-2-yl)oxycarbonyl tert-butyl carbonate (1.6 g, 1.1 eq) and DMAP (80 mg, 0.1 eq), and the resulting yellow solution was stirred at ambient temperature for 2 hrs. The reaction mixture was diluted with ethyl acetate and the organic solution was washed sequentially with water and brine, dried (MgSO₄) and evaporated to give a yellow foam which was used in the next step without purification, ¹H NMR (300.073 MHz, DMSO-d₆) δ 1.38 (s, 9H), 1.55-1.95 (m, 4H), 2.25 (s, 3H), 2.75-3.23 (m, 3H), 3.50-3.90 (m, 4H), 4.46-4.81 (m, 1H), 4.88-5.07 (m, 2H), 7.35 (s, 1H), 7.41 (s, 2H), 7.50 (d, 2H), 7.76 (d, 2H), m/z 556 (M+H)⁺, 85% purity.

### Step 2

### Methyl 2-[[5-[4-(4-cyanophenyl)piperidine-1-carbonyl]-2-methyl-phenyl]-[(2-methylpropan-2-yl)oxycarbonyl]sulfamoyl]propanoate

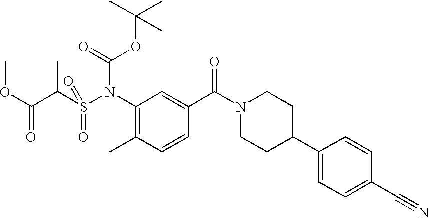

A solution of methyl 2-[[5-[4-(4-cyanophenyl)piperidine-1-carbonyl]-2-methyl-phenyl]-[(2-methylpropan-2-yl)oxycarbonyl]sulfamoyl]acetate (Step 1) (2.0 g, 3.6 mmol) in anhydrous DMF (20 ml) was placed under a under nitrogen atmosphere and treated with potassium hydroxide (222 mg, 3.96 mmol, 1.1 eq). Iodomethane (0.246 mL, 3.96 mmol, 1.1 eq) was then added and the resulting orange mixture was heated to 60° C. for 3½ hrs. The reaction mixture was allowed to cool and then diluted with water and DCM. The layers were separated and the organic phase was washed sequentially with water and brine, dried (MgSO₄) and evaporated to a yellow oil. This contained some dimethylated compound in addition to the monomethyl and two attempts at separation were unsuccessful, so the crude compound was carried through to the next step, m/z 570 (M+H)⁺, retention time 2.81 min., 86% purity.

### Intermediate T

### N-(5-(4-(4-cyanophenyl)piperidine-1-carbonyl)-2-methylphenyl)-1-(oxiran-2-yl)methanesulfonamide

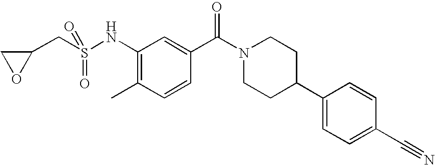

3-chloroperoxybenzoic acid (537 mg, 2.15 mmol) was added to N-(5-(4-(4-cyanophenyl)piperidine-1-carbonyl)-2-methylphenyl)prop-2-ene-1-sulfonamide (Example 146) (455 mg, 1.07 mmol) in DCM (10 mL). The resulting solution was stirred at 40° C. for 24 hours. The reaction was incomplete and further 3-chloroperoxybenzoic acid (1.074 g, 4 eq.) was added and the solution was stirred at 40° C. for a further 18 hours. The reaction mixture was diluted with DCM and washed with water. The organic phase was dried (phase separating cartridge) and evaporated to give crude product. This was purified by flash silica chromatography, elution gradient 0 to 100% EtOAc in isohexane, to give the title compound (141 mg, 29.9%) as a yellow solid, ¹H NMR (300.073 MHz, DMSO-d₆) δ 1.51-1.95 (4H, m), 2.34 (3H, s), 2.59-2.65 (1H, m), 2.76-2.83 (1H, m), 2.84-3.20 (3H, m), 3.41-3.57 (1H, m), 3.61-3.82 (1H, m), 4.37-4.80 (1H, m), 7.18-7.41 (3H, m), 7.50 (2H, d), 7.77 (2H, d), 9.40 (1H, s), m/z 440 (M+H)⁺.

### Intermediate U

### N-(5-(4-(4-cyanophenyl)piperidine-1-carbonyl)-2-methylphenyl)pyrrolidine-3-sulfonamide hydrochloride

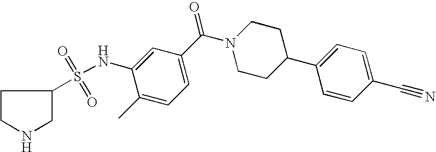

### Step 1

### (RS)-benzyl 3-(acetylthio)pyrrolidine-1-carboxylate

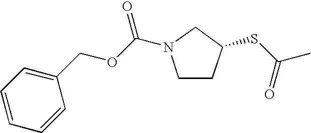

The title compound may be prepared as described in U.S. Pat. Appl. US 2007/072882

### Step 2

### (RS)-benzyl 3-(chlorosulfonyl)pyrrolidine-1-carboxylate

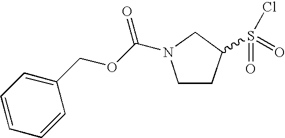

A solution of (RS)-benzyl 3-(acetylthio)pyrrolidine-1-carboxylate (Step 1) (6 g, 21.48 mmol) and acetic acid (2 mL, 34.94 mmol) in water (150 mL) was stirred at 20° C. and chlorine gas was bubbled through the reaction mixture for 1 hour. Nitrogen was bubbled through the reaction mixture to remove excess chlorine. The reaction mixture was then diluted with EtOAc (125 mL), and the solution washed with saturated brine (100 mL). The organic layer was dried over MgSO₄, filtered and evaporated to afford crude product (3.2 g). This was purified by flash silica chromatography, elution gradient 5 to 30% EtOAc in isohexane to give the title compound as a colourless oil (4.00 g, 61.3%), ¹H NMR (300.072 MHz, CDCl₃) δ 2.42-2.55 (1H, m), 2.59-2.72 (1H, m), 3.55-3.67 (1H, m), 3.70-3.80 (1H, m), 3.89-3.99 (1H, m), 4.05-4.17 (1H, m), 4.25-4.34 (1H, m), 5.15 (2H, s), 7.33-7.38 (5H, m), m/z 302 (M−H)⁻.

### Step 3

### (RS)-benzyl 3-(N-(tert-butoxycarbonyl)-N-(5-(4-(4-cyanophenyl)piperidine-1-carbonyl)-2-methylphenyl)sulfamoyl)pyrrolidine-1-carboxylate

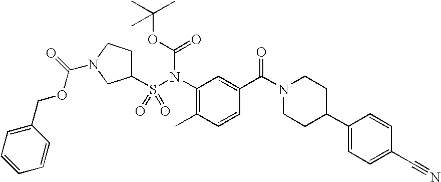

Lithium bis(trimethylsilyl)amide (1M in THF) (2.86 mL, 2.86 mmol) was added to tert-butyl 5-(4-(4-cyanophenyl)piperidine-1-carbonyl)-2-methylphenylcarbamate (Intermediate V) (1.2 g, 2.86 mmol) in THF (40 mL) at 0° C. The resulting solution was stirred at 0° C. for 30 minutes and (RS)-benzyl 3-(chlorosulfonyl)pyrrolidine-1-carboxylate (Step 2) (1.043 g, 3.43 mmol) was added and the reaction mixture was stirred at 20° C. for 5 hrs under nitrogen. The reaction mixture was diluted with EtOAc (20 mL), and washed sequentially with water (25 mL) and saturated brine (25 mL). The organic layer was dried over MgSO₄, filtered and evaporated to afford crude product. The crude product was purified by flash silica chromatography, elution gradient 20 to 80% EtOAc in isohexane, to give the title compound (0.460 g, 23.4%), ¹H NMR (300.072 MHz, CDCl₃) δ 1.41 (9H, s), 1.64-1.92 (4H, m), 2.37 (3H, d), 2.44-2.50 (2H, m), 2.53-2.60 (1H, m), 2.76-2.91 (2H, m), 2.95-3.10 (1H, m), 3.46-3.59 (1H, m), 3.65-3.73 (1H, m), 3.86-3.99 (2H, m), 4.74-4.85 (2H, m), 5.05-5.14 (2H, m), 7.29-7.37 (10H, m), 7.56-7.63 (2H, m), m/z (ESI+) (M+H)⁺=687; HPLC tR=3.05 min.

### Step 4

### (RS)-tert-butyl 3-(N-(5-(4-(4-cyanophenyl)piperidine-1-carbonyl)-2-methylphenyl) sulfamoyl)pyrrolidine-1-carboxylate

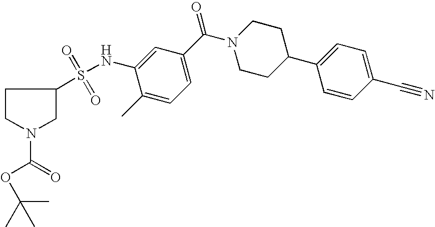

Ammonium formate (0.184 g, 2.91 mmol) was added to (RS)-benzyl 3-(N-(tert-butoxycarbonyl)-N-(5-(4-(4-cyanophenyl)piperidine-1-carbonyl)-2-methylphenyl) sulfamoyl)pyrrolidine-1-carboxylate (Step 3) (1 g, 1.46 mmol) and palladium on carbon (10%) (0.1 g, 0.09 mmol) in MeOH (10 mL) at 20° C. under nitrogen. The resulting suspension was stirred at 20° C. for 2 hours and at 60° C. for 20 hrs. The reaction was incomplete and further ammonium formate (0.184 g, 2.91 mmol) and palladium on carbon (10%) (0.1 g, 0.09 mmol) were added and the mixture was stirred at 60° C. for a further 2 hours. The reaction mixture was filtered through celite and evaporated to dryness; it was then redissolved in EtOAc (25 mL) and the solution washed with saturated brine (20 mL). The organic layer was dried over Na2SO4, filtered and evaporated to afford crude product which was purified by flash silica chromatography, elution gradient 30 to 100% EtOAc in isohexane, to give the title compound as a colourless solid (0.260 g, 32.3%), ¹H NMR (300.072 MHz, CDCl₃) δ 1.44 (9H, s), 1.64-1.96 (4H, m), 2.17-2.27 (1H, m), 2.33 (3H, s), 2.38-2.46 (1H, m), 2.79-2.89 (2H, m), 3.01-3.20 (1H, m), 3.33-3.45 (1H, m), 3.52-4.00 (5H, m), 4.78-4.97 (1H, m), 6.85-6.96 (1H, m), 7.16-7.25 (2H, m), 7.33 (2H, d), 7.47 (1H, s), 7.61 (2H, d), m/z (ESI+) (M+H)⁺=553; HPLC tR=2.54 min.

### Step 5

### (RS)—N-(5-(4-(4-cyanophenyl)piperidine-1-carbonyl)-2-methylphenyl)pyrrolidine-3-sulfonamide hydrochloride

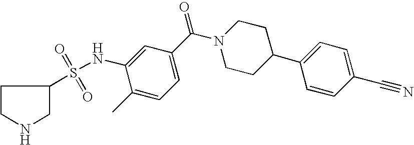

The title compound was prepared by a method analogous to that described in Example 115, starting from (RS)-tert-butyl 3-(N-(5-(4-(4-cyanophenyl)piperidine-1-carbonyl)-2-methylphenyl) sulfamoyl)pyrrolidine-1-carboxylate (Step 4), to give the title compound as the hydrochloride salt, which was used without purification, ¹H NMR (400.132 MHz, DMSO-d₆) δ 1.64-1.93 (4H, m), 2.36 (2H, q), 2.42 (3H, s), 2.93-3.05 (2H, m), 3.12-3.21 (1H, m), 3.31-3.37 (2H, m), 3.59-3.69 (3H, m), 4.14 (1H, quintet), 4.57-4.79 (1H, m), 7.31-7.43 (3H, m), 7.57 (2H, d), 7.84 (2H, d), 9.43 (1H, s), 9.59 (1H, s), m/z (ESI+) (M+H)⁺=453; HPLC tR=1.27 min.

### Intermediate V

### tert-butyl 5-(4-(4-cyanophenyl)piperidine-1-carbonyl)-2-methylphenylcarbamate

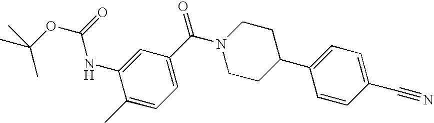

The title compound may be prepared as described in Synthetic Communications 31(21) p 3273 (2001), ¹H NMR (300.073 MHz, DMSO-d₆) δ 1.45 (s, 9H), 1.53-1.70 (m, 2H), 1.70-1.91 (m, 2H), 2.21 (s, 3H), 2.68-3.00 (m, 1H), 3.00-3.23 (m, 1H), 3.61-3.94 (m, 1H), 4.21-4.83 (m, 1H), 7.04-7.12 (m, 1H), 7.17-7.25 (m, 1H), 7.38 (s, 1H), 7.50 (d, 2H), 7.75 (d, 2H), 8.59 (s, 1H); NB signal due to piperidyl 4-H very broad and obscured by surrounding signals, m/z 420 (M+H)⁺.

### Intermediate W

### 3-methanesulfonamido-4-methyl-benzoic acid

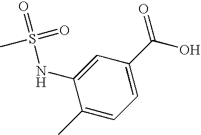

### Step 1

### Methyl 3-methanesulfonamido-4-methyl-benzoate

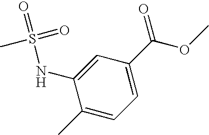

Methanesulfonyl chloride (6.68 mL, 86.26 mmol) was added to a solution of methyl 3-amino-4-methyl benzoate (9.5 g, 57.51 mmol) and pyridine (9.30 mL, 115.02 mmol) in DCM (150 mL) and the resulting solution was stirred at room temperature for 18 hours. The reaction mixture was diluted with water and poured onto a phase separating cartridge. The organic layer was evaporated to give crude product which was purified by crystallisation from DCM (with a little methanol) to give the title compound as a colourless crystalline solid (7.07 g, 50.5%). The liquors were concentrated and triturated with DCM to give a second batch of the title compound as a colourless solid (3.08 g, 22%), ¹H NMR (300.073 MHz, DMSO-d₆) δ 2.37 (3H, s), 2.99 (3H, s), 3.83 (3H, s), 7.39 (1H, d), 7.72 (1H, d), 7.86 (1H, s), 9.24 (1H, s), m/z (ESI−) (M−H)⁻=242.23; HPLC tR=1.62 min.

### Step 2

### 3-methanesulfonamido-4-methyl-benzoic acid

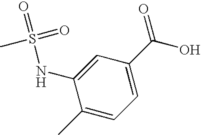

The title compound was prepared by hydrolysis of methyl 3-methanesulfonamido-4-methyl-benzoate (Step 1) using lithium hydroxide, as described in Example 79, ¹H NMR (300.073 MHz, DMSO-d₆) δ 2.36 (3H, s), 2.98 (3H, s), 7.35 (1H, d), 7.65-7.75 (1H, m), 7.84 (1H, s), m/z (ESI+) (M−H)⁻=228.24; HPLC tR=1.23 min.

### Intermediate X

### 4-[1-(3-amino-4-methylsulfanylbenzoyl)piperidin-4-yl]benzonitrile

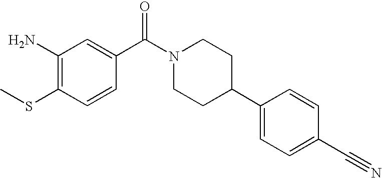

### Step 1

### 4-[1-(4-methylsulfanyl-3-nitrobenzoyl)piperidin-4-yl]benzonitrile

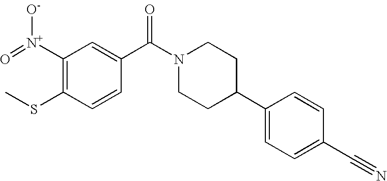

Oxalyl chloride (2.5 mL, 28 mmol) was added to a stirred suspension of 4-methylsulfanyl-3-nitro-benzoic acid (5 g, 23.45 mmol) in dichloromethane (50 mL), followed by the addition of DMF (2 drops), and the reaction mixture was stirred at ambient temperature for 2 hrs. The volatiles were removed in vacuo and the residue redissolved in dichloromethane (25 mL). This solution was added to a stirred solution of 4-(4′-cyanophenyl)piperidine (4.36 g, 23.45 mmol) and DIPEA (8.99 mL, 51.59 mmol) in DCM (25 mL) and the reaction mixture stirred for 20 hrs. It was then diluted with DCM and the resulting solution was washed sequentially with 0.5M HCl solution, saturated NaHCO₃ solution, and brine. The organic phase was dried and concentrated in vacuo to give a yellow solid which was purified by chromatography on silica (120 g column, eluting with 10-100% EtOAc in isohexane to give the title compound as a yellow solid (2.6 g), ¹H NMR (300.072 MHz, CDCl₃) [symbol] 1.59-2.04 (m, 4H), 2.54 (s, 3H), 2.80-2.93 (m, 2H), 3.01-3.18 (m, 1H), 3.77-4.20 (m, 1H), 4.44-5.03 (m, 1H), 7.33 (d, 2H), 7.44 (d, 1H), 7.63 (d, 2H), 7.68-7.72 (m, 1H), 8.34 (d, 1H), m/z (ESI+) (M+H)⁺=382; HPLC tR=2.54 min.

### Step 2

### 4-[1-(3-amino-4-methylsulfanylbenzoyl)piperidin-4-yl]benzonitrile

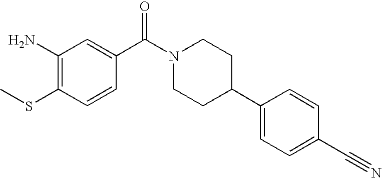

A mixture of 4-[1-(4-methylsulfanyl-3-nitrobenzoyl)piperidin-4-yl]benzonitrile (Step 1) (2.6 g, 6.82 mmol), iron (III) chloride hexahydrate (5.53 g, 20.45 mmol) and zinc dust (4.46 g, 68.2 mmol) in DMF (70 ml) and water (35 ml) was heated at 100° C. for 4 hrs. The reaction mixture was filtered through celite and evaporated in vacuo. Ethyl acetate (30 ml) was added to the filtrate and the resulting mixture was washed sequentially with water (2×30 mL) and saturated brine (30 mL). A beige solid impurity was removed by filtration and the organic filtrate was dried (MgSO₄) and evaporated in vacuo to give a yellow foam which was purified by chromatography on silica (40 g column, eluting with 20-80% EtOAc in isohexane) to give the title compound as a colourless solid (0.83 g), ¹H NMR (300.072 MHz, CDCl₃) δ 1.52-1.98 (m, 4H), 2.36 (s, 3H), 2.79-2.90 (m, 2H), 2.94-3.05 (m, 1H), 3.86-4.11 (m, 1H), 4.30 (s, 2H), 4.67-5.06 (m, 1H), 6.71-6.78 (m, 2H), 7.29-7.36 (m, 3H), 7.61 (d, 2H), m/z (ESI+) (M+H)⁺=352; HPLC tR=2.27 min.

### Intermediate Y

### 4-[1-[3-amino-4-(methoxymethyl)benzoyl]piperidin-4-yl]benzonitrile

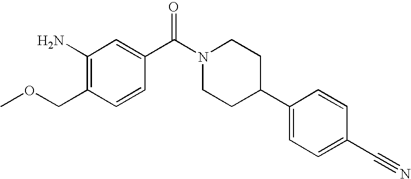

### Step 1

### 4-(methoxymethyl)-3-nitrobenzoic acid

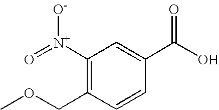

A solution of sodium methoxide in methanol (0.5 M, 115 mL, 57.68 mmol) was added dropwise to a stirred mixture of 4-(bromomethyl)-3-nitrobenzoic acid (5 g, 19.23 mmol) in methanol (100 mL) over a period of 5 minutes. The resulting mixture was stirred at 62° C. for 1 hour and was then quenched with water (100 mL) and the bulk of the methanol removed under reduced pressure. The reaction mixture was acidified with 2M HCl. The resulting precipitate was collected by filtration, washed with water (150 mL) and dried in the vacuum oven to give the title compound as a pale orange solid (2.96 g, 72.9%), which was used without further purification, ¹H NMR (300.073 MHz, DMSO-d₆) δ 3.39 (3H, s), 4.82 (2H, s), 7.86 (1H, d), 8.22-8.28 (1H, m), 8.47 (1H, d), 13.58 (1H, s), m/z (ESI−) (M−H)−=210.25; HPLC tR=1.69 min.

### Step 2

### 4-[1-[4-(methoxymethyl)-3-nitrobenzoyl]piperidin-4-yl]benzonitrile

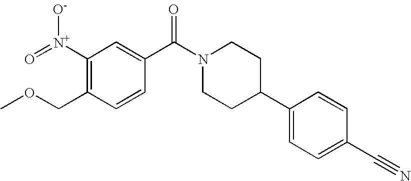

The title compound was prepared by the method described for Intermediate W, Step 1, starting from 4-(methoxymethyl)-3-nitrobenzoic acid and 4-(4′-cyanophenyl)piperidine (Step 1), ¹H NMR (300.073 MHz, DMSO-d₆) δ 1.59-1.97 (4H, m), 2.74-3.02 (2H, m), 3.06-3.24 (1H, m), 3.37 (3H, s), 3.50-3.80 (1H, m), 4.50-4.72 (1H, m), 4.77 (2H, s), 7.51 (2H, d), 7.73-7.85 (4H, m), 8.09 (1H, s), m/z—no mass ion observed; HPLC tR=2.45 min.

### Step 3

### 4-[1-[3-amino-4-(methoxymethyl)benzoyl]piperidin-4-yl]benzonitrile

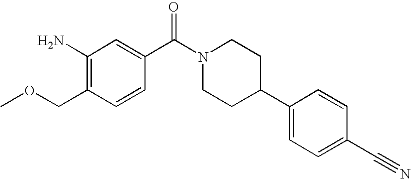

The title compound was prepared by the method described for Intermediate D, Step 2, starting from 4-[1-[4-(methoxymethyl)-3-nitrobenzoyl]piperidin-4-yl]benzonitrile (Step 2), and using a MeOH and THF mixture (1:1.5 by volume) as solvent, ¹H NMR (300.073 MHz, DMSO-d₆) δ 1.48-1.67 (2H, m), 1.68-1.92 (2H, m), 2.65-3.22 (3H, m), 3.27 (3H, s), 3.58-3.96 (1H, m), 4.32 (2H, s), 4.40-4.75 (1H, m), 5.09 (2H, s), 6.55 (1H, d), 6.67 (1H, s), 7.07 (1H, d), 7.49 (2H, d), 7.76 (2H, d), m/z (ESI+) (M+H)⁺=350.28; HPLC tR=2.01 min.

### Intermediate Z

### N-[5-[4-[4-(aminomethyl)phenyl]piperidine-1-carbonyl]-2-methylphenyl]methanesulfonamide

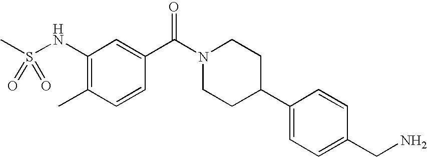

Cobalt chloride hexahydrate (2.370 g, 9.96 mmol) was added portionwise to a solution of N-(5-(4-(4-cyanophenyl)piperidine-1-carbonyl)-2-methylphenyl)methanesulfonamide (Example 1) (1.32 g, 3.32 mmol) in MeOH (25 mL), cooled to 0° C., over a period of 2 minutes. The resulting mixture was stirred at 0-5° C. for 5 minutes. Sodium borohydride (1.256 g, 33.21 mmol) was added portionwise over 5 minutes (vigorous effervescence and the colour changed from violet to black) and the mixture was stirred at 0-5° C. for a further 35 minutes. The reaction mixture was quenched with 2M HCl (125 mL) and the bulk of the methanol removed under reduced pressure. The resultant aqueous mixture was extracted with DCM (3×50 mL portions) and the combined organic layers were dried by passing through a phase separating cartridge and evaporated to give a yellow solid. The aqueous phase was made basic with aqueous sodium hydrogen carbonate solution and extracted with DCM (3×50 mL portions). The combined organic layers were dried by passing through a phase separating cartridge and evaporated to give a beige foam (0.52 g, 38%) which was used without further purification, ¹H NMR (400.132 MHz, DMSO-d₆) δ 1.41-1.87 (4H, m), 2.23 (3H, s), 2.66-2.82 (2H, m), 2.87 (3H, s), 2.98-3.14 (1H, m), 3.56-3.78 (3H, m), 4.45-4.61 (1H, m), 4.62-5.42 (2H, m), 7.05 (1H, d), 7.12-7.26 (6H, m) (signal due sulfonamide proton apparently missing), m/z (ESI+) (M+H)⁺=402.48; HPLC tR=1.00 min.

### Intermediate AA

### Methyl 4-[1-(3-methanesulfonamido-4-methylbenzoyl)piperidin-4-yl]benzoate

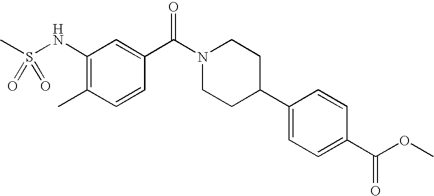

The title compound was prepared by a method analogous to that described in Example 155 starting from 3-methanesulfonamido-4-methyl-benzoic acid (Intermediate W) and using methyl 4-piperidin-4-ylbenzoate hydrochloride in place of 4-(4-chlorophenyl)piperidine hydrochloride, ¹H NMR (400.132 MHz, DMSO-d₆) d1.49-1.65 (m, 2H), 1.65-1.86 (m, 2H), 2.27 (s, 3H), 2.73-2.90 (m, 2H), 2.94 (s, 3H), 2.99-3.18 (m, 1H), 3.59-3.73 (m, 1H), 3.77 (s, 3H), 4.46-4.63 (m, 1H), 7.14-7.19 (m, 1H), 7.26 (d, 2H), 7.38 (d, 2H), 7.84 (d, 2H), 9.09 (s, 1H), m/z (ESI+) (M+H)⁺=431.46; HPLC tR=2.12 min.

### Intermediate BB

### (Z)-N′-hydroxy-4-(1-(4-methyl-3-(methylsulfonamido)benzoyl)piperidin-4-yl)benzimidamide

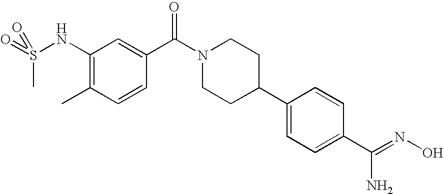

Hydroxylamine (0.033 mL, 0.55 mmol) was added to a solution of N-(5-(4-(4-cyanophenyl)piperidine-1-carbonyl)-2-methylphenyl)methanesulfonamide (Example 1) (0.2 g, 0.50 mmol) in EtOH (10 mL) under nitrogen. The resulting suspension was stirred for 24 hours under reflux. The reaction was incomplete and further hydroxylamine (0.033 mL, 0.55 mmol) was added and the suspension was stirred for a further 6 hours under reflux. The reaction mixture was evaporated to dryness to give the title compound as a colourless solid (0.182 g, 84%) which was used without further purification, ¹H NMR (400.132 MHz, DMSO-d₆) δ 1.54-1.93 (4H, m), 2.35 (3H, s), 2.78-3.20 (6H, m), 3.64-3.83 (1H, m), 4.52-4.72 (1H, m), 5.73 (2H, s), 7.21-7.37 (5H, m), 7.61 (2H, d), 9.16 (1H, s), 9.52 (1H, s), m/z (ESI+) (M+H)⁺=431.43; HPLC tR=1.05 min.

### Intermediate CC

### 4-[1-[3-amino-4-(methylsulfanylmethyl)benzoyl]piperidin-4-yl]benzonitrile

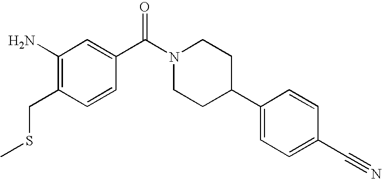

### Step 1

### 4-[1-[4-(chloromethyl)-3-nitrobenzoyl]piperidin-4-yl]benzonitrile

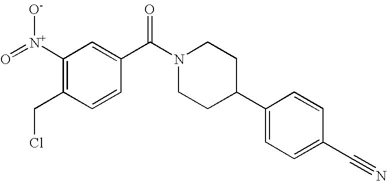

The title compound was prepared by an amide coupling reaction starting from 4-(chloromethyl)-3-nitrobenzoic acid and 4-(4-piperidyl)benzonitrile as described for Intermediate A, ¹H NMR (300.072 MHz, CDCl₃) δ 1.50-2.10 (4H, m), 2.84-3.30 (3H, m), 3.85 (1H, m), 4.90 (1H, m), 5.00 (2H, s), 7.33 (2H, d), 7.61-7.64 (2H, m), 7.76 (2H, m), 8.13 (1H, d), m/z (EI+) (M+H)⁺=384.11; HPLC tR=2.55 min.

### Step 2

### 4-[1-[4-(methylsulfanylmethyl)-3-nitrobenzoyl]piperidin-4-yl]benzonitrile

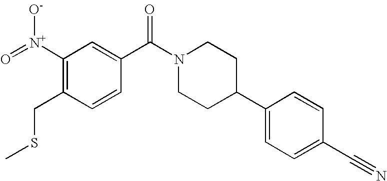

Sodium thiomethoxide (0.362 g, 5.17 mmol) was added in one portion to sodium borohydride (0.196 g, 5.17 mmol) and 4-(1-(4-(chloromethyl)-3-nitrobenzoyl)piperidin-4-yl)benzonitrile (Step 1) (1.984 g, 5.17 mmol) in MeOH (30.0 mL) at room temperature. The resulting pale yellow suspension was stirred for 22 hours. The reaction mixture was then evaporated to dryness, dissolved in DCM (150 mL), and washed sequentially with water (150 mL×2) and saturated brine (150 mL), and the aqueous washings extracted with DCM (100 mL). The combined organic layers were dried over MgSO₄, filtered and evaporated to afford crude product. This was purified by flash silica chromatography (elution gradient 10 to 50% EtOAc in isohexane). Impure fractions were combined and concentrated then re-purified by flash silica chromatography (elution gradient 10 to 30% EtOAc in isohexane). Pure fractions were evaporated to dryness to give the title compound as a yellow solid (1.027 g, 50.2%), ¹H NMR (400.132 MHz, CDCl₃) δ 2.03-1.56 (4H, m), 2.05 (3H, s), 2.94-2.83 (2H, m), 3.40-2.94 (1H, m), 4.03-3.56 (1H, m), 4.05 (2H, s), 5.11-4.86 (1H, m), 7.33 (2H, d), 7.56 (1H, d), 7.63 (2H, d), 7.66 (1H, d), 8.04 (1H, d), m/z (EI+) (M+H)⁺=396; HPLC tR=2.56 min. m/z (EI−) (M−H)−=394; HPLC tR=2.56 min.

### Step 3

### 4-[1-[3-amino-4-(methylsulfanylmethyl)benzoyl]piperidin-4-yl]benzonitrile

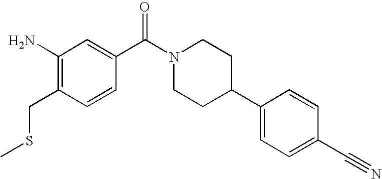

The title compound was prepared by an iron (III) chloride and zinc dust reduction, as described in Intermediate X, Step 2, starting from 4-[1-[4-(methylsulfanylmethyl)-3-nitrobenzoyl]piperidin-4-yl]benzonitrile (Step 2), ¹H NMR (400.132 MHz, DMSO-d₆) δ 1.66-1.53 (2H, m), 1.95-1.67 (2H, m), 1.97 (3H, s), 2.98-2.89 (2H, m), 3.16-2.98 (1H, m), 3.62 (2H, s), 3.71-3.65 (1H, m), 4.71-4.46 (1H, m), 6.53 (1H, d), 6.68 (1H, s), 7.03 (1H, d), 7.51 (2H, d), 7.79 (2H, d), m/z (EI+) (M+H)⁺=365.46; HPLC tR=2.29 min.

### Intermediate DD

### (3-amino-4-methyl-phenyl)-[4-[4-(trifluoromethyl)phenyl]-1-piperidyl]methanone

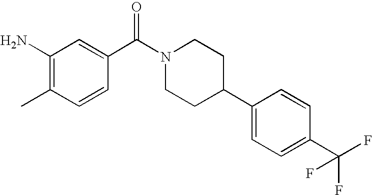

The title compound was prepared by the method described for Intermediate A, starting from 3-amino-4-methyl-benzoic acid and 4-[4-(trifluoromethyl)phenyl]piperidine hydrochloride,

¹H NMR (300.073 MHz, DMSO-d₆) d1.48-1.68 (m, 2H), 1.69-1.94 (m, 2H), 2.06 (s, 3H), 2.84-3.02 (m, 3H), 3.49-4.20 (m, 1H), 4.23-4.82 (m, 1H), 4.91-5.03 (m, 2H), 6.49 (d, 1H), 6.62 (s, 1H), 6.95 (d, 1H), 7.51 (d, 2H), 7.65 (d, 2H), m/z (ESI+) (M+H)⁺=363.37; HPLC tR=2.53 min.

### Intermediate EE

### 2-methoxy-4-(piperidin-4-yl)benzonitrile

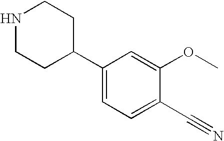

### Step 1

### tert-butyl 4-(4-cyano-3-methoxyphenyl)piperidine-1-carboxylate

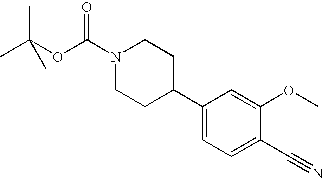

The title compound was prepared by the method described in Journal of Organic Chemistry 69 pp 5120-5123 (2004), starting from tert-butyl 4-hydroxy-1-piperidinecarboxylate and 4-bromo-2-methoxy-benzonitrile. The product was used in the next step without purification, ¹H NMR (400.132 MHz, CDCl₃) δ 1.47 (9H, s), 1.54-1.64 (2H, m), 1.80-1.86 (2H, m), 2.65-2.86 (3H, m), 3.94 (3H, s), 4.23-4.32 (2H, m), 6.79 (1H, s), 6.85 (1H, d), 7.49 (1H, d), HPLC tR=2.79 min.

### Step 2

### 2-methoxy-4-(piperidin-4-yl)benzonitrile

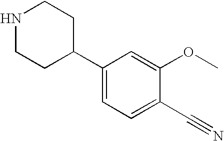

A saturated solution of hydrogen chloride in EtOAc (1.580 mL, 4.74 mmol) was added to tert-butyl 4-(4-cyano-3-methoxyphenyl)piperidine-1-carboxylate (Step 1) (0.15 g, 0.47 mmol, of 50% pure material) in DCM (5 mL) at 20° C. The resulting solution was stirred at 20° C. for 2 hours and the solvent then evaporated. The residue was triturated with Et2O to give the title compound as the hydrochloride salt (0.100 g, 83%; 50% pure due to impure starting material), ¹H NMR (400.132 MHz, DMSO-d₆) δ 1.82-2.00 (4H, m), 2.90-3.04 (3H, m), 3.20-3.31 (2H, m), 3.93 (3H, s), 6.96 (1H, d), 7.08 (1H, s), 7.70 (1H, d), 8.94 (1H, s), m/z (ESI+) (M+H)⁺=217; HPLC tR=0.72 min.

### Intermediate FF

### 2-fluoro-4-piperidin-4-ylbenzonitrile

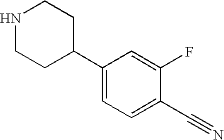

### Step 1

### tert-butyl 4-(4-cyano-3-fluorophenyl)piperidine-1-carboxylate

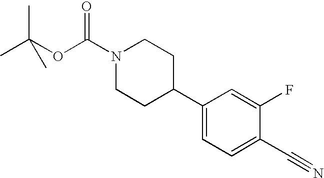

The title compound was prepared by the method described for Intermediate EE, Step 1, starting from tert-butyl 4-hydroxy-1-piperidinecarboxylate and 4-bromo-2-fluoro-benzonitrile, ¹H NMR (400.132 MHz, CDCl₃) δ 1.46 (9H, s), 1.51-1.61 (2H, m), 1.77-1.86 (2H, m), 2.66-2.85 (3H, m), 4.20-4.40 (2H, m), 7.04-7.12 (2H, m), 7.54-7.58 (1H, m), HPLC tR=2.80 min.

### Step 2

### 2-fluoro-4-piperidin-4-ylbenzonitrile

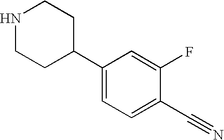

The title compound was prepared as the hydrochloride salt by the method described for Intermediate EE, Step 2, starting from tert-butyl 4-(4-cyano-3-fluorophenyl)piperidine-1-carboxylate (Step 1), ¹H NMR (400.132 MHz, DMSO-d₆) δ 1.79-1.94 (4H, m), 2.85-2.99 (3H, m), 3.24-3.29 (2H, m), 7.23-7.26 (1H, m), 7.32-7.38 (1H, m), 7.85 (1H, t), 9.09 (1H, s), m/z (ESI+) (M+H)⁺=205; HPLC tR=0.67 min.

### Intermediate GG

### 4-[4-(trifluoromethoxy)phenyl]piperidine

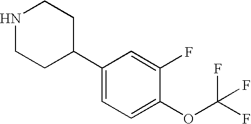

### Step 1

### tert-butyl 4-[4-(trifluoromethoxy)phenyl]piperidine-1-carboxylate

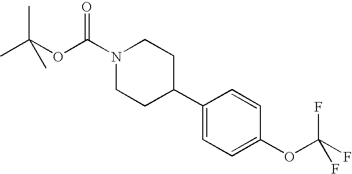

The title compound was prepared by the method described for Intermediate EE, Step 1, starting from tert-butyl 4-hydroxy-1-piperidinecarboxylate and 1-iodo-4-(trifluoromethoxy)benzene, ¹H NMR (400.132 MHz, CDCl₃) δ 1.48 (9H, s), 1.55-1.61 (2H, m), 1.77-1.85 (2H, m), 2.62-2.71 (1H, m), 2.78-2.89 (2H, m), 4.16-4.34 (2H, m), 7.14-7.17 (2H, m), 7.20-7.23 (2H, m); (NB. 50% purity estimated by NMR).

### Step 2

### 4-[4-(trifluoromethoxy)phenyl]piperidine

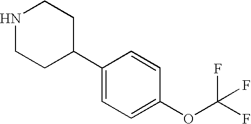

The title compound was prepared as the hydrochloride salt by the method described for Intermediate EE, Step 2, starting from tert-butyl 4-[4-(trifluoromethoxy)phenyl]piperidine-1-carboxylate (Step 1), m/z (ESI+) (M+H)⁺=246; tR=1.18 min.

### Intermediate HH

### 2-(4-piperidin-4-ylphenoxy)acetonitrile

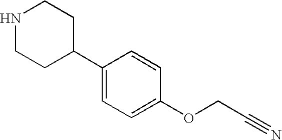

### Step 1

### tert-butyl 4-[4-(cyanomethoxy)phenyl]piperidine-1-carboxylate

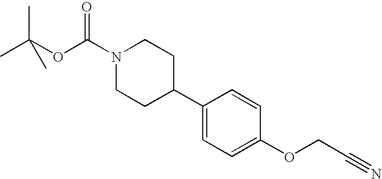

The title compound was prepared by the method described for Intermediate EE, Step 1, starting from tert-butyl 4-hydroxy-1-piperidinecarboxylate and 2-(4-bromophenoxy)acetonitrile, ¹H NMR (400.132 MHz, CDCl₃) δ 1.48 (9H, s), 1.54-1.64 (2H, m), 1.76-1.84 (2H, m), 2.58-2.66 (1H, m), 2.74-2.85 (2H, m), 4.18-4.32 (2H, m), 4.76 (2H, s), 6.93 (2H, d), 7.18 (2H, d), m/z (ESI+) (M−H)−=315; HPLC tR=2.73 min.

### Step 2

### 2-(4-piperidin-4-ylphenoxy)acetonitrile

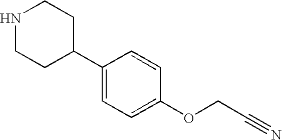

The title compound was prepared as the hydrochloride salt by the method described for Intermediate EE, Step 2, starting from tert-butyl 4-[4-(cyanomethoxy)phenyl]piperidine-1-carboxylate, m/z (ESI+) (M+H)⁺=217; HPLC tR=0.8 min; estimated to be 60% pure.

### Intermediate II

### 4-(4-methylsulfinylphenyl)piperidine

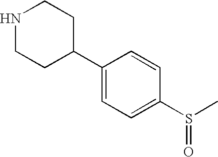

### Step 1

### tert-butyl 4-(4-methylsulfinylphenyl)piperidine-1-carboxylate

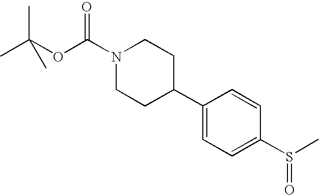

The title compound was prepared by the method described for Intermediate EE, Step 1, starting from tert-butyl 4-hydroxy-1-piperidinecarboxylate and 1-bromo-4-methanesulfinyl-Benzene, ¹H NMR (400.132 MHz, CDCl₃) δ 1.48 (9H, s), 1.59-1.68 (2H, m), 1.79-1.89 (2H, m), 2.72 (3H, s), 2.73-2.89 (3H, m), 4.20-4.35 (2H, m), 7.37 (2H, d), 7.59 (2H, d), m/z (ESI+) (M+Na)+=346; HPLC tR=1.78 min.

### Step 2

### 4-(4-methylsulfinylphenyl)piperidine

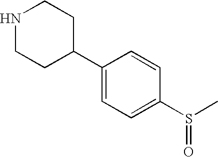

The title compound was prepared as the hydrochloride salt by the method described for Intermediate EE, Step 2, starting from tert-butyl 4-(4-methylsulfinylphenyl)piperidine-1-carboxylate (Step 1), HPLC tR=0.90 min, estimated to be 75% pure.

### Intermediate JJ

### N-methyl-4-piperidin-4-ylbenzenesulfonamide

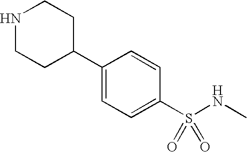

### Step 1

### tert-butyl 4-[4-(methylsulfamoyl)phenyl]piperidine-1-carboxylate

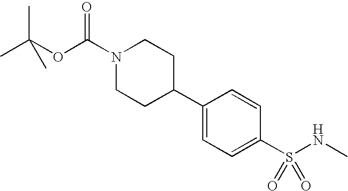

The title compound was prepared by the method described for Intermediate EE, Step 1, starting from tert-butyl 4-hydroxy-1-piperidinecarboxylate and 4-bromo-N-methyl-benzenesulfonamide 1H NMR (400.132 MHz, CDCl₃) δ 1.49 (9H, s), 1.60-1.68 (2H, m), 1.80-1.86 (2H, m), 2.67 (3H, d), 2.70-2.76 (1H, m), 2.78-2.87 (2H, m), 4.23-4.35 (2H, m), 4.41 (1H, q), 7.36 (2H, d), 7.80 (2H, d), m/z (ESI+) (M−H)−=353; HPLC tR=2.14 min.

### Step 2

### N-methyl-4-piperidin-4-ylbenzenesulfonamide

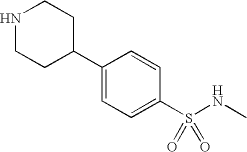

The title compound was prepared as the hydrochloride salt by the method described for Intermediate EE, Step 2, starting from tert-butyl 4-[4-(methylsulfamoyl)phenyl]piperidine-1-carboxylate (Step 1), ¹H NMR (400.132 MHz, DMSO-d₆) δ 1.82-2.00 (4H, m), 2.41 (3H, d), 2.91-3.05 (3H, m), 3.34-3.39 (2H, m), 7.45-7.49 (3H, m), 7.75 (2H, d), 8.85 (1H, s), m/z (ESI+) (M+H)⁺=255; HPLC tR=1.15 min; estimated to be 70% pure.

### Intermediate KK

### N-cyclopropyl-4-piperidin-4-ylbenzamide

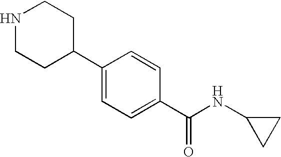

### Step 1

### tert-butyl 4-[4-(cyclopropylcarbamoyl)phenyl]piperidine-1-carboxylate

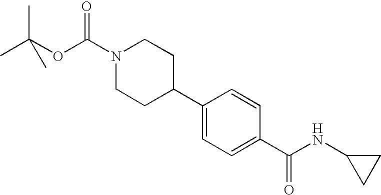

The title compound was prepared by the method described for Intermediate EE, Step 1, starting from tert-butyl 4-hydroxy-1-piperidinecarboxylate and N-cyclopropyl-4-iodo-benzamide, ¹H NMR (400.132 MHz, CDCl₃) δ 0.58-0.63 (2H, m), 0.82-0.90 (2H, m), 1.48 (9H, s), 1.56-1.67 (2H, m), 1.78-1.85 (2H, m), 2.65-2.71 (1H, m), 2.74-2.82 (2H, m), 2.87-2.93 (1H, m), 4.19-4.31 (2H, m), 6.27 (1H, s), 7.24 (2H, d), 7.68 (2H, d),), m/z (ESI+) (M+Na)+=367; HPLC tR=2.38 min.

### Step 2

### N-cyclopropyl-4-piperidin-4-ylbenzamide

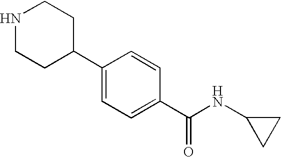

The title compound was prepared as the hydrochloride salt by the method described for Intermediate EE, Step 2, starting from tert-butyl 4-[4-(cyclopropylcarbamoyl)phenyl]piperidine-1-carboxylate (Step 1), m/z (ESI+) (M+H)⁺=245; HPLC tR=0.69 min.

Intermediate LL (4-piperidin-4-ylphenyl)methanesulfonate

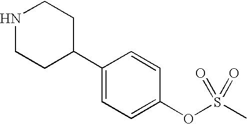

### Step 1

### tert-butyl 4-(4-methylsulfonyloxyphenyl)piperidine-1-carboxylate

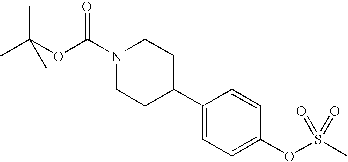

The title compound was prepared by the method described for Intermediate EE, Step 1, starting from tert-butyl 4-hydroxy-1-piperidinecarboxylate and 4-iodophenyl methanesulfonate, m/z (ESI−) (M−H)⁻=354; HPLC tR=2.76 min, HPLC indicates 76% purity.

### Step 2

### (4-piperidin-4-ylphenyl)methanesulfonate

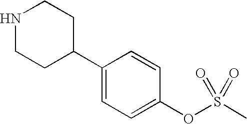

The title compound was prepared as the hydrochloride salt by the method described for Intermediate EE, Step 2, starting from tert-butyl 4-(4-methylsulfonyloxyphenyl)piperidine-1-carboxylate (Step 1), m/z (ESI+) (M+H)⁺=256; HPLC tR=0.74 min.

1) A compound of formula I 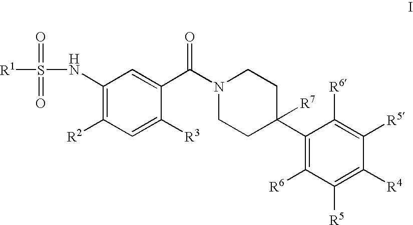
or a pharmaceutically acceptable salt thereof, in which
R¹ represents 1) a C₁₋₆alkyl group optionally substituted by one or two groups selected from A-X below and/or by one to five groups selected from Y below:
A) phenyl optionally substituted by one or more of the following i) halo; ii) cyano; iii) a C₁₋₄alkoxy group optionally substituted by one or more halo iv) hydroxy; v) a C₁₋₄alkyl group optionally substituted by one or more halo; vi) a group CONR^{e}R^{f} in which R^{e} and R^{f} are as defined below; vii) C₁₋₆alkanoyl; viii) benzoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl or benzyloxy; xix) nitro; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 10 membered heterocyclic group as defined in c) below; xxvii) phenylsulfonyl; xxviii) heteroarylsulfonyl;
xxix) a group of formula NR^{c}R^{d} in which R^{c} and R^{d} independently represent:
a) H;
b) C₁₋₆alkanoyl optionally substituted by carboxy or by a C₁₋₆ alkoxycarbonyl group;
c) a carbon linked saturated or partially unsaturated 4 to 10 membered heterocyclic group containing one or more N, S or O, wherein the S may be in its oxidised form of SO or SO₂, which is optionally fused to a benz ring and any ring is optionally substituted by one or more of the following: hydroxy, halo, a C₁₋₆ alkoxycarbonyl group, oxo, carboxy, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulphonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 10 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulphonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl or nitro;
k) heteroaryl optionally substituted by one or more carboxy; fluoro; hydroxy; a C₁₋₆alkyl group (which is optionally substituted by one or more of the following: a C₁₋₆alkoxy group, hydroxy or a group of formula NR^{e}R^{f} in which R^{e} and R^{f} are as defined above); a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group optionally substituted by phenyl;
n) heteroarylcarbonyl;
o) sulfamoyl optionally substituted by one or two independently selected C₁₋₆alkyl groups or the terminal nitrogen is included in a 5 or 6 membered saturated or partially unsaturated heterocyclic ring optionally containing an additional N, S or O, wherein the S may be in its oxidised form of SO or SO₂;
B) a heteroaryl group which is optionally substituted by groups i) to xxix) as described for phenyl above;
C) a group of formula NR^{c}R^{d} in which R^{c} and R^{d} are as defined above;
D) a C₃₋₁₀cycloalkyl group optionally substituted by one or more hydroxy or a group of formula NR^{e}R^{f} in which R^{e} and R^{f} are as defined above;
E) a carbon linked saturated or partially unsaturated 4 to 10 membered heterocyclic group containing one or more N, S or O, wherein the S may be in its oxidised form of SO or SO₂, which is optionally fused to a benz ring or a heteroaryl ring and/or is optionally substituted by one or more of the following: hydroxy; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; a C₁₋₆alkyl optionally substituted by one or more hydroxy or C₁₋₆alkoxy; or a C₁₋₆ alkoxycarbonyl group;
F) a C₁₋₆ alkoxycarbonyl group;
G) a C₂₋₆alkynyl group;
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulphonyl group;
M) phenylsulfonyl;
N) heteroarylsulfonyl;
O) benzoyl;
P) a C₁₋₆alkanoyl group
Q) C₁₋₆alkylthio;
R) ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
S) phenoxy;
T) hydroxy;
U) oxo;
V) carboxy;
W) cyano;
X) sulfamoyl optionally substituted by one or two independently selected C₁₋₆alkyl groups or the nitrogen is included in a 4 or 7 membered saturated or partially unsaturated heterocyclic ring optionally containing an additional N, S or O, wherein the S may be in its oxidised form of SO or SO₂;
Y) sulfamoylamino optionally substituted by one or two independently selected C₁₋₆alkyl groups or the terminal nitrogen is included in a 4 or 7 membered saturated or partially unsaturated heterocyclic ring optionally containing an additional N, S or O, wherein the S may be in its oxidised form of SO or SO₂;
Z) fluoro or chloro;
or R¹ represents
2) a C₃₋₁₀cycloalkyl group optionally substituted by one or two groups selected from A to Y above and/or by one to five groups selected from Z above;
3) a C₂₋₆alkynyl group optionally substituted by one or two groups selected from A to Y above and/or by one to five groups selected from Z above;
4) a carbon linked saturated or partially unsaturated 4 to 10 membered heterocyclic group containing one or more N, S or O, wherein the S may be in its oxidised form of SO or SO₂, which is optionally fused to a benz ring and any ring is optionally substituted by one or two groups A to Y as defined above and/or by one to five groups selected from Z above;
5) a C₂₋₆alkenyl group optionally substituted by one or two groups selected from A to Y above and/or by one to five groups selected from Z above;
6) optionally substituted phenyl including optional fusion of the phenyl ring to a saturated or partially unsaturated 5 to 6 membered heterocyclic ring optionally containing one, two or three hetero atoms selected from oxygen, sulphur optionally in its oxidised forms of SO or SO₂ or nitrogen wherein the heterocyclic ring is optionally substituted by one or more of the following: a C₁₋₆alkoxy group; a C₁₋₆alkanoyl group; carboxy; a C₁₋₆alkylsulfonyl group; a C₁₋₆alkoxycarbonyl group; carbamoyl; N—C₁₋₆alkylcarbamoyl; N,N-diC₁₋₆alkylcarbamoyl; hydroxy; oxo; a C₁₋₆alkyl group (which is optionally substituted by one or more of the following: a C₁₋₆alkoxy group, hydroxy or a group of formula NR^{c}R^{d} in which R^{c} and R^{d} are as defined above) and wherein the phenyl ring is optionally substituted by one or more of the groups i to xxix listed above or by a heteroaryl group optionally substituted by one or more groups i) to xxix) above or by an ureido group of formula R^{m}RⁿN—C(O)—NH— in which R^{m} and Rⁿ independently represent H, a C₁₋₆alkyl group optionally substituted by a C₁₋₆alkoxy group, or R^{m} and Rⁿ together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and/or optionally substituted by one or more of the following: a C₁₋₆alkoxy group; hydroxy; oxo; carboxy; a C₁₋₆alkylsulfonyl group; or a C₁₋₆alkyl group optionally substituted by one or more hydroxy or C₁₋₆alkoxy;
7) optionally substituted heteroaryl including N-oxides and S-oxides thereof optionally substituted by one or more of the groups i to xxix listed above;
wherein any alkyl chain mentioned in any of the definitions from A to Z above or in any of the definitions i to xxix above is optionally substituted by 1) one group or two groups selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 10 membered heterocyclic group in the list of optional substituents from A to Y above or in any of the definitions i to xxix above, for which specific substitution has not been previously mentioned, is optionally substituted by one, two or three groups selected from: carboxy; hydroxy; a C₁₋₃alkoxy group optionally substituted on C2 or C3 by carboxy; a group NR^{c}R^{d} din which R^{c} and R^{d} are as defined above; or a group CONR^{e}R^{f} in which R^{e} and R^{f} are as defined above; a C₁₋₄alkanoyloxy group or a C₁₋₄alkyl optionally substituted by one or more hydroxy, C₁₋₃alkoxy or a group —NR^{e}R^{f} in which R^{e} and R^{f} are as defined above; and/or is optionally substituted by one to five fluoro;
R² represents H, cyano, halo, a C₁₋₃alkoxy group, a group C₁₋₆alkylS(O)ₐ(O)_{b}— wherein the C₁₋₆alkyl is optionally substituted by one or more fluoro and a is 0, 1 or 2 and b is 0 except when a is 2 then b may also be 1 or R² represents a C₁₋₃alkyl group optionally substituted by a C₁₋₃alkoxy group or by a group C₁₋₃alkylS(O)ᵤ— which is optionally substituted by one or more fluoro and in which u is 0, 1 or 2;
R³ represents H, cyano, halo, a C₁₋₃alkoxy group, a group C₁₋₆alkylS(O)_{c}(O)_{d}— wherein the C₁₋₆alkyl is optionally substituted by one or more fluoro and c is 0, 1 or 2 and d is 0 except when c is 2 then d may also be 1 or R² represents a C₁₋₃alkyl group optionally substituted by a C₁₋₃alkoxy group or by a group C₁₋₃alkylS(O)ₜ— which is optionally substituted by one or more fluoro and in which t is 0, 1 or 2;
R⁴ represents
i) a C₁₋₃alkyl group optionally substituted by cyano, hydroxy, a C₁₋₃alkoxy group or by one or more halo
ii) a C₁₋₃alkoxy group optionally substituted by one or more halo or optionally substituted by cyano, hydroxy, a C₁₋₃alkoxy group, an amino group of formula NR^{u}R^{v} in which R^{u} and R^{v} independently represent H, a C₁₋₃alkylsulphonyl group, a C₁₋₃alkanoyl group, a C₁₋₃alkoxycarbonyl group, or a C₁₋₃alkyl group optionally substituted by hydroxy or R^{u} and R^{v} together with the nitrogen atom to which they are attached represent azetidinyl, pyrrolidinyl, piperidinyl, piperazinyl or morpholinyl each of which is optionally substituted by one or more of the following: oxo, C₁₋₃alkyl or hydroxy; or
iii) halo, iv) nitro, v) cyano,
vi) a C₁₋₆alkylS(O)_{y}(O)_{z}— optionally substituted by one or more fluoro wherein y is 0, 1 or 2 and z is 0 except when y is 2 then z may also be 1
vii) a group -L-R^{g} in which L represents a bond, a C₃₋₆cycloalkylene group, a C₃₋₆cycloalkylidene group, a C₁₋₆alkylene group or a C₁₋₆alkoxyC₁₋₆alkylene group wherein each group is optionally substituted by one or more of the following: carboxy, hydroxy, a C₁₋₃alkyl group optionally substituted by hydroxy;
and R^{g} represents carboxy or a group NR^{u}R^{v} in which R^{u} and R^{v} are as defined above and additionally R^{v} represents cyano or R^{g} represents a group CO₂R^{w} in which R^{w} is a C₁₋₃alkyl group; or R^{g} represents a group CONR^{x}R^{y} in which R^{x} and R^{y} independently represent H, a C₁₋₃alkylsulphonyl group, a C₁₋₃alkyl group or a C₃₋₆cycloalkyl group wherein the alkyl and cycloalkyl groups are optionally substituted by one or more hydroxy, carboxy or NR^{u}R^{v} in which R^{u} and R^{v} are as previously defined, or R^{x} and R^{y} together with the nitrogen atom to which they are attached represent azetidinyl; pyrrolidinyl, piperidinyl or morpholinyl; or R^{g} represents tetrazolyl or thiazolidin-2,4-dion-5-yl; or R^{g} represents ureido optionally independently substituted by one, two or three C₁₋₆alkyl or the terminal nitrogen is included in a 5 or 6 membered saturated or partially unsaturated heterocyclic ring optionally containing an additional N, S or O, wherein the S may be in its oxidised form of SO or SO₂;
viii) a group -L₁-N(R^{h})SO₂-L₂-Rⁱ in which L₁ and L₂ independently represent a bond or a C₁₋₆alkylene optionally substituted by one or more C₁₋₃alkyl groups, R^{h} is H or C₁₋₃alkyl and Rⁱ represents cyano or NR^{u}R^{v} in which R^{u} and R^{v} are as previously defined or Rⁱ represents a group CO—R^{j} in which R^{j} represents hydroxy, C₁₋₃alkoxy or a group NR^{u}R^{v} in which R^{u} and R^{v} are as previously defined;
ix) phenyl(O)_{f}— wherein f is 0 or 1 optionally substituted by one or more halo, C₁₋₃alkyl optionally substituted by one or more halo or C₁₋₃alkoxy optionally substituted by one or more halo;
x) phenylthio optionally substituted by one or more halo, C₁₋₃alkyl optionally substituted by one or more halo or C₁₋₃alkoxy optionally substituted by one or more halo;
xi) monocyclic heteroaryl(O)_{g}— wherein g is 0 or 1 optionally substituted by one or more halo, C₁₋₃alkyl optionally substituted by one or more halo or C₁₋₃alkoxy optionally substituted by one or more halo;
xii) a nitrogen containing 5 or 6 membered heteroarylCO— wherein the heteroaryl is linked through N to the carbonyl group and is optionally substituted by one or more halo, C₁₋₃alkyl optionally substituted by one or more halo or C₁₋₃alkoxy optionally substituted by one or more halo;
xiii) a C₂₋₆alkynyl group optionally substituted by one or more C₁₋₃alkyl, hydroxy, C₁₋₃alkoxy, C₁₋₃alkoxyC₁₋₃alkoxy, or a group —NR^{u}R^{v} as defined above;
xiv) a group -L₃-S(O)ₑC₁₋₆alkyl in which L₃ is a C₁₋₆alkylene optionally substituted by one or more of the following: hydroxy or a C₁₋₃alkyl group, and e is 0, 1 or 2;
xv) a group SO₂NR^{o}R^{p} in which R^{o} and R^{p} independently represent H; a C₁₋₆alkyl group optionally substituted by one or more of the following: hydroxy, C₁₋₆alkoxy or a group —NR^{u}R^{v} in which R^{k} and R^{l} are as defined above, or R^{o} and R^{p} together with the nitrogen atom to which they are attached represent a saturated or partially unsaturated 4 to 10 membered heterocyclic ring optionally containing an additional sulphur including oxidised as SO or SO₂, oxygen or nitrogen and/or optionally fused to a benz ring and any ring is optionally substituted by one or more of the following: a C₁₋₃alkoxy group; carboxy; a C₁₋₃alkylsulfonyl group; C₁₋₃alkanoyl; benzoyl; hydroxy; oxo; carboxy; or by a C₁₋₃alkyl group optionally substituted by one or more of the following: hydroxy, C₁₋₃alkoxy or carboxy; or
xvi) —C(NH₂)═N—OH
R⁵ and R^{5′} independently represent H, halo, cyano, C₁₋₃alkyl optionally substituted by one or more halo or C₁₋₃alkoxy optionally substituted by one or more halo;
R⁶ and R^{6′} independently represent H, halo, cyano, C₁₋₃alkyl optionally substituted by one or more halo or C₁₋₃alkoxy optionally substituted by one or more halo; and
R⁷ is H or OH with the proviso that the compound is not one of the following:
4-chloro-N-[2-methyl-5-(4-phenylpiperidine-1-carbonyl)phenyl]benzenesulfonamide
N-[2-chloro-5-[4-(4-cyanophenyl)piperidine-1-carbonyl]phenyl]methanesulfonamide
N-[5-[4-(4-cyanophenyl)piperidine-1-carbonyl]-2-(trifluoromethoxy)phenyl]methanesulfonamide
N-[5-[4-(4-cyanophenyl)piperidine-1-carbonyl]-2-(trifluoromethoxy)phenyl]-1-phenylmethanesulfonamide
N-[5-[4-(4-cyanophenyl)piperidine-1-carbonyl]-2-fluorophenyl]methanesulfonamide
N-[3-[4-(4-cyanophenyl)piperidine-1-carbonyl]-4-fluorophenyl]methanesulfonamide
4-[1-(3-methanesulfonamido-4-methylbenzoyl)piperidin-4-yl]-N,N-dimethylbenzamide
2-[[5-[4-(4-cyanophenyl)piperidine-1-carbonyl]-2-methylphenyl]sulfamoyl]benzoic acid
2-[[5-[4-(4-cyanophenyl)piperidine-1-carbonyl]-2-methylphenyl]sulfamoyl]-N-methylbenzamide
2-[[5-[4-(4-cyanophenyl)piperidine-1-carbonyl]-2-methylphenyl]sulfamoyl]-N-[(2,4-dimethoxyphenyl)methyl]benzamide
N-[5-[4-(4-cyanophenyl)piperidine-1-carbonyl]-2-methylsulfinylphenyl]methanesulfonamide
Benzyl 3-[[5-[4-(4-cyanophenyl)piperidine-1-carbonyl]-2-methylphenyl]sulfamoyl]pyrrolidine-1-carboxylate
N-[5-(4-hydroxy-4-phenylpiperidine-1-carbonyl)-2-methylphenyl]methanesulfonamide
N-[2-methyl-5-(4-phenylpiperidine-1-carbonyl)phenyl]methanesulfonamide
N-[5-[4-(4-chlorophenyl)-4-hydroxypiperidine-1-carbonyl]-2-methylphenyl]methanesulfonamide
N-[5-[4-(2-methoxyphenyl)piperidine-1-carbonyl]-2-methylphenyl]methanesulfonamide
N-[5-[4-hydroxy-4-[3-(trifluoromethyl)phenyl]piperidine-1-carbonyl]-2-methylphenyl]methanesulfonamide
N-[5-[4-(2-fluorophenyl)-4-hydroxypiperidine-1-carbonyl]-2-methylphenyl]methanesulfonamide
N-[5-[4-(3-cyanophenyl)piperidine-1-carbonyl]-2-methylphenyl]methanesulfonamide
N-[5-[4-(3-fluorophenyl)piperidine-1-carbonyl]-2-methylphenyl]methanesulfonamide
N-[5-[4-(3-chlorophenyl)piperidine-1-carbonyl]-2-methylphenyl]methanesulfonamide
N-[5-[4-(3-methoxyphenyl)piperidine-1-carbonyl]-2-methylphenyl]methanesulfonamide
N-[5-[4-(2-cyanophenyl)piperidine-1-carbonyl]-2-methylphenyl]methanesulfonamide
Methyl 4-[1-(3-methanesulfonamido-4-methylbenzoyl)piperidin-4-yl]benzoate
N-[5-[4-(3-bromophenyl)piperidine-1-carbonyl]-2-methylphenyl]methanesulfonamide
N-[5-[4-[4-(aminomethyl)phenyl]piperidine-1-carbonyl]-2-methylphenyl]methanesulfonamide
N-[2-methyl-5-[4-(3-phenylphenyl)piperidine-1-carbonyl]phenyl]methanesulfonamide
N-[2-methyl-5-[4-[3-(1,3-thiazol-5-yl)phenyl]piperidine-1-carbonyl]phenyl]methanesulfonamide
N-[5-[4-(3-ethynylphenyl)piperidine-1-carbonyl]-2-methylphenyl]methanesulfonamide
N-[5-[4-(4-cyanophenyl)piperidine-1-carbonyl]-2-(methylsulfonylmethyl)phenyl]methanesulfonamide
N-[2-methyl-5-[4-(4-pyrimidin-2-ylphenyl)piperidine-1-carbonyl]phenyl]methanesulfonamide
N-[5-[4-(4-cyano-2-methylphenyl)piperidine-1-carbonyl]-2-methylphenyl]methanesulfonamide
N-[5-[4-(4-cyano-3,5-difluorophenyl)piperidine-1-carbonyl]-2-methylphenyl]methanesulfonamide
N-[5-[4-(3,4-dicyanophenyl)piperidine-1-carbonyl]-2-methylphenyl]methanesulfonamide
N-[5-[4-[4-cyano-3-(trifluoromethyl)phenyl]piperidine-1-carbonyl]-2-methylphenyl]methanesulfonamide
N-[2-methyl-5-[4-[4-(2-oxopyrrolidin-1-yl)phenyl]piperidine-1-carbonyl]phenyl]methanesulfonamide
4-[1-(3-methanesulfonamido-4-methylbenzoyl)piperidin-4-yl]-N,N-dimethylbenzenesulfonamide
N-[5-[4-[4-[(3R)-3-hydroxypyrrolidin-1-yl]sulfonylphenyl]piperidine-1-carbonyl]-2-methylphenyl]methanesulfonamide
N-[2-methyl-5-[4-[4-(4-methylpiperazin-1-yl)sulfonylphenyl]piperidine-1-carbonyl]phenyl]methanesulfonamide and
N-[2-methyl-5-[4-[4-(4-methyl1,4-diazepane-1-carbonyl)phenyl]piperidine-1-carbonyl]phenyl]methanesulfonamide.
2-[4-[1-(3-methanesulfonamido-4-methylbenzoyl)piperidin-4-yl]phenyl]acetic acid
2-[4-[1-(3-methanesulfonamido-4-methylbenzoyl)piperidin-4-yl]phenyl]acetamide
2-[4-[1-(3-methanesulfonamido-4-methylbenzoyl)piperidin-4-yl]phenyl]-N,N-dimethylacetamide
2-hydroxy-N-[[4-[1-(3-methanesulfonamido-4-methylbenzoyl)piperidin-4-yl]phenyl]methyl]-2-methylpropanamide
N-[2-methyl-5-[4-(4-pyrrolidin-1-ylsulfonylphenyl)piperidine-1-carbonyl]phenyl]methanesulfonamide
2-[4-[1-[3-(cyclohexylsulfonylamino)-4-methylbenzoyl]piperidin-4-yl]phenyl]acetic acid or N-[5-[4-[4-(3-amino-3-methylbut-1-ynyl)phenyl]piperidine-1-carbonyl]-2-methylphenyl]methanesulfonamide.

2) A compound according to claim 1 as represented by formula II 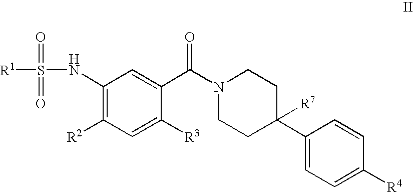
R¹ represents 1) a C₁₋₆alkyl group optionally substituted by one or two groups selected from A-S below and/or by one to five groups selected from T below:
A) phenyl optionally substituted by one or more of the following i) halo; ii) cyano; iii) a
C₁₋₄alkoxy group optionally substituted by one or more halo iv) hydroxy; v) a C₁₋₄alkyl group optionally substituted by one or more halo; vi) carbamoyl; vii) N—C₁₋₆alkylcarbamoyl; viii) N,N-diC₁₋₆alkylcarbamoyl; ix) carboxy; x) C₁₋₆ alkoxycarbonyl; xi) C₁₋₆alkylthio; xii) C₁₋₆alkylsulfinyl; xiii) C₁₋₆alkylsulfonyl; xiv) C₁₋₆alkylsulfonyloxy; xv) sulphamoyl; xvi) N—C₁₋₆alkylsulphamoyl; xvii) N,N-diC₁₋₆alkylsulphamoyl; xviii) benzyl xix) benzyloxy; xx) heteroaryl; xxi) heteroaryloxy; xxii) phenyl xxiii) phenoxy xxiv) phenylsulphamoyl; xxv) heteroarylsulphamoyl; xxvi) a carbon linked saturated or partially unsaturated 4 to 8 membered heterocyclic group as defined in c) below; xxvii) phenylsulfonyl; xxviii) heteroarylsulfonyl;
xxix) a group of formula NR^{c}R^{d} in which R^{c} and R^{d} independently represent:
a) H;
b) C₁₋₆alkanoyl;
c) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by one or more of the following: hydroxy, oxo, carboxy, a C₁₋₆alkoxy group optionally substituted by one or more hydroxy or C₁₋₆alkoxy, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
d) a C₁₋₆alkyl group optionally substituted by one or more of the following: hydroxy; carboxy; a C₁₋₆alkoxycarbonyl group; a C₁₋₆alkoxy group; heteroaryl; a group of formula NR^{e}R^{f} in which R^{e} and R^{f} independently represent H; a C₁₋₆alkanoyl group; a C₁₋₆alkylsulfonyl group; a C₁₋₆alkoxycarbonyl group; a C₁₋₆alkyl group optionally substituted by one or more hydroxy or C₁₋₆alkoxy, or R^{e} and R^{f} together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and any ring is optionally substituted by one or more of the following: a C₁₋₆alkoxy group; carboxy; a C₁₋₆alkylsulfonyl group; C₁₋₄alkanoyl; benzoyl; hydroxy; oxo; carboxy; or a C₁₋₆alkyl group optionally substituted by one or more hydroxy or by one or more C₁₋₆alkoxy or by one or more carboxy;
e) R^{c} and R^{d} together with the nitrogen atom to which they are attached represent a saturated or partially unsaturated 4 to 8 membered heterocyclic ring optionally containing an additional oxygen, sulphur, SO, SO₂ or nitrogen and/or optionally fused to a benz ring and/or optionally substituted by one or more of the following: a C₁₋₆alkoxy group; C₁₋₄alkanoyl group; benzoyl; a C₁₋₆alkoxycarbonyl group; a C₁₋₆alkylsulfonyl group; carbamoyl; N—C₁₋₆alkylcarbamoyl; N,N-diC₁₋₆alkylcarbamoyl; hydroxy; oxo; carboxy; a C₁₋₆alkyl group (which is optionally substituted by one or more of the following: a C₁₋₆alkoxy group, hydroxy or a group of formula NR^{e}R^{f} in which R^{e} and R^{f} are as defined above) or a group of formula NR^{e}R^{f} in which R^{e} and R^{f} are as defined above;
f) a C₁₋₆alkylsulfonyl group;
g) phenylsulfonyl;
h) heteroarylsulfonyl;
i) benzoyl;
j) phenyl optionally substituted by one or more of the following: halo; C₁₋₃alkyl; C₁₋₃alkoxy; a C₁₋₆alkanoylamino group; carbamoyl; N—C₁₋₆alkylcarbamoyl; N,N-diC₁₋₆alkylcarbamoyl;
k) heteroaryl optionally substituted by one or more carboxy; fluoro; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above;
l) a C₃₋₁₀cycloalkyl group which may be monocyclic, bicyclic or tricyclic and optionally may be bridged and is optionally substituted by one or more carboxy; fluoro; hydroxy; a C₁₋₃alkoxy group optionally substituted on C2 or C3 by carboxy; a group NR^{e}R^{f} in which R^{e} and R^{f} are as defined above; or a group CONR^{e}R^{f} in which R^{e} and R^{f} are as defined above;
m) a C₁₋₆alkoxycarbonyl group;
B) a heteroaryl group which is optionally substituted by groups i) to xxix) as described for phenyl above;
C) a group of formula NR^{c}R^{d} in which R^{c} and R^{d} are as defined above;
D) a C₃₋₇cycloalkyl group optionally substituted by one or more hydroxy or a group of formula NR^{e}R^{f} in which R^{e} and R^{f} are as defined above;
E) a carbon linked saturated or partially unsaturated 4 to 8 membered heterocyclic group containing one or more N, S or O, wherein the S may be in its oxidised form of SO or SO₂, which is optionally fused to a benz ring and/or is optionally substituted by one or more of the following: hydroxy; oxo; a C₁₋₆alkoxy group; carboxy; hydroxy; C₁₋₄alkanoyl; a C₁₋₆alkylsulfonyl group; amino; C₁₋₃alkylamino; di(C₁₋₃ alkyl)amino; or a C₁₋₆alkyl optionally substituted by one or more hydroxy or C₁₋₆alkoxy;
F) a C₁₋₆ alkoxycarbonyl group;
G) a C₂₋₆alkynyl group:
H) a group —CONR^{c}R^{d} in which R^{c} and R^{d} are as defined above;
I) a C₁₋₆alkoxy group;
J) a C₂₋₆alkenyl group:
K) a C₁₋₆alkyl group;
L) a C₁₋₆alkylsulfonyl group;
M) phenylsulfonyl;
N) heteroarylsulfonyl;
O) benzoyl;
P) a C₁₋₆alkanoyl group
Q) hydroxy;
R) oxo;
S) carboxy;
T) fluoro
or R¹ represents
2) a C₃₋₇cycloalkyl group optionally substituted by one or two groups selected from A to T above;
3) a C₂₋₆alkynyl group optionally substituted by one or two groups selected from A to T above;
4) a carbon linked saturated or partially unsaturated 4 to 8 membered heterocyclic group containing one or more N, S or O, wherein the S may be in its oxidised form of SO or SO₂, which is optionally fused to a benz ring and any ring is optionally substituted by a group A to T as defined above one or more of the following: hydroxy, oxo, carboxy, a C₁₋₆alkoxy group, hydroxy, a C₁₋₆alkylsulfonyl group, C₁₋₄alkanoyl, benzoyl, amino, C₁₋₃alkylamino, di(C₁₋₃ alkyl)amino or a C₁₋₆alkyl optionally substituted by one or more hydroxy or C₁₋₆alkoxy;
5) a C₂₋₆alkenyl group optionally substituted by one or two groups selected from A to T above;
6) optionally substituted phenyl including optional fusion of the phenyl ring to a saturated or partially unsaturated 5 to 6 membered heterocyclic ring optionally containing one, two or three hetero atoms selected from oxygen, sulphur optionally in its oxidised forms of SO or SO₂ or nitrogen wherein the heterocyclic ring is optionally substituted by one or more of the following: a C₁₋₆alkoxy group; a C₁₋₆alkanoyl group; carboxy; a C₁₋₆alkylsulfonyl group; a C₁₋₆alkoxycarbonyl group; carbamoyl; N—C₁₋₆alkylcarbamoyl; N,N-diC₁₋₆alkylcarbamoyl; hydroxy; oxo; a C₁₋₆alkyl group (which is optionally substituted by one or more of the following: a C₁₋₆alkoxy group, hydroxy or a group of formula NR^{c}R^{d} in which R^{c} and R^{d} are as defined above) and wherein the phenyl ring is optionally substituted by one or more of the groups i to xxix listed above or by a heteroaryl group optionally substituted by one or more groups i) to xxix) above or by an ureido group of formula R^{m}RⁿN—C(O)—NH— in which R^{m} and Rⁿ independently represent H, a C₁₋₆alkyl group optionally substituted by a C₁₋₆alkoxy group, or R^{m} and Rⁿ together with the nitrogen atom to which they are attached represent a saturated or partially unsaturated 4 to 8 membered heterocyclic ring optionally containing an additional sulphur including oxidised as SO or SO₂, oxygen or nitrogen and/or optionally fused to a benz ring and/or optionally substituted by one or more of the following: a C₁₋₆alkoxy group; hydroxy; oxo; carboxy; a C₁₋₆alkylsulfonyl group; or a C₁₋₆alkyl group optionally substituted by one or more hydroxy or C₁₋₆alkoxy;
7) optionally substituted heteroaryl including N-oxides and S-oxides thereof optionally substituted by one or more of the groups i to xxix listed above;
wherein any alkyl chain mentioned in any of the definitions from A to P above or in any of the definitions i to xxix above is optionally substituted by 1) one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or by 2) from one to five fluoro;
and further wherein any cycloalkyl, phenyl, heteroaryl ring or carbon linked saturated or partially saturated 4 to 8 membered heterocyclic group in the list of optional substituents from A to P above or in any of the definitions i to xxix above, for which specific substitution has not been previously mentioned, is optionally substituted by one group selected from: carboxy; hydroxy; a C₁₋₃alkoxy group optionally substituted on C2 or C3 by carboxy; a group NR^{c}R^{d} in which R^{c} and R^{d} are as defined above; or a group CONR^{e}R^{f} in which R^{e} and R^{f} are as defined above; and/or is optionally substituted by one to five fluoro;
R² represents H, a C₁₋₃alkyl group; a C₁₋₃alkoxy group, cyano or halo;
R³ represents H, a C₁₋₃alkyl group; a C₁₋₃alkoxy group; cyano or halo;
R⁴ represents halo, cyano or a C₁₋₄alkylsulfonyl group;
R⁷ is H or OH.

3) A compound according to claim 1 as represented by formula IIA 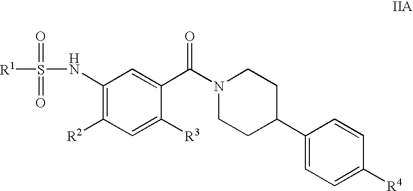
or a pharmaceutically acceptable salt thereof, in which
R¹ represents 1) a C₁₋₆alkyl group optionally substituted by one or more of the following: a) halo b) a C₁₋₆alkoxycarbonyl c) a C₃₋₆cycloalkyl group d) phenyl optionally substituted by one or more of the following: halo or a C₁₋₄alkylsulfonyl group; e) a C₁₋₄alkylsulfonyl group or f) an amino group of formula NR^{u}R^{v} in which R^{u} and R^{v} are as defined above;
2) C₃₋₆cycloalkyl group; or
3) phenyl optionally substituted by one or more of the following: a) halo; b) cyano; c) a C₁₋₆alkanoylamino group or d) a C₁₋₆alkoxy group;
4) thienyl optionally substituted by one or more halo;
5) 2-oxo-1,3-dihydroindol-5-yl;
6) 5-methyl-1,2-oxazol-4-yl;
R² represents H, a C₁₋₃alkyl group; a C₁₋₃alkoxy group, cyano or halo;
R³ represents H, a C₁₋₃alkyl group; a C₁₋₃alkoxy group; cyano or halo; and
R⁴ represents cyano or a C₁₋₄alkylsulfonyl group.

4) A compound according to any previous claim in which one of R² and R³ is other than H.

5) A compound according to any one of claims 1 to 3 in which R² is methyl and R³ is H.

6) A compound according to any one of claims 1 to 3 in which R³ is methyl and R² is H.

7) A compound according to any one of claims 1 to 3 in which R² is methyl and R³ is methyl.

8) A compound selected from one or more of the compounds in List 1 or a pharmaceutically-acceptable salt thereof.

9) A compound selected from one or more of the compounds in List 2 or a pharmaceutically-acceptable salt thereof.

10) A method of treating obesity or being overweight, eating disorders, dyslipidaemia and type 2 diabetes mellitus comprising administering a pharmacologically effective amount of a compound of formula I as defined in any one of claims 1 to 9 to a patient in need thereof.

11) A method of treating cancer comprising administering a pharmacologically effective amount of a compound of formula I as defined in any one of claims 1 to 9 to a patient in need thereof.

12) A method of treating infection comprising administering a pharmacologically effective amount of a compound of formula I as defined in any one of claims 1 to 9 to a patient in need thereof.

13) A pharmaceutical formulation comprising a compound of formula I as defined in any one of claims 1 to 9, or pharmaceutically acceptable salt thereof, in admixture with pharmaceutically acceptable adjuvants, diluents and/or carriers.

14) A process for preparing a compound of formula I according to claim 1 comprising
(a) reacting a compound of formula VI 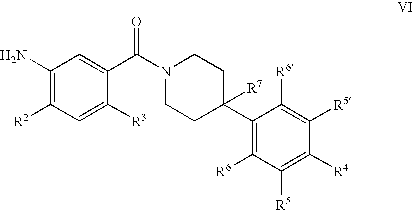
with a compound of formula VII
R₁SO₂X VII
in which X represents a leaving group in the presence of a diluent and optionally in the presence of a base at a temperature in the range of 0-150° C.; or
b) reacting a compound of formula IX 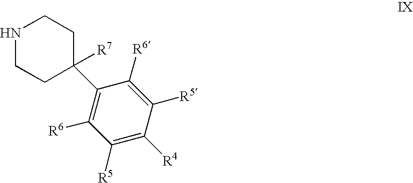
with a compound of formula X 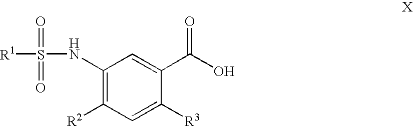
optionally in the presence of a coupling agent and optionally in the presence of a diluent at a temperature in the range of 0-150° C.; or
c) reacting a compound of formula IX 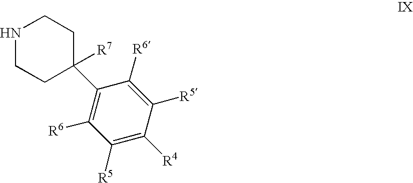
with a compound of formula XI 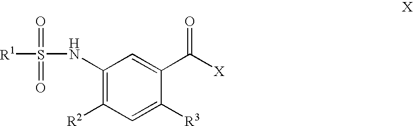
in which X represents a leaving group in the presence of a diluent and optionally in the presence of a base at a temperature in the range of 0-150° C.; or
d) reacting a compound of formula XII 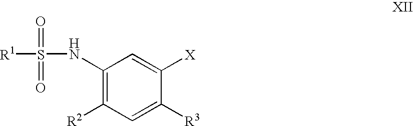
in which X represents a replaceable group with a compound of formula X in the presence of carbon monoxide and in the presence of a metal catalyst and in a solvent and in the temperature range 0-150° C. wherein in each of a), b), c), or d), R¹, R², R³, R⁴, R⁵, R^{5′}, R⁶, R^{6′} and R⁷ are, unless otherwise specified, as defined in claim 1.

15) A compound of formula VI as described in the preceding claim.
